(19) United States
(12) Patent Application Publication
(10) Pub. No.: US 2005/0004134 A1
(43) Pub. Date: Jan. 6, 2005

(54) Thiazole derivative and pharmaceutical use thereof

(76) Inventors: Hideo Tsutsumi, Osaka-shi Osaka (JP); Seiichiro Tabuchi, Osaka (JP); Atsushi Akahane, Osaka (JP); Hironobu Yasuda, Osaka (JP); Hiroki Omori, Osaka (JP); Kiyoshi Temmaru, Osaka (JP); Atsuhiko Zanka, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/494,033

(22) PCT Filed: Nov. 8, 2002
(86) PCT No.: PCT/JP02/11639

(30) Foreign Application Priority Data
Nov. 8, 2001 (AU) PR 8749
Nov. 23, 2001 (AU) PR 9048

(51) Int. Cl. C07D 417/02; A61K 31/501

(52) U.S. Cl. 514/252.05; 544/238

(57) ABSTRACT

A thiazole derivative of the formula (I): wherein R is a 1-optionally substituted-6-oxo-1,6-dihydro-3-pyridazinyl, R′ is an optionally substituted phenyl, and R² is hydrogen, a group of the formula (i): wherein R⁴ is hydrogen, lower alkyl or lower alkenyl, and R⁵ is hydrogen, optionally substituted lower alkyl, acyl, cyclo(lower)alkyl, lower alkenyl, optionally substituted aryl or heterocyclic, or a group of the formula (ii): wherein X is oxygen or sulfur, R⁸ is hydrogen or lower alkyl, R⁹ is hydrogen, optionally substituted lower alkyl, cyclo(lower)alkyl, lower alkoxy or mono- or di-lower alkylamino or R⁸ and R⁹ may combine together to form optionally substituted saturated N-containing heterocyclic, or a salt thereof.

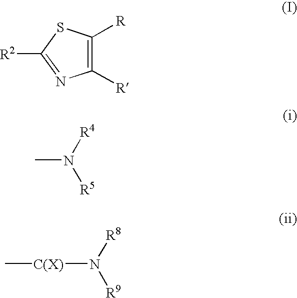

### TECHNICAL FIELD

The present invention relates to a novel thiazole derivative which are useful as medicaments, a process for preparing an intermediate 2-allyl-6-hydroxy-3(2H)-pyridazinone for their production and a pharmaceutical composition containing the same.

### BACKGROUND ART

Adenosine is a ubiquitous biochemical messenger. Adenosine binds to and activates seven-transmembrane spanning G-protein coupled receptors, eliciting a variety of physiological responses. Adenosine receptors are divided into four known subtypes (i.e., A₁, A₂ₐ, A_{2b}, and A₃). These receptor subtypes mediate different, and sometimes opposing, effects. Activation of the adenosine A₁ receptor, for example, elicits an increase in renal vascular resistance, while activation of the adenosine A₂, receptor elicits a decrease in renal vascular resistance. Accordingly, adenosine antagonists are useful in the prevention and/or treatment of numerous diseases, including cardiac and circulatory disorders, degenerative disorders of the central nervous system, respiratory disorders, and many diseases for which diuretic treatment is suitable.

Some 4-aryl-5-(pyridin-4-yl)thiazole derivatives having adenosine A₃ or A_{2b} inhibitory activities are known (e.g. WO-9964418A, JP-2001-114779A, etc.). However, 4-aryl-5-(6-oxo-1,6-dihydro-pyridazin-3-yl)thiazole derivatives are not known, so far. In addition, any thiazole derivatives having both of adenosine A₁ and A₂ₐ inhibitory activities are not known.

It is known that it is generally difficult to selectively allate 3,6-dihydroxypyridazine to give 2-alkyl-6-hydroxy-3(2H)-pyridazinone (see “Pyridazine” ed. by R. N. Castle, John Wiley & Sons, 1973). For example, 3,6-dihydroxypyridazine is methylated with dimethyl sulfate to give 2-methyl-6-hydroxy-3(2H)-pyridazinone derivative, 1,2-dimethyl-3(2H), 6(1H)-pyridazinedione and/or 2-methyl-6-methoxy-3(2H)-pyridazinone depending the reaction condition (K. Eichenberger et al., Helv. Chin Acta, 37, 837 (1954)). With diazomethane, 1,3-dihydroxypyridazine is alkylated to give 6-methoxy-3(2H)-pyridazinone (F. Arndt, Angew. Chem., 61, 397 (1949)). With an alkyl halide, 3,6-dihydroxypyridazine is alkylated to give 2-alkyl-6-alkoxy-3 (2H), 6(1H)-pyrdazinedinone, 2-alkyl-6-hydroxy-3(2H)-pyridazinone or 6-alkoxy-3(2H)-pyridazinone depending the reaction pH condition (R. Sch nbeck, Monatsh Chem., 90, 284 (1959)). Besides, 3,6-dihydroxypyridazine is hardly reactive nor soluble in an usual solvent. R. H. Mizzoni et al reported the preparation of 6-hydroxy-2-alkyl-3(2H)-pyridazinone by reacting maleic anhydride with alkyl hydrazine (J. Amer. Chem. Soc., 76, 2201 (1954)). However, alkylhydrazine is too explosive to prepare or obtain commercially. Therefore, it is desired to develop a safe and convenient process for preparing 2-alkyl-6-hydroxy-3(2H)-pyridazinone, which is useful intermediate for preparing thiazole derivatives.

### DISCLOSURE OF INVENTION

The present invention relates to a novel thiazole derivative and a pharmaceutically acceptable salt thereof, which are useful as medicaments; processes for preparing an intermediate 2-alkyl-6-hydroxy-3(2H)-pyridazinone for the production of said thiazole derivative and a salt thereof; a pharmaceutical composition comprising, as an active ingredient, said thiazole derivative or a pharmaceutically acceptable salt thereof; a use of said thiazole derivative or a pharmaceutically acceptable salt thereof as a medicament; and a method for using said thiazole derivative or a pharmaceutically acceptable salt thereof for therapeutic purposes, which comprises administering said thiazole derivative or a pharmaceutically acceptable salt thereof to a human being or an animal.

The thiazole derivatives of this invention are represented by the following formula (I): 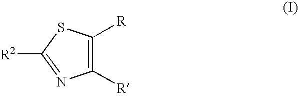
R is a 1-optionally substituted-6-oxo-1,6-dihydro-3-pyridazinyl,
R′ is an optionally substituted phenyl,
R² is a hydrogen atom,
   a group represented by the formula (i): 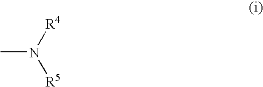
   wherein
   R⁴ is hydrogen atom,
      a lower alkyl group or
      a lower alkenyl group, and
   R⁵ is hydrogen atom,
      an optionally substituted lower allyl group,
      an acyl group,
      a cyclo(lower)alkyl group,
      a lower alkenyl group,
      an optionally substituted aryl group or
      a heterocyclic group, or
         a group represented by the formula (ii): 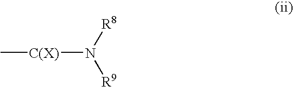
   wherein
   X is an oxygen or sulfur atom,
   R⁸ is a hydrogen atom or
      a lower alkyl group,
   R⁹ is a hydrogen atom,
      an optionally substituted lower alkyl group,
      a cyclo(lower)alkyl group,
      a lower alkoxy group or
      a mono- or di-lower alkylamino group or
   R⁸ and R⁹ may combine together to form an optionally substituted saturated N-containing heterocyclic group.

In the above and subsequent description of the present specification, suitable examples and illustrations of the various definitions, which the present invention includes within the scope, are explained in detail as follows.

The term “one or more” means 1 to 6, among which the preferred one is a number of 1 to 3, and the most preferred one is 1 or 2.

The term “lower” means a group having 1 to 6 carbon atom(s) unless otherwise indicated.

Suitable examples of the lower alkyl group and the lower alky moieties in the mono- or di-lower alkylamino, halo(lower)alkyl, di(lower)alkylamino, hydroxy(lower)alkyl, lower alkoxy(lower)alkyl, saturated or unsaturated heterocyclic(lower)alkyl, mono- or di-lower alkylamino(lower)alkyl, lower alkanoylamino(lower)allyl, ar(lower)alkyl, ar(lower)alkylamino, pyrrolidon-1-yl(lower)alkyl, halo(lower)alkoxy, lower alkylsulfonyl, mono- or di-lower alkylcarbamoyl and ar(lower)alkylcarbamoyl groups are straight or branched ones having 1 to 6 carbon atoms such as methyl, ethyl, propyl, isopropyl, n-butyl, isobutyl, tert-butyl, n-pentyl, n-hexyl, etc., in which the preferred one may be methyl, n-butyl, tert-butyl or hexyl.

Suitable examples of the halogen atom and halogen moieties in the halo(lower)alkyl and halo(lower)alkoxy groups are fluorine, chlorine, bromine or iodine.

Suitable examples of the lower alkenyl group are straight or branched ones having 1 to 6 carbon atom(s), such as ethenyl, 1- or 2-propenyl, butenyl, pentenyl, hexenyl, etc.

Suitable examples of the cyclo(lower)alkyl group and cyclo(lower)alkyl moiety in the cyclo(lower)aylcarbonyl group are cyclo(C₃-C₈)alkyl such as cyclopropyl, cyclobutyl, cyclopentyl, cyclohexyl, cycloheptyl, cyclooctyl, etc., in which the preferred one may be cyclohexyl.

Suitable examples of the lower alkoxy group and the lower alkoxy moieties in the lower alkoxy(lower)alkyl, lower alkoxycarbonyl and lower alkoxy-substituted aryl groups are straight or branched ones having 1 to 6 carbon atoms such as methoxy, ethoxy, n-propoxy, isopropoxy, n-butoxy, 2-ethylbutoxy, isobutoxy, tert-butoxy, pentyloxy, n-hexyloxy, etc., in which the preferred one may be ones having 1 to 4 carbon atoms and the more preferred one may be methoxy.

Suitable examples of the acyl group include optionally substituted lower alkanoyl, cyclo(lower)alkylcarbonyl, lower alkoxycarbonyl, optionally substituted aroyl, aryloxycarbonyl, heterocyclic carbonyl, mono- or di-lower alkylcarbamoyl, ar(lower)alkylcarbamoyl, optionally substituted arylcarbamoyl and optionally substituted arylsulfonylcarbamoyl.

Suitable aryl and aryl moieties in the ar(lower)alkylamino, ar(lower)alkyl, aryloxy, arylamino, arylsulfonylamino, aroyl, aryloxycarbonyl, ar(lower)alkylcarbamoyl, arylcarbamoyl and arylsulfonylcarbamoyl groups are the ones having 6 to 18 carbon atoms such as phenyl, naphthyl, indenyl, anthryl, etc., in which the preferred one may be the one having 6 to 10 carbon atoms, and the more preferred one may be phenyl.

Suitable examples of the mono-lower alkylamino group are methylamino, ethylamino, propylamino and butylamino.

Suitable examples of the di-lower alkylamino group are dimethylamino, methyl(ethyl)amino, diethylamino, ethyl(propyl)amino and dipropylamino.

Suitable examples of the heterocyclic group and the heterocyclyl moieties in the saturated or unsaturated heterocyclic(lower)alkyl and heterocyclic carbonyl groups are saturated or unsaturated, monocyclic or condensed heterocyclic group containing 1 to 4 heteroatoms selected from nitrogen, oxygen and sulfur atoms.

Preferable examples of the heterocyclic group and the heterocyclyl moieties are described in the following.
(1) unsaturated 3 to 7-membered, preferably 5- or 6-membered heteromonocyclic group containing 1 to 4 nitrogen atoms, for example, pyrrolyl, pyrrolinyl, imidazolyl, pyrazolyl, pyridyl, tetrahydropyridyl, pyrimidinyl, tetrahydropyrimidinyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g., 1H-tetrazolyl, 2H-tetrazolyl, etc.), etc.;
(2) saturated 3 to 7-membered, preferably 5- or 6-membered heteromonocyclic group containing 1 to 4 nitrogen atoms (e.g., pyrrolidinyl, imidazolidinyl, piperidyl, piperidino, piperazinyl, etc.);
(3) unsaturated 3 to 7-membered, preferably 5- or 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.), etc.;
(4) saturated 3 to 7-membered, preferably 5- or 6-membered heteromonocyclic group containing 1 to 2 oxygen atoms and 1 to 3 nitrogen atoms (e.g., morpholinyl, etc.);
(5) unsaturated 3 to 7-membered, preferably 5- or 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.), etc.;
(6) saturated 3 to 7-membered preferably 5- or 6-membered heteromonocyclic group containing 1 to 2 sulfur atoms and 1 to 3 nitrogen atoms (e.g., thiomorpholinyl, thiazolidinyl, etc.);
(7) unsaturated 3 to 7-membered, preferably 5- or 6-membered heteromonocyclic group containing 1 to 2 oxygen atoms (e.g., furyl, pyranyl, etc);
(8) saturated 3 to 7-membered, preferably 5- or 6-membered heteromonocyclic group containing 1 to 2 oxygen atoms (e.g., 1,4-dioxanyl, etc);
(9) unsaturated 3 to 7-membered, preferably 5- or 6-membered heteromonocyclic group containing 1 to 2 sulfur atoms (e.g., thienyl, etc);
(10) saturated 3 to 7-membered, preferably 5- or 6-membered heteromonocyclic group containing 1 to 2 sulfur atoms (e.g., tetrahydrothienyl, etc);
(11) unsaturated condensed heterocyclic group containing 1 to 3 nitrogen atoms (e.g., benzopyrrolyl, benzimidazolyl, benzopyrazolyl, benzotriazolyl, quinolyl, isoquinolyl, indolyl, indolinyl, carbazolyl, 1,2,3,4-tetrahydroquinolyl, etc);
(12) unsaturated condensed heterocyclic group containing 1 to 2 oxygen atoms (e.g., benzofuryl, benzodioxolyl, etc);
(13) unsaturated condensed heterocyclic group containing 1 to 2 sulfur atoms (e.g., benzo[b]thienyl, etc.)
(14) unsaturated condensed heterocyclic group containing 1 to 2 oxygen atoms and 1 to 3 nitrogen atoms (e.g., benzoxazolyl, benzoxadiazolyl, phenoxazinyl, etc); or
(15) unsaturated condensed heterocyclic group containing 1 to 2 sulfur atoms and 1 to 3 nitrogen atoms (e.g., benzothiazolyl, benzisothiazolyl, phenothiazinyl, etc).

The N-containing heterocyclic group includes the ones described in (1), (2), (3), (4), (5), (6), (11), (14) and (15).

The saturated N-containing heterocyclic group includes the ones described in (2), (4) and (6).

Suitable examples of the substituent of the optionally substituted lower alkyl group are amino, imino, lower alkoxy, lower alkoxycarbonyl, lower alkanoyl, cyclo(lower)alkyl, aryl, optionally substituted, saturated or unsaturated heterocycle, carbamoyl, mono- or di-lower alkylamino and lower alkanoyl amino.

Suitable examples of the substituent of the optionally substituted aryl group are halo(lower)alkyl and di(lower)alkylamino.

Suitable examples of the substituent of the optionally substituted saturated N-containing heterocyclic group are lower alkyl, lower alkanoyl, aryl and ar(lower)alkyl.

Suitable examples of the substituent of the optionally substituted aroyl group are halogen, lower alkyl, halo(lower)alkyl, lower alkoxy, halo(lower)alkoxy and a group represented by the formula: —CH₂—NR¹²R¹³ wherein R¹² and R¹³ are defined in the below.

Suitable examples of the substituent of the optionally substituted arylcarbamoyl group are lower alkyl, etc.

Suitable examples of the substituent of the optionally substituted arylsulfonylcarbamoyl group are lower aLklr, etc.

Suitable examples of the lower alkanoyl group and lower alkanoyl moieties in the lower alkanoylamino and lower alkanoylamino(lower)alkyl groups are formyl, acetyl, propionyl, butyryl, isobutyryl, valeryl, isovaleryl, pivaloyl, hexanoyl, etc., in which the preferred one may be (C₁-C₄)alkanoyl and the more preferred one may be acetyl.

Suitable examples of halo(lower)alkyl group are C₁₋₄, preferably C₁₋₂ alkyl group containing 1 to 9, preferably 1 to 5 halogen atoms, preferably fluorine, chlorine and/or bromine atom(s), more preferably fluorine and/or chlorine atom(s). Preferable examples the halo(lower)alkyl group are chloromethyl, bromomethyl, 2-chloroethyl, 1-fluoroethyl, 2-fluoroethyl, trifluoromethyl, trichloromethyl, chlorodifluoromethyl, dichlorofluoromethyl, 2,2-difluoroethyl, 2,2,2-trifluoroethyl, 2,2,2-trichloroethyl and pentafluoroethyl.

Suitable examples of halo(lower)alkoxy group are C₁₋₄, preferably C₁₋₂ alkoxy group containing 1 to 9, preferably 1 to 5 halogen atoms, preferably fluorine, chlorine and/or bromine atom(s), more preferably fluorine and/or chlorine atom(s). Preferable examples are chloromethoxy, bromomethoxy, 1-fluoroethoxy, 2-fluoroethoxy, trifluoromethoxy, trichloromethoxy, chlorodifluoromethoxy, dichlorofluoromethoxy, 2,2-difluoroethoxy, 2,2,2-trifluoroethoxy, 2,2,2-trichloroethoxy and pentafluoroethoxy.

Suitable examples of the ar(lower)alkyl group and ar(lower)alkyl moieties in the ar(lower)alkylamino and ar(lower)alkylcarbamoyl groups are benzyl, phenethyl, phenylpropyl, phenylbutyl, phenylpentyl, phenylhexyl, benzhydryl, trityl and naphthylmethyl.

Suitable examples of the lower alkoxy-substituted aryl are 2-, 3- or 4-methoxyphenyl, 2-, 3- or 4-ethoxyphenyl, 2-, 3- or 4-propoxyphenyl, 2-, 3- or 4-methoxynaphthyl and 2-, 3- or 4-ethoxynaphthyl.

Suitable examples of the hydroxy(lower)alkyl group are hydroxymethyl, 1- or 2-hydroxyethyl, 1,2-dihydroxyethyl, 1-, 2- or 3-propyl, 1,2-, 2,3- or 1,3-dihydroxypropyl, 1-, 2-, 3- or 4-hydroxybutyl and 1,2-, 2,3-, 3,4-, 1.3-, 1,4- or 2,4-dihydroxybutyl.

Suitable examples of the lower alkoxy(lower)alkyl group are methoxymethyl, 1- or 2-methoxyethyl, 1- or 2-ethoxyethyl, 1-, 2- or 3-methoxypropyl and 1-, 2- or 3-ethoxypropyl.

Suitable examples of the saturated or unsaturated heterocyclic(lower)alkyl group are piperidylmethyl, 1- or 2-piperidylethyl, morpholinylmethyl, 1- or 2-morpholinylethyl, 1-, 2- or 3-morpholinylpropyl, pyridylmethyl, and 1- or 2-pyridylethyl, Suitable examples of the mono- or di-lower alkylamino(lower)alkyl group are methylaminomethyl, dimethylaminomethyl, 1- or 2-methylaminoethyl, 1- or 2-dimethylaminoethyl, 1- or 2-ethylaminoethyl, 1- or 2-diethylaminoethyl, 1-, 2- or 3-methylaminopropyl and 1-, 2- or 3-dimethylaminopropyl.

Suitable examples of the lower alkanoylamino(lower) alkyl group are acetylaminomethyl, 1- or 2-acetylaminoethyl, propionylaminomethyl and 1- or 2-butyrylaminoethyl.

Suitable examples of the hydroxy- or sulfamoyl-substituted ar(aower)alllyl group are 2-, 3- or 4-hydroxyphenylmethyl, 2-, 3- or 4-sulfamoylphenylmethyl, 2-, 3- or 4-hydroxyphenylethyl, 2-, 3- or 4-sulfamoylphenylethyl, 2-hydroxy-2-phenylethyl and 1-hydroxy-2-phenylethyl.

Suitable examples of the lower alkyl-substituted, saturated or unsaturated heterocyclic group are 3-, 4-, 5- or 6-methylpyrid-2-yl, 3-, 5- or 6-methylpyrazin-2-yl and 2- or 3-methylpyrid-4-yl.

It is to be noted that the object compound (I) may include stereo isomer(s) due to the asymmetric carbon atom(s).

Suitable salts of the object compound (I) are conventional pharmaceutically acceptable ones and include a metal salt such as an alkali metal salt (e.g. sodium salt, potassium salt, etc.) and an alkaline earth metal salt (e.g. calcium salt, magnesium salt, etc.), an ammonium salt, an organic base salt (e.g. trimethylamine salt, triethylamine salt, pyridine salt, picoline salt, dicyclohexylamine salt, N,N′-dibenzylethylenediamine salt, etc.), an organic acid salt (e.g. acetate, trifluoroacetate, maleate, tartrate, fumarate, methanesulfonate, benzenesulfonate, formate, toluenesulfonate, etc.), an inorganic acid salt (e.g. hydrochloride, hydrobromide, hydriodide, sulfate, phosphate, etc.), a salt with an amino acid (e.g. arginine, aspartic acid, glutamic acid, etc.), etc.

The compound of the formula (I) and its salt can be in a form of a solvate, which is included within the scope of the present invention. The solvate preferably include a hydrate and an ethanolate.

Also included in the scope of invention are radiolabelled derivatives of compounds of formula (1) which are suitable for biological studies.

Preferred embodiments of the object compounds (I) are the one represented by the formula (I-1): 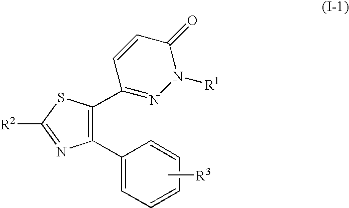
wherein
R¹ is a hydrogen atom,
   an optionally substituted lower alkyl group,
   a lower alkenyl group, or
   a cyclo(lower)alkyl,
R² is as defined in the above, and
R³ is a hydrogen atom, a halogen atom, a hydroxy group, a lower alkyl group or a lower alkoxy group.

More preferred embodiments of the object compounds (1-1) are the one wherein
R¹ is a hydrogen atom;
   a lower alkyl group which may be substituted with lower alkoxy, lower alkoxycarbonyl, lower alkanoyl, cyclo(lower)alkyl or aryl;
   a lower alkenyl group; or
   a cyclo(lower)alkyl;
R² is a hydrogen atom,
   a group represented by the formula (ia): 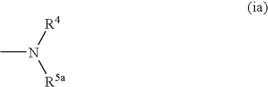
      wherein
      R⁴ is a hydrogen atom,
         a lower alkyl group or
         a lower alkenyl group, and
      R^{5a} is a hydrogen atom;
         a lower alkyl group which may be substituted with one or more substituents selected from amino, imino, lower alkoxy, aryl and saturated or unsaturated heterocyclic group;
         a lower alkyl sulfonyl group;
         a cyclo(lower)alkyl group;
         a lower alkenyl group;
         an aryl group which may be substituted with halo(lower)alkyl or di(lower)alkylamino;
         an unsaturated heterocyclic group,
   a group represented by the formula (iii): 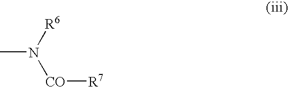
      wherein
      R⁶ is a hydrogen atom or
         a lower alkyl group, and
      R⁷ is a hydrogen atom;
         a cyclo(lower)alkyl group;
         a lower alkoxy group;
         an aryloxy group;
         a saturated or unsaturated heterocyclic group;
         a mono- or di-lower alkylamino group;
         an ar(lower)alkylamino group;
         a lower alkyl group which may be substituted with halogen, aryl, lower alkoxy-substituted aryl, aryloxy, or a group of the formula (Iv): 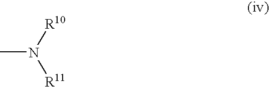
            wherein
            R¹⁰ is a hydrogen atom or a lower alkyl group,
            R¹¹ is a lower alkyl group, a cyclo(lower)alkyl group, a hydroxy(lower)alkyl group, a lower alkoxy(lower)alkyl group, a saturated or unsaturated heterocyclic(lower)alkyl group, a mono- or di-lower alkylamino(lower)alkyl group, a lower alkanoylamino(lower)alkyl group, an ar(lower)alkyl group, a hydroxy- or sulfamoyl-substituted ar(lower)alkyl group or a pyrrolidonyl(lower)alkyl group,
            or R¹⁰ and R¹¹ may combine together to form a N-containing heterocyclic group which may be substituted with lower alkyl or lower alkanoyl;
         an arylamino group which may be substituted with lower allyl;
         an arylsulfonylamino group which may be substituted with lower alkyl; or
         an aryl group which may be substituted with one or more of substituent(s) selected from the group consisting of halogen, lower alkyl, halo(lower)allyl, lower alkoxy, halo(lower)alkoxy, and
         a group of the formula (v): 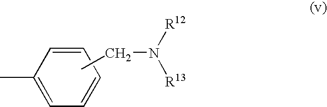
            wherein
            R¹² is a hydrogen atom or a lower alkyl group,
            R¹³ is a lower alkyl group, a hydroxy(lower)alkyl group, a lower alkoxy(lower)alkyl group, a saturated or unsaturated heterocyclic(lower)alkyl group, or a mono- or di-lower alkylamino(lower)alkyl group,
            or R¹² and R¹³ may combine together to form a N-containing heterocyclic group which may be substituted with lower alkyl, and
   a group represented by the formula (ii): 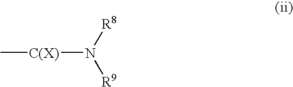
      wherein
      X is an oxygen or sulfur atom,
      R⁸ is a hydrogen atom or
         a lower alkyl group,
      R⁹ is a hydrogen atom;
         a lower alkyl group which may be substituted with carbamoyl, lower alkoxy, mono- or di-lower alkylamino, lower alkanoylamino, aryl, or unsubstituted or lower alkyl-substituted, saturated or unsaturated heterocyclic group;
         a cyclo(lower)alkyl group;
         a lower alkoxy group; or
         a mono- or di-lower alkylamino group; or
      R⁸ and R⁹ may combine together to form a saturated N-containing heterocyclic group which may be substituted with lower alkyl, lower alkanoyl, aryl or ar(lower)alkyl; and
R³ is a hydrogen atom, a halogen atom, a hydroxy group, a lower alkyl group or a lower alkoxy group.

Further preferred embodiments of the object compounds (I-1) are the one wherein
R¹ is a hydrogen atom;
   a lower alkyl group which may be substituted with lower alkoxy, lower alkoxycarbonyl, lower alkanoyl, cyclo(lower)alkyl or phenyl;
   a lower alkenyl group; or
   a cyclo(lower)alkyl;
R² is a hydrogen atom,
   a group represented by the formula (ia): 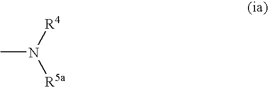
      wherein
      R⁴ is a hydrogen atom,
         a lower alkyl group or
         a lower alkenyl group, and
      R^{5a} is a hydrogen atom;
         a lower alkyl group which may be substituted with one or more substituents selected from amino, imino, lower alkoxy, phenyl, piperidyl, morpholinyl, pyridyl or furyl;
         a lower alkyl sulfonyl group;
         a cyclo(lower)alkyl group;
         a lower alkenyl group;
         a phenyl or naphthyl group which may be substituted with halo(lower)alkyl or di(lower)alkylamino;
         a pyridyl group,
   a group represented by the formula (iii): 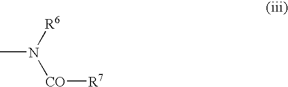
      wherein
      R⁶ is a hydrogen atom or
         a lower alkyl group, and
      R⁷ is a hydrogen atom;
         a cyclo(lower)alkyl group;
         a lower alkoxy group;
         a phenoxy group;
         a piperidyl, morpholinyl, pyridyl or carbazolyl group;
         a mono- or di-lower alkylamino group;
         a phenyl(lower)alkylamino group;
         a lower allyl group which may be substituted with halogen, phenyl, lower alkoxy-substituted phenyl, phenoxy, or a group of the formula (iv): 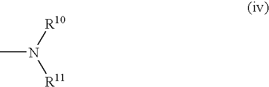
            wherein
            R¹⁰ is a hydrogen atom or a lower alkyl group,
            R¹¹ is a lower alkyl group, a cyclo(lower)alkyl group, a hydroxy(lower)alkyl group, a lower alkoxy(lower)alkyl group, a piperidyl(lower)alkyl, a morpholinyl(lower)alkyl or a pyridyl(lower)alkyl group, a mono- or di-lower alkylamino(lower)alkyl group, a lower alkanoylamino(lower)alkyl group, a phenyl(lower)alkyl group, a hydroxy- or sulfamoyl-substituted phenyl(lower) alkyl group or a pyrrolidonyl(lower)alkyl group,
            or R¹⁰ and R¹¹ may combine together to form a imidazolyl, pyrrolidinyl, piperidyl, morpholinyl or piperazinyl group which may be substituted with lower alkyl or lower alkanoyl;
         an phenylamino group which may be substituted with lower alkyl;
         an phenylsulfonylamino group which may be substituted with lower alkyl; or
         a phenyl or naphthyl group which may be substituted with one or more of
            substituent(s) selected from the group consisting of halogen, lower alkyl, halo(lower)alkyl, lower alkoxy, halo(lower)alkoxy, and
         a group of the formula (v): 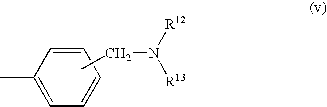
            wherein
            R¹² is a hydrogen atom or a lower alkyl group,
            R¹³ is a lower alkyl group, a hydroxy(lower)alkyl group, a lower alkoxy(lower)alkyl group, a piperidyl(lower)alkyl, a morpholinyl(lower)alkyl or a pyridyl(lower)alkyl group, or a mono- or di-lower alkylamino(lower)alkyl group,
            or R¹² and R¹³ may combine together to form a imidazolyl, pyrrolidinyl, piperidyl, morpholinyl or piperazinyl group which may be substituted with lower alkyl, and
   a group represented by the formula (ii): 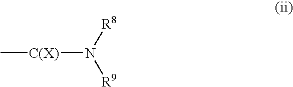
      wherein
      X is an oxygen or sulfur atom,
      R⁸ is a hydrogen atom or
         a lower alkyl group,
      R⁹ is a hydrogen atom;
         a lower alkyl group which may be substituted with carbamoyl, lower alkoxy, mono- or di-lower alkylamino, lower alkanoylamino, phenyl, morpholinyl, pyridyl or pyrazinyl which may be substituted with lower alkyl;
         a cyclo(lower)alkyl group;
         a lower alkoxy group; or
         a mono- or di-lower alkylamino group; or
      R⁸ and R⁹ may combine together to form a pyrrolidinyl, piperidyl, morpholinyl or piperazinyl group which may be substituted with lower alkyl, lower alkanoyl, phenyl or phenyl(lower)alkyl and
R³ is a hydrogen atom, a halogen atom, a hydroxy group, a lower allcyl group or a lower alkoxy group.

The object compounds (I) and (I-1) and a salt thereof of the present invention can be prepared by the following processes.
Process 1 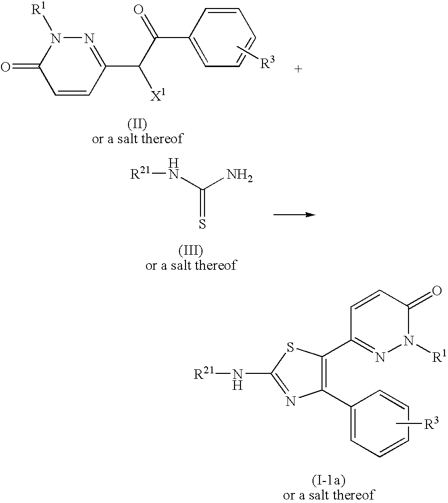
Process 2 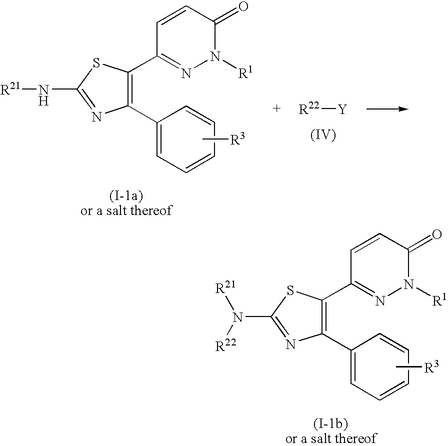
Process 3 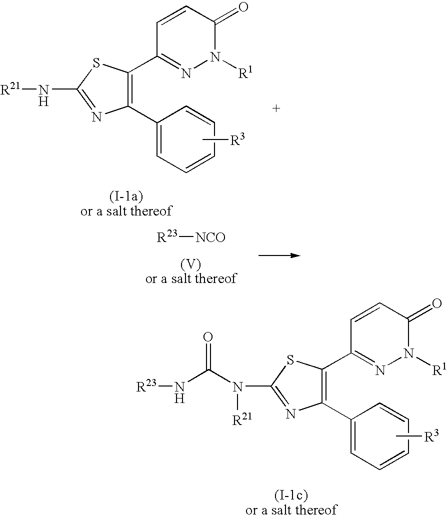
Process 4 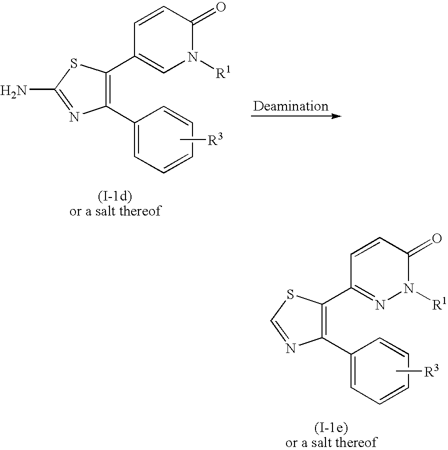
Process 5 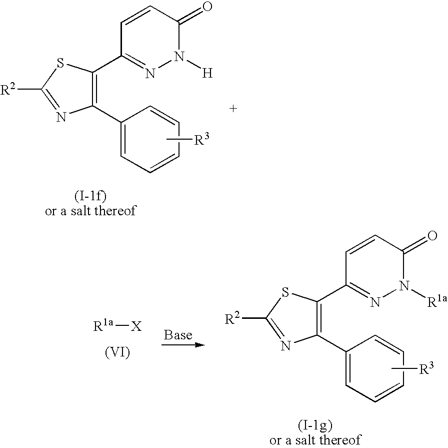
Process 6 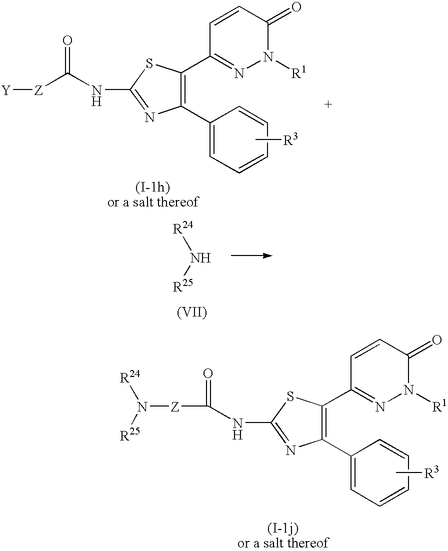
Process 7 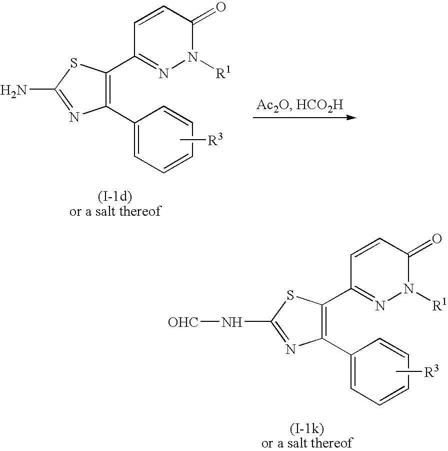
Process 8 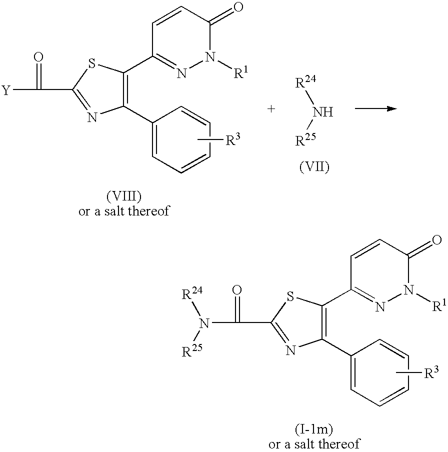
Process 9 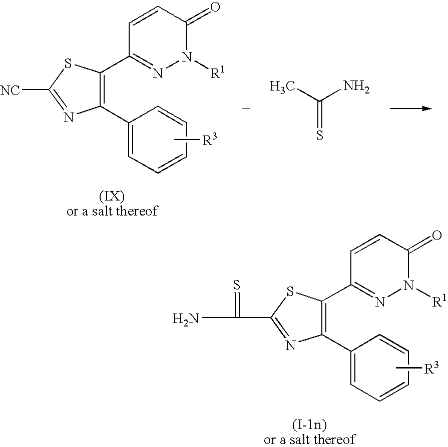 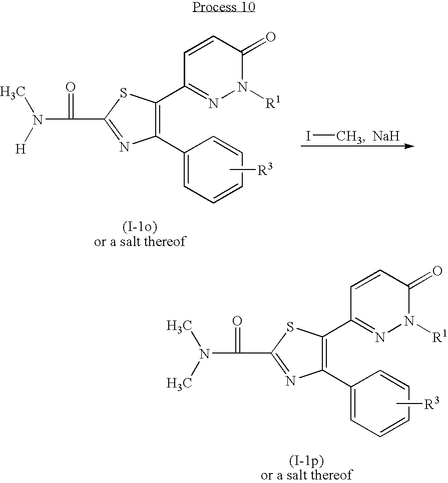
wherein
R¹, R² and R³ are as defined above,
R^{1a} is an optionally substituted lower alkyl, lower alkenyl or cyclo(lower)alkyl group,
R²¹ is a hydrogen atom or an optionally substituted lower alkyl, optionally substituted aryl, cyclo(lower)alkyl, heterocycle or acyl group,
R²² is an optionally substituted lower alkyl, acyl or lower alkenyl group,
R²³ is a hydrogen atom, an optionally substituted aryl, optionally substituted lower alkyl, acyl or heterocyclic group,
R²⁴ is a hydrogen atom or a lower alkyl group,
R²⁵ is an optionally substitutted lower alkyl, cyclo(lower)alkyl, pyrrolidonyl(lower)alkyl, optionally substituted lower alkanoyl, or di-lower alkylamino group, or
R²⁴ and R²⁵ may combine together to form an optionally substituted heterocyclic group,
X¹ is a halogen atom,
Y is a leaving group.
Z is —(CH₂)ₙ—, 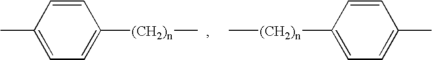
   or phenylene, and
n is 1 or 2.

Suitable leaving group are halogen as mentioned above, hydroxy, acyloxy such as alkanoyloxy (e.g. acetoxy, propionyloxy, etc.), lower alkoxy (e.g., ethoxy etc.), sulfonyloxy (e.g. mesyloxy, tosyloxy, etc.), etc.

Suitable salt of the compounds (I-1a), (I-1b), (I-1c), (I-1d), (I-1e), (VIII) and (IX) can be referred to the ones as exemplified for the compound (I).

The processes for preparing the object thiazole derivative(I) are explained in detail in the following.

Process 1

The compound (I-1a) or a salt thereof can be prepared by reacting the compound (II) or a salt thereof with the thiourea derivative (III) or a salt thereof.

The reaction is preferably conducted in the presence of a base, for example, inorganic base such as alkali metal hydroxide (e.g. sodium hydroxide, potassium hydroxide, etc.), alkali metal carbonate(e.g. sodium carbonate, potassium carbonate, etc.), alkali metal bicarbonate(e.g. sodium hydrogen carbonate, potassium hydrogen carbonate, etc.), alkali metal hydride (e.g. sodium hydride), alkali metal alkoxide (e.g. EtONa, t-BuOK, etc.) organic base such as trialkylamine, etc.

The reaction may be carried out in a conventional solvent such as water, alcohol (e.g. methanol, ethanol, etc.), acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction. These conventional solvents may also be used in a mixture with water.

The reaction temperature is not critical, and the reaction is usually carried out at ambient temperature, under warming or under heating.

Process 2

The compound (I-1b) or a salt thereof can be prepared by reacting the compound (I-1a) or a salt thereof with a compound (IV).

The reaction is preferably conducted in the presence of a base, for example, inorganic base such as alkali metal hydroxide (e.g. sodium hydroxide, potassium hydroxide, etc.), alkali metal carbonate, alkali metal bicarbonate, alkali metal hydride (e.g. sodium hydride), alkali metal alkoxide (e.g. EtONa, t-BuOK, etc.) organic base such as trialkylamine (e.g. triethylamine, etc.), etc.

Alternatively, the present reaction is preferably carried out in the presence of alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.) etc.

When Y is —OH, activation of OH with triphenylphosphine and the like may be necessary.

The present reaction may be carried out in a solvent such as water, phosphate buffer, acetone, chloroform, acetonitrile, nitrobenzene, methylene chloride, ethylene chloride, formamide, N,N-dimethylformamide, methanol, ethanol, sec-butanol, amyl alcohol, diethyl ether, dioxane, tetrahydrofuran, dimethyl sulfoxide, pyridine or any other organic solvent which does not adversely affect the reaction, preferably in ones having strong polarities. Among the solvents, hydrophilic solvents may be used in a mixture with water. When the compound (IV) is in liquid, it can also be used as a solvent.

The reaction temperature is not critical, and the reaction is usually carried out at ambient temperature, under warming or under heating.

Process 3

The compound (I-1c) or a salt thereof can be prepared by reacting the compound (I-1a) or a salt thereof with the compound (V) or a salt thereof.

The reaction is preferably conducted in the presence of a base, for example, inorganic base such as alkali metal hydroxide (e.g. sodium hydroxide, potassium hydroxide, etc.), alkali metal carbonate, alkali metal bicarbonate, alkali metal hydride (e.g. sodium hydride), alkali metal alkoxide (e.g. EtONa, t-BuOK, etc.) organic base such as trialkylamine (e.g. triethylamine, etc.), etc.

Alternatively, the present reaction is preferably carried out in the presence of alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.) etc.

The present reaction may be carried out in a solvent such as acetone, chloroform, acetonitrile, nitrobenzene, methylene chloride, ethylene chloride, formamide, N,N-dimethylformamide, diethyl ether, dioxane, tetrahydrofuran, dimethyl sulfoxide, pyridine or any other organic solvent which does not adversely affect the reaction, preferably in ones having strong polarities. When the compound (V) is in liquid, it can also be used as a solvent.

The reaction temperature is not critical, and the reaction is usually carried out at ambient temperature, under warming or under heating.

Process 4

The compound (I-1e) or a salt thereof can be prepared by subjecting the compound (1-d) or a salt thereof to deamination reaction.

The deamination reaction can be carried out in the presence of isoamyl nitrate in a solvent such as chloroform, acetonitrile, methylene chloride, diethyl ether, dioxane, tetrahydrofuran or any other organic solvent which does not adversely affect the reaction. The reaction temperature is not critical, and the reaction is usually carried out at ambient temperature, under warming or under heating.

Process 5

The compound (I-1g) or a salt thereof can be prepared by reacting the compound (I-1f) or a salt thereof with a compound (VI).

The reaction is usually conducted in the presence of a base, for example, inorganic base such as alkali metal hydroxide (e.g. sodium hydroxide, potassium hydroxide, etc.), alkali metal carbonate(e.g. sodium carbonate, potassium carbonate, etc.), alkali metal bicarbonate(e.g. sodium hydrogen carbonate, potassium hydrogen carbonate, etc.), alkali metal hydride (e.g. sodium hydride), alkali metal alkoxide (e.g. EtONa, t-BuOK, etc.) organic base such as trialkylamine (e.g., triethylamine), and the like.

The reaction may be carried out in a conventional solvent such as acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction.

The reaction temperature is not critical, and the reaction is usually carried out at ambient temperature, under warming or under heating.

Process 6

The compound (I-1j) or a salt thereof can be prepared by reacting the compound (I-1 h) or a salt thereof with amine derivative (VII).

The reaction is preferably conducted in the presence of a base, for example, inorganic base such as alkali metal hydroxide (e.g. sodium hydroxide, potassium hydroxide, etc.), alkali metal carbonate(e.g. sodium carbonate, potassium carbonate, etc.), alkali metal bicarbonate(e.g. sodium hydrogen carbonate, potassium hydrogen carbonate, etc.), alkali metal hydride (e.g. sodium hydride), alkali metal alkoxide (e.g. EtONa, t-BuOK, etc.) organic base such as trialkylamine, and the like.

The reaction may be carried out in a conventional solvent such as acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction.

The reaction temperature is not critical, and the reaction is usually carried out at ambient temperature, under warming or under heating.

Process 7

The compound (I-1k) or a salt thereof can be prepared by reacting the compound (I-1d) or a salt thereof with acetic anhydride and formic acid.

The reaction may be carried out in a conventional solvent such as acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction.

The reaction temperature is not critical, and the reaction is usually carried out at ambient temperature, under warming or under heating.

Process 8

The compound (I-1m) or a salt thereof can be prepared by reacting the compound (VIII) or a salt thereof with the amine (VII).

The reaction of this process can be carried out in the manner similar to that of Process 6.

Process 9

The compound (I-1n) or a salt thereof can be prepared by reacting the compound (IX) or a salt thereof with thioacetamide.

The reaction is preferably conducted in the presence of an acid, for example, organic acid such as acetic acid or inorganic acid such as hydrochloric acid, hydrobromic acid, etc.

The reaction may be carried out in a conventional solvent such as acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction.

The reaction temperature is not critical, and the reaction is usually carried out at ambient temperature, under warming or under heating.

Process 10

The compound (I-1p) or a salt thereof can be prepared by reacting the compound (I-1o) or a salt thereof with methyl idodide and base.

The reaction of this process can be carried out in the manner similar to that of Process 5.

The starting compounds (II), (II-1), (VIII), (VIII-2) and (IX) or a salt thereof are novel and can be prepared, for example, by the following reaction schemes. 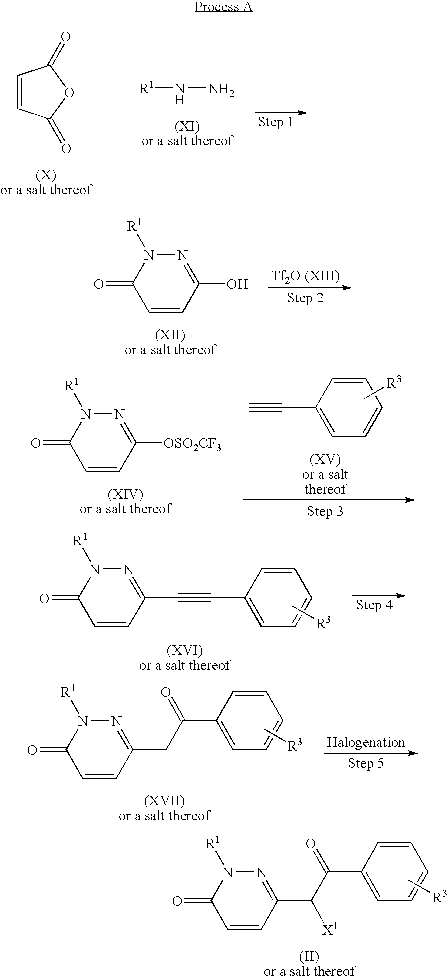 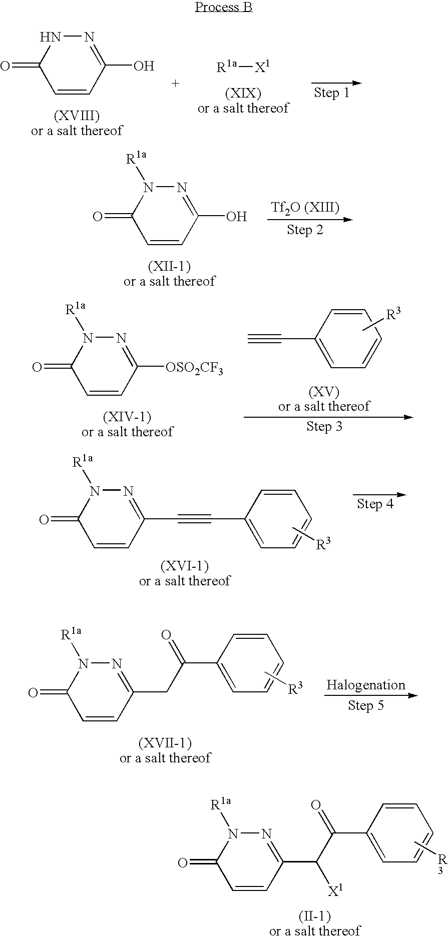 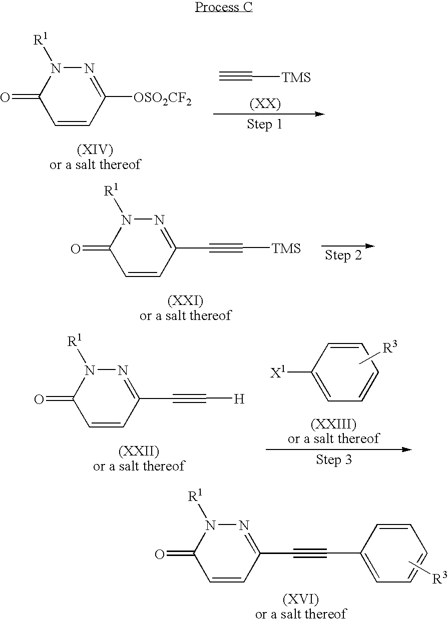 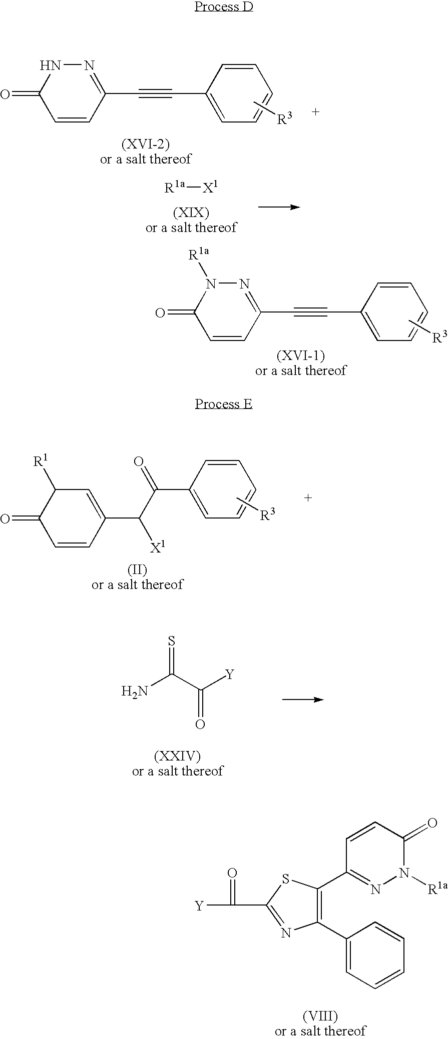 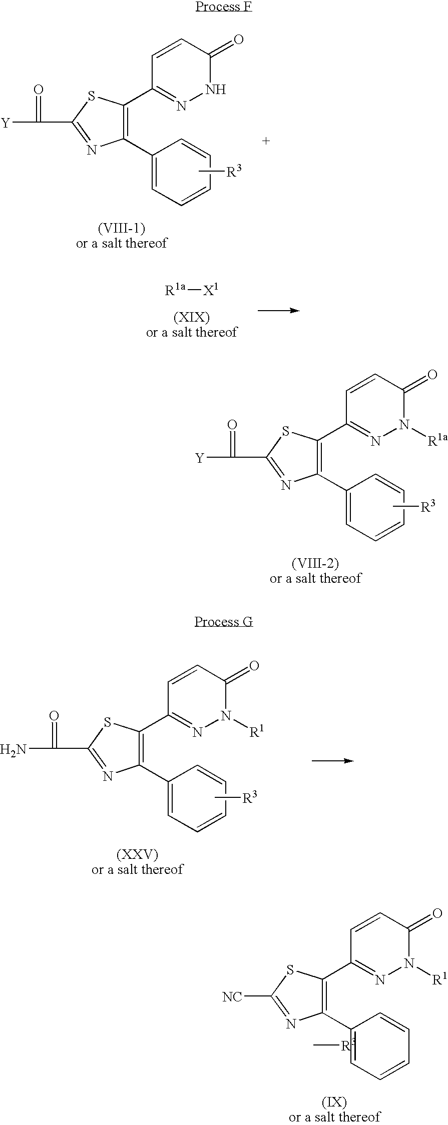
wherein R¹, R³, Y, R^{1a} and X¹ are as defined above,
Tf₂O is trifluoromethanesulfonic anhydride,
TMS is trimethylsilyl and
Steps 2 to 5 of Process B are as same as those of Process A.

Suitable salt of the compounds (II), (II-1), (VIII), (VIII-1), (VIII-2), (IX), (X), (XI), (XII), (XII-1), (XIV), (XIV-1), (XV), (XVI), (XVI-1), (XVI-2), (XVII), (XVII-1), (XVIII), (XIX), (XXI), (XXII), (XXIII), (XXV) and (XXV) can be referred to the ones as examplified for the compound (I).

Process A

Step 1: The compound (XII) or a salt thereof can be prepared by reacting the compound (X) or a salt thereof and the compound (XI) or a salt thereof. The reaction is usually carried out in the presence of an acid, for example, organic acid such as acetic acid or inorganic acid such as hydrochloric acid, hydrobromic acid, etc.

This reaction is usually carried out in a conventional solvent such as alcohol (e.g. methanol, ethanol, etc.), acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction. The acid can be used as the solvent if it is liquid.

The reaction temperature is not critical, and the reaction is usually carried out at ambient temperature, under warming or under heating, preferebly under heating.

Step 2: The compound (XIV) or a salt thereof can be prepared by reacting the compound (XII) or a salt thereof with trifluoromethane sulfonic acid anhydride (XIII). The reaction is usually carried out in the presence of a base, for example, inorganic base such as alkali metal hydroxide (e.g. sodium hydroxide, potassium hydroxide, etc.), alkali metal carbonate(e.g. sodium carbonate, potassium carbonate, etc.), alkali metal bicarbonate(e.g. sodium hydrogen carbonate, potassium hydrogen carbonate, etc.), alkali metal hydride (e.g. sodium hydride), alkali metal alkoxide (e.g. EtONa, t-BuOK, etc.) organic base such as trialkylamine, pyridine and the like.

The reaction may be carried out in a conventional solvent such as dioxane, acetonitrile, chloroform, methylene chloride, ethylene chloride, tetrahydrofuran, ethyl acetate, pyridine or any other organic solvent which does not adversely affect the reaction.

The reaction temperature is not critical, and the reaction is usually carried out at ambient temperature, under warming or under heating, preferebly under heating.

Step 3: The compound (XVI) or a salt thereof can be prepared bycoupling the compound (XI) or a salt thereof and the compound (XV) or a salt thereof.

The reaction is usually conducted in the presence of palladium and copper catalyst such as dichlorobis(triphenylphosphine)palladium (II) and copper a) iodide.

Besides, the reaction is usually carried out in the presence of a base, for example, inorganic base such as alkali metal hydroxide (e.g. sodium hydroxide, potassium hydroxide, etc.), alkali metal carbonate(e.g. sodium carbonate, potassium carbonate, etc.), alkali metal bicarbonate(e.g. sodium hydrogen carbonate, potassium hydrogen carbonate, etc.), alkali metal hydride (e.g. sodium hydride), alkali metal alkoxide (e.g. EtONa, t-BuOK, etc.) organic base such as trialkylamine, pyridine and the like.

The reaction may be carried out in a conventional solvent such as acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction.

The reaction temperature is not critical, and the reaction is usually carried out at ambient temperature, under warming or under heating.

Step 4: The compound (XVII) or a salt thereof can be prepared by reacting the compound (XVI) or a salt thereof with sulfuric acid and acetic acid.

The reaction may be carried out in a conventional solvent such as acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction.

The reaction temperature is not critical, and the reaction is usually carried out at ambient temperature, under warming or under heating.

Step 5: The compound (II) or a salt thereof can be prepared by subjecting the compound (XVII) or a salt thereof to halogenation. Halogenation reaction can be carried out in the presence of pyridinium tribromide or sulfuryl chloride.

The reaction may be carried out in a conventional solvent such as acetone, dioxane, acetonitrile, chloroform, methylene chloride, ethylene chloride, tetrahydrofuran, ethyl acetate, N,N-dimethylformamide, acetic acid or any other organic solvent which does not adversely affect the reaction.

The reaction temperature is not critical, and the reaction is usually carried out at ambient temperature, under warming or under heating.

Process B

The compound (II-1) or a salt thereof can be prepared by reacting the compound (XVIII) or a salt thereof with the compound (XIX) by Steps 1 to 5.

Step 1: The compound (XII-1) or a salt thereof can be prepared by reacting the compound (XVIII) or a salt thereof with a silylation reagent and then reacting with a halide compound (XIX) or a salt thereof.

The silylation usually proceeds in the presence of a silylating reagent such as N,N′-bis(trimethylsilyl)urea (BSU), 1,1,1,3,3,3-hexamethyldisilazane (HMDS), etc. and optionally a catalyst such as sulfuric acid. The amount of the silylating reagent is preferably more than 2 equivalent of the compound (XVIII) or a salt thereof. The silylation may be carried out in a conventional solvent such as dioxane, acetonitrile, chloroform, methylene chloride, ethylene chloride, tetrahydrofuran, benzene, toluene or any other organic solvent which does not adversely affect the reaction.

The reaction temperature of the silylation is not critical, and the reaction is preferably carried out under heating.

After silylation, both the silylating reagent and the solvent are preferably removed such as evaporation. Then, the silylated compound can be reacted with the halide compound MIX) or a salt thereof in a solvent such as the one having the high inductivity, for example o-dichlorobenzene, nitrobenzene, ethylene carbonate, propylene carbonate, etc. The amount of the halide compound (XIX) is at least 1 equivalent, preferably more than 1 equivalent of the compound (XVIII).

The reaction temperature is not critical, and the reaction is preferably carried out under heating.

Silylation of 3,6-dihydroxypyridazine improves its reactivity and solubility and using the solvent having the high inductivity for the alkylation with the compound p) can facilitate preparing the compound (XII-1).

The Steps 2 to 5 can be respectively carried out in a manner similar to Steps 2 to 5 of Process A.

Process C

Step 1: The compound (XVI) or a salt thereof can be prepared by reacting the compound (XIV) or a salt with the compound (XX). The Step 1 can be carried out in a manner similar to Step 3 of Process A.

Step 2: The compound (XXI) or a salt thereof can be prepared by subjecting the compound (X) or a salt thereof to a base, for example, inorganic base such as alkali metal hydroxide (e.g. sodium hydroxide, potassium hydroxide, etc.), alkali metal carbonate(e.g. sodium carbonate, potassium carbonate, etc.), alkali metal bicarbonate(e.g. sodium hydrogen carbonate, potassium hydrogen carbonate, etc.), alkali metal hydride (e.g. sodium hydride), alkali metal alkoxide (e.g. EtONa, t-BuOK, etc.) organic base such as triallkylamine, pyridine and the like.

The reaction may be carried out in a conventional solvent such as water, alcohol (e.g. methanol, ethanol, etc.), acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction. These conventional solvents may also be used in a mixture with water.

The reaction temperature is not critical, and the reaction is usually carried out at ambient temperature, under warming or under heating.

Step 3: The Step 3 can be carried out in a manner similar to Step 3 of Process A.

Process D

The compound (XVI-1) or a salt thereof can be prepared by reacting the compound (XVI-2) or a salt thereof with the compound (XIX) or a salt thereof.

The reaction of this process can be carried out in a manner similar to Process 5.

Process E

The compound (VIII) or a salt thereof can be prepared by reacting the compound (II) or a salt thereof with the compound (XXIV) or a salt thereof.

The reaction may be carried out in a conventional solvent such as acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction.

The reaction temperature is not critical, and the reaction is usually carried out at ambient temperature, under warming or under heating.

Process F

The compound (VII-2) or a salt thereof can be prepared by reacting the compound (VIII-1) or a salt thereof with the compound (XIX) or a salt thereof.

The reaction of this process can be carried out in a manner similar to Process 5.

Process G

The compound (IX) or a salt thereof can be prepared by reacting the compound (XXV) or a salt thereof with trifluoroacetic anhydride and pyridine.

The reaction may be carried out in a conventional solvent such as acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction.

The reaction temperature is not critical, and the reaction is usually carried out at ambient temperature, under warming or under heating.

In order to show the usefulness of the compound (I) of the present invention, the pharmacological test result of the representative compound of the present invention is shown in the following.

Test 1: Adenosine Antagonistic Activity

[I] Test Method

The adenosine antagonistic activity [Ki(nM)] of the test compound was examined by radioligand binding techniques using 8-cyclopentyl-1,3-dipropylxanthine, [dipropyl-2,3⁻³H(N)] ([3H]DPCPX, 4.5 nM) for human A₁ receptor and [3H]CGS 21680 (20 nM) for human A₂ₐ receptor.

[II] Test Compound

N-[5-(1-Isopropyl-6-oxo-1,6-dihydro-3-pyridazinyl)-4-phenyl-1,3-thiazol-2-yl]hexanamide (Example 3)

N-[5-(1-Isopropyl-6-oxo-1,6-dihydro-3-pyrndazinyl)-4-phenyl-1,3-thiazol-2-yl]-2-(4-methoxy-phenyl)acetamide (Example 9)

N-[5-(1-Isopropyl-6-oxo-1,6-dihydro-3-pyridazinyl)-4-phenyl-1,3-thiazol-2-yl]-N′-(3-methylphenyl)urea (Example 10)

2-Isopropyl-6-[2-(methylamino)-4-phenyl-1,3-thiazol-5-yl]-3(2H)-pyridazinone (Example 15)

[III] Test Result

TABLE 1

| | |
|---|---|
| | Adenosine receptor |
| Test compound | binding (Ki:nM) |

| (Example No.) | A₁ | A₂ₐ |
|---|---|---|
| 3 | 0.27 | 1.46 |
| 9 | 0.28 | 1.22 |
| 10 | 0.38 | 3.08 |
| 15 | 0.12 | 1.63 |
| 78 | 0.45 | 1.23 |
| 100 | 0.61 | 1.22 |
| 214 | 1.14 | 1.52 |
| 233 | 1.03 | 1.14 |

Test 2: Anticatalepsy Activity in Mouse

The test compound (3.2 mg/kg) was administered orally with ddY mice(n=7). Then, haloperidol (0.32 mg/kg) was injected intraperitoneally 30 min. after the administration of the compound. Thirty minutes after the injection, the cataleptic responses of mice were measured. The forelimbs of each mouse were placed on a 3 cm high, 3 mm wide horizontal bar, and the duration of cataleptic posture was measured for up to 30 sec.

[II] Test Compound

N-[5-(1-Isopropyl-6-oxo-1,6-dihydro-3-pyridazinyl)-4-phenyl-1,3thiazol-2-yl]hexanamide (Example 3)

N-[5-(1-Isopropyl-6-oxo-1,6-dihydro-3-pyridazinyl)₄— phenyl-1,3-thiazol-2-yl]-2-(4-methoxyphenyl)acetaride (Example 9)

2-Isopropyl-6-[2-(methylamino)-4-phenyl-1,3-thiazol-5-yl]-3(2H)-pyridazinone (Example 15)

[III] Test Result

TABLE 2

| | Manifestation rate of catalepsy |
|---|---|
| Test compound (Example No.) | (number of mouse) |
| 3 | 0/7 |
| 9 | 0/7 |
| 15 | 0/7 |
| 78 | 0/7 |
| 100 | 0/7 |
| 214 | 0/7 |
| 233 | 0/7 |

The thiazole derivatives of the present invention have an adenosine antagonistic activity and pharmacological action such as anticatelepsy activity as shown in the above.

The thiazole derivative and a salt thereof of the present invention are useful as adenosine antagonists (especially, A₁ receptor and A₂ (particularly A₂ₐ) receptor dual antagonists) and possess various pharmacological actions such as anticatalepsy action, cognitive enhancing action, analgesic action, locomotor action, antidepressant action, diuretic action, cardioprotective action, cardiotonic action, vasodilating action (e.g. cerebral vasodilating action, etc.), the action of increasing the renal blood flow, renal protective action, improvement action of renal function, enhancing action of lipolysis, inhibition action of anaphylactic bronchoconstriction, acceleration action of the insulin release, the aciton of increasing the production of erythropoietin, inhibiting action of platelet aggregation, etc.

Therefore, the thiazole derivative (I) and a salt thereof of this invention are useful as cognitive enhancer, antianxietry drug, antidementia drug, psychostimulant, analgesic, cardioprotective agent, antidepressant, ameliorants of cerebral circulation, tranquilizer, drug for heart failure, cardiotonic agent, antihypertensive agent, drug for renal failure (renal insufficiency), drug for renal toxicity, renal protective agent, drug for improvement of renal function, diuretic, drug for edema, antiobesity, antiasthmatic, bronchodilator, drug for apnea, drug for gout, drug for hyperuricemia, drug for sudden infant death syndrome (SDS), ameliorants of immunosuppressive action of adenosine, antidiabetic agent, drug for ulcer, drug for pancreatitis, drug for Meniere's syndrome, drug for anemia;
drug for thrombosis, drug for myocardial infarction, drug for obstruction, drug for arteriosclerosis obliterans, drug for thrombophlebitis, drug for cerebral infarction, drug for transient ischemic attack, drug for angina pectoris, etc.;
and useful for the prevention and/or treatment of depression, dementia (e.g. Alzheimer's disease, cerebrovascular dementia, dementia accompanying Parkinson's disease, etc.), Parkinson's disease, anxiety, pain, cerebrovascular disease (e.g. stroke, etc.), heart failure; hypertension (e.g. essential hypertension, nephrogenous hypertension, etc.);
circulatory insufficiency (acute circulatory insufficiency) cuased by, for example, ischemia/reperfusion injury (e.g. myocardial ischemia/reperfusion injury, cerebral ischentia/reperfusion injury, peripheral ischemia/reperfusion injury, etc.), shock (e.g. endotoxin shock, hemorrhagic shock, etc.), surgical procedure, etc.;
post-resuscitation asystole;
bradyarrhythmia;
electro-mechanical dissociation;
hemodynamic collapse;
SIRS (systemic inflammatory response syndrome);
multiple organ failure;
renal failure (renal insufficiency) (e.g. acute renal failure, etc.), renal toxicity [e.g. renal toxicity induced by a drug such as cisplatins, gentamicin, FR-900506 (disclosed in EP-0184162), cyclosporin (e.g. cyclosporin A) etc.; glycerol, etc.], nephrosis, nephritis, edema (e.g. cardiac edema, nephrotic edema, hepatic edema, idiopathic edema, drug edema, acute angioneurotic edema, hereditary angioneurotic edema, carcinomatous ascites, gestational edema, etc.);
obesity, bronchial asthma, gout, hyperuricemia, sudden infant death syndrome, immunosuppression, diabetes, ulcer such as peptic ulcer (e.g. gastric ulcer, duodenal ulcer, etc.), pancreatitis, Meniere's syndrome, anemia, dialysis-induced hypotension, constipation, ischemic bowel disease, ileus (e.g. mechanical ileus, adynamic ileus, etc.); and
myocardial infarction, thrombosis (e.g. arterial thrombosis, cerebral thrombosis, etc.), obstruction, arteriosclerosis obliterans, thrombophlebitis, cerebral infarction, transient ischemic attack, angina pectoris, etc.

The present invention provides a pharmaceutical composition which contains the thiazole derivative (I) or a pharmaceutically acceptable salt thereof as an active ingredient in admixture with an organic or inorganic carrier or excipient suitable for rectal, pulmonary (nasal or buccal inhalation), nasal, ocular, external (topical), oral or parenteral (including subcutaneous, intravenous and intramuscular) administrations or insufflation. The pharmaceutical composition of this invention can be formulated in the form of a pharmaceutical preparation, for example, in a solid, semisolid or liquid form. The examples of the carrier or excipient are non-toxic, pharmaceutically acceptable carriers for tablets, pellets, troches, capsules, suppositories, creams, ointments, aerosols, powders for insufflation, solutions, emulsions, suspensions, and any other form suitable for use. In addition, auxiliary, stabilizing agents, thickening agents, coloring agents and perfumes may be used where it is necessary. The thiazole derivative (I) or a pharmaceutically acceptable salt thereof is included in a pharmaceutical composition in an amount sufficient to produce the desired aforesaid pharmaceutical effect upon the process or condition of diseases.

For applying the composition to a human being or an animal, it is preferable to apply it by intravenous, intramuscular, pulmonary or oral administration, or insufflation. While the dosage of therapeutically effective amount of the thiazole derivative (I) varies depending on the age and condition of each individual patient to be treated, in the case of intravenous administration, a daily dose of 0.01-100 mg of the thiazole derivative (I) per kg weight of a human being or an animal, in the case of intramuscular administration, a daily dose of 0.1-100 mg of the thiazole derivative (I) per kg weight of a human being or an animal, and in case of oral administration, a daily dose of 0.5-100 mg of the thiazole derivative (I) per kg weight of a human being or an animal is generally given for the prevention and/or treatment of the aforesaid diseases.

The following Preparations and Examples are given for the purpose of illustrating the present invention in more detail.

Preparation 1

To a solution of maleic anhydride (41.57 g) in glacial acetic acid (310 ml) was added 1-isopropyl-hydrazine (31.43 g) at ambient temperature. The mixture was heated under reflux for 5 hours and then concentrated under reduced pressure to give a solid. The solid was triturated by diisopropyl ether, collected by filtration and recrystalized from a mixture of methanol and isopropyl ether to give 6-hydroxy-2-isopropyl-3(2H)-pyridazinone (60.27 g).

mp: 162-164° C.

IR(KBr): 1504 cm⁻¹

¹H NMR(CDCl₃, δ): 1.22(6H,d,J=6.66 Hz), 5.03(1H, 7-plet,J=6.65 Hz), 6.85(1H,d,J=9.62 Hz), 7.01(1H,d,J=9.62 Hz), 10.95(1H,br.s)

APCI/MS: 155[M+H]⁺

Elemental Analysis for C₇H₁₀N₂O₂

Calcd.: C, 54.54; H, 6.54; N, 18.17

Found: C, 54.72; H, 6.61; N, 18.13

Preparation 2

To a solution of 6-hydroxy-2-isopropyl-3(2H)-pyridazinone (5.00 g) in pyridine (32 ml) was dropwise added tifluoromethanesulfonic anhydride (5.51 ml) under ice-cooling. The mixture was stirred for one hour under ice-cooling and for 3 hours at ambient temperature. Pyridine was removed under reduced pressure to give a residue. The residue was dissolved in a mixture of ethyl acetate and water. An organic layer was washed with brine, dried over magnesium sulfate, and concentrated under reduced pressure to give a residue. The residue was purified by a column chromatography on silica gel (n-hexane:ethyl acetate=8:2,v/v) to give 1-isopropyl-6-oxo-1,6-dihydro-3-pyridazinyl trifluoromethanesulfonate as a solid (8.66 g).

mp: 45-46° C.

IR(KBr): 1660, 1587 cm⁻¹

¹H NMR(CDCl₃, δ): 1.34(6H,d,J=6.62 Hz), 5.23(1H, 7-plet,J=6.61 Hz), 7.04(1H,d,J=9.83 Hz), 7.16(1H,d,J=9.83 Hz)

APCI/MS: 287[M+H]⁺

Elemental Analysis for C₈H₉F₃N₂O₄S

Calcd.: C, 33.57; H, 3.17; N, 9.79

Found: C, 33.80; H, 2.96; N, 9.79

Preparation 3

In the presence of dichlorobis(triphenylphosphine)palladium (II)(0.49 g) and copper(1)iodide (0.133 g), a solution of triethylamine (11.7 ml) in dioxane (10 ml) was added dropwise to a mixture of 1-isopropyl-6-oxo-1,6-dihydro-3-pyridazinyl trifluoromethanesulfonate (20.00 g), ethynylbenzene (8.56 g) in dioxane (70 ml) at 75-80° C. for 0.5 hour. The mixture was stirred for 1.5 hours at 75-80° C. After cooling, a mixture of water and chloroform was added to the mixture. The separated organic layer was washed with brine, dried over magnesium sulfate, and concentrated under reduced pressure to give a residue. The residue was purified by a column chromatography on silica gel (n-hexane:ethyl acetate=85:15, v/v) to give 2-isopropyl-6-(phenylethynyl)-3(2H)-pyridazinone as a solid (16.17 g).

mp: 75.5-77° C.

IR(KBr): 2218, 1669, 1583 cm⁻¹

¹H NMR(CDCl₃, δ): 1.40(6H,d,J=6.65 Hz), 5.33(1H, 7-plet,J=6.65 Hz), 6.87(1H,d,J=9.57 Hz), 5.13(1H,d,J=9.57 Hz), 7.34-7.42(3H,m), 7.52-7.60(2H,m)

APCI/MS: 239[M+H]⁺, 197

Elemental Analysis for C₁₅H₁₄N₂O

Calcd.: C, 75.61; H, 5.92; N, 11.76

Found: C, 75.79; H, 5.88; N, 11.74

Preparation 4

To a mixture of sulfuric acid (1 ml) and acetic acid (3 ml) was added 2-isopropyl-6-(phenyl-ethynyl)-3(2H)-pyridazinone (479 mg), and the mixrture was heated for 2 hours at 100-105° C. The solution was poured into ice-water (80 ml) and extracted with ethyl acetate (30 ml×3). The organic layer was dried over magnesium sulfate and concentrated under reduced pressure to give a residue. The residue was purified by a column chromatography on silica gel (n-hexane:ethyl acetate=1:3, v/v) to give 2-isopropyl-6-(2-oxo-2-phenylethyl)-3(2H)-pyridazinone as a solid (451 mg).

mp: 50-53° C.

IR(KBr): 1687, 1660, 1595 cm⁻¹

¹H NMR(CDCl₃, δ): 1.32(6H,d,J=6.66 Hz), 4.32(2H,s), 5.29(1H, 7-plet, J=6.66 Hz), 6.88(1H,d,J=9.50 Hz), 7.18(1H,d,J=9.50 Hz), 7.45-7.62(3H,m), 8.01-8.07(2H,m)

APCI/MS: 257[M+H]⁺, 215

Elemental Analysis for C₁₅H₁₆N₂O₂

Calcd.: C, 70.29; H, 6.29; N, 10.93

Found: C, 69.17; H, 6.32; N, 10.74

Preparation 5

To a solution of 2-isopropyl-6-(2-oxo-2-phenylethyl)-3(2H)-pyridazinone (610 mg) in acetic acid (5 ml) was added 30% hydrogen bromide solution in acetic acid (0.5 ml). Under ice-cooling, pyridinium tribromide (915 mg) was added. The mixture was stirred for 30 minutes at the same temperature and for 3 hours at ambient temperature. The solution was poured into ice-water(50 ml) and extracted with chloroform (20 ml×3). The organic layer was dried over magnesium sulfate and concentrated under reduced pressure to give a residue. The residue was purified by a column chromatography on silica gel(n-hexane:ethyl acetate=4:1, v/v) to give 6-(1-bromo-2-oxo-2-phenylethyl)-2-isopropyl-0.3(2H)-pyridazinone as a solid (690 mg).

mp: 98-100° C.

IR(KBr): 1707, 1660, 1587 cm⁻¹

¹H NMR(CDCl₃, δ): 1.19(3H,d,J=6.64 Hz), 1.34(3H,d,J=6.64 Hz), 5.27(1H, 7-plet,J=6.64 Hz), 6.25(1H,s), 6.95(1H,d,J=9.70 Hz), 7.26-7.69(4H,m), 8.05-8.10(2H,m)

APCI/MS: 336 and 334[M+H]⁺, 295 and 293, 257, 215

Elemental Analysis for C₁₅H₁₅BrN₂O₂

Calcd.: C, 53.75; H, 4.51; N, 8.36

Found: C, 53.65; H, 4.53; N, 8.31

Preparation 6

To a mixture of maleic hydrazide (200 g) and HMDS (1,1,1,3,3,3-hexamethyldisilazane, 576 g) in toluene (800 ml) as solvent was added dropwise sulfuric acid (17.5 g). The mixture was heated to reflux over 1.5 hours. After cooling to 20° C., the mixture was evaporated under reduced pressure. To the residue were added propylene carbonate (400 ml) and 2-propyl iodide (607 g), and then the mixture was heated to 95° C. The reaction continued for 3 hours maintaining the temperature of 95-110° C. for 3 hours. Ethyl acetate (200 ml) was added to the mixture after the mixture was cooled to 30° C., and then the mixture was quenched by water (2000 ml) in one portion. The resulting mixture was stirred for 15 minutes at the ambient temperature then below 10° C. After stirring for 1 hour at 3-10° C., the precipitate was collected, washed with ethyl acetate (cooled, 300 ml) and dried under reduced pressure to give 6-hydroxy-2-isopropyl-3(2H)-pyridazinone as a yellowish solid (225.6 g).

¹H NMR(200 MHz, DMSO-d₆, δ): 1.24(6H,d,J=6.6 Hz), 4.98-5.12(1H,m), 6.87(1H,d,J=9.7 Hz), 7.03(1H,d,J=9.7 Hz)

Preparation 7

To a mixture of maleic hydrazide (10 g) and HMDS (21.6 g) in toluene (30 ml) as solvent was added dropwise sulfuric acid (0.88 g). The mixture was heated for one and a half hours at 100° C. After cooling, the mixture was evaporated under reduced pressure. To the residue were added propylene carbonate (20 ml) and methyl iodide (25.32 g), and then the mixture was refluxed for 2 hours. Ethyl acetate (40 ml) and water (100 ml) were added to the mixture after the mixture was cooled to room temperature. The resulting mixture was stirred for 30 minutes at the ambient temperature. The resulting precipitate was collected, washed with ethyl acetate (20 ml) and dried under reduced pressure to give 1-methyl-1,2-dihydro-3,6-pyridazinedione as a brown crystalline (9.18 g).

¹H NMR(200 MHz, DMSO-d₆, δ): 3.49(1H,s), 6.91 (1H, d, J=9.6 Hz), 7.08 (1H, d, J=9.7 Hz)

API-ES/MS: 127.3 [M+1]⁺

Preparation 8

To a mixture of maleic hydrazide (10 g) and HMDS (21.6 g) in toluene (30 ml) as solvent was added dropwise sulfuric acid (0.88 g). The mixture was heated for one and a half hours at 100° C. After cooling, the mixture was evaporated under reduced pressure. To the residue were added propylene carbonate (20 ml) and n-butyl iodide (32.83 g), and then the mixture was refluxed for 3 hours. Ethyl acetate (100 ml) and water (100 ml) were added to the mixture after the mixture was cooled to room temperature. The resulting mixture was stirred at the ambient temperature. The separated organic layer was added with n-heptane (100 ml) and the resulting mixture was stirred under cooling to 5° C. The resulting precipitate was collected and washed with a mixture of ethyl acetate (10 ml) and n-heptane (10 ml), then dried under reduced pressure to give 2-n-butyl-6-hydroxy-3(2H)-pyridazinone as a yellowish white crystalline (11.86 g).

¹H NMR(200 MHz, DMSO-d₆, δ): 0.89(3H,t, J=7.2 Hz), 1.19-1.37 (2H, m), 1.56-1.71 (2H, m), 3.86 (2H, t, J=7.3 Hz), 6.87 (1H, d, J=9.8 Hz), 7.03 (1H, d, J=13.9 Hz), 11.07 (1H, s) API/MS: 169.3 [M+1]⁺

Preparation 9

To a mixture of maleic hydrazide (10 g) and HMDS (21.6 g) in toluene (30 ml) as solvent was added dropwise sulfuric acid (0.88 g). The mixture was heated for one and a half hours at 100 CC. After cooling, the mixture was evaporated under reduced pressure. To the residue were added propylene carbonate (20 ml) and benzyl bromide (30.5 g), and then the mixture was refluxed for 2 hours. Water (100 ml) was added to the mixture after the mixture was cooled to room temperature, and then the mixture was cooled to 5° C. The resulting precipitate was collected, washed with a mixture of water (30 ml) and acetone (20 ml), then dried under reduced pressure to give 2-benzyl-6-hydroxy-3(2H)-pyridazinone as a yellowish white crystalline (17.64 g).

¹H NMR(200 MHz, DMSO-d₆, δ): 5.08 (2H, s), 6.96 (1H, d, J=9.8 Hz), 7.09 (1H, d, J=9.8 Hz), 11.18 (1H, s)

API-ES/MS: 203.2 [M+1]⁺

Preparation 10

To a mixture of maleic hydrazide (10 g) and HMDS (21.6 g) in toluene (30 ml) as solvent was added dropwise sulfuric acid (0.88 g). The mixture was heated for one and a half hours at 100° C. After cooling, the mixture was evaporated under reduced pressure. To the residue were added propylene carbonate (20 ml) and ethyl bromoacetate (29.80 g), and then the mixture was refluxed for 2 hours. Water (100 ml) was added to the mixture after the mixture was cooled to room temperature, and then the mixture was cooled to 5° C. The resulting precipitate was collected, washed with a mixture of water (30 ml) and acetone (20 ml), then dried under reduced pressure to give ethyl 3-hydroxy-6-oxo-[(6H)-pyridazinylacetate as a white crystalline (14.48 g).

¹H NMR(200 MHz, DMSO-d₆, δ): 1.20 (3H, t, J=7.2 Hz), 4.14 (2H, q, J=7.1 Hz), 4.64 (2H, s), 6.95 (1H, d, J=9.7 Hz), 7.13 (1H, d, J=9.9 Hz), 11.23 (1H, s)

API-ES/MS: 199.1 [M+1]⁺

Preparation 11

To a solution of 2-isopropyl-6-(2-oxo-2-phenylethyl)-3(2H)-pyridazinone (15.12 g) in acetic acid (90 mL) was added 30% hydrogen bromide solution in acetic acid (9 mL). Under ice-cooling, pyridinium tribromide (22.64 g) was added to the mixture. The mixture was stirred for 30 minutes at the same temperature and for 3 hours at ambient temperature. The mixture was poured into ice-water and extracted with chloroform. The organic layer was washed with water, aqueous sodium hydrogen carbonate solution and brine, dried over magnesium sulfate, and concentrated under reduced pressure to give a residue. The residue was purified by a column chromatography on silica gel (n-hexane:ethyl acetate=80:20 v/v) to give 6-(1-bromo-2-oxo-2-phenylethyl)-2-isopropyl-3(2H)-pyridazinone as a solid (16.27 g).

m.p.: 98-100° C. (diisopropyl ether-n-hexane)

IR (KBr): 1707, 1660, 1587 cm⁻¹

APCI/MS: 336 and 334(M+Na)⁺

¹H NMR (CDCl₃, δ): 1.19(3H, d, J=6.64 Hz), 1.34(3H, d, J=6.64 Hz), 5.27(1H, 7-plet, J=6.64 Hz), 6.25(1H, s), 6.95(1H, d, J=9.70 Hz), 7.26-7.69(4H, m), 8.05-8.10(2H, m)

Elemental Analysis for C₁₅H₁₅BrN₂O₂

Calcd. C: 53.73; H: 4.51; N: 8.36

Found C: 53.65; H: 4.53; N: 8.31

Preparation 12

A mixture of 6-(1-bromo-2-oxo-2-phenylethyl)-2-isopropyl-3(2H)-pyridazinone (11.93 g) and ethyl amino(thioxo)acetate (7.11 g) in ethanol (150 mL) was refluxed for 80 hours. After evaporation of ethanol, the mitxure was dissolved in chloroform and washed with water, an aqueous sodium hydrogen carbonate solution and brine. The organic layer was dried over magnesium sulfate and concentrated under reduced pressure to give a residue. The residue was purified by a column chromatography on silica gel (n-hexane:ethyl acetate=60:40, v/v) to give ethyl 5-(1-isopropyl-6-oxo-1,6-dihydro-3-pyridazinyl)-4-phenyl-1,3-thiazole-2-carboxylate as a solid (5.52 g).

m.p.: 153-154° C. (acetone-n-hexane)

IR (KBr): 1711, 1668, 1589 cm⁻¹

ESI/MS: 392(M+Na)⁺, 370(M+H)⁺

¹H NMR (CDCl₃, δ): 1.40(6H, d, J=6.64 Hz), 1.46(3H, t, J=7.12 Hz), 4.52(2H, q, J=6.64 Hz), 5.32(1H, 7-plet, J=6.64 Hz), 6.71(1H, d, J=9.70 Hz), 6.95(1H, d, J=9.70 Hz), 7.40-7.62(3H, m), 7.51-7.58(2H, m)

Elemental Analysis for C₁₉H₁₉N₃O₃S

Calcd. C: 61.77; H: 5.18; N: 11.37

Found C: 61.61; H: 5.16; N: 11.35

Preparation 13

Ethyl 5-(1-isopropyl-6-oxo-1,6-dihydro-3-pyridazinyl)-4-phenyl-1,3-thiazole-2-carboxylate was prepared as a solid (69.28 g), from 6-(1-chloro-2-oxo-2-phenylethyl)-2-isopropyl-3 (2H)-pyridazinone (90.0 g) and ethyl amino(thioxo)acetate (53.5 g) in a manner similar to

Preparation 12

m.p.: 153-154° C. (acetone-n-hexane)

IR (KBr): 1711, 1668, 1589 cm⁻¹

ESI/MS: 392(M+Na)⁺, 370(M+H)⁺

¹H NMR (CDCl₃, 3): 1.40(6H, d, J=6.64 Hz), 1.46(3H, t, J=7.12 Hz), 4.52(2H, q, J=6.64 Hz), 5.32(1H, 7-plet, J=6.64 Hz), 6.71(1H, d, J=9.70 Hz), 6.95(1H, d, J=9.70 Hz), 7.40-4762(3H, m), 7.51-7.58(2H, m)

Elemental Analysis for C₁₉H₁₉N₃O₃S

Calcd. C: 61.77; H: 5.18; N: 11.37

Found C: 61.61; H: 5.16; N: 11.35

Preparation 14

Ethyl 4-(4-fluorophenyl)-5-(1-isopropyl-6-oxo-1,6-dihydro-3-pyridazinyl)-1,3-thiazole-2-carboxylate wase obtained in a manner similar to Preparation 12.

m.p.: 193-194° C. (acetone-diisopropyl ether)

IR (KBr): 1689, 1649, 1585, 1535 cm⁻¹

APCI/MS: 797(2M+Na)⁺, 410(M+Na)⁺, 388(M+H)⁺,

¹H NMR (CDCl₃, δ): 1.39(6H, d, J=6.66 Hz), 1.46(3H, t, J=7.14 Hz), 4.52(2H, q, J=7.14 Hz), 5.33(1H, 7-plet, J=6.66 Hz), 6.75(1H, d, J=9.60 Hz), 6.96(1H, d, J=9.60 Hz), 7.08-7.19(2H, m), 7.51-7.59(2H, m)

Elemental Analysis for C₁₉H₁₈FN₃O₃S

Calcd. C: 58.90; H: 4.68; N: 10.85

Found C: 59.04; H: 4.68; N: 10.90

Preparation 15

Ethyl 4-(2-fluorophenyl)-5-(1-isopropyl-6-oxo-1,6-dihydro-3-pyridazinyl)-1,3-thiazole-2-carboxylate wase obtained in a manner similar to Preparation 12.

m.p.: 139.5-141° C. (acetone-n-hexane)

IR (KBr): 1712, 1668, 1589 cm⁻¹

ESI/MS: 797(2M+Na)⁺, 410(M+Na)⁺, 388(M+H)⁺

¹H NMR (CDCl₃, δ): 1.33(6H, d, J=6.66 Hz), 1.46(3H, t, J=7.12 Hz), 4.52(2H, q, J=7.12 Hz), 5.29(1H, 7-plet, J=6.66 Hz), 6.76(1H, d, J=9.58 Hz), 7.00(1H, d, J=9.58 Hz), 7.07-7.17(1H, m), 7.24-7.32(1H, m), 7.39-7.50(1H, m), 7.57-7.67(1H, m)

Elemental Analysis for C₁₉H₁₈FN₃O₃S

Calcd. C: 58.90; H: 4.68; N: 10.85

Found C: 59.15; H: 4.72; N: 10.78

Preparation 16

Ethyl 4-(3-fluorophenyl)-5-(1-isopropyl-6-oxo-1,6-dihydro-3-pyridazinyl)-1,3-thiazole-2-carboxylate wase obtained in a manner similar to Preparation 12.

m.p.: 154-155° C. (acetone-n-hexane)

IR (KBr): 1712, 1668, 1587 cm⁻¹

ESI/MS: 797(2M+Na)⁺, 410(M+Na)⁺, 388(M+H)⁺

¹H NMR (CDCl₃, δ): 1.39(6H, d, J=6.62 Hz), 1.47(3H, t, J=7.90 Hz), 4.52(2H, q, J=7.90 Hz), 5.33(1H, 7-plet, J=6.62 Hz), 6.76(1H, d, J=9.70 Hz), 6.99(1H, d, J=9.70 Hz), 7.09-7.19(1H, m), 7.26-7.42(3H, m)

Elemental Analysis for C₁₉H₁₈FN₃O₃S

Calcd. C: 58.90; H: 4.68; N: 10.85

Found C: 59.13; H: 4.72; N: 10.88

Preparation 17

Ethyl 4-(3-chlorophenyl)-5-(1-isopropyl-6-oxo-1,6-dihydro-3-pyridazinyl)-1,3-thiazole-2-carboxylate wase obtained in a manner similar to Preparation 12.

m.p.: 134-136° C. (acetone-n-hexane)

IR (KBr): 1728, 1668, 1591 cm⁻¹

ESI/MS: 831 and 829(2M+Na)⁺, 428 and 426(M+Na)⁺

¹H NMR (CDCl₃, δ): 1.39(6H, d, J=6.61 Hz), 1.47(3H, t, J=7.08 Hz), 4.53(2H, q, J=7.08 Hz), 5.33(1H, 7-plet, J=6.61 Hz), 6.77(1H, d, J=9.62 Hz), 7.00(1H, d, J=9.62 Hz), 7.30-7.46(3H, m), 7.61-7.63(1H, m)

Elemental Analysis for C₁₉H₁₈ClN₃O₃S

Calcd. C: 56.50; H: 4.49; N: 10.40

Found C: 56.59; H: 4.50; N: 10.48

Preparation 18

Ethyl 5-(6-oxo-1,6-diydro-3-pyridazinyl)-4-phenyl-1,3-thiazole-2-carboxylate wase obtained in a manner similar to Preparation 12.

m.p.: >250° C. (ethanol)

IR (KBr): 1711, 1678, 1657, 1583 cm⁻¹

ESI/MS: 350(M+Na)⁺, 328(M+H)⁺

¹H NMR (DMSO-d₆, δ): 1.35(3H, t, J=7.08 Hz), 4.42(2H, q, J=7.08 Hz), 6.84(1H, d, J=9.90 Hz), 7.06(1H, d, J=9.90 Hz), 7.46-7.59(5H, m), 13.44(1H, br.s)

Elemental Analysis for C₁₆H₁₃N₃O₃S.0.4H₂O

Calcd. C: 57.44; H: 4.16; N: 12.56

Found C: 57.25; H: 3.87; N: 12.52

Preparation 19

To a solution of 2-isopropyl-6-(2-oxo-2-phenylethyl)-3(2H)-pyridazinone (20.01 g) in dichloromethane (4.8 mL) was dropwise added sulfuryl chloride (6.59 mL) under reflux, and the mixture was refluxed for 30 minutes. The solution was poured into dichloromethane (40 mL). The resulting mixture was washed with water, an aqueous sodium hydrgencarbonate solution and brine, dried over magnesium sulfate and concentrated under reduced pressure to give a residue. The residue was purified by a column chromatography on silica gel (n-hexane:ethyl acetate=75:25, v/v) to give 6-(1-chloro-2-oxo-2-phenylethyl)-2-isopropyl-3(2H)-pyridazinone as a solid (21.38 g).

m.p.: 86.5-87.5° C. (n-hexane)

IR (KBr): 1711, 1660, 1589 cm⁻¹

ESI/MS: 603 and 605(2M+Na)⁺, 313 and 315(M+Na)⁺, 291 and 293(M+H)⁺

¹H NMR (CDCl₃, δ): 1.28(3H, d, J=6.63 Hz), 1.32(3H, d, J=6.63 Hz), 5.26(1H, 7-plet, J=6.63 Hz), 6.24(1H, s), 6.94(1H, d, J=9.66 Hz), 7.26-7.68(4H, m), 8.03-8.09(2H, m)

Elemental Analysis for C₁₅H₁₅CN₂O₂

Calcd. C: 61.97; H: 5.20; N: 9.63

Found C: 62.15; H: 5.17; N: 9.70

Preparation 20

In the presence of dichlorobis(triphenylphosphine)palladium(II) (1.47 g) and copper(I) iodide (1.47 g), triethylamine (14.67 mL) was added dropwise to a mixture of 1-isopropyl-6-oxo-1,6-dihydro-3-pyridazinyl trifluoromethanesulfonate (20.10 g) and ethynyl(trimethyl)silane (24.81 mL) in tetrahydrofuran (300 mL) under ice-cooling for 2 hour. The mixture was stirred for 3 hours at ambient temperature. The reaction mixture was poured into a mixture of water and ethyl acetate. An organic layer was washed with brine, dried over magnesium sulfate and concentrated under reduced pressure to give a residue. The residue was purified by a column chromatography on silica gel (n-hexane:ethyl acetate=90:10, v/v) to give 2-isopropyl-6-[(trimethylsilyl)ethynyl]-3(2H)-pyridazinone as a solid (16.10 g).

mp: 61-62.5° C. (n-hexane)

IR (KBr): 2160, 1664, 1587 cm⁻¹

ESI/MS: 491(2M+Na)⁺, 257(M+Na)⁺, 235(M+H)⁺

¹H NMR (CDCl₃, δ): 0.27(9H, s), 1.37(6H, d, J=6.64 Hz), 5.29(1H, 7-plet, J=6.64 Hz), 6.81(1H, d, J=9.54 Hz), 7.21(1H, d, J=9.54 Hz), 7.51-7.61(2H, m)

Elemental Analysis for C₁₂H₁₈N₂OSi

Calcd. C: 61.50; H: 7.74; N: 11.95

Found C: 61.25; H: 7.82; N: 12.00

Preparation 21

To a solution of 2-isopropyl-6-[(trimethylsilyl)ethynyl]-3(2H)-pyridazinone and benzyltriethyl-ammonium chloride (0.52 g) in a mixture of tetrahydrofuran (45 mL) and acetonitrile (45 mL) was added dropwise 12N aqueous sodium hydroxide solution (60 mL) under ice-cooling. After stirring for 30 minutes, the mixture was acidified with concentrated hydrochloric acid under ice-cooling. The mixture was extracted with chloroform, dried over magnesium sulfate and concentrated under reduced pressure to give a residue. The residue was purified by a column chromatography on silica gel (n-hexane:ethyl acetate=80:20, v/v) to give 6-ethynyl-2-isopropyl-3(2H)-pyridazinone as a solid (10.42 g).

mp: 103-104° C. (acetone-n-hexane)

IR (KBr): 3194, 2108, 1655, 1587 cm⁻¹

ESI/MS: 185(M+Na)⁺, 163(M+H)⁺

¹H NMR (CDCl₃, δ): 1.38(6H, d, J=6.64 Hz), 3.19(1H, s), 5.31(1H, 7-plet, J=6.64 Hz), 6.85(1H, d, J=9.52 Hz), 7.22(1H, d, J=9.52 Hz)

Elemental Analysis for C₉H₁₀N₂O

Calcd. C: 66.65; H: 6.21; N: 17.27

Found C: 66.92; H: 6.28; N: 17.36

Preparation 22

In the presence of dichlorobis(triphenylphosphine)palladium(II) (0.42 g) and copper(I) iodide (0.42 g), triethylamine (3.9 mL) was added dropwise to a mixture of 6-ethynyl-2-isopropyl-3(2H)-pyridazinone (3.25 g) and 1-fluoro-4-iodobenzene (6.67 g) in dioxane (60 mL) for 0.5 hour at 75-80° C. The mixture was stirred for 1.5 hours at 75-80° C. After cooling, a mixture of water and ethyl acetate was added to the reaction mixture. An organic layer was washed with brine, dried over magnesium sulfate and concentrated under reduced pressure to give a residue. The residue was purified by a column chromatography on silica gel (n-hexane:ethyl acetate=70:30, v/v) to give 6-[(4-fluorophenyl)-ethynyl]-2-isopropyl-3(2H)-pyridazinone as a solid (3.81 g).

mp: 105.5-106.5° C. (n-hexane)

IR (KBr): 2208, 1664, 1587 cm⁻¹

ESI/MS: 535(2M+Na)⁺, 279(M+Na)⁺, 257(M+H)⁺

¹H NMR (CDCl₃, δ): 1.40(6H, d, J=6.64 Hz), 5.33(1H, 7-plet, J=6.64 Hz), 6.87(1H, d, J=9.57 Hz), 7.01-7.14(2H, m), 7.28(1H, d, J=9.57 Hz), 7.51-7.61(2H, m)

Elemental Analysis for C₁₅H₁₃FN₂O

Calcd. C: 70.30; H: 5.11; N: 10.93

Found C: 70.33; H: 5.34; N: 11.05

Preparation 23

6-[(2-Fluorophenyl)-ethynyl]-2-isopropyl-3(2H)-pyridazinone was obtained in a manner similar to Preparation 22.

m.p.: 84.5-86° C. (diisopropyl ether-n-hexane)

IR (KBr): 2224, 1660, 1644, 1583 cm⁻¹

ESI/MS: 535(2M+Na)⁺, 279(M+Na)⁺, 257(M+H)⁺

¹H NMR (CDCl₃, δ): 1.41(6H, d, J=6.62 Hz), 5.34(1H, 7-plet, J=6.62 Hz), 6.88(1H, d, J=9.52 Hz), 7.12-7.20(2H, m), 7.32(1H, d, J=9.52 Hz), 7.33-7.41(1H, m), 7.52-7.60(1H, m)

Elemental Analysis for C₁₅H₁₃FN₂O

Calcd. C: 70.30; H: 5.11; N: 10.93

Found C: 70.38; H: 5.14; N: 10.95

Preparation 24

6-[(3-Fluorophenyl)-ethynyl]-2-isopropyl-3(2H)-pyridazinone was obtained in a manner similar to Preparation 22.

m.p.: 95.5-96.5° C. (acetone-n-hexane)

IR (KBr): 2220, 1660, 1606, 1585 cm⁻¹

ESI/MS: 535(2M+Na)⁺, 279(M+Na)⁺, 257(M+H)⁺

¹H NMR (CDCl₃, δ): 1.41(6H, d, J=6.62 Hz), 5.34(1H, 7-plet, J=6.62 Hz), 6.88(1H, d, J=9.52 Hz), 7.12-7.20(2H, m), 7.32(1H, d, J=9.52 Hz), 7.33-7.41(1H, m), 7.52-7.60(1H, m)

Elemental Analysis for C₁₅H₁₃FN₂O

Calcd. C: 70.30; H: 5.11; N: 10.93

Found C: 70.22; H: 5.16; N: 10.94

Preparation 25

In the presence of dichlorobis(triphenylphosphine)palladium(II) (0.42 g) and copper(I) iodide (0.42 g), triethylamine (3.9 mL) was added dropwise to a mixture of 1-isopropyl-6-oxo-1,6-dihydro-3-pyridazinyl trifluoromethanesulfonate (5.73 g) and 1-ethynyl-4-fluorobenzene (3.65 g) in dioxane (60 mL) for 0.5 hour at 75-80° C. The mixture was stirred for 1.5 hours at 75-80° C. After cooling, water and chloroform were added to the reaction mixture. An organic layer was washed with brine, dried over magnesium sulfate and concentrated under reduced pressure to give a residue. The residue was purified by a column chromatography on silica gel (n-hexane:ethyl acetate=70:30, v/v) to give 6-[(4-fluorophenyl)ethynyl]-2-isopropyl-3(2H)-pyridazinone as a solid (4.22 g).

mp: 105.5-106.5° C. (n-hexane)

IR (KBr): 2208, 1664, 1587 cm⁻¹

ESI/MS: 535(2M+Na)⁺, 279(M+Na)⁺, 257(M+H)⁺

¹H NMR (CDCl₃, δ): 1.40(6H, d, J=6.64 Hz), 5.33(1H, 7-plet, J=6.64 Hz), 6.87(1H, d, J=9.57 Hz), 7.01-7.14(2H, m), 7.28(1H, d, J=9.57 Hz), 7.51-7.61(2H, m)

Elemental Analysis for C₁₅H₁₃FN₂O

Calcd. C: 70.30; H: 5.11; N: 10.93

Found C: 70.33; H: 5.34; N: 11.05

Preparation 26

6-[(3-Chlorophenyl)-ethynyl]-2-isopropyl-3(2H)-pyridazinone was obtained in a manner similar to preparation 25.

m.p.: 94-95° C. (heptane)

IR (KBr): 1664, 1589 cm⁻¹

ESI/MS: 569 and 567(2M+Na)⁺, 297 and 295(M+Na)⁺, 275 and 273(M+H)⁺

¹H NMR (CDCl₃, δ): 1.40(6H, d, J=6.65 Hz), 5.33(1H, 7-plet, J=6.65 Hz), 6.88(1H,d, J=9.54 Hz), 7.25-7.48(4H, m), 7.55-7.58(1H, m)

Elemental Analysis for C₁₅H₁₃ClN₂O

Calcd. C: 66.06; H: 4.80; N: 10.27

Found C: 66.10; H: 4.83; N: 10.27

Preparation 27

To a mixture of sulfuric acid (6 mL) and acetic acid (15 mL) was added 6-[(4-fluorophenyl)-ethynyl]-2-isopropyl-3(2H)-pyridazinone (3.00 g), and the mixture was heated for 40 minutes at 100-105° C. The solution was poured into a mixuture of ice (90 g) and sodium carbonate (25.4 g). The resulting mixture was extracted with ethyl acetate (24 mL×2), dried over magnesium sulfate and concentrated under reduced pressure to give a residue. The residue was purified by a column chromatography on silica gel (n-hexane:ethyl acetate=30:70, v/v) to give 6-[2-(4-fluorophenyl)-2-oxoethyl]-2-isopropyl-3(2H)-pyridazinone as a solid (451 mg).

mp: 67-68° C. (n-hexane)

IR (KBr): 1689, 1660, 1596 cm⁻¹

APCI/MS: 275(M+H)⁺, 233

¹H NMR (CDCl₃, δ): 1.32(6H, d, J=6.62 Hz), 4.28(2H, s), 5.29(1H, 7-plet, J=6.62 Hz), 6.89(1H, d, J=9.50 Hz), 7.11-7.23(3H, m), 8.04-8.13(2H, m)

Elemental Analysis for C₁₅H₁₅FN₂O₂

Calcd. C: 65.68; H: 5.51; N: 10.21

Found C: 65.72; H: 5.65; N: 10.21

Preparation 28

6-[2-(2-Fluorophenyl)-2-oxoethyl]-2-isopropyl-3(2H)-pyridazinone was obtained in a manner similar to Preparation 27.

IR (Neat): 1685, 1664, 1593 cm⁻¹

ESI/MS: 571(2M+Na)⁺, 297(M+Na)⁺, 275(M+H)⁺

¹H NMR (CDCl₃, δ): 1.32(6H, d, J=6.65 Hz), 4.28(2H, s), 5.29(1H, 7-plet, J=6.65 Hz), 6.89(1H, d, J=9.50 Hz), 7.17(1H, d, J=9.50 Hz), 7.40-7.49(1H, m), 7.55-7.62(1H, m), 7.89-7.95(1H, m), 8.02-8.04(1H, m)

Preparation 29

6-[2-(3-Fluorophenyl)-2-oxoethyl]-2-isopropyl-3(2H)-pyridazinone was obtained in a manner similar to Preparation 27.

m.p.: 80-81° C. (diisopropyl ether-n-hexane)

IR (KBr): 1680, 1658, 1591 cm⁻¹

ESI/MS: 274(2M+Na)⁺, 297(M+Na)⁺, 275(M+H)⁺

¹H NMR (CDCl₃, δ): 1.32(6H, d, J=6.60 Hz), 4.29(2H, s), 5.29(1H, 7-plet, J=6.60 Hz), 6.89(1H, d, J=9.48 Hz), 7.18(1H, d, J=9.48 Hz), 7.26-7.33(1H, m), 7.43-7.53(1H, m), 7.70-7.77(1H, m), 7.80-7.86(1H, m)

Elemental Analysis for C₁₅H₁₅FN₂O₂

Calcd. C: 65.68; H: 5.51; N: 10.21

Found C: 65.73; H: 5.61; N: 10.24

Preparation 30

6-[2-(3-Chlorophenyl)-2-oxoethyl]-2-isopropyl-3(2H)-pyridazinone was obtained in a manner similar to Preparation 27.

m.p.: 85-86° C. (diisopropyl ether-n-hexane)

IR (KBr): 1676, 1658, 1591 cm⁻¹

ESI/MS: 605 and 603(2M+Na)⁺, 315 and 313(M+Na)⁺, 293 and 291 (M+H)⁺

¹H NMR (CDCl₃, d): 1.32(6H, d, J=6.65 Hz), 4.28(2H, s), 5.29(1H, 7-plet, J=6.65 Hz), 6.89(1H, d, J=9.50 Hz), 7.17(1H, d, J=9.50 Hz), 7.40-7.49(1H, m), 7.55-7.62(1H, m), 7.89-7.95(1H, m), 8.02-8.04(1H, m)

Elemental Analysis for C₁₅H is ClN₂O₂

Calcd. C: 61.97; H: 5.20; N: 9.63

Found C: 62.10; H: 5.25; N: 9.68

Preparation 31

To a solution of 6-[2-(4-fluorophenyl)-2-oxoethyl]-2-isopropyl-3(2H)-pyridazinone (2.40 g) in dichloromethane (4.8 mL) was dropwise added a solution of sulfuryl chloride (1.24 g) in dichloromethane (0.8 mL) under reflux, and the mixture was refluxed for 30 minutes. The solution was poured into dichloromethane (40 mL). The mixture was washed with water, an aqueous sodium hydrgencarbonate solution and brine, dried over magnesium sulfate and concentrated under reduced pressure to give a residue. The residue was purified by a column chromatography on silica gel (n-hexane:ethyl acetate=80:20, v/v) to give 6-[1-chloro-2-(4-fluorophenyl)-2-oxoethyl]-2-isopropyl-3(2H)-pyridazinone as a solid (2.17 g).

mp: 86.5-88° C. (n-hexane)

IR (KBr): 1709, 1658, 1592 cm⁻¹

ESI/MS: 641 and 639(2M+Na)⁺, 333 and 331(M+Na)⁺

¹H NMR (CDCl₃, δ).: 1.28(3H, d, J=6.60 Hz), 1.37(3H, d, J=6.60 Hz), 5.26(1H, 7-plet, J=6.60 Hz), 6.17(1H, s), 6.94(1H, d, J=9.70 Hz), 6.96-7.27(2H, m), 7.48(1H, d, J=9.70 Hz), 8.05-8.15(2H, m)

Elemental Analysis for C₁₅H₁₄ClFN₂O₂

Calcd. C: 58.36; H: 4.57; N: 9.07

Found C: 58.54; H: 4.59; N: 9.07

Preparation 32

6-(1-Chloro-2-(2-fluorophenyl)-2-oxoethyl]-2-isopropyl-3(2H)-pyridazinone was obtained in a manner similar to Preparation 31.

IR (Neat): 1666, 1595 cm⁻¹

ESI/MS: 641 and 639(2M+Na)⁺, 333 and 331(M+Na)⁺

¹H NMR (CDCl₃, δ): 1.14(3H, d, J=6.62 Hz), 1.23(3H, d, J=6.62 Hz), 5.19(1H, 7-plet, J=6.62 Hz), 6.19(1H, s), 6.94(1H, d, J=9.60 Hz), 7.09-7.20(1H, m), 7.25-7.34(1H, m), 7.43(1H, d, J=9.60 Hz), 7.52-7.75(1H, m), 7.92-7.82(1H, m)

Elemental Analysis for C₁₅H₁₄ClFN₂O₂

Calcd. C: 58.36; H: 4.57; N: 9.07

Found C: 58.09; H: 4.68; N: 9.01

Preparation 33

6-[1-Chloro-2-(3-fluorophenyl)-2-oxoethyl]-2-isopropyl-3(2H)-pyridazinone was obtained in a manner similar to Preparation 31.

m.p.: 65.5-66.5° C. (n-hexane)

IR (KBr): 1714, 1664, 1589 cm⁻¹

ESI/MS: 641 and 639(2M+Na)⁺, 333 and 331(M+Na)⁺

¹H NMR (CDCl₃, δ): 1.27(3H, d, J=6.68 Hz), 1.32(3H, d, J=6.68 Hz), 5.26(1H, 7-plet, J=6.68 Hz), 6.18(1H, s), 6.95(1H, d, J=9.68 Hz), 7.27-7.52(3H, m), 7.72-7.88(2H, m)

Elemental Analysis for C₁₅H₁₄ClFN₂O₂

Calcd. C: 58.36; H: 4.57; N: 9.07

Found C: 58.44; H: 4.42; N: 9.09

Preparation 34

6-[1-Chloro-2-(3-chlorophenyl)-2-oxoethyl]-2-isopropyl-3(2H)-pyridazinone was obtained in a manner similar to Preparation 31.

IR (Neat): 1697, 1670, 1593 cm⁻¹

ESI/MS: 673 and 671(2M+Na)⁺, 349 and 347(M+Na)⁺

¹H NMR (CDCl₃, δ): 1.30(3H, d, J=6.64 Hz), 1.33(3H, d, J=6.64 Hz), 5.26(1H, 7-plet, J=6.64 Hz), 6.19(1H, s), 6.95(1H, d, J=9.70 Hz), 7.41-7.50(2H, m), 7.57-7.63(1H, m), 7.91-7.97(1H, m), 8.03-8.06(1H, m)

Elemental Analysis for C₁₅H₁₄Cl₂N₂O₂

Calcd. C: 55.40; H: 4.34; N: 8.61

Found C: 55.47; H: 4.53; N: 8.48

Preparation 35

Trifluoromethanesulfonic anhydride (3.55 mL) was added dropwise to a solution of 3,6-dihydroxypyridazine (2.25 g) in pyridine (50 mL) under ice-cooling. The mixture was stirred for one hour under ice-cooling and for 2 hours at ambient temperature. After addition of methanol (1 mL) under ice-cooling, pyridine was evaporated under reduced pressure to give a syrup. The syrup was dissolved in ethyl acetate. The mixture was washed with water, 1N-hydrochloric acid, an aqueous sodium hydrogencarbonate solution and brine. The mixture was dried over magnesium sulfate and concentrated under reduced pressure to give a residue. The residue was purified by a column chromatography on silica gel (n-hexane:ethyl acetate=60:40 and 40:60, v/v) to give 6-oxo-1,6-dihydro-3-pyridazinyl trifluoromethane-sulfonate as a solid (4.10 g).

m.p.: 130-131.5° C. (acetone-n-hexane)

IR (KBr): 3080, 2985, 2881, 1703, 1641, 1597 cm⁻¹

ESI/MS: 243(M−H)⁻

¹H NMR (DMSO-d₆, δ): 7.18(1H, d, J=10.05 Hz), 7.76(1H, d, 10.05 Hz), 13.27(1H, s)

Elemental Analysis for C₅H₃F₃N₂O₄S

Calcd. C: 24.60; H: 1.24; N: 11.47

Found C: 24.63; H: 1.16; N: 11.43

Preparation 36

Under nitrogen atmosphere, bis(trimethylsilyl)acetamide (5.0 mL) was added to a suspension of 6-oxo-1,6-dihydro-3-pyridazinyl trifluoromethanesulfonate (5.00 g) in tetrahydrofuran (10 mL), and the mixture was stirred at ambient temperature for 15 minutes. To the mixture were added ethynylbenzene (2.30 g), dichlorobis(triphenylphosphine)palladium(II) (72 mg) and coppers) iodide (20 mg). A solution of triethylamine (3.14 mL) in tetrahydrofuran (2.5 mL) was added dropwise to the mixture under reflux. The reaction mixture was refluxed for one hour. After cooling, the mixture was poured into water (100 mL) to afford a solid. The solid was collected by filtration, dried over phosphorous petoxide under reduced pressure and recrystallized from a mixture of methanol and diisopropyl ether to give 6-(phenylethynyl)-3(2H)-pyridazinone as a solid (2.48 g).

m.p.: 190-192° C. (methanol-diisopropyl ether)

IR (KBr): 2222, 1664, 1647 cm⁻¹

ESI/MS: 415(2M+Na)⁺, 219(M+Na)⁺, 197(M+H)⁺

¹H NMR (DMSO-d₆, δ): 6.94(1H, d, J=8.64 Hz), 7.42-7.50(3H, m), 7.55-7.63(3H, m), 13.36(1H, br.s)

Elemental Analysis for C₁₂H₈N₂O

Calcd. C: 73.46; H: 4.11; N: 14.28

Found C: 73.33; H: 4.10; N: 14.13

Preparation 37

To a mixture of sulfuric acid (11.0 mL) and acetic acid (27.5 mL) was added 6-(phenylethynyl)-3(2H)-pyridazinone (5.50 g), and the mixture was heated for 30 minutes at 100-105° C. The solution was poured into a mixuture of ice (37.3 g) and sodium carbonate (165 g) and warmed at 30° C. to obtain a solid. The solid was collected by filtration, dried over phosphorous pentoxide and purified by a column chromatography on silica gel (methanol:chloroform ==2:98, v/v) to give 6-(2-oxo-2-phenylethyl)-3(2H)-pyridazinone as a solid (3.86 g).

m.p.: 178-179° C. (chloroform-n-hexane)

IR (KBr): 1678, 1660, 1603 cm⁻¹

ESI/MS: 451(2M+Na)⁺, 237(M+Na)⁺, 215(M+H)⁺

¹H NMR (CDCl₃, δ): 4.30(2H, s), 6.95(1H, d, J=9.76 Hz), 7.29(1H, d, J=9.76 Hz), 7.49-7.54(2H, m), 7.60-7.65(1H, m), 7.97-8.06(2H, m), 10.52(1H, br.s)

¹H NMR (DMSO-d₆, δ): 4.43(2H, s), 6.86(1H, d, J=9.75 Hz), 7.38(1H, d, J=9.75 Hz), 7.51-7.60(2H, m), 7.64-7.73(1H, m), 8.00-8.05(2H, m)

Elemental Analysis for C₁₂H₁₀N₂O₂

Calcd. C: 67.28; H: 4.70; N: 13.08

Found C: 67.36; H: 4.69; N: 13.23

Preparation 38

To a solution of 6-(2-oxo-2-phenylethyl)-3(2H)-pyridazinone (1.00 g) in acetic acid (9 raL) was added 30% hydrogen bromide solution in acetic acid (1 mL). Under ice-cooling, pyridinium tribromide (1.79 g) was added to the mixture. The mixture was stirred for 30 minutes at the same temperature and for 20 hours at ambient temperature to obtain a solid. The solid was collected by filtration and dissolved in chloroform (30 mL). The mixture was washed with an aqueous sodium hydrogencarbonate solution, dried over magnesium sulfate and concentrated under reduced pressure to give a residue. The residue was recrystallized from a mixture of acetone and diisopropyl ether to give 6-(1-bromo-2-oxo-2-phenylethyl)-3(2H)-pyridazinone as a solid (1.01 g).

m.p.: 140-141.5° C. (acetone-diisopropyl ether)

IR (KBr): 1682, 1666, 1595 cm⁻¹

ESI/MS: 315 and 317(M+Na)⁺

¹H NMR (CDCl₃, δ): 6.21(1H, s), 7.03(1H, d, J=9.94 Hz), 7.48-7.66(3H, m), 7.78(1H, d, J=9.94 Hz), 8.02-8.08(2H, m), 11.81(1H, br.s)

¹H NMR (DMSO-d₆, δ): 6.98(1H, d, J=10.08 Hz), 7.03(1H, s), 7.51-7.77(4H, m), 8.02-8.07(2H, m), 13.14(1H, br.s)

Elemental Analysis for C₁₂H₉BrN₂O₂

Calcd. C: 49.17; H: 3.09; N: 9.56

Found C: 49.53; H: 3.08; N: 9.64

Preparation 39

To a solution of 6-(phenylethynyl)-3(2H)-pyridazinone (100 mg) in dimethylformamide (0.5 mL) was added sodium hydride (60% in oil) (21 mg), and the mixture was stirred for 30 minutes at 50-55° C. 2-Iodopropane (0.056 mL) was added to the mixture, and the mixture was stirred for 3 hours at 50-55° C. The reaction mixture was diluted with ethyl acetate. The mixture was washed with water and brine, dried over magnesium sulfate and concetrated under reduced pressure to obtain a residue. The residue was purified by a preparative TLC on silica gel (n-hexane:ethyl acetate=60:40 v/v) to give 2-isopropyl-6-(phenylethynyl)-3(2H)-pyridazinone as a solid (93 mg).

mp: 75.5-77° C. (heptane)

IR (KBr): 2218, 1669, 1583 cm⁻¹

APCI/MS: 239(M+H)⁺, 197

¹H NMR (CDCl₃, δ): 1.40(6H, d, J=6.65 Hz), 5.33(1H, 7-plet, J=6.65 Hz), 6.87(1H, d, J=9.57 Hz), 7.26-7.42(4H, m), 7.52-7.60(2H, m)

Elemental Analysis for C₁₅H₁₄N₂O

Calcd. C: 75.61; H: 5.92; N: 11.76

Found C: 75.79; H: 5.88; N: 11.74

Preparation 40

To a solution of ethyl 5-(6-oxo-1,6-dihydro-3-pyridazinyl)-4-phenyl-1,3-thiazole-2-carboxylate (1.64 g) in dimethylformamide (5 mL) was added sodium hydride (60% in oil) (210 mg), and the mixture was stirred at 50-55° C. for 30 minutes. Iodomethane (0.374 mL) was added to the mixture, and the mixture was stirred for 20 hours at ambient temperature. The mixture was poured into water (20 mL) to give a solid. The solid was collected by filtration, dried over phosphorous pentoxide and purified by a column chromatography on silica gel (n-hexane:ethyl acetate=50:50, v/v) to give ethyl 5-(1-methyl-6-oxo-1,6-dihydro-3-pyridazinyl)-4-phenyl-1,3-thiazole-2-carboxylate as a solid (1.56 g).

m.p.: 157.5-159° C. (chloroform-diisopropyl ether)

IR (KBr): 1707, 1668 cm⁻¹

ESI/MS: 705(2M+Na)⁺, 364(M+Na)⁺, 342(M+H)⁺

¹H NMR (CDCl₃, δ): 1.46(3H, t, J=7.12 Hz), 3.85(3H, s), 4.52(2H, q, J=7.12 Hz), 6.73(1H, d, J=9.72 Hz), 6.96(1H, d, J=9.72 Hz), 7.41-7.45(3H, m), 7.53-7.57(2H, m)

Elemental Analysis for Cl₇H is N₃O₃S

Calcd. C: 59.81; H: 4.43; N: 12.31

Found C: 59.72; H: 4.35; N: 12.28

Preparation 41

To a solution of ethyl 5-(6-oxo-1,6-dihydro-3-pyridazinyl)-4-phenyl-1,3-thiazole-2-carboxylate (1.64 g) in dimethylformamide (5 mL) was added sodium hydride (60% in oil) (210 mg), and the mixture was stirred for 30 minutes at 50-55° C. Iodoethane (0.481 mL) was added to the mixture, and the mixture was stirred for 3 hours at 50-55° C. The mixture was poured into water (20 mL) to obtain a solid. The solid was collected by filtration, dried over phosphorous pentoxide and purified by a column chromatography on silica gel (n-hexane:ethyl acetate=70:30, v/v) to give ethyl 5-(1-ethyl-6-oxo-1,6-dihydro-3-pyridazinyl)-4-phenyl-1,3-thiazole-2-carboxylate as a solid (1.62 g).

m.p.: 144-146° C. (chloroform-diisopropyl ether)

IR (KBr): 1707, 1666 cm⁻¹

ESI/MS: 733(2M+Na)⁺, 378(M+Na)⁺, 356(M+H)⁺

¹H NMR (CDCl₃, δ): 1.44(3H, t, J=7.20 Hz), 1.46(3H, t, J=7.12 Hz), 4.26(2H, q, J=7.20 Hz), 4.52(2H, q, J=7.12 Hz), 6.73(1H, d, J=9.72 Hz), 6.96(1H, d, J=9.72 Hz), 7.41-7.45(3H, m), 7.54-7.57(2H, m)

Elemental Analysis for C₁₈H₁₇N₃O₃S

Calcd. C: 60.83; H: 4.82; N: 11.82

Found C: 60.91; H: 4.73; N: 11.89

Preparation 42

Ethyl 5-(6-oxo-1-propyl-1,6-dihydro-3-pyridazinyl)-4-phenyl-1,3-thiazole-2-carboxylate was obtained in a manner similar to Preparation 41.

m.p.: 124.5-126° C. (chloroform-diisopropyl ether)

IR (KBr): 1709, 1664 cm⁻¹

ESI/MS: 761(2M+Na)⁺, 392(M+Na)⁺

¹H NMR (CDCl₃, δ): 1.03(3H, t, J=7.20 Hz), 1.46(3H, t, J=7.12 Hz), 1.84-1.92(2H, m), 4.17(2H, t, J=7.20 Hz), 4.51(2H, q, J=7.12 Hz), 6.73(1H, d, J=9.72 Hz), 6.95(1H, d, J=9.72 Hz), 7.41-7.45(3H, m), 7.53-7.57(2H, m)

Elemental Analysis for C₁₆H₁₃N₃O₃S.0.5H₂O

Calcd. C: 60.30; H: 5.33; N: 11.10

Found C: 60.29; H: 5.03; N: 11.07

Preparation 43

Ethyl 5-(1-allyl-6-oxo-1,6-dihydro-3-pyridazinyl)-4-phenyl-1,3-thiazole-2-carboxylate was obtained in a manner similar to Preparation 41.

m.p.: 94-95° C. (chloroform-diisopropyl ether)

IR (KBr): 1711, 1668 cm⁻¹

ESI/MS: 757(2M+Na)⁺, 390(M+Na)⁺, 368(M+H)⁺

¹H NMR (CDCl₃, δ): 1.46(3H, t, J=7.12 Hz), 4.51(2H, q, J=7.12 Hz), 4.79-4.82(2H, m), 5.29-5.36(2H, m), 6.00-6.11(1H, m), 6.74(1H, d, J=9.72 Hz), 6.96(1H, d, J=9.72 Hz), 7.42-7.45(3H, m), 7.53-7.57(2H, m)

Elemental Analysis for C₁₉H₁₇N₃O₃S.0.2H₂O

Calcd. C: 61.51; H: 4.73; N: 11.33

Found C: 61.31; H: 4.51; N: 11.20

Preparation 44

Ethyl 5-(1-benzyl-6-oxo-1,6-dihydro-3-pyridazinyl)-4-phenyl-1,3-thiazole-2-carboxylate was obtained in a manner similar to Preparation 41.

m.p.: 137-139° C. (chloroform-diisopropyl ether)

IR (KBr): 1712, 1670 cm⁻¹

ESI/MS: 857(2M+Na)⁺, 440(M+Na)⁺, 418(M+H)⁺

¹H NMR (CDCl₃, δ): 1.46(3H, t, J=7.12 Hz), 4.52(2H, q, J=7.12 Hz), 5.35(2H, s), 6.72(1H, d, J=9.72 Hz), 6.93(1H, d, J=9.72 Hz), 7.26-7.43(3H, m), 7.48-7.55(2H, m)

Elemental Analysis for C₂₃H₁₉N₃O₃S.0.2H₂O

Calcd. C: 65.61; H: 4.64; N: 9.98

Found C: 65.64; H: 4.56; N: 9.80

Preparation 45

Ethyl 5-[1-(2-methoxyethyl)-6-oxo-1,6-dihydro-3-pyridazinyl]-4-phenyl-1,3-thiazole-2-carboxylate was obtained in a manner similar to Preparation 41.

m.p.: 111-112° C. (chloroform-diisopropyl ether)

IR (KBr): 1739, 1674 cm⁻¹

ESI/MS: 793(2M+Na)⁺, 408(M+Na)⁺, 386(M+H)⁺

¹H NMR (CDCl₃, δ): 1.46(3H, t, J=7.12 Hz), 3.40(3H, s), 3.84(2H, t, J=5.58 Hz), 4.41(2H, t, J=5.58 Hz), 4.52(2H, q, J=7.12 Hz), 6.73(1H, d, J=9.76 Hz), 6.96(1H, d, J=9.76 Hz), 7.42-7.45(3H, m), 7.54-7.57(2H, m)

Elemental Analysis for C₁₉H₁₉N₃O₄S

Calcd. C: 59.21; H: 4.97; N: 10.90

Found C: 59.25; H: 4.93; N: 10.91

Preparation 46

Under ice-cooling, trifluoroacetic anhydride (0.163 mL) was added dropwise to a mixture of 5-(1-isopropyl-6-oxo-1,6-dihydro-3-pyridazinyl)-4-phenyl-1,3-thiazole-2-carboxamide (342 mg) and pyridine (0.163 mL) in dioxane (2 mL). The mixture was stirred for one hour at the same temperature and for 2 hours at ambient temperature. Water was added to the mixture to give a solid. The solid collected by filtration was dissolved in chloroform, dried over magnesium sulfate and concentrated under reduced pressure to give a residue. The residue was crysatallized from a mixture of acetone and n-hexane to give 5-(1-isopropyl-6-oxo-1,6-dihydro-3-pyridazinyl)-4-phenyl-1,3-thiazole-2-carbonitrile as a solid (271 mg).

m.p.: 135-136° C. (acetone-n-hexane)

IR (KBr): 2229, 1670, 1589 cm⁻¹

ESI/MS: 345(M+Na)⁺

¹H NMR (CDCl₃, δ): 1.40(6H, d, J=6.60 Hz), 5.32(1H, 7-plet, J=6.60 Hz), 6.74(1H, d, J=9.62 Hz), 6.97(1H, d, J=9.62 Hz), 7.43-7.57(5H, m)

Elemental Analysis for C₁₇H₁₄N₄OS

Calcd. C: 63.34; H: 4.38; N: 17.38

Found C: 63.23; H: 4.34; N: 17.26

### EXAMPLE 1

A mixture of 6-(1-bromo-2-oxo-2-phenylethyl)-2-isopropyl-3(2H)-pyridazinone (140 mg) and thiourea (48 mg) in ethanol (1.5 ml) was refluxed for 60 hours. The mixture was poured into a mixture of chloroform (5 ml), a saturated sodium hydrogencarbonate solution (0.5 ml) and water (0.5 ml). The organic solution was washed with brine, dried over magnesium sulfate and concentrated under reduced pressure to give a residue. The residue was purified by a column chromatography on silica gel (n-hexane:ethyl acetate=1:4, v/v) to give 6-(2-amino-4-phenyl-1,3-thiazol-5-yl)-2-isopropyl-3(2H)-pyridazinone as a solid (97 mg).

mp: >250° C.

IR(KBr): 1641, 1583, 1525 cm⁻¹

¹H NMR(CDCl₃, δ): 1.36(6H,d,J=6.62 Hz), 5.17(2H,br.s), 5.29(1H, 7-plet,J=6.62 Hz), 6.61(1H,d,J=9.70 Hz), 6.88(1H,d,J=9.70 Hz), 7.26-7.43(3H,m), 7.45-7.53(2H,m)

APCI/MS: 345[M+Na]⁺, 313[M+H]⁺, 282, 257

Elemental Analysis for C₁₆H₁₆N₄OS

Calcd.: C, 60.47; H, 5.26; N, 17.63

Found: C, 60.45; H, 5.05; N, 17.58

### EXAMPLE 2

A mixture of 6-(2-amino-4-phenyl-1,3-thiazol-5-yl)-2-isopropyl-3(2H)-pyridazinone (150 mg), benzoyl chloride (81 mg) and triethylamine (63.2 mg) in dimethylformamide (3 ml) was stirred overnight at ambient temperature. After 1N-hydrochloric acid was poured into the reaction mixture, the resulting mixture was extracted with ethyl acetate. The organic layer was washed with an aqueous sodium hydrogencarbonate solution and dried over magnesium sulfate. The solvent was removed in vacuo to give an oil, which was subjected to a column chromatography on silica gel eluting with a mixture of chloroform and methanol. The solvent was removed in vacuo to afford an oil, which was suspended in diisopropyl ether with stirring. The powder was collected by filtration to afford N-[5-(1-isopropyl-6-oxo-1,6-dihydro-3-pyridazinyl)-4-phenyl-1,3-thiazol-2-yl]-benzamide (30 mg).

mp: 126-129° C.

IR(KBr): 3432, 1660, 1583 cm⁻¹

¹H NMR (DMSO-d₆, δ): 1.30(6H,d,J=6.6 Hz), 5.15(1H, 7-plet,J=6.6 Hzt, 6.81(1H,d,J=9.7 Hz), 7.04(1H,d,J=9.7 Hz), 7.35-7.7(8H,m), 8.1-8.2(2H,m), 12.96(1H,brs)

APCI/MS: 417[M+H]⁺, 439[M+Na]⁺

Elemental Analysis for C₂₋₃H₂₀N₄O₂S.0.8H₂O

Calcd.: C, 64.11; H, 5.05; N, 13.00

Found: C, 64.32; H, 5.01; N, 12.59

### EXAMPLE 3

N-[5-(1-Isopropyl-6-oxo-1,6-dihydro-3-pynidazinyl)-4-phenyl-1,3-thiazol-2-yl]hexanamide was obtained in a manner similar to Example 2.

mp: 129-132° C.

IR(KBr): 3432, 1660, 1583 cm⁻¹

¹H NMR(DMSO-d₆, δ): 0.8-0.95(3H,m), 1.15-1.4(10H,m), 1.5-1.75(2H,m), 2.4-2.6(2H,m), 5.14(1H, 7-plet,J=6.6 Hz), 6.80(1H, d,J=9.7 Hz), 7.01 (1H,d,J=9.7 Hz), 7.35-7.6(5H,m), 12.39(1H,brs)

APCI/MS: 411[M+H]⁺, 433[M+Na]⁺

### EXAMPLE 4

N-[5-(1-Isopropyl-6-oxo-1,6-dihydro-3-pyridazinyl)-4-phenyl-1,3-thiazol-2-yl]-2-phenylacetamide was obtained in a manner similar to Example 2.

mp: 250-252° C.

IR(KBr): 3166, 1650, 1583 cm⁻¹

¹H NMR(DMSO-d₆, δ): 1.25(6H,d,J=6.6 Hz), 3.81(2H,s), 5.12(1H, 7-plet,J=6.6 Hz), 6.80(1H,d,J=9.7 Hz), 7.00(1H,d,J=9.7 Hz), 7.2-7.6(10H,m), 12.68(1H,brs)

APCI/MS: 431[M+H]⁺, 453[M+NaJ+

Elemental.Analysis for C₂₄H₂₂N₄O₂S 0.2H₂O

Calcd.: C, 66.40; H, 5.20; N, 12.91

Found: C, 66.77; H, 5.28; N, 12.55

### EXAMPLE 5

N-[5-(1-Isopropyl-6-oxo-1,6-dihydro-3-pyridazinyl)-4-phenyl-1,3-thiazol-2-yl]-2,2-dimethylpropanamide was obtained in a manner similar to Example 2.

mp: 224-226° C.

IR(KBr): 3230, 1654, 1585 cm⁻¹

¹H NMR(DMSO-d₆, δ): 1.2-1.3(15H,m), 5.13(1H, 7-plet,J=6.6 Hz), 6.79(1H,d,J=9.7 Hz), 6.99(1H,d,J=9.7 Hz), 7.35-7.6(5H,m), 12.11(1H,brs)

ESI/MS: 397[M+H]⁺, 419[M+Na]⁺

Elemental Analysis for C₂₁H₂₄N₄O₂S

Calcd.: C, 63.61; H, 6.10; N, 14.13

Found: C, 63.31; H, 6.14; N, 13.90

### EXAMPLE 6

A mixture of 6-(2-amino-4-phenyl-1,3-thiazol-5-yl)-2-isopropyl-3(2H)-pyridazinone (200 mg), acetyl chloride (60.3 mg) and triethylamine (97.2 mg) in dimethylformamide (2 ml) was stirred overnight at ambient temperature. After 1N-hydrochloric acid was poured into the reaction mixture, the resulting mixture was extracted with ethyl acetate. The organic layer was washed with an aqueous sodium hydrogencarbonate solution, and dried over magnesium sulfate. The solvent was removed in vacuo to give an oil, which was subjected to a column chromatography on silica gel eluting with a mixture of chloroform and methanol. The solvent was removed in vacuo to afford an oil, which was suspended in diisopropyl ether with stirring. The powder was collected by filtration to afford N-[5-(1-isopropyl-6-oxo-1,6-dihydro-3-pyridazinyl)-4-phenyl-1,3-thiazol-2-yl]-acetamide (30 mg).

mp: 202-204° C.

IR(KBr): 3432, 1648, 1579 cm⁻¹

¹H NMR(DMSO-dr,3): 1.26(6H,d,J=6.6 Hz), 2.19(3H,s), 5.13(1H, 7-plet,J=6.6 Hz), 6.80(1H,d,J=9.7 Hz), 7.02(1H,d,J=9.7 Hz), 7.3-7.6(5H,m)

ESI/MS: 355[M+H]⁺, 377[M+Na]⁺

Elemental Analysis for C₁₈H₁₈N₄O₂S

Calcd.: C, 61.00; H, 5.12; N, 15.81

Found: C, 61.03; H, 5.12; N, 15.84

### EXAMPLE 7

N-[5-(1-Isopropyl-6-oxo-1,6-dihydro-3-pyridazinyl)-4-phenyl-1,3-thiazol-2-yl]cyclohexanecarboxamide was obtained in a manner similar to Example 2.

mp: 234-236° C.

IR(KBr): 3178, 1646, 1579 cm⁻¹

¹H NMR(DMSO-d₆, δ): 1.1-1.55(11H,m), 1.55-1.9(5H,m), 5.13(1H, 7-plet,J=6.6 Hz), 6.80(1H,d,J=9.7 Hz), 7.00(1H,d,J=9.7 Hz), 7.3-7.6(5H,m), 12.33(1H,brs)

ESI/MS: 423[M+H]⁺, 445[M+Na]⁺

Elemental Analysis for C₂₃H₂₆N₄O₂S.0.1H₂O

Calcd.: C, 65.10; H, 6.22; N, 13.20

Found: C, 65.26; H, 6.42; N, 12.85

### EXAMPLE 8

N-[5-(1-Isopropyl-6-oxo-1,6-dihydro-3-pyridazinyl)-4-phenyl-1,3-thiazol-2-yl]-2-phenoxyacetamide was obtained in a manner similar to Example 2.

mp: 243-244° C.

IR(KBr): 3399, 1697, 1666, 1589 cm⁻¹

¹H NMR(DMSO-d₆, δ): 1.26(6H,d,J=6.6 Hz), 4.90(2H,s), 5.13(1H, 7-plet,J=6.6 Hz), 6.80(1H,d,J=9.7 Hz), 6.9-7.1(4H,m), 7.2-7.6(7H,m), 12.70(1H,brs)

ESI/MS: 447[M+H]⁺, 469[M+Na]⁺

Elemental Analysis for C₂₄H₂₂N₄O₃S.0.7H₂O

Calcd.: C, 62.78; H, 5.14; N, 12.20

Found: C, 62.89; H, 4.86; N, 12.03

### EXAMPLE 9

N-[5-(1-Isopropyl-6-oxo-1,6-dihydro-3-pyridazinyl)-4-phenyl-1,3-thiazol-2-yl]-2-(4-methoxyphenyl)acetamide was obtained in a manner sinilar to Example 2.

mp: 188-190° C.

IR(KBr): 3191, 1648, 1581 cm⁻¹

¹H NMR(DMSO-d₆, δ): 1.26(6H,d,J=6.6 Hz), 3.31(2H,s), 3.74(3H,s), 5.12(1H, 7-plet,J=6.6 Hz), 6.79(1H,d,J=9.7 Hz), 6.85-6.95(2H,m), 6.99(1H,d,J=9.7 Hz), 7.26(2H,d,J=8.7 Hz), 7.35-7.55(5H,m), 12.62(1H,brs)

ESI/MS: 461[M+H]⁺, 483[M+Na]⁺

Elemental Analysis for C₂₅H₂₄N₄O₃S

Calcd.: C, 65.20; H, 5.25; N, 12.17

Found: C, 65.21; H, 5.28; N, 12.01

### EXAMPLE 10

A mixture of 6-(2-amino-4-phenyl-1,3-thiazol-5-yl)-2-isopropyl-3(2H)-pyridazinone (200 mg) and m-tolylisocyanate (93.8 mg) in dioxane (5 ml) was stirred for 6 hours at ambient temperature. The solvent was removed in vacuo to give a pale yellow powder. The powder was recrystallized from ethanol to give N-[5-(1-isopropyl-6-oxo-1,6-dihydro-3-pyridazinyl)-4-phenyl-1,3-thiazol-2-yllN′-(3-methylphenyl)urea as pale yellow crystals (100 mg).

mp: 242-243° C.

IR(KBr): 3357, 1710, 1639, 1616 cm⁻¹

¹H NMR(DMSO-d₆, δ): 1.29(6H,d,J=6.6 Hz), 2.31(3H,s), 5.14(1H, 7-plet,J=6.6 Hz), 6.79(1H,d,J=9.8 Hz), 6.8-6.95(1H,m), 6.99(1H,d,J=9.8 Hz), 7.1-7.6(8H,m), 8.88(1H,s), 10.8(1H,s)

ESI/MS: 446[M+H]⁺, 468[M+Na]⁺

Elemental Analysis for C₂₄H₂₃NsO₂S

Calcd.: C, 64.44; H, 5.23; N, 15.66

Found: C, 64.69; H, 5.14; N, 15.77

### EXAMPLE 11

A mixture of 6-(2-amino-4-phenyl-1,3-thiazol-5-yl)-2-isopropyl-3(2H)-pyridazinone (200 mg) and benzylisocyanate (93.8 mg) in dioxane (5 ml) was stirred for 6 hours at ambient temperature. The solvent was removed in vacuo to give a pale yellow powder. The powder was recrystallized from ethanol to give N-benzyl-N′-[5-(1-isopropyl-6-oxo-1,6-dihydro-3-pyridazinyl)-4-phenyl-1,3-thiazol-2-yl]urea as a pale yellow crystal (50 mg).

mp: 200-201° C.

IR(KBr): 3307, 1698, 1639, 1575 cm⁻¹

¹H NMR(DMSO-d₆, δ): 1.27(6H,d,J=6.6 Hz), 4.37(2H,d,J=5.9 Hz), 5.13(1H, 7-plet,J=6.6 Hz), 6.77(1H,d,J=9.7 Hz), 6.97(1H,d, J=9.7 Hz), 7.0-7.15(1H,m), 7.2-7.55(10H,m), 10.88(1H,br)

APCI/MS: 446[M+H]⁺

Elemental Analysis for C₂₄H₂₃N₅O₂S

Calcd.: C, 64.44; H, 5.23; N, 15.66

Found: C, 64.58; H, 5.29; N, 15.66

### EXAMPLE 12

N-({[5-(1-Isopropyl-6-oxo-1,6-dihydro-3-pyridazinyl)-4-phenyl-1,3-thiazol-2-yl]amino}carbonyl)-4-methylbenzene-sulfonamide was obtained in a marner similar to Example 11.

mp: 172-174° C.

IR(KBr): 3430, 1650, 1579 cm⁻¹

¹H NMR(DMSO-d₆, δ): 1.25(6H,d,J=6.6 Hz), 2.36(3H,s), 5.10(1H, 7-plet,J=6.6 Hz), 6.73(1H,d,J=9.7 Hz), 6.92(1H,d,J=9.7 Hz), 7.2-7.5(7H,m), 7.7-7.85(2H,m), 10.70(1H,br) Negative ESI/MS: 508[M−H]

### EXAMPLE 13

N-[5-(1-Isopropyl-6-oxo-1,6-dihydro-3-pyridazinyl)-4-phenyl-1,3-thiazol-2-yl]methanesulfonamide was prepared as a brown oil in a manner similar to Example 2.

¹H NMR(DMSO-d₆, δ): 1.27(6H,d,J=6.6 Hz), 3.73(3H,s), 5.14(1H, 7-plet,J=6.6 Hz), 6.88(1H,d,J=9.8 Hz), 7.14(1H,d,J=9.8 Hz), 7.4-7.65(5H,m) Negative ESI/MS: 389[M−H]⁻

### EXAMPLE 14

A mixture of 6-(1-bromo-2-oxo-2-phenylethyl)-2-isopropyl-3(2H)-pyridazinone (150 mg) and 1-hexyl-2-thiourea (108 mg) in dioxane (1 ml) was stirred overnight at 80° C. Chloroform and an aqueous sodium hydrogencarbonate solution were added to the reaction mixture at ambient temperature. The separated organic layer was dried over sodium sulfate. The solvent was removed in vacuo to give a yellow powder, which was subjected to a column chromatography on silica gel eluting with a mixture of chloroform and methanol (20:1).

The solvent was removed in vacuo to afford a yellow powder, which was suspended in diisopropyl ether with stirring. The powder was collected by filtration to afford 6-[2-(hexylamino)-4-phenyl-1,3-thiazol-5-yl]-2-isopropyl-3(2H)-pyridazinone as yellow powder (50 mg).

mp: 90-92° C.

IR(KBr): 3199, 1662, 1585 cm⁻¹

¹H NMR(DMSO-d₆, δ): 0.8-0.95(3H,m), 1.24(6H,d,J=6.6 Hz), 1.15-1.4(6H,m), 1.45-1.65(2H,m), 3.15-3.35(2H,m), 5.10(1H, 7-plet,J=6.6 Hz), 6.70(1H,d,J=9.7 Hz), 6.87(1H,d,J=9.7 Hz), 7.35-7.5(5H,m), 7.99(1H,t,J=5.5 Hz)

APCI/MS: 397[M+H]⁺, 419[M+Na]⁺, 815[2M+H]⁺

Elemental Analysis for C₂₂H₂₈N₄OS 0.2H₂O

Calcd.: C, 66.04; H, 7.15; N, 14.00

Found: C, 66.10; H, 7.25; N, 14.24

### EXAMPLE 15

2-Isopropyl-6-[2-(methylamino)-4-phenyl-1,3-thiazol-5-yl]-3(2H)-pyridazinone was obtained in a manner similar to Example 14.

mp: 234-236° C.

IR(KBr): 3203, 1664, 1581 cm⁻¹

¹H NMR(DMSO-d₆, δ): 1.25(6H,d,J=6.6 Hz), 2.87(3H,d,J=4.7 Hz), 5.10(1H, 7-plet,J=6.6 Hz), 6.70(1H,d,J=9.7 Hz), 6.86(1H,d,J=9.7 Hz), 7.3-7.5(5H,m), 7.8-8.0(1H,m)

APCI/MS: 327[M+H]⁺, 349[M+Na]⁺

Elemental Analysis for C₁₇H₁₈N₄OS.0.2H₂O

Calcd.: C, 61.87; H, 5.62; N, 16.98

Found: C, 62.02; H, 5.59; N, 17.02

### EXAMPLE 16

2-Isopropyl-6-[4-phenyl-2-(3-pyridinylamino)-1,3-thiazol-5-yl]-3(2H)-pyridazinone was obtained in a manner similar to Example 14.

mp: 226-228° C.

IR(KBr): 3045, 1660, 1581 cm⁻¹

¹H NMR(DMSO-d₆, δ): 1.27(6H,d), 5.13(1H, 7-plet), 6.80(1H,d,J=9.7 Hz), 7.01(1H,d,J=9.7 Hz), 7.35-7.7(6H,m), 8.2-8.4(2H,m), 8.9-9.0(1H,m), 10.91(1H,brs)

APCI/MS: 390[M+H]⁺, 412[M+Na]⁺

### EXAMPLE 17

A mixture of 6-(1-bromo-2-oxo-2-phenylethyl)-2-isopropyl-3(2H)-pyridazinone (150 mg) and N-methylthiourea (74.9 mg) in dioxane (1 ml) was stirred overnight at 80° C. The precipitate was collected by filtration to afford a yellow powder. The powder was recrystallized from ethanol to give 2-isopropyl-6-[2-(methylamino)-4-phenyl-1,3-thiazol-5-yl]-3(2H)-pyridazinone hydrobromide as pale yellow crystals (95 mg).

mp: 226-228° C.

IR(KBr): 3054, 1662, 1623, 1583 cm⁻¹

¹H NMR (DMSO-d₆, δ): 1.26(6H,dJ=3.3 Hz), 2.98(3H,s), 5.10(1H, 7-plet,J=3.3 Hz), 6.75(1H,d,J=8.4 Hz), 6.78(1H,d,J=8.4 Hz), 7.45-7.6(5H,m), 8.93(1H,br)

APCI/MS: 327[M+H]⁺

Elemental Analysis for C₁₇H₁₈N₄OS-HBr

Calcd.: C, 49.91; H, 4.73; N, 13.69

Found: C, 50.45; H, 4.73; N, 13.83

### EXAMPLE 18

6-(2-Anilino-4-phenyl-1,3-thiazol-5-yl)-2-isopropyl-3 (2H)-pyridazinone hydrobromide was obtained in a manner similar to Example 17.

mp: 127-129° C.

IR(KBr): 3419, 1666, 1623, 1579 cm⁻¹

¹H NMRPMSO-d₆, δ): 1.27(6H,d,J=3.3 Hz), 5.11(1H, 7-plet,J=3.3 Hz), 6.76(1H,d,J=4.9 Hz), 6.97(1H,d,J=4.9 Hz), 6.9-7.05(1H,m), 7.3-7.4(2H,m), 7.4-7.5(3H,m), 7.5-7.6(2H,m), 7.6-7.7(2H,m), 10.46(1H,br)

APCI/MS: 389[M+H]⁺

Elemental Analysis for C₂₂H₂₀N₄OS-HBr-1.4H₂O

Calcd.: C, 53.42; H, 4.85; N, 11.33

Found: C, 53.40; H, 4.79; N, 11.21

### EXAMPLE 19

6-[2-(Butylamino)-4-phenyl-1,3-thiazol-5-yl]-2-isopropyl-3(2H)-pyridazinone hydrobromide was obtained in a manner similar to Example 17.

mp: 204-205° C.

IR(KBr): 3415, 1668, 1633, 1585 cm⁻¹

¹H NMRPMSO-d₆, δ): 0.91(3H,t,J=3.7 Hz), 1.25(6H,d,J=3.3 Hz), 1.3-1.45(2H,m), 1.5-1.65(2H,m), 5.10(1H, 7-plet,J=3.3 Hz), 6.72(1H,d,J=4.9 Hz), 6.82(1H,d,J=4.9 Hz), 7.4-7.55(5H,m), 8.55(1H,br)

APCI/MS: 369[M+H]⁺

Elemental Analysis for C₂₀H₂₄N₄OS-HBr

Calcd.: C, 53.24; H, 5.63; N, 12.42

Found: C, 53.64; H, 5.60; N, 12.50

### EXAMPLE 20

2-Isopropyl-6-{4-phenyl-2-[(2-pyridinylmethyl)amino]-1,3-thiazol-5-yl}-3(2H)-pyridazinone was obtained in a manner similar to Example 14.

mp: 182-184° C.

IR(KBr): 3201, 1660, 1585 cm⁻¹

¹H NMR(DMSO-d₆, δ): 1.24(6H,d,J=6.6 Hz), 4.61(2H,d,J=5.9 Hz), 5.09(1H, 7-plet,J=6.6 Hz), 6.70(1H,d,J=9.6 Hz), 6.88(1H,d, J=9.6 Hz), 7.2-7.5(6H,m), 7.7-7.9(1H,m), 8.5-8.65(2H,m)

APCI/MS: 404[M+H]⁺

Elemental Analysis for C₂₂H₂₁NsOS

Calcd.: C, 65.20; H, 5.27; N, 17.28

Found: C, 65.18; H, 5.25; N, 17.33

### EXAMPLE 21

2-Isopropyl-6-(4-phenyl-2-{[2-(2-pyridinyl)ethyl]amino}-1,3-thiazol-5-yl)-3(2H)-pyridazinone hydrobrornide was obtained in a manner similar to Example 17.

mp: 126-127° C.

IR(KBr): 3205, 1660, 1581 cm⁻¹

¹H NMR(DMSO-d₆, δ): 1.24(6H,d,J=3.3 Hz), 3.06(2H,t,J=3.6 Hz), 3.66(2H,q,J=3.6 Hz), 5.10(1H, 7-plet,J=3.3 Hz), 6.70(1H,d, J=4.9 Hz), 6.87(1H,d,J=4.9 Hz), 7.2-7.35(2H,m), 7.35-7.5(5H,m), 7.65-7.75(1H,m), 8.0-8.1(1H,m)

APCI/MS: 418[M+H]⁺

### EXAMPLE 22

A mixture of 2-isopropyl-6-[2-(methylamino)-4-phenyl-1,3-thiazol-5-yl]-3(2H)-pyridazinone hydrobromide (150 mg) and acetyl chloride (43.3 mg) in pyridine (3 ml) was stirred overnight at ambient temperature. The solvent was removed in vacuo to give an oil, which was subjected to a column chromatography on silica gel eluting with a mixture of chloroform and methanol. The solvent was removed in vacuo to afford an oil, which was suspended in diisopropyl ether with stirring. The powder was collected by filtration to afford N-[5-(1-isopropyl-6-oxo-1,6-dihydro-3-pyridazinyl)-4-phenyl-1,3-thiazol-2-yl]-N-methylacetamide (60 mg).

mp: 165-167° C.

IR(KBr): 1666, 1585 cm⁻¹

¹H NMR(DMSO-d₆, δ): 1.26(6H,d,J=6.6 Hz), 2.43(3H,s), 3.69(3H,s), 5.13(1H, 7-plet,J=6.6 Hz), 6.81(1H,d,J=9.6 Hz), 7.03(1H,d, J=9.6 Hz), 7.35-7.6(5H,m)

APCI/MS: 369[M+H]⁺

Elemental Analysis for C₁₉H₂₀N₄O₂S

Calcd.: C, 61.64; H, 5.50; N, 15.13

Found: C, 61.82; H, 5.46; N, 15.06

### EXAMPLE 23

A mixture of 2-isopropyl-6-[2-(methylamino)-4-phenyl-1,3-thiazol-5-yl]-3(2H)-pyridazinone hydrobromide (100 mg), sodium hydride (61.3 mg) and methyl iodide (217 mg) in dimethylformamide (4 ml) was stirred for 3 hours at ambient temperature. Water and ethyl acetate were added to the reaction mixtureat ambient temperature. The separated organic layer was dried over sodium sulfate. The solvent was removed in vacuo to give a yellow powder, which was subjected to a colurn chromatography on silica gel eluting with a mixture of chloroform and methanol. The solvent was removed in vacuo to afford a yellow powder, which was suspended in diisopropyl ether with stirring.

The powder was collected by filtration to afford 6-[2-(dimethylamino)-4-phenyl-1,3-thiazol-5-yl]-2-isopropyl-3(2H)-pyridazinone as a yellow powder (44 mg).

mp: 158-160° C.

IR(KBr): 1668, 1565 cm⁻¹

¹H NMR(DMSO-d₆, δ): 1.26(6H,d,J=6.6 Hz), 3.10(6H,s), 5.10(1H, 7-plet,J=6.6 Hz), 6.70(1H,d,J=9.8 Hz), 6.85(1H,d,J=9.8 Hz), 7.35-7.55(5H,m)

APCI/MS: 341[M+Hq+

Elemental Analysis for C₁₈H₂₀N₄OS

Calcd.: C, 63.17; H, 5.95; N, 16.37

Found: C, 62.89; H, 5.88; N, 16.15

### EXAMPLE 24

A mixture of 6-(2-amino-4-phenyl-1,3-thiazol-5-yl)-2-isopropyl-3(2H)-pyridazinone (200 mg) and isoamyl nitrate (150 mg) in tetrahydrofuran (5 ml) was refluxed for 3 hours with stirring. The solvent was removed in vacuo to give a yellow oil, which was subjected to a column chromatography on silica gel eluting with a mixture of chloroform and methanol (20:1). The solvent was removed in vacuo to afford 2-isopropyl-6-(4-phenyl-1,3-thiazol-5-yl)-3(2H)-pyridazinone as an oil.

IR(KBr): 1670, 1662, 1652, 1589 cm⁻¹

¹H NMR(DMSO-d₆, δ): 1.24(6H,d,J=6.6 Hz), 5.13(1H, 7-plet,J=6.6 Hz), 6.86(1H,d,J=9.6 Hz), 7.13(1H,d,J=9.6 Hz), 7.4-7.6(5H,m), 9.23(1H,s)

APCI/MS: 298[M+H]⁺

### EXAMPLE 25

Phenyl 5-(1-isopropyl-6-oxo-1,6-dihydro-3-pyridazinyl)-4-phenyl-1,3-thiazol-2-ylcarbamate was obtained in a manner similar to Example 2.

mp: 205-207° C. (ethanol)

IR (KBr): 3432, 1732, 1643 cm⁻¹

¹H NMR (DMSO-d₆, δ): 1.25(6H, d, J=6.6 Hz), 5.12(1H, 7-plet, J=6.6 Hz), 6.79(1H, d, J=9.8 Hz), 6.99(1H, d, J=9.8 Hz), 7.2-7.6(10H, m), 12.64(1H, br)

ESI/MS: 433(M+1)⁺, 455(M+Na)⁺

### EXAMPLE 26

N-[5-(1-Isopropyl-6-oxo-1,6-dihydro-3-pyridazinyl)-4-phenyl-1,3-thiazol-2-yl]-2-pyridinecarboxamide was obtained in a manner similar to Example 2.

mp 245-246° C. (ethanol)

IR (KBr): 3340, 1664, 1587 cm⁻¹

¹H NMR (PMSO-dc, 8): 1.30(6H, d, J=6.6 Hz), 5.15(1H, 7-plet, J=6.6 Hz), 6.83(1H, d, J=9.7 Hz), 7.05(1H, d, J=9.7 Hz), 7.3-7.6(5H, m), 7.65-7.8(1H, m), 8.0-8.25(2H, m), 8.7-8.8(1H, m), 12.29(1H, brs)

ESI/MS: 418 (M+H)⁺, 440 (M+Na)⁺

Elemental Analysis for C₂₂H₁₉N₅O₂S

Calcd. C: 63.29, H: 4.59, N: 16.78

Found C: 63.25, H: 4.65, N: 16.73

### EXAMPLE 27

N-[5-(1-Isopropyl-6-oxo-1,6-dihydro-3-pyridazinyl)-4-phenyl-1,3-thiazol-2-yl]-4-(4-morpholinylmethyl)benzamide was obtained in a manner similar to Example 2.

mp: 222-224° C. (diisopropyl ether)

IR (KBr): 3442, 1648 cm⁻¹

¹H NMR (DMSO-d₆, δ): 1.30(6H, d, J=6.6 Hz), 2.3-2.45(4H, m), 3.5-3.7(6H, m), 5.15(1H, 7-plet, J=6.6 Hz), 6.82(1H, d, J=9.7 Hz), 7.03(1H, d, J=9.7 Hz), 7.3-7.6(7H, m), 8.0-8.2(2H, m), 12.91(1H, br)

ESI/MS: 516(M+H)⁺, 538 (M+Na)⁺

Elemental Analysis for C₂₈H₂₉N₅O₃S 0.3H₂O

Calcd. C: 64.55, H: 5.73, N: 13.44

Found C: 64.72, H: 5.90, N: 12.97

### EXAMPLE 28

4-[(Dimethylamino)methyl]-N-[5-(1-isopropyl-6-oxo-1,6-dihydro-3-pyridazinyl)-4-phenyl-1,3-thiazol-2-yl]benzamide was obtained in a manner similar to Example 2.

mp: 246-248° C. (diisopropyl ether)

IR (KBr): 3421, 1648 cm⁻¹

¹H NMR PMSO-d₆, δ): 1.30(6H, d, J=6.6 Hz), 2.17(6H, s), 3.48(2H, s), 5.15(1H, 7-plet, J=6.6 Hz), 6.82(1H, d, J=9.7 Hz), 7.04(1H, d, J=9.7 Hz), 7.3-7.6(7H, m), 8.0-8.2(2H, m), 12.89(1H, br)

ESI/MS: 474(M+H)⁺, 496(M+Na)⁺

Elemental Analysis for C₂₆H₂₇N₅O₂S.0.1H₂O

Calcd. C: 65.69, H: 5.77, N: 14.73

Found C: 65.57, H: 5.73, N: 14.73

### EXAMPLE 29

N-[5-(1-Isopropyl-6-oxo-1,6-dihydro-3-pyrndazinyl)-4-phenyl-1,3-thiazol-2-yl]-2-methylpropanamide was obtained in a manner similar to Example 2.

mp: 231-232° C. (ethyl acetate)

IR (KBr): 3181, 1689, 1648, 1581 cm⁻¹

¹H NMR (DMSO-d₆, δ): 1.14(6H, d, J=6.8 Hz), 1.27(6H, d, J=6.6 Hz), 2.77(1H, 7-plet, J=6.8 Hz), 5.13(1H, 7-plet, J=6.6 Hz), 6.80(1H, d, J=9.6 Hz), 7.01(1H, d, J=9.6 Hz), 7.3-7.6(5H, m), 12.38(1H, brs)

ESI/MS: 383(M+H)⁺, 405 (M+Na)⁺

Elemental Analysis for C₂₀H₂₂N₄O₂S

Calcd. C: 62.81, H: 5.80, N: 14.65

Found C: 62.71, H: 5.77, N: 14.73

### EXAMPLE 30

N-[5-(1-Isopropyl-6-oxo-1,6-dihydro-3-pyridazinyl)-4-phenyl-1,3-thiazol-2-yl]-2-naphthamide was obtained in a manner similar to Example 2.

mp: 227-229° C. (ethanol-ethyl acetate)

IR (KBr): 3151, 1679, 1643, 1579 cm⁻¹

¹H NMR (DMSO-d₆, δ): 1.31(6H, d, J=6.6 Hz), 5.16(1H, 7-plet, J=6.6 Hz), 6.83(1H, d, J=9.6 Hz), 7.06(1H, d, J=9.6 Hz), 7.3-7.8(7H, m), 7.9-8.2(4H, m), 8.85(1H, s), 13.10(1H, brs)

ESI/MS Nega: 465(M−H)⁺

Elemental Analysis for C₂₇H₂₂N₄O₂S

Calcd. C: 69.51, H: 4.75, N: 12.01

Found C: 69.21, H: 4.91, N: 11.98

### EXAMPLE 31

N-[5-(1-Isopropyl-6-oxo-1,6-dihydro-3-pyridazinyl)-4-phenyl-1,3-thiazol-2-yl]-1-naphthamide was obtained in a manner similar to Example 2.

mp: 223-224° C. (ethanol-ethyl acetate)

IR (KBr): 3141, 1681, 1643, 1577 cm⁻¹

¹H NMR (DMSO-d₆, δ): 1.32(6H, d, J=6.6 Hz), 5.17(1H, 7-plet, J=6.6 Hz), 6.84(1H, d, J=9.6 Hz), 7.06(1H, d, J=9.6 Hz), 7.3-7.7(7H, m), 7.8-8.2(4H, m), 8.2-8.4(1H, m), 13.10(1H, brs)

ESI/MS Nega: 465(M−H)⁺

Elemental Analysis for C₂₇H₂₂N₄O₂S 0.2H₂₀

Calcd. C: 68.98, H: 4.80, N: 11.92

Found C: 69.07, H: 4.73, N: 11.96

### EXAMPLE 32

N-[5-(1-Isopropyl-6-oxo-1,6-dihydro-3-pyridazinyl)-4-phenyl-1,3-thiazol-2-yl]-4-morpholinecarboxamide was obtained in a manner similar to Example 2.

mp: 231-232° C. (ethyl acetate)

IR (KBr): 3440, 1668 1590 cm⁻¹

¹H NMR (DMSO-d₆, δ): 1.27(6H, d, J=6.6 Hz), 3.4-3.7(8H, m), 5.12(1H, 7-plet, J=6.6 Hz), 6.77(1H, d, J=9.8 Hz), 6.96(1H, d, J=9.8 Hz), 7.3-7.6(5H, m), 11.25(1H, brs)

ESI/MS: 448(M+Na)⁺

Elemental Analysis for C₂₁H₂₃N,O₃S

Calcd. C: 59.28, H: 5.45 N: 16.45

Found C: 59.04, H: 5.49, N: 16.36

### EXAMPLE 33

N-[5-(1-Isopropyl-6-oxo-1,6-dihydro-3-pyridazinyl)-4-phenyl-1,3-thiazol-2-yl]cyclopropanecarboxamide was obtained in a manner similar to Example 2.

mp: 226-227° C. (ethyl acetate)

IR (KBr): 3392, 1687 1639 cm⁻¹

¹H NMR (PMSO-d₆, δ): 0.8-1.0(4H, m), 1.26(6H, d, J=6.6 Hz), 1.85-2.05(1H, m), 5.13(1H, 7-plet, J=6.6 Hz), 6.80(1H, d, J=9.8 Hz), 7.01(1H, d, J=9.8 Hz), 7.3-7.6(5H, m), 12.69(1H, brs)

ESI/MS: 381(M+H)⁺, 403(M+Na)⁺

Elemental Analysis for C₂₀H₂₀N₄O₂S 0.2H₂O

Calcd. C: 62.55, H: 5.35, N: 14.59

Found C: 62.50, H: 5.30, N: 14.60

### EXAMPLE 34

N-[5-(1-Isopropyl-6-oxo-1,6-dihydro-3-pyridazinyl)-4-phenyl-1,3-thiazol-2-yl]-2-methylbenzamide was obtained in a manner similar to Example 2.

mp: 221-222° C. (ethyl acetate)

IR (KBr): 3135, 1681 1641 cm⁻¹

¹H NMR (DMSO-d₆, δ): 1.30 (6H, d, J=6.6 Hz), 2.44(3H, s), 5.13(1H, 7-plet, J=6.6 Hz), 6.82(1H, d, J=9.8 Hz), 7.04(1H, d, J=9.8 Hz), 7.2-7.7(9H, m), 12.81(1H, brs)

ESI/MS: 431(M+H)⁺, 453(M+Na)⁺

Elemental Analysis for C₂₄H₂₂N₄O₂S

Calcd. C: 66.96, H: 5.15, N: 13.01

Found C: 67.11, H: 5.22, N: 13.04

### EXAMPLE 35

3-Chloro-N-[5-(1-isopropyl-6-oxo-1,6-dihydro-3-pyridazinyl)-4-phenyl-1,3-thiazol-2-yl]benzamide was obtained in a manner similar to Example 2.

mp: 173-174° C. (ethanol)

IR (KBr): 3426, 1649, 1579 cm⁻¹

¹H NMR (DMSO-d₆, δ): 1.30 (6H, d, J=6.6 Hz), 5.15(1H, 7-plet, J=6.6 Hz), 6.82(1H, d, J=9.7 Hz), 7.04(1H, d, J=9.7 Hz), 7.4-7.8(7H, m), 8.0-8.1(1H, m), 8.15-8.25(1H, m), 13.07(1H, brs)

ESI/MS Nega: 449(M−H)⁺

Elemental Analysis for C₂₃H₁₉ClN₄O₂S

Calcd. C: 61.26, H: 4.25, N: 12.43

Found C: 61.03, H: 4.04, N: 12.55

### EXAMPLE 36

3-Fluoro-N-[5-(1-isopropyl-6-oxo-1,6-dihydro-3-pyridazinyl)-4-phenyl-1,3-thiazol-2-yl]benzamide was obtained in a manner similar to Example 2.

mp: 183-184° C. (ethanol)

IR (KBr): 3421, 1639, 1575 cm⁻¹

¹H NMR (PMSO-d₆, δ): 1.30 (6H, d, J=6.6 Hz), 5.15(1H, 7-plet, J=6.6 Hz), 6.82(1H, d, J=9.7 Hz), 7.04(1H, d, J=9.7 Hz), 7.4-7.7(7H, m), 7.9-8.1(2H, m), 13.05(1H, brs)

ESI/MS: 435(M+H)⁺, 457(M+Na)⁺

Elemental Analysis for C₂₃H₁₉FN₄O₂S

Calcd. C: 63.58, H: 4.41, N: 12.89

Found C: 63.49, H: 4.40, N: 12.94

### EXAMPLE 37

2-Fluoro-N-[5-(1-isopropyl-6-oxo-1,6-dihydro-3-pyridazinyl)-4-phenyl-1,3-thiazol-2-yl]benzamide was obtained in a manner similar to Example 2.

mp: 251-252° C. (ethanol-ethyl acetate)

IR (KBr): 3421, 1666, 1587 cm⁻¹

¹H NMR (DMSO-d₆, δ): 1.30 (6H, d, J=6.6 Hz), 5.15(1H, 7-plet, J=6.6 Hz), 6.82(1H, d, J=9.7 Hz), 7.04(1H, d, J=9.7 Hz), 7.2-7.9(9H, m), 12.91(1H, brs)

ESI/MS: 435(M+H)⁺, 457(M+Na)⁺

Calcd. C: 63.58, H: 4.41, N: 12.89

Found C: 63.39, H: 4.70, N: 12.89

### EXAMPLE 38

N-[5-(1-Isopropyl-6-oxo-1,6-dihydro-3-pyridazinyl)-4-phenyl-1,3-thiazol-2-yl]-3-(trifluoromethyl)benzamide was obtained in a manner similar to Example 2.

mp: 237-238° C. (ethanol)

IR (KBr): 1646, 1581 cm⁻¹

¹H NMR (DMSO-dr, 6): 1.30 (6H, d, J=6.6 Hz), 5.15(1H, 7-plet, J=6.6 Hz), 6.82(1H, d, J=9.6 Hz), 7.04(1H, d, J=9.6 Hz), 7.35-7.6(5H, m), 7.80(1H, t, J=8 Hz), 8.02(1H, t, J=8 Hz), 8.42(1H, t, J=8 Hz), 8.53(1H, s), 13.23(1H, brs)

ESI/MS: 485(M+H)⁺, 507(M+Na)⁺

Elemental Analysis for C₂₄H₁₉F₃N₄O₂S

Calcd. C: 59.50, H: 3.95, N: 11.56

Found C: 59.476, H: 3.97, N: 11.54

### EXAMPLE 39

N-[5-(1-Isopropyl-6-oxo-1,6-dihydro-3-pyridazinyl)-4-phenyl-1,3-thiazol-2-yl]-4-(trifluoromethyl)benzamide was obtained in a manner similar to Example 2.

mp 162-167° C. (ethanol)

IR (KBr): 1648, 1579 cm⁻¹

¹H NMR (DMSO-d₆, δ): 1.30 (6H, d, J=6.6 Hz), 5.15(1H, 7-plet, J=6.6 Hz), 6.83(1H, d, J=9.6 Hz), 7.04(1H, d, J=9.6 Hz), 7.3-7.6(5H, m), 7.95(2H, d, J=8.4 Hz), 8.32(2H, t, J=8.4 Hz), 13.22(1H, brs)

ESI/MS: 485(M+H)⁺, 507(M+Na)⁺

Elemental Analysis for C₂₄H₁₉F₃N₄O₂S.0.1H₂O

Calcd. C: 59.50, H: 3.95 N: 11.56

Found C: 59.28, H: 3.98, N: 11.52

### EXAMPLE 40

N-[5-(1-Isopropyl-6-oxo-1,6-dihydro-3-pyridazinyl)-4-phenyl-1,3-thiazol-2-yl]-2-(trifluoromethyl)benzamide was obtained in a manner similar to Example 2.

mp: 219-220° C. (ethanol)

IR (KBr): 3174, 1650, 1583 cm⁻¹

¹H NMR (DMSO-d₆, δ): 1.29 (6H, d, J=6.6 Hz), 5.15(1H, 7-plet, J=6.6 Hz), 6.83(1H, d, J=9.6 Hz), 7.04(1H, d, J=9.6 Hz), 7.3-7.6(5H, m), 7.7-7.95(4H, m), 13.13(1H, brs)

ESI/MSnega: 483(M−H)⁺

Elemental Analysis for C₂₄H₁₉F₃N₄O₂S

Calcd. C: 59.50, H: 3.95, N: 11.56

Found C: 59.44, H: 4.03, N: 11.70

### EXAMPLE 41

N-[5-(1-Isopropyl-6-oxo-1,6-dihydro-3-pyridazinyl)-4-phenyl-1,3-thiazol-2-yl]-2-methoxybenzamide was obtained in a manner similar to Example 2.

mp: >250° C. (ethanol)

IR (KBr): 3315, 1658, 1585 cm⁻¹

¹H NMR (DMSO-d₆, δ): 1.30 (6H, d, J=6.6 Hz), 3.93(3H, s), 5.15(1H, 7-plet, J=6.6 Hz), 6.81(1H, d, J=9.6 Hz), 6.9-7.25(3H, m), 7.4-7.65(6H, m), 7.65-7.8(1H, m), 12.09(1H, brs)

ESI/MS: 447(M+H)⁺, 469(M+Na)⁺

Elemental Analysis for C₂₄H₂₂O₃S

Calcd. C: 64.56, H: 4.97, N: 12.55

Found C: 64.56, H: 4.96, N: 12.60

### EXAMPLE 42

N-[5-(1-Isopropyl-6-oxo-1,6-dihydro-3-pyridazinyl)-4-phenyl-1,3-thiazol-2-yl]-3-methylbenzamide was obtained in a manner similar to Example 2.

mp: 198-199° C. (ethyl acetate)

IR (KBr): 3349, 1646, 1579 cm⁻¹

¹H NMR (DMSO-d₆, δ): 1.30 (6H, d, J=6.6 Hz), 2.40(3H, s), 5.15(1H, 7-plet, J=6.6 Hz), 6.82(1H, d, J=9.8 Hz), 7.04(1H, d, J=9.8 Hz), 7.3-7.6(7H, m), 7.8-8.05(2H, m), 12.88(1H, brs)

ESI/MS: 431(M+H)⁺, 453(M+Na)⁺

Elemental Analysis for C₂₄H₂₂O₂S 0.2H₂O

Calcd. C: 66.40, H: 5.20, N: 12.91

Found C: 66.50, H: 5.32, N: 12.73

### EXAMPLE 43

N-[5-(1-Isopropyl-6-oxo-1,6-dihydro-3-pyridazinyl)-4-phenyl-1,3-thiazol-2-yL]-3-methylbenzamide was obtained in a manner similar to Example 2.

mp: 198-199° C. (ethyl acetate)

IR (KBr): 3388, 1660, 1581 cm⁻¹

¹H NMR (DMSO-d₆, δ): 1.30 (6H, d, J=6.6 Hz), 2.40(3H, s), 5.15(1H, 7-plet, J=6.6 Hz), 6.81(1H, d, J=9.8 Hz), 7.03(1H, d, J=9.8 Hz), 7.3-7.6(7H, m), 8.05(1H, d, J=8.2 Hz), 12.87(1H, brs)

ESI/MS: 431(M+H)⁺, 453(M+Na)⁺

Elemental Analysis for C₂₄H₂₂O₂S.0.2H₂O

Calcd. C: 66.40, H: 5.20 N: 12.91

Found C: 66.50, H: 5.32, N: 12.73

### EXAMPLE 44

N-[5-(1-Isopropyl-6-oxo-1,6-dihydro-3-pyridazinyl)-4-phenyl-1,3-thiazol-2-yl]-3,5-bis(trifluoromethyl)benzamide was obtained in a manner similar to Example 2.

mp: 207-208° C. (ethanol)

IR (KBr): 1646, 1575 m⁻¹

¹H NMR (DMSO-d₆, δ): 1.30 (6H, d, J=6.6 Hz), 5.15(1H, 7-plet, J=6.6 Hz), 6.83(1H, d, J=9.6 Hz), 7.05(1H, d, J=9.6 Hz), 7.3-7.6(5H, m), 8.43(1H, s), 8.79(2H, s), 13.44(1H, brs)

ESI/MSNega: 551(M−H)⁺

Elemental Analysis for C₂₅H₁₈N₄O₂S

Calcd. C: 54.35, H: 3.28, N: 10.14

Found C: 54.41, H: 3.30, N: 10.36

### EXAMPLE 45

N-[5-(1-Isopropyl-6-oxo-1,6-dihydro-3-pyridazinyl)-4-phenyl-1,3-thiazol-2-yl]-4-methoxybenzamide was obtained in a manner similar to Example 2.

mp: 219-221° C. (ethanol)

IR (KBr): 3421, 1646, 1577 cm⁻¹

¹H NMR (DMSO-d₆, δ): 1.30 (6H, d, J=6.6 Hz), 3.86(3H, s), 5.15(1H, 7-plet, J=6.6 Hz), 6.81(1H, d, J=9.6 Hz), 6.95-7.15(3H, m), 7.35-7.65(5H, m), 8.05-8.2(2H, m)

ESI/MS: 447(M+H)⁺, 469(M+Na)⁺

Elemental Analysis for C₂₅H₁₈N₄O₂S

Calcd. C: 64.30, H: 4.99, N: 12.50

Found C: 64.17, H: 4.93, N: 12.80

### EXAMPLE 46

2-Chloro-N-[5-(1-isopropyl-6-oxo-1,6-dihydro-3-pyridazinyl)-4-phenyl-1,3-thiazol-2-yl]benzamide was obtained in a manner similar to Example 2.

mp: 220-221° C. (ethanol)

IR (EBr): 3421, 1641, 1573 cm⁻¹

¹H NMR (DMSO-dr, 3): 1.30 (6H, d, J=6.6 Hz), 5.15(1H, 7-plet, J=6.6 Hz), 6.83(1H, d, J=9.8 Hz), 7.04(1H, d, J=9.8 Hz), 7.3-7.7(9H, m), 13.03(1H, brs)

ESI/MS: 473(M+Na)+

Elemental Analysis for C₂₃HigC1N₄O₂S

Calcd. C: 61.26, H: 4.25, N: 12.42

Found C: 61.16, H: 4.22, N: 12.38

### EXAMPLE 47

4-Chloro-N-[5-(1-isopropyl-6-oxo-1,6-dihydro-3-pyridazinyl)-4-phenyl-1,3-thiazol-2-yl]benzamide was obtained in a manner similar to Example 2.

mp: 205-206° C. (ethanol)

IR (KBr): 3178, 1641, 1575 cm⁻¹

¹H NMR (DMSO-d₆, δ): 1.30 (6H, d, J=6.6 Hz), 5.15(1H, 7-plet, J=6.6 Hz), 6.82(1H, d, J=9.8 Hz), 7.04(1H, d, J=9.8 Hz), 7.3-7.7(7H, m), 8.15(2H, dd, J=2 Hz and 9.1 Hz), 13.04(1H, brs)

ESI/MS Nega: 449(M−H)⁺

Elemental Analysis for C₂₃H₁₉ClN₄O₂S

Calcd. C: 61.26, H: 4.25, N: 12.42

Found C: 61.27, H: 4.26, N: 12.41

### EXAMPLE 48

4-fluoro-N-[5-(1-isopropyl-6-oxo-1,6-dihydro-3-pyridazinyl)-4-phenyl-1,3-thiazol-2-yl]benzamide was obtained in a manner similar to Example 2.

mp: 225-226° C. (ethanol)

IR (KBr): 3180, 1679, 1641, 1575 cm⁻¹

¹H NMR (DMSO-d₆, δ): 1.30 (6H, d, J=6.6 Hz), 5.15(1H, 7-plet, J=6.6 Hz), 6.82(1H, d, J=9.8 Hz), 7.04(1H, d, J=9.8 Hz), 7.3-7.6(7H, m), 8.1-8.3(2H, m), 12.98(1H, brs)

ESI/MS: 435(M+H)⁺, 457(M+Na)⁺

Elemental Analysis for C₂₃HigFN₄O₂S

Calcd. C: 63.58, H: 4.41, N: 12.89

Found C: 63.57, H: 4.44, N: 12.94

### EXAMPLE 49

2,6-Dichloro-N-[5-(1-isopropyl-6-oxo-1,6-dihydro-3-pyridazinyl)-4-phenyl-1,3-thiazol-2-yl]benzamide was obtained in a manner similar to Example 2.

mp: 248-249° C. (ethyl acetate)

IR (KBr): 3428, 1679, 1646, 1581 cm⁻¹

¹H NMR (DMSO-d₆, δ): 1.29 (6H, d, J=6.6 Hz), 5.15(1H, 7-plet, J=6.6 Hz), 6.83(1H, d, J=9.7 Hz), 7.05(1H, d, J=9.7 Hz), 7.3-7.7(8H, m), 13.28(1H, brs)

ESI/MS: 485(M)⁺

### EXAMPLE 50

N′-[5-(1-Isopropyl-6-oxo-1,6-dihydro-3-pyridazinyl)-4-phenyl-1,3-thiazol-2-yl]-N,N-dimethylurea was obtained in a manner similar to Example 2.

mp: 199-200° C. (ethyl acetate)

IR (KBr): 3239, 1673, 1648, 1583 cm⁻¹

¹H NMR (DMSO-di, 6): 1.27(6H, d, J=6.6 Hz), 2.98(6H, s), 5.13(1H, 7-plet, J=6.6 Hz), 6.77(1H, d, J=9.6 Hz), 6.96(1H, d, J=9.6 Hz), 7:3-7.6(5H, m), 11.03(1H, brs)

ESI/MS: 384(M+H)⁺, 406 (M+Na)⁺

### EXAMPLE 51

4-Iodo-N-[5-(1-isopropyl-6-oxo-1,6-dihydro-3-pyridazinyl)-4-phenyl-1,3-thiazol-2-yl]benzamide was obtained in a manner similar to Example 2.

mp: 253-254° C. (ethanol)

IR (KBr): 1673, 1643, 1579 cm⁻¹

¹H NMR (DMSO-d₆, δ): 1.29(6H, d, J=6.6 Hz), 5.15(1H, 7-plet, J=6.6 Hz), 6.82(1H, d, J=9.7z), 7.04(1H, d, J=9.7 Hz), 7.3-7.6(5H, m), 7.8-8.0(4H, m), 13.02(1H, br)

ESI/MS: 543(M+H)⁺, 565 (M+Na)⁺

### EXAMPLE 52

N-[5-(1-Isopropyl-6-oxo-1,6-dihydro-3-pyridazinyl)-4-phenyl-1,3-thiazol-2-yl]-1-piperidinecarboxamide was obtained in a manner similar to Example 2.

mp: 138-140° C. (ethyl acetate-isopropyl ether)

IR (KBr): 3224, 1652, 1581 cm⁻¹

ESI/MS: 424(M+H)⁺, 446 (M+Na)⁺

### EXAMPLE 53

N-[5-(1-Isopropyl-6-oxo-1,6-dihydro-3-pyridazinyl)-4-phenyl-1,3-thiazol-2-yl]-2-(trifluoromethoxy)benzamide was obtained in a manner similar to Example 2.

mp: 212-213° C. (ethanol)

IR (KBr): 3141, 1646, 1579 cm⁻¹

¹H NMR (DMSO-d₆, B): 1.30(6H, d, J=6.6 Hz), 5.15(1H, 7-plet, J=6.6 Hz), 6.82(1H, d, J=9.7z), 7.04(1H, d, J=9.7 Hz), 7.3-7.8(7H, m), 8.0-8.25(2H, m), 13.18(1H, br)

ESI/MS Nega: 499(M−H)⁻

### EXAMPLE 54

N-[5-(1-Isopropyl-6-oxo-1,6-dihydro-3-pyridazinyl)-4-phenyl-1,3-thiazol-2-yl]-9H-carbazole-9-carboxamide was obtained in a manner similar to Example 2.

mp: 241-242° C. (ethanol)

IR (KBr): 3089, 1652, 1579 cm⁻¹

¹H NMR PMSO-d₆, δ): 1.2-1.4 (6H, m), 5.15(1H, 7-plet, J=6.6 Hz), 6.79(1H, d, J=9.7z), 6.87(1H, d, J=9.7 Hz), 7.3-7.7(10H, m), 8.0-8.2(2H, m), 8.7-9.0(2H, br)

ESI/MS: 504 (M+H)⁺

### EXAMPLE 55

N-[5-(1-Isopropyl-6-oxo-1,6-difiydro-3-pyridazinyl)-4-phenyl-1,3-thiazol-2-yl]isonicotinamide was obtained in a manner similar to Example 2.

mp: 223-224° C. (ethanol)

IR (KBr):3432, 1668, 1583 cm⁻¹

¹H NMR (DMSO-d₆, δ): 1.30(6H, d, J=6.6 Hz), 5.15(1H, 7-plet, J=6.6 Hz), 6.83(1H, d, J=9.8 Hz), 7.04(1H, d, J=9.8 Hz), 7.35-7.6(5H, m), 8.03(2H, dd, J=1.4 and 4.6 Hz), 8.83(2H, dd, J=1.4 and 4.6 Hz), 13.28(1H, brs).

ESI/MS: 418 (M+H)⁺, 440 (M+Na)⁺

### EXAMPLE 56

4-(Chloromethyl)-N-[5-(1-isopropyl-6-oxo-1,6-dihydro-3-pyridazinyl)-4-phenyl-1,3-thiazol-2-yl]benzamide was obtained in a manner similar to Example 2.

mp: >250° C. (ethanol)

IR (KBr):3419, 1650, 1579 cm⁻

¹H NMR (DMSO-d₆, δ): 1.30(6H, d, J=6.6 Hz), 4.86(2H, s), 5.15(1H, 7-plet, J=6.6 Hz), 6.83(1H, d, J=9.8 Hz), 7.04(1H, d, J=9.8 Hz), 7.3-7.7(7H, m), 8.0-8.2(2H, m), 12.99(1H, brs)

ESI/MS: 465 (M+H)⁺, 487 (M+Na)⁺

### EXAMPLE 57

N-[4-(4-Fluorophenyl)-5-(1-isopropyl-6-oxo-1,6-dihydro-3-pyridazinyl)-1,3-thiazol-2-yl]cyclopropanecarboxamide was obtained in a manner similar to Example 2,

mp: 250-252° C. (ethanol)

IR (KBr): 3154, 1689, 1646, 1579 cm⁻¹

¹H NMR (PMSO-d₆, δ): 0.8-1.0(4H, m), 1.25(6H, d, J=6.6 Hz), 1.9-2.1(1H, m), 5.12(1H, 7-plet, J=6.6 Hz), 6.82(1H, d, J=9.6z), 7.04(1H, d, J=9.6 Hz), 7.2-7.35(25H, m), 7.5-7.6(2H, m), 12.72(1H, br)

ESI/MS: 399(M+H)⁺, 421(M+Na)⁺

### EXAMPLE 58

N-[5-(1-Isopropyl-6-oxo-1,6-dihydro-3-pyridazinyl)-4-phenyl-1,3-thiazol-2-yl]-3-methylbutanamide was obtained in a manner similar to Example 2.

mp: 198-199° C. (ethyl acetate-isopropyl ether)

mp: >250° C. (diisopropyl ether)

IR (KBr):3154, 1689, 1646, 1579 cm⁻¹

¹H NMR (DMSO-d₆, δ): 0.94(6H, d, J=6.6 Hz), 1.28(6H, d, J=6.6 Hz), 2.11(1H, m), 2.36(2H, d, J=7.1 Hz), 5.14(1H, 7-plet, J=6.6 Hz), 6.80(1H, d, J=9.7 Hz), 7.01(1H, d, J=9.7 Hz), 7.35-7.6(5H, m), 12.39(1H, brs)

ESI/MS: 397 (M+H)⁺, 419 (M+Na)⁺

### EXAMPLE 59

2-Chloro-N-[5-(1-isopropyl-6-oxo-1,6-dihydro-3-pyridazinyl)-4-(4-fluorophenyl)-1,3-thiazol-2-yl]acetamide was obtained in a manner similar to Example 2.

¹H NMR (CDCl₃, δ): 1.40(6H, d, J=6.6 Hz), 4.24(2H, s), 5.32(1H, 7-plet, J=6.6 Hz), 6.76(1H, d, J=9.6 Hz), 6.94(1H, d, J=9.6 Hz), 7.0-7.2(2H, m), 7.4-7.6(2H, m), 10.13(1H, br)

ESI/MS: 429(M+Na)⁺

Elemental Analysis for C₂₂H₁₉N₅O₂S

Calcd. C: 63.29, H: 4.59, N: 16.78

Found C: 63.25, H: 4.65, N: 16.73

### EXAMPLE 60

2-Chloro-N-[5-(1-isopropyl-6-oxo-1,6-dihydro-3-pyridazinyl)-4-phenyl-1,3-thiazol-2-yl]acetamide was obtained in a manner similar to Example 2.

¹H NMR (DMSO-d₆, δ): 1.28(6H, d, J=6.6 Hz), 4.44(2H, s), 5.14(1H, 7-plet, J=6.6 Hz), 6.81(1H, d, J=9.6 Hz), 7.03(1H, d, J=9.6 Hz), 7.3-7.6(5H, m), 12.81(1H, br)

ESI/MS: 389(M+H)⁺, 411(M+Na)⁺

### EXAMPLE 61

6-[2-(tert-Butylamino)-4-phenyl-1,3-thiazol-5-yl]-2-isopropyl-3(2H)-pyridazinone was obtained in a manner similar to Example 14.

mp: 189-190° C. (ethanol)

IR (KBr): 3288, 3257, 1648, 1581 cm⁻¹

¹H NMR (DMSO-dr, 6): 1.23(6H, d, J=6.6 Hz), 1.40(9H, s), 5.10(1H, 7-plet, J=6.6 Hz), 6.72(1H, d, J=9.7 Hz), 6.94(1H, d, J=9.7 Hz), 7.3-7.55(5H, m), 7.72(1H, s)

ESI/MS: 369(M+H)⁺, 391(M+Na)⁺

Elemental Analysis for C₂₀H₂₄N₄OS

Calcd. C: 65.19; H:6.56; N: 15.20

Found C: 65.12; H: 6.59; N: 15.20

### EXAMPLE 62

6-[2-(Ethylamino)-4-phenyl-1,3-thiazol-5-yl]-2-isopropyl-3(2H)-pyridazinone was obtained in a manner similar to Example 14.

mp: 167-169° C. (ethanol)

IR (KBr): 3203, 1664, 1575 cm⁻¹

¹H NMR PMSO-d₆, δ): 1.18(3H, t, J=7.3 Hz), 1.25(6H, d, J=6.7 Hz), 3.15-3.4(2H, m), 5.10(1H, 7-plet, J=6.7 Hz), 6.70(1H, d, J=9.6 Hz), 6.87(1H, d, J=9.6 Hz), 7.3-7.55(5H, m), 7.97(1H, t, J=5.3 Hz)

ESI/MS: 341(M+H)⁺, 363 (M+Na)⁺

Elemental Analysis for ClaH₂-ON₄OS 0.2H₂O

Calcd. C: 62.84, H: 5.98, N: 16.28

Found C: 62.85, H: 5.97, N: 16.31

### EXAMPLE 63

N-[5-(1-Isopropyl-6-oxo-1,6-dihydro-3-pyridazinyl)-4-phenyl-1,3-thiazol-2-yl]guanidine was obtained in a manner similar to Example 14.

mp: >250° C. (ethanol)

IR (KBr): 3405, 1656 cm⁻¹

¹H NMR (DMSO-ds, 6): 1.22(6H, t, J=6.6 Hz), 5.09(1H, 7-plet, J=6.6 Hz), 6.72(1H, d, J=9.6 Hz), 6.93(1H, d, J-9.6 Hz), 6.9-7.1(4H, br), 7.3-7.55(5H, m)

ESI/MS: 355(M+H)⁺, 377 (M+Na)⁺

Elemental Analysis for C₁₇H₁₈N₆OS.0.2H₂O

Calcd. C: 57.03, H: 5.18, N: 23.47

Found C: 56.99; H: 5.22, N: 23.29

### EXAMPLE 64

2-Isopropyl-6-[2-(isopropylamino)-4-phenyl-1,3-thiazol-5-yl]-3(2H)-pyridazinone was obtained in a manner similar to Example 14.

mp 138-139° C. (ethanol)

IR (KBr): 3259, 1650, 1585 cm⁻¹

¹H NMR PMSO-d₆, δ): 1.0-1.3(12H, m), 3.7-3.95(1H, m), 5.10(1H, 7-plet, J=6.6 Hz), 6.70(1H, d, J=9.6 Hz), 6.87(1H, d, J=9.6 Hz), 7.3-7.6(4H, m), 7.8-8.0(1H, m)

ESI/MS: 355(M+H)⁺, 377 (M+Na)⁺

### EXAMPLE 65

6-[2-(Benzylamino)-4-phenyl-1,3-thiazol-5-yl]-2-isopropyl-3 (2H)-pyridazinone was obtained in a manner simniar to Example 14.

mp 157-158° C. (ethanol)

IR (KBr): 3201, 1662, 1583 cm⁻¹

¹H NMR (DMSO-d₆, δ): 1.23(6H, d, J=6.6 Hz), 4.52(2H, d, J=5.9 Hz), 5.10(1H, 7-plet, J=6.6 Hz), 6.70(1H, d, J=9.7 Hz), 6.88(1H, d, J=9.7 Hz), 7.1-7.6(10H, m), 8.50(1H, t, J=5.9 Hz)

ESI/MS: 403(M+H)⁺, 425 (M+Na)⁺

### EXAMPLE 66

6-{2-[(2-Furylmethyl)amino]-4-phenyl-1,3-thiazol-5-yl}-2-isopropyl-3(2H)-pyridazinone was obtained in a manner similar to Example 14.

mp: 115-116° C. (ethanol)

IR (KBr): 3201, 1658, 1583 cm⁻¹

¹H NMR (DMSO-d₆, δ): 1.24(6H, d, J=6.6 Hz), 4.50(2H, d, J=5.6 Hz), 5.10(1H, 7-plet, J=6.6 Hz), 6.3-6.45(2H, m), 6.71(1H, d, J=9.7 Hz), 6.89(1H, d, J=9.7 Hz), 7.3-7.7(6H, m), 8.40(1H, t, J=5.6 Hz)

ESI/MS: 393(M+H)⁺, 415 (M+Na)⁺

### EXAMPLE 67

2-Isopropyl-6-[4-phenyl-2-(2-pyridinylamino)-1,3-thiazol-5-yl]-3(2H)-pyridazinone was obtained in a manner similar to Example 14.

mp: 194-195° C. (ethanol)

IR (KBr): 3444, 1646, 1577 cm⁻¹

¹H NMR (DMSO-d₆, δ): 1.29(6H, d, J=6.6 Hz), 5.14(1H, 7-plet, J=6.6 Hz), 6.78(1H, d, J=9.7 Hz), 6.9-7.15(3H, m), 7.3-7.7(5H, m), 7.6-7.8(1H, m), 8.25-8.4(1H, m), 11.6(1H, br)

ESI/MS: 390(M+H)⁺, 412 (M+Na)⁺

### EXAMPLE 68

2-I sopropyl-6-(2-{[3-(4-morpholinyl)propyl]amino}-4-phenyl-1,3-thiazol-5-yl)-3(2H)-pyridazinone was obtained in a manner similar to Example 14.

mp: 194-195° C. (ethanol)

IR (KBr): 3444, 1646, 1577 cm⁻¹

¹H NMR (DMSO-d₆, δ): 1.25(6H, d, J=6.6 Hz), 1.6-1.85(2H, m), 2.2-2.45(6H, m), 3.2-3.4(2H, m), 3.5-3.7(4H, m), 5.10(1H, 7-plet, J=6.6 Hz), 6.70(1H, d, J=9.8 Hz), 6.87(1H, d, J=9.8 Hz), 7.3-7.6(5H, m), 8.01(1H, t, J=5.5 Hz)

ESI/MS: 440(M+H)⁺, 462 (M+Na)+

### EXAMPLE 69

2-Isopropyl-6-(2-{[2-(4-morpholinyl)ethyl]amino}-4-phenyl-1,3-thiazol-5-yl)-3(2H)-pyridazinone was obtained in a manner similar to Example 14.

IR (KBr): 3444, 1646, 1577 cm⁻¹

¹H NMR (DMSO-d₆, δ): 1.24(6H, d, J=6.6 Hz), 2.3-2.6(6H, m), 3.2-3.7(6H, m), 5.10(1H, 7-plet, J=6.6 Hz), 6.70(1H, d, J-9.6 Hz), 6.87(1H, d, J=9.6 Hz), 7.3-7.6(5H, m), 7.85-8.0(1H, m)

ESI/MS: 426(M+H)⁺, 448 (M+Na)⁺

### EXAMPLE 70

6-[2-(Cyclohexylamino)-4-phenyl-1,3-thiazol-5-yl]-2-isopropyl-3(2H)-pyridazinone was obtained in a manner siar to Example 14.

mp: 149-151° C. (ethanol)

IR (KBr): 3203, 1668, 1569 cm⁻¹

¹H NMR (DMSO-d₆, δ): 1.24(6H, d, J=6.6 Hz), 1.1-1.4(5H, m), 1.45-1.8(3H, m), 1.85-2.05(2H, m), 3.4-3.6(1H, br), 5.10(1H, 7-plet, J=6.6 Hz), 6.69(1H, d, J=9.8 Hz), 6.87(1H, d, J=9.8 Hz), 7.3-7.55(5H, m), 7.94(1H, d, J=7.6 Hz)

ESI/MS: 395 (M+H)⁺, 417 (M+Na)⁺

### EXAMPLE 71

2-Isopropyl-6-{2-[(2-methoxyethyl)amino]-4-phenyl-1,3-thiazol-5-yl}-3(2H)-pyiidazinone was obtained in a manner similar to Example 14.

mp: 112-114° C. (isopropyl ether)

IR (KBr): 3363, 1664, 1587 cm⁻¹

¹H NMR (DMSO-d₆, δ): 1.24(6H, d, J=6.6 Hz), 3.29(3H, s), 3.35-3.6(4H, m), 5.10(1H, 7-plet, J=6.6 Hz), 6.70(1H, d, J=9.6 Hz), 6.87(1H, d, J=9.6 Hz), 7.3-7.55(5H, m), 8.0-8.2(1H, m)

ESI/MS: 371 (M+H)⁺, 393 (M+Na)⁺

### EXAMPLE 72

2-Isopropyl-6-[2-(1-naphthylamino)-4-phenyl-1,3-thiazol-5-yl]-3(2H)-pyridazinone was obtained in a manner similar to Example 14.

mp: 239-240° C. (ethanol)

IR (KBr): 1664, 1579 cm⁻¹

¹H NMR (DMSO-d₆, δ): 1.24(6H, d, J=6.6 Hz), 5.10(1H, 7-plet, J=6.6 Hz), 6.76(1H, d, J=9.7 Hz), 6.96(1H, d, J=9.7 Hz), 7.3-7.8(9H, m), 7.85-8.3(3H, m), 10.38(1H, br)

ESI/MS: 439 (M+H)⁺, 461 (M+Na)⁺

### EXAMPLE 73

2-Isopropyl-6-[4-phenyl-2-(propylamino)-1,3-thiazol-5-yl]-3(2H)-pyridazinone was obtained in a manner similar to Example 14.

mp: 165-166° C. (ethanol)

IR (KBr): 3205, 1666, 1577 cm⁻¹

¹H NMR (DMSO-d₆, δ): 0.92(3H, t, J=7.4 Hz), 1.24(6H, d, J=6.6 Hz), 1.59(2H, m), 3.1-3.4(2H, m), 5.10(1H, 7-plet, J=6.6 Hz), 6.70(1H, d, J=9.7 Hz), 6.87(1H, d, J=9.7 Hz), 7.3-7.55(5H, m), 8.01(1H, t, J=5.4 Hz)

ESI/MS: 355 (M+H)⁺, 377 (M+Na)⁺

### EXAMPLE 74

2-Isopropyl-6-(4-phenyl-2-{[2-(1-piperidinyl)ethyl]amino}-1,3-thiazol-5-yl)-3(2H)-pyridazinone was obtained in a manner similar to Example 14.

mp: 165-166° C. (isopropyl ether)

IR (KBr): 3205, 1666, 1577 cm⁻¹

¹H NMR (DMSO-d₆, δ): 1.24(6H, d, J=6.6 Hz), 1.3-1.6(6H, m), 2.3-2.6(4H, m), 3.2-3.5(4H, m), 5.10(1H, 7-plet, J=6.6 Hz), 6.70(1H, d, J=9.8 Hz), 6.87(1H, d, J=9.7 Hz), 7.3-7.55(5H, m), 7.8-7.9(1H,m)

ESI/MS: 424 (M+H)⁺

### EXAMPLE 75

6-(2-{[4-(Dimethylamino)phenyl]amino4-phenyl-1,3-thiazol-5-yl)-2-isopropyl-3(2H)-pyridazinone was obtained in a manner similar to Example 14.

mp: 234-236° C. (ethanol)

¹H NMR (DMSO-d₆, δ): 1.25(6H, d, J=6.6 Hz), 2.85(3H,s), 5.10(1H, 7-plet, J=6.6 Hz), 6.6-6.8(3H, m), 6.93(1H, d, J=9.7 Hz), 7.3-7.6(7H, m), 10.07(1H, brs)

ESI/MS: 432(M+H)⁺, 454 (M+Na)⁺

Elemental Analysis for C₂₄H₂₅N₅OS

Calcd. C: 66.80, H: 5.84, N: 16.23

Found C: 66.90, H: 5.87 N: 16.32

### EXAMPLE 76

A solution of 6-[1-chloro-2-(4-fluorophenyl)-2-oxoethyl]-2-isopropyl-3(2H)-pyridazinone (300 mg) and thiourea (88.8 mg) in dimethylformamide (0.6 mL) was heated for 35 hours at 80-85° C. After cooling, a mixture of a saturated sodium hydrogencarbonate (1.5 mL) and water (5 mL) was added to the reaction mixture and the resalting mixture was stirred for one hour. The precipitates were collected by filtration and dried over phosphorus petoxide under reduced pressure to give 4-(4-fluorophenyl)-5-(1-isopropyl-6-oxo-1,6-dihydro-3-pyridazinyl)-1,3-thiazol-2-ylformamide as a solid (324 mg).

m.p.: 230-231° C. (ethanol)

IR (KBr): 1736, 1668, 1587 cm⁻¹

APCI/MS: 358(M+H)⁺, 331

¹H NMR (CDCl₃, δ): 1.38(6H, d, J=6.62 Hz), 5.31(1H, 7-plet, J=6.62 Hz), 6.72(1H, d, J=9.67 Hz), 6.85(1H, d, J=9.67 Hz), 7.13-7.26(2H, m), 7.46-7.57(2H, m), 7.68(1H, s), 12.08(1H, s)

Elemental Analysis for C₁₇HisFN₄O₂S

Calcd. C: 56.97; H: 4.22; N: 15.63

Found C: 57.01; H: 4.26; N: 15.68

### EXAMPLE 77

A solution of 6-[1-chloro-2-(4-fluorophenyl)-2-oxoethyl]-2-isopropyl-3(2H)-pyridazinone (300 mg) and thiourea (88.8 mg) in dioxane (0.6 mL) was heated for 20 hours at 80-85° C. After cooling, a mixture of a saturated sodium hydrogencarbonate (1.5 mL) and water (5 mL) was added to the reaction mixture and the resulting mixture was stirred for one hour. The precipitates were collected by filtration and dried over phosphorus petoxide under reduced pressure to give 6-[2-amino-4-(4-fluorophenyl)-1,3-thiazol-5-yl]-2-isopropyl-3(2H)-pyridazinone as a solid (301 mg).

m.p.: 255.5-257° C. (ethanol)

IR (KBr): 3384, 1650, 1582, 1523 cm⁻¹

ESI/MS: 353(M+Na)⁺, 331(M+H)⁺

¹H NMR (DMSO-d₆, δ): 1.23(6H, d, J=6.60 Hz), 5.09(1H, 7-plet, J=6.60 Hz), 6.73(1H, d, J=9.70 Hz), 6.92(1H, d, J=9.70 Hz), 7.18-7.27(2H, m), 7.41(2H, s), 7.44-7.54(2H, m)

Elemental Analysis for C₁₆H₁₅FN₄OS

Calcd. C: 58.17; H: 4.58; N: 16.96

Found C: 58.42; H: 4.65; N: 17.05

### EXAMPLE 78

N-[4-(4-Fluorophenyl)-5-(1-isopropyl-6-oxo-1,6-dihydro-3-pyridazinyl)-1,3-thiazol-2-yl]-benzamide was obtained in a manner similar to Example 77.

m.p.: 228-230° C. (ethanol-n-hexane)

IR (KBr): 3224, 1648, 1579, 1529 cm⁻¹

ESI/MS: 891(2M+Na)⁺, 457(M+Na)⁺, 435(M+H)⁺

¹H NMR (CDCl₃, δ): 1.41(6H, d, J=6.64 Hz), 5.33(1H, 7-plet, J=6.64 Hz), 6.72(1H, d, J=9.71 Hz), 6.95(1H, d, J=9.71 Hz), 7.05-7.15(2H, m), 7.45-7.64(5H, m), 7.91-7.97(2H, m), 9.87(1H, br.s)

Elemental Analysis for C₂₃H₁₉FN₄O₂S

Calcd. C: 63.58; H: 4.41; N: 12.89

Found C: 63.62; H: 4.39; N: 12.89

### EXAMPLE 79

6-[2-Amino-4-(2-fluorophenyl)-1,3-thiazol-5-yl]-2-isopropyl-3(2H)-pyridazinone was obtained in a manner similar to Example 77.

m.p.: 233-235° C. (ethanol)

IR (KBr): 3361, 3280, 3130, 1655, 1587, 1523 cm⁻¹

ESI/MS: 683(2M+Na)⁺, 353(M+Na)⁺, 331(M+H)⁺

¹H NMR (DMSO-d, 6): 1.17(6H, d, J=6.60 Hz), 5.06(1H, 7-plet, J=6.60 Hz), 6.75(1H, d, J=9.90 Hz), 6.88(1H, d, J=9.90 Hz), 7.21-7.32(2H, m), 7.42-7.55(4H, m)

Elemental Analysis for C₁₆HisFN₄OS

Calcd. C: 58.17; H: 4.58; N: 16.96

Found C: 58.06; H: 4.79; N: 16.61

### EXAMPLE 80

6-[2-Amino-4-(3-fluorophenyl)-1,3-thiazol-5-yl]-2-isopropyl-3(2H)-pyridazinone was obtained in a manner similar to Example 77.

m.p.: 237-238° C. (ethanol)

IR (KBr): 3384, 3294, 3134, 1653, 1635, 1581, 1522 cm⁻¹

ESI/MS: 683(2M+Na)⁺, 353(M+Na)⁺, 331(M+H)⁺

¹H NMR (DMSO-ds, 3): 1.23(6H, d, J=6.62 Hz), 5.10(1H, 7-plet, J=6.62 Hz), 6.76(1H, d, J=9.62 Hz), 6.97(1H, d, J=9.62 Hz), 7.21-7.32(3H, m), 7.38-7.50(3H, m)

Elemental Analysis for C₁₆H₁₅FN₄OS

Calcd. C: 58.17; H: 4.58; N: 16.96

Found C: 58.19; H: 4.62; N: 16.95

### EXAMPLE 81

6-[2-Amino-4-(3-chlorophenyl)-1,3-thiazol-5-yl]-2-isopropyl-3(2H)-pyridazinone was obtained in a manner similar to Example 77.

m.p.: 235.5-237° C. (ethanol)

IR (KBr): 3334, 3296, 3091, 1647, 1576, 1533 cm⁻¹

ESI/MS: 371 and 369(M+Na)⁺, 349 and 347(M+H)⁺

¹H NMR (DMSO-d₆, δ): 1.22(6H, d, J=6.62 Hz), 5.10(1H, 7-plet, J=6.62 Hz), 6.77(1H, d, J=9.60 Hz), 7.00(1H, d, J=9.60 Hz), 7.38-7.52(6H, m)

Elemental Analysis for C₁₆H₁₅ClN₄OS

Calcd. C: 55.41; H: 4.36; N: 16.15

Found C: 55.48; H: 4.43; N: 16.10

### EXAMPLE 82

Acetyl chloride (0.855 mL) was added to a solution of 6-[2-amino-4-(4-fluorophenyl)-1,3-thiazol-5-yl]-2-isopropyl-3(2H)-pyridazinone (331 mg) in pyridine (6 mL) at ambient temperature and stirred at the same temperature for 2 hours. Pyridine was removed under reduced pressure to give a syrup. The syrup was dissolved in chloroform, washed with 1N-hydrochloric acid, aqueous sodium hydrogen carbonate solution and brine, dried over magnesium sulfate and concentrated under reduced pressure to give a residue. The residue was purified by column chromatography on silica gel (methanol-dichloromethane 2:98 v/v) to give N-[4-(4-fluorophenyl)-5-(1-isopropyl-6-oxo-1,6-dihydro-3-pyridazinyl)-1,3-thiazol-2-yl]acetamide as a solid (273 mg).

m.p.: 236-237.5° C. (ethanol)

IR (KBr): 1649, 1577, 1550 cm⁻¹

ESI/MS: 767(2M+Na)⁺, 395(M+Na)⁺, 373(M+H)⁺

¹H NMR (DMSO-d₆, δ): 1.26(6H, d, J=6.64 Hz), 2.19(3H, s), 5.13(1H, 7-plet, J=6.64 Hz), 6.82(1H, d, J=9.70 Hz), 7.06(1H, d, J=9.70 Hz), 7.21-7.32(2H, m), 7.50-7.59(2H, m), 12.42(1H, br.s)

Elemental Analysis for C₁₈H₁₇FN₄OS

Calcd. C: 58.05; H: 4.60; N: 15.04

Found C: 58.07; H: 4.61; N: 14.98

### EXAMPLE 83

N-[4-(4-Fluorophenyl)-5-(1-isopropyl-6-oxo-1,6-dihydro-3-pyridazinyl)-1,3-thiazol-2-yl]-benzamide was obtained in a manner similar to Example 82.

m.p.: 202-203.5° C. (ethanol-diisopropyl ether)

IR (KBr): 3234, 3187, 1670, 1583, 1549 cm⁻¹

ESI/MS: 457(M+Na)⁺, 435(M+H)⁺

¹H NMR (CDCl₃, δ): 1.42(6H, d, J=6.58 Hz), 5.33(1H, 7-plet, J=6.58 Hz), 6.73(1H, d, J=9.70 Hz), 6.91(1H, d, J=9.70 Hz), 7.12-7.21(2H, m), 7.46-7.63(5H, m), 8.05-8.18(3H, m)

Elemental Analysis for C₂₃HigFN₄O₂S

Calcd. C: 63.58; H: 4.41; N: 12.89

Found C: 63.62; H: 4.39; N: 12.89

### EXAMPLE 84

A mixture of 6-(1-bromo-2-oxo-2-phenylethyl)-3(2H)-pyridazinone (1.00 g) and thiourea (311 mg) in 1-methyl-2-pyrrolidinone (2 mL) was heated for 6 hours at 80-85° C. The mixture was poured into a saturated sodium hydrogencarbonate solution (3 mL) and the mixture was stirred for one hour to give a solid. The solid was collected by filtration, dried over phosphorous pentoxide and triturated with diisopropyl ether to give 6-(2-amino-4-phenyl-1,3-thiazol-5-yl)-3(2H)-pyridazinone as a solid (0.84 g).

m.p.: >250° C. (ethanol)

IR (KBr): 3311, 3151, 1668, 1647, 1593, 1547, 1510 cm⁻¹

ESI/MS: 563(2M+Na)⁺, 293(M+Na)⁺, 271(M+H)⁺

¹H NMR (DMSO-d₆, o): 6.66(1H, dd, J=1.59, 9.98 Hz), 6.86(1H, d, J=9.98 Hz), 7.37-7.49(7H, m), 12.93(1H, br.s)

Elemental Analysis for C₁₃H₁₀N₄OS

Calcd. C: 57.76; H: 3.73; N: 20.73

Found C: 57.48; H: 3.66; N: 20.55

### EXAMPLE 85

To a solution of 6-(2-amino-4-phenyl-1,3-thiazol-5-yl)-3(2H)-pyridazinone (1010 mg) in dimethylformamide (10 mL) was added sodium hydride (60% in oil) (157 mg), and the mixture was stirred for 30 minutes at 50-55° C. Iodomethane (0.279 mL) was added to the mixture and the resulting mixture wvas stirred for 8 hours at 50-55° C. The mixture was poured into water (100 mL) to give a solid. The solid was collected by filtration, dried over phosphorous pentoxide and purified by a column chromatography on silica gel (n-hexane:ethyl acetate=60:40 and then 20:80, v/v) to give 6-(2-amino-4-phenyl-1,3-thiazol-5-yl)-2-methyl-3(2H)-pyridazinone as a solid (185 mg).

m.p.: 238-241° C. (ethanol-diisopropyl ether)

IR (KBr): 3344, 3122, 1657, 1581, 1522 cm⁻¹

ESI/MS: 591(2M+Na)⁺, 307(M+Na)⁺, 285(M+H)⁺

¹H NMR (DMSO-d₆, δ): 3.62(3H, s), 6.72(1H, d, J=9.76 Hz), 6:86(1H, d, J=9.76 Hz), 7.37-7.47(7H, m)

Elemental Analysis for C₁₄H₁₂N₄OS

Calcd. C: 59.14; H: 4.25; N: 19.70

Found C: 58.95; H: 4.18; N: 19.54

### EXAMPLE 86

6-(2-Amino-4-phenyl-1,3-thiazol-5-yl)-2-propyl-3(2H)-pyridazinone was obtained in a manner similar to Example 85.

m.p.: 224-226° C. (ethanol)

IR (KBr): 3444, 3280, 1649, 1579, 1535 cm⁻¹

ESI/MS: 647(2M+Na)⁺, 335(M+Na)⁺, 313(M+H)⁺

¹H NMR (DMSO-ds, 8): 0.88(3H, t, J=7.38 Hz), 1.65-1.75(2H, m), 3.97 (2H, t, J=7.08 Hz), 6.72(1H, d, J=9.72 Hz), 6.88(1H, d, J=9.72 Hz), 7.38-7.47(7H, m)

Elemental Analysis for C₁₅H₁₄N₄OS.0.1H₂O

Calcd. C: 61.17; H: 5.20; N: 17.83

Found C: 61.21; H: 5.11; N: 17.69

### EXAMPLE 87

6-(2-Amino-4-phenyl-1,3-thiazol-5-yl)-2-(2-methoxyethyl)-3(2H)-pyridazinone was obtained in a manner similar to Example 85.

m.p.: 208-209.5° C. (ethanol)

IR (KBr): 3361, 3097, 1668, 1589, 1522 cm⁻¹

ESI/MS: 679(2M+Na)⁺, 351(M+Na)⁺, 329(M+H)⁺

¹H NMR (DMSO-d₆, δ): 3.25(3H, s), 3.67(2H, t, J=5.64 Hz), 4.18(2H, t, J=5.64 Hz), 6.73(1H, d, J=9.75 Hz), 6.87(1H, d, J=9.75 Hz), 7.39-7.47(7H, m)

Elemental Analysis for C₁₆H₁₆N₄O₂S 0.2H₂O

Calcd. C: 57.89; H: 4.98; N: 16.88

Found C: 57.87; H: 4.81; N: 16.90

### EXAMPLE 88

6-(2-Amino-4-phenyl-1,3-thiazol-5-yl)-2-(cyclopropylmethyl)-3(2H)-pyridazinone was obtained in a manner similar to Example 85.

m.p.: 204-206° C. (ethanol-diisopropyl ether)

IR (KBr): 3354, 3132, 1653, 1581, 1520 cm⁻¹

ESI/MS: 671(2M+Na)⁺, 347(M+Na)⁺, 325(M+H)⁺

¹H NMR (DMSO-d₆, δ): 0.34-0.39(2H, m), 0.46-0.52(2H, m), 1.19-1.23(1H, m), 3.87(2H, d, J=7.16 Hz), 6.73(1H, d, J=9.74 Hz), 6.89(1H, d, J=9.74 Hz), 7.39-7.48(7H, m)

Elemental Analysis for C₁₇H₁₆N₄OS.0.15H₂O

Calcd. C: 62.42; H: 5.02; N: 17.13

Found C: 62.93; H: 5.12; N: 16.82

### EXAMPLE 89

Methyl [3-(2-amino-4-phenyl-1,3-thiazol-5-yl)-6-oxo-1 (6H)-pyridazinyl]acetate was obtained in a manner similar to Example 85.

m.p.: 190-193° C. (ethanol-diisopropyl ether)

IR (KBr): 3427, 3103, 1734, 1672, 1591, 1522 cm⁻¹

ESI/MS: 365(M+Na)⁺, 343(M+H)⁺

¹H NMR (DMSO-d₆, δ): 3.69(3H, s), 4.83(2H, s), 6.79(1H, d, J=9.80 Hz), 6.92(1H, d, J=9.80 Hz), 7.40-7.51(7H, m)

### EXAMPLE 90

6-(2-Amino-4-phenyl-1,3-thiazol-5-yl)-2-(2-oxopropyl)-3(2H)-pyridazinone was obtained in a manner similar to Example 85.

m.p.: 216-219° C. (ethanol-diisopropyl ether)

IR (KBr): 3417, 3093, 1728, 1672, 1593, 1522 cm⁻¹

ESI/MS: 349(M+Na)⁺, 327(M+H)⁺

¹H NMR (DMSO-d₆, δ): 2.20(3H, s), 4.94(2H, s), 6.77(1H, d, J=9.80 Hz), 6.91(1H, d, J=9.80 Hz), 7.39-7.47(7H, m)

Elemental Analysis for C₁₆H₁₄N₄O₂S 0.2H₂O

Calcd. C: 58.24; H: 4.40; N: 16.98

Found C: 58.14; H: 4.26; N: 16.79

### EXAMPLE 91

To a solution of 6-(2-amino-4-phenyl-1,3-thiazol-5-yl)-3(2H)-pyridazinone (300 mg) in dimethylformamide (1.8 mL) was added sodium hydride (60% in oil) (46.6 mg), and the mixture was stirred for 30 minutes at 50-55° C. Iodoethane (0.259 mL) was added to the mixture, and the resulting mixture was stirred for 10 hours at 50-55° C. The mixture was poured into water (15 mL) to give a solid. The solid was collected by filtration, dried over phosphorous pentoxide and purified by a column chromatography on silica gel with eluting with a mixture of n-hexane and ethyl acetate (80:20, v/v) to give 6-[2-(diethylamino)-4-phenyl-1,3-thiazol-5-yl]-2-ethyl-3(2H)-pyridazinone as a syrup (6 mg) and eluting with a mixture of n-hexane and ethyl acetate (60:40 v/v) to give 2-ethyl-6-[2-(ethylamino)-4-phenyl-1,3-thiazol-5-yl]-3(2H)-pyridazinone as a solid (11 mg), and eluting wih a mixture of n-hexane and ethyl acetate (20:80 v/v) to give 6-(2-amino-4-phenyl-1,3-thiazol-5-yl)-2-ethyl-3(2H)-pyridazinone as a solid (213 mg). 6-[2-(diethylamino)-4-phenyl-1,3-thiazol-5-yl]-2-ethyl-3(2H)-pyridazinone

ESI/MS: 731(2M+Na)⁺, 377(M+Na)⁺, 355(M+H)⁺

¹H NMR (CDCl₃, δ): 1.26(6H, t, J=7.10 Hz), 1.40(3H, t, J=7.20 Hz), 3.55(4H, q, J=7.10 Hz), 4.19(2H, q, J=7.20 Hz), 6.59(1H, d, J=9.72 Hz), 6.84(1H, d, J=9.72 Hz), 7.37-7.41(3H, m), 7.51-7.54(2H, m) 2-ethyl-6-[2-(ethylamino)-4-phenyl-1,3-thiazol-5-yl]-3(2H)-pyridazinone

m.p.: 0.160-163° C. (diisopropyl ether)

IR (KBr): 3199, 2968, 166, 1583 cm⁻¹

ESI/MS: 675(2M+Na)⁺, 349(M+Na)⁺, 327(M+H)⁺

¹H NMR (CDCl₃, δ): 1.20(3H, t, J=6.68 Hz), 1.40(3H, t, J=7.20 Hz), 3.21-3.26(2H, m), 4.19(2H, q, J=7.20 Hz), 6.15(1H, br.s), 6.60(1H, d, J=9.72 Hz), 6.84(1H, d, J=9.72 Hz), 7.37-7.41(3H, m), 7.46-7.51(2H, m) 6-(2-amino-4-phenyl-1,3-thiazol-5-yl)-2-ethyl-3(2H)-pyridazinone

m.p.: 232-235° C. (ethanol-diisopropyl ether)

IR (KBr): 3357, 3124, 1657, 1583, 1522 cm⁻¹

ESI/MS: 619(2M+Na)⁺, 321(M+Na)⁺, 299(M+H)⁺

¹H NMR PDMSO-d₆, δ): 1.25(3H, t, J=7.16 Hz), 4.00-4.07(2H, m), 6.71(1H, d, J=9.72 Hz), 6.87(1H, d, J=9.72 Hz), 7.38-7.47(7H, m)

Elemental Analysis for C₁₅H₁₄N₄OS.0.2H₂O

Calcd. C: 59.66; H: 4.81; N: 18.55

Found C: 59.77; H: 4.61; N: 18.47

### EXAMPLE 92

6-(2-Amino-4-phenyl-1,3-thiazol-5-yl)-2-benzyl-3(2H)-pyridazinone and 2-benzyl-6-[2-(benzylamino)-4-phenyl-1,3-thiazol-5-yl]-3(2H)-pyridazinone were obtained in a manner simialar to Example 91.

6-(2-amino-4-phenyl-1,3-thiazol-5-yl)-2-benzyl-3(2H)-pyridazinone

m.p.: 225-228° C. (ethanol-diisopropyl ether)

IR (KBr): 1653, 1585 cm⁻¹

ESI/MS: 743(2M+Na)⁺, 383(M+Na)⁺, 361(M+H)⁺

¹H NMR PMSO-d₆, δ): 5.20(2H, s), 6.77(1H, d, J=9.76 Hz), 6.89(1H, d, J=9.76 Hz), 7.29-7.51(12H, m)

Elemental Analysis for C₂₀Ht6N₄OS.0.5H₂O

Calcd. C: 65.02; H: 4.64; N: 15.17

Found. C: 65.37; H: 4.39; N: 14.75

2-benzyl-6-[2-(benzylamino)-4-phenyl-1,3-thiazol-5-yl]-3(2H)-pyridazinone

m.p.: 163.5-165° C. (ethanol-diisopropyl ether)

IR (KBr): 3188, 1657, 1576 cm⁻¹

ESI/MS: 923(2M+Na)⁺, 473(M+Na)⁺, 451(M+H)⁺

¹H NMR (DMSO-d₆, δ): 4.47(2H, d, J=5.20 Hz), 5.27(2H, s), 6.10(1H, br.s), 6.60(1H, d, J=9.76 Hz), 6.84(1H, d, J=9.76 Hz), 7.30-7.48(15H, m)

Elemental Analysis for C₂₇H₂₂N₄OS.0.4H₂O

Calcd. C: 70.84; H: 5.02; N: 12.24

Found C: 70.86; H: 4.76; N: 12.26

### EXAMLE 93

2-Allyl-6-(2-amino-4-phenyl-1,3-thiazol-5-yl)-3(2H)-pyridazinone, 2-allyl-6-[2-(allylamino)-4-phenyl-1,3-thiazol-5-yl]-3(2H)-pyridazinone and 2-allyl-6-[2-(diallylamino)-4-phenyl-1,3-thiazol-5-yl]-3(2H)-pyridazinone were obtained in a manner similar to Example 91.

2-allyl-6-(2-amino-4-phenyl-1,3-thiazol-5-yl)-3 (2H)-pyridazinone

m.p.: 212-215° C. (ethanol)

IR (KBr): 3373, 3097, 1655, 1581, 1520 cm⁻¹

ESI/MS: 643(2M+Na)⁺, 333(M+Na)⁺, 311(M+H)⁺

¹H NMR (DMSO-d₆, δ): 4.62(2H, d, J=5.60 Hz), 5.12-5.23(2H, m), 5.89-5.99(1H, m), 6.75(1H, d, J=9.78 Hz), 6.89(1H, d, J=9.78 Hz), 7.38-7.48(7H, m)

Elemental Analysis for C₁₆H₁₄N₄OS.0.1H₂O

Calcd. C: 61.56; H: 4.58; N: 17.95

Found C: 61.43; H: 4.38; N: 17.87

2-allyl-6-[2-(allylamino)-4-phenyl-1,3-thiazol-5-yl]-3 (2H)-pyridazinone

m.p.: 146-147° C. (ethanol-diisopropyl ether)

IR (KBr): 3190, 1672, 1574 cm⁻¹

ESI/MS: 732(2M+Na)⁺, 373(M+Na)⁺, 351(M+H)⁺

¹H NMR (CDCl₃, δ): 3.89-3.92(2H, m), 4.75(2H, d, J=6.00 Hz), 5.22-5.37(4H, m), 5.76(1H, br.s), 5.87-6.07(2H, m), 6.62(1H, d, J=9.72 Hz), 6.87(1H, d, J=9.72 Hz), 7.38-7.41(3H, m), 7.47-7.51(2H, m)

Elemental Analysis for CigHlsN₄OS.0.2H₂O

Calcd. C: 64.46; H: 5.24; N: 15.82

Found C: 64.61; H: 5.07; N: 15.87

2-allyl-6-[2-(diallylamino)-4-phenyl-1,3-thiazol-5-yl]-3(2H)-pyridazinone

ESI/MS: 803(2M+Na)⁺, 413(M+Na)⁺, 391(M+H)⁺

¹H NMR (CDCl₃, δ): 4.12(4H, d, J=5.76 Hz), 4.73-4.76(2H, m), 5.24-5.32(4H, m), 5.81-6.12(3H, m), 6.62(1H, d, J=9.72 Hz), 6.87(1H, d, J=9.72 Hz), 7.37-7.39(3H, m), 7.50-7.54(2H,m)

### EXAMPLE 94

Formic acid (66 mg) was added to a solution of acetic anhydride (74 mg) in dichloromethane (3 ml) under ice-bath cooling. After 30 minutes, 6-(2-amino-4-phenyl-1,3-thiazol-5-yl)-2-isopropyl-3 (2H)-pyridazinone (150 mg) was added to the reaction mixture. The mixture was stirred for 30 minutes with ice-bath cooling, and then stirred for 1 hour at ambient temperature. Formic acid (0.16 ml) and acetic anhydride (0.2 ml) were added to the mixture. The reaction mixture was stirred overnight at ambient temperature. An aqueous sodium hydrogencarbonate solution was added to the reaction mixture, the resulting mixture was extracted with ethyl acetate. The separated organic layer was dried over sodium sulfate. The solvent was removed in vacuo to give a yellow powder, which was collected by filtration to afford 5-(1-isopropyl-6-oxo-1,6-dihydro-3-pyridazinyl)-4-phenyl-1,3-thiazol-2-ylformamide as yellow powder (80 mg).

mp 232-234° C. (ethanol)

IR (KBr): 3451, 3033, 1695, 1662, 1585 cm⁻¹

¹H NMR (DMSO-d₆, δ): 1.28(6H, d, J=6.6 Hz), 5.13(1H, 7-plet, J=6.6 Hz), 6.81(1H, d, J=9.6 Hz), 7.02(1H, d, J=9.6 Hz), 7.3-7.55(5H, m), 8.59(1H, s), 12.2-13.0 (1H, br)

ESI/MS: 341(M+H)⁺, 363 (M+Na)⁺

Elemental Analysis for C₁₇H₁₆N₄O₂S

Calcd. C: 59.98, H: 4.74, N: 16.46

Found C: 60.06, H: 4.78, N: 16.48

### EXAMPLE 95

A mixture of 6-(2-amino-4-phenyl-1,3-thiazol-5-yl)-2-isopropyl-3(2H)-pyridazinone (232 mg), di-t-butyloxycarbonate (170 mg) and triethylamine (90 mg) in dichloromethane (5 ml) was stirred at ambient temperature. 4-Dimethylaminopyridine (50 mg) was added to the reaction mixture under same conditions. After 12 hours, water and ethyl acetate were added to the mixture. The separated organic layer was dried over diatomaceous earth. The solvent was removed in vacuo to give a yellow powder, which was objected to a column chlomatography on silica gel eluting with a mixture of n-hexane and ethyl acetate. The solvent was removed in vacuo to afford a yellow powder, which was suspended in diisopropyl ether with stiring. The powder was collected by filtration to afford tert-butyl 5-(1-isopropyl-6-oxo-1,6-dihydro-3-pyridazinyl)-4-phenyl-1,3-thiazol-2-ylcarbamate (91 mg).

mp 198-199° C. (ethanol)

IR (KIr): 3154, 1710, 1648, 1581 cm⁻¹

¹H NMR (CDCl₃, δ): 1.39(6H, d, J=6.7 Hz), 1.52(9H, s), 5.31(1H, 7-plet, J=6.7 Hz), 6.66(1H, d, J=9.6 Hz), 6.92(1H, d, J=9.6 Hz), 7.3-7.55(5H, m), 8.51(1H, br)

ESI/MS: 435 (M+Na)⁺

Elemental Analysis for C₂₁H₂₄N₄O₃S

Calcd. C: 61.15, H: 5.86, N: 13.58

Found C: 60.83, H: 6.21, N: 13.29

### EXAMPLE 96

A mixture of 4-(chloromethyl)-N-[5-(1-isopropyl-6-oxo-1,6-dihydro-3-pyridazinyl)-4-phenyl-1,3-thiazol-2-yl]benzamide (100 mg) and 2-methoxyethylamine (50 mg) in dioxane (1 ml) was stirred overnight at ₈₀° C. Ethyl acetate and an aqueous sodium hydrogencarbonate solution were added to the reaction mixture at ambient temperature. The separated organic layer was dried over sodium sulfate. The solvent was removed in vacuo to give a yellow powder, which was objected to a column chlomatography on silica gel eluting with a mixture of chloroform and methanol. The solvent was removed in vacuo to afford a yellow powder, which was suspended in diisopropyl ether with stirring. The powder was collected by filtration to afford N-[5-(1-isopropyl-6-oxo-1,6-dihydro-3-pyridazinyl)-4-phenyl-1,3-thiazol-2-yl]-4-([(2-methoxyethyl)amino]methyl}benzamide as white powder (10 mg).

mp 192-194° C. (diisopropyl ether)

IR (KBr): 3421, 1648, 1577 cm⁻¹

¹H NMR (DMSO-d₆, δ): 1.30(6H, d, J=6.6 Hz), 2.3-3.8(7H, m), 4.07(2H, s), 5.12(1H, 7-plet, J=6.6 Hz), 6.83(1H, d, J=9.7 Hz), 7.04(1H, d, J=9.7 Hz), 7.3-7.7(7H, m), 8.0-8.2(2H, m)

APCI/MS: 504(M+H)⁺, 526 (M+Na)⁺

### EXAMPLE 97

N-(5-(1-Isopropyl-6-oxo-1,6-dihydro-3-pyridazinyl)-4-phenyl-1,3-thiazol-2-yl]-4-[(4-methyl-1-piperazinyl)methyl]benzamide was obtained in a manner similar to Example 96.

mp 224-227° C. (diisopropyl ether)

IR (KBr): 3444, 1648 cm⁻¹

¹H NMR (DMSO-d₆, δ): 1.30(6H, d, J=6.6 Hz), 2.16(3H, s), 2.2-2.5(8H, m), 3.54(2H,s), 5.15(1H, 7-plet, J=6.6 Hz), 6.82(1H, d, J=9.6 Hz), 7.03(1H, d, J=9.6 Hz), 7.3-7.6(7H, m), 8.0-8.15(2H, m), 12.6-13.2(1H, brs)

ESI/MS: 529 (M+H)⁺, 551 (M+Na)⁺

Elemental Analysis for C₂₉H₃₂N₆O₂S

Calcd. C: 64.78, H: 6.18, N: 15.63

Found C: 64.76, H: 6.17, N: 15.53

### EXAMPLE 98

N-[5-(1-isopropyl-6-oxo-1,6-dihydro-3-pyridazinyl)-4-phenyl-1,3-thiazol-2-yl]-4-(1-pyrrolidinylmethyl)benzamide was obtained in a manner simnilar to Example 96.

mp 221-222° C. (diisopropyl ether)

IR (KBr): 3421, 1650 cm⁻¹

¹H NMR (PMSO-d₆, δ): 1.30(6H, d, J=6.6 Hz), 1.7-1.9(4H, m), 2.6-2.8(4H, m), 3.91(2H,s), 5.15(1H, 7-plet, J=6.6 Hz), 6.82(1H, d, J=9.6 Hz), 7.04(1H, d, J=9.6 Hz), 7.3-7.7(7H, m), 8.0-8.15(2H, m), 10-13(1H, br)

ESI/MS: 500(M+H)⁺, 522 (M+Na)⁺

Elemental Analysis for C₂₈H₂₉NsO₂S 2.6H₂O

Calcd. C: 61.54, H: 6.31, N: 12.82

Found C: 61.47, H: 6.06, N: 13.00

### EXAMPLE 99

A mixture of 2-isopropyl-6-[2-(methylamino)-4-phenyl-1,3-thiazol-5-yl]-3(2H)-pyridazinone hydrobromide (111 mg), 3-tolylisocyanate (40 mg) and triethylamine (33 mg) in dioxane (3 ml) was stirred for 3 hours at ambient temperature. Water and ethyl acetate were added to the reaction mixture at ambient temperature. The separated organic layer was dried over diatomaceous earth. The solvent was removed in vacuo to give a yellow powder, which was objected to a column chlomatography on silica gel eluting with a mixture of chloroform and methanol. The solvent was removed in vacuo to afford a yellow powder, which was suspended in diisopropyl ether with stirring. The powder was collected by filtration to afford N-[5-(1-isopropyl-6-oxo-1,6-dihydro-3-pyridazinyl)-4-phenyl-1,3-thiazol-2-yl]-N-methyl-N′-(3-methylphenyl)urea as yellow white powder (11 mg).

mp 157-158° C. (diisopropyl ether)

IR (KBr):3565, 1683, 1656 cm⁻¹

¹H NMR (DMSO-d₆, δ): 1.28(6H, d, J=6.6 Hz), 2.32(3H, s) 3.73(3H, s), 5.14(1H, 7-plet, J=6.6 Hz), 6.80(1H, d, J=9.7 Hz), 7.01(1H, d, J=9.7 Hz), 7.0-7.6(9H, m), 9.37(1H, brs)

ESI/MS: 460(M+H)⁺, 482 (M+Na)⁺

Elemental Analysis for C₂₅H₂₅NsO₂S.0.1H₂O

Calcd. C: 65.08, H: 5.51, N: 15.18

Found C: 65.28, H: 5.56, N: 14.80

### EXAMPLE 100

A mixture of N-[5-(1-isopropyl-6-oxo-1,6-dihydro-3-pyridazinyl)-4-phenyl-1,3-thiazol-2-yl]-2-chloroacetamide (200 mg) and 4-aminomethylpyridine (278 mg) in dioxane (4 ml) was stirred overnight at 50° C. Water and ethyl acetate were added to the reaction niixture at ambient temperature. The separated organic layer was dried over diatomaceous earth. The solvent was removed in vacuo to give a yellow powder, which was objected to a column chiomatography on silica gel eluting with a mixture of chloroform and methanol. The solvent was removed in vacuo to afford a yellow powder, which was suspended in diisopropyl ether with stirring. The powder was collected by filtration to give N-[5-(1-isopropyl-6-oxo-1,6-dihydro-3-pyridazinyl)-4-phenyl-1,3-thiazol-2-yl]-2-[(4-pyridinylmethyl)amino]acetamide as a yellow white powder (105 mg).

mp: 187-188° C. (diisopropyl ether)

IR (KBr): 3336, 1658, 1581 cm⁻¹

¹H NMR (DMSO-d₆, δ): 1.28(6H, d, J=6.6 Hz), 3.51(2H, s), 3.81(2H, s), 5.14(1H, 7-plet, J=6.6 Hz), 6.81(1H, d, J=9.7 Hz), 7.02(1H, d, J=9.7 Hz), 7.3-7.6(8H, m), 8.05(2H, dd, J.=1.5 Hz and 4.5 Hz)

ESI/MS: 461(M+H)⁺, 483(M+Na)⁺

### EXAMPLE 101

N-[5-(1-Isopropyl-6-oxo-1,6-dihydro-3-pyridazinyl)-4-phenyl-1,3-thiazol-2-yl]-2-[(2-pyridinylmethyl)aininolacetamide dihydrochloride was obtained in a manner similar to Example 100.

mp: 252-254° C. (diisopropyl ether)

IR (KBr): 1648 cm⁻¹

¹H NMR (DMSO-d₆, δ): 1.28(6H, d, J=6.6 Hz), 4.23(2H, brs), 4.49(2H, brs), 5.15(1H, 7-plet, J=6.6 Hz), 6.83(1H, d, J=9.7 Hz), 7.04(1H, d, J=9.7 Hz), 7.3-7.6(7H, m), 7.85-8.0(1H, m), 8.67 (1H, dd, J=0.8 Hz and 4.2 Hz), 9.6-10.2(1H, br), 12.6-13.4(1H, br)

ESI/MS: 461(M−2HCl+H)⁺, 483 (M−2HCl+Na)⁺

Elemental Analysis for C₂₈H₂₅N₅O₃S.0.3H₂O

Calcd. C: 64.55, H: 5.73, N: 13.44

Found C: 64.72, H: 5.90, N: 12.97

### EXAMPLE 102

2-(1H-Imidazol-1-yl)-N-[5-(1-isopropyl-6-oxo-1,6-dihydro-3-pyridazinyl)-4-phenyl-1,3-thiazol-2-yl]acetamide was obtained in a manner similar to Example 100.

mp: 160-161° C. (ethanol)

IR (KBr): 3451, 1698, 1656 ca-r

¹H NMR (DMSO-d₆, δ): 1.25(6H, d, J=6.6 Hz), 5.08(2H, s), 5.15(1H, 7-plet, J=6.6 Hz), 6.80(1H, d, J=9.7 Hz), 6.92(1H, s), 7.02(1H, d, J=9.7 Hz), 7.19(1H, s), 7.3-7.6(5H, m), 7.66 (1H, s), 12.81(1H, br)

ESI/MS: 421(M+H)⁺, 443 (M+Na)⁺

Elemental Analysis for C₂₁H₂₀N₆O₂S 0.8H₂O

Calcd. C: 58.00, H: 5.01, N: 19.32

Found C: 58.05, H: 5.05, N: 19.26

### EXAMPLE 103

2-(Benzylamino)-N-[5-(1-isopropyl-6-oxo-1,6-dihydro-3-pyridazinyl)-4-phenyl-1,3-thiazol-2-yl]acetamide was obtained in a manner similar to Example 100.

mp: 144-145° C. (ethanol)

IR (KBr): 3286, 1677, 1658 cm⁻¹

¹H NMR (DMSO-d₆, δ): 1.28(6H, d, J=6.6 Hz), 3.48(2H, s), 3.76(2H, s), 5.14(1H, 7-plet, J=6.6 Hz), 6.81(1H, d, J=9.7 Hz), 7.02(1H, d, J=9.7 Hz), 7.1-7.6(12H, m)

ESI/MS: 460(M+H)⁺, 482 (M+Na)⁺

Elemental Analysis for C₂₅H₂₅NSO₂S

Calcd. C: 65.34, H: 5.48, N: 15.24

Found C: 65.24, H: 5.50 N: 15.24

### EXAMPLE 104

N-[5-(1-Isopropyl-6-oxo-1,6-dihydro-3-pyridazinyl)-4-phenyl-1,3-thiazol-2-yl]-2-[(2-methoxyethyl)amino]acetamide hydrochloride was obtained in a manner similar to Example 100.

mp: 252-253° C. (ethyl acetate)

IR (KBr): 3444, 1668, 1658 cm⁻¹

¹H NMR PMSO-d₄, δ): 1.28(6H, d, J=6.6 Hz), 3.2-3.3(2H, br), 3.4-3.7(5H, m), 4.13(2H, s), 5.14(1H, 7-plet, J=6.6 Hz), 6.83(1H, d, J=9.7 Hz), 7.04(1H, d, J=9.7 Hz), 7.4-7.6(5H, m), 9.44(2H, br), 13.01(1H, brs)

ESI/MS: 428(M−HCl+H)⁺, 450 (M−HCl+Na)⁺

Elemental Analysis for C₂₁H₂₆ClN₅O₃S.1.0H₂O

Calcd. C: 52.33, H: 5.86, N: 14.53

Found C: 52.39, H: 5.77, N: 14.60

### EXAMPLE 105

N-[5-(1-Isopropyl-6-oxo-1,6-dihydro-3-pyridazinyl)-4-phenyl-1,3-thiazol-2-yl]-2-(4-methyl-1-piperazinyl)acetamide dihydrochloride was obtained in a manner similar to Example 100.

mp: 244-246° C. (diisopropyl ether)

IR (KBr): 3428, 1648, cm⁻¹

¹H NMR (DMSO-d₆, δ): 1.28(6H, d, J=6.6 Hz), 2.82(3H, s), 3.3-3.7(8H, m), 4.20(2H, s), 5.14(1H, 7-plet, J=6.6 Hz), 6.82(1H, d, J=9.67 Hz), 7.02(1H, d, J=9.6 Hz), 6.8-7.3(2H, m), 7.3-7.6(5H, m), 13.01(1H, brs)

ESI/MS: 453(M−2HCl+H)⁺, 475 (M−2HCl+Na)⁺

### EXAMPLE 106

N-[5-(1-Isopropyl-6-oxo-1,6-dihydro-3-pyridazinyl)-4-phenyl-1,3-thiazol-2-yl]-2-(4-morpholinyl)acetamide hydrochloride was obtained in a manner similar to Example 100.

mp: 252-253° C. (ethyl acetate)

IR (KBr): 3426, 1670, 1658 cm⁻¹

¹H NMR (DMSO-d₆, δ): 1.28(6H, d, J=6.6 Hz), 3.2-3.5(4H, br), 3.8-4.0(4H, m), 4.2-4.4(2H, brs), 5.14(1H, 7-plet, J=6.6 Hz), 6.83(1H, d, J=9.7 Hz), 7.03(1H, d, J=9.7 Hz), 7.4-7.55(5H, m), 11.15(1H, brs), 13.13(1H, brs)

ESI/MS: 440(M−HCl+H)⁺, 462 (M−HCl+Na)⁺

Elemental Analysis for C₂₂H₂₆ClN₅O₃S.6.9H₂O

Calcd. C: 53.69, H: 5.69, N: 14.23

Found C: 53.69, H: 5.67, N: 14.13

### EXAMPLE 107

N-[5-(1-Isopropyl-6-oxo-1,6-dihydro-3-pyridazinyl)-4-phenyl-1,3-thiazol-2-yl]-2-(1-pyrrolidinyl)acetamide hydrochloride was obtained in a manner similar to Example 100.

mp: >250° C. (ethyl acetate)

IR (KBr): 3423, 1668, 1656 cm⁻¹

¹H NMR (DMSO-d₆, δ): 1.28(6H, d, J=6.6 Hz), 1.8-2.1(4H, m), 3.0-3.3(2H, m), 3.4-3.8(2H, m), 4.42 (2H, s), 5.14(1H, 7-plet, J=6.6 Hz), 6.82(1H, d, J=9.7 Hz), 7.03(1H, d, J=9.7 Hz), 7.35-7.6(5H, m), 10.92(1H, brs), 13.09(1H, brs)

ESI/MS: 424(M−HCl+H)⁺, 446(M−HCl+Na)⁺

Elemental Analysis for C₂₂H₂₆ClNs0₂S.0.8H₂O

Calcd. C: 55.70, H: 5.86, N: 14.76

Found C: 55.79, H: 5.78, N: 14.76

### EXAMPLE 108

2-(Dimethylamino)-N-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: 232-234° C. (ethyl acetate)

IR (KBr): 3421, 1662 cm⁻¹

¹H NMR (DMSO-d₆, δ): 1.28(6H, d, J=6.6 Hz), 2.92(6H, s), 4.33(2H, s), 5.14(1H, 7-plet, J=6.6 Hz), 6.83(1H, d, J=9.7 Hz), 7.03(1H, d, J=9.7 Hz), 7.4-7.6(5H, m), 10.57(1H, brs), 13.13(1H, brs)

ESI/MS: 398(M−HCl+H)⁺, 420(M−HCl+Na)⁺

Elemental Analysis for C₂₀H₂₄ClN₅O₂S.2.2H₂O

Calcd. C: 50.72, H: 6.04, N: 14.79

Found C: 50.61, H: 5.96, N: 14.70

### EXAMPLE 109

N-[5-(1-Isopropyl-6-oxo-1,6-dihydro-3-pyridazinyl)-4-phenyl-1,3-thiazol-2-yl]-2-{[3-(2-oxo-1-pyrrolidinyl)propyl]amino}acetamide hydrochloride was obtained in a manner similar to Example 100.

mp: 207-209° C. (diisopropyl ether)

IR (KBr): 3424, 1698, 1646 cm⁻¹

¹H NMR (DMSO-d₆, δ): 1.27(6H, d, J=6.6 Hz), 1.8-2.0(4H, m), 2.2-2.3(2H, m), 2.9-3.05(2H, br), 3.2-3.3(2H, m), 3.3-3.4(2H, m), 4.05-4.2(2H, m), 5.14(1H, 7-plet, J=6.6 Hz), 6.83(1H, d, J=9.7 Hz), 7.04(1H, d, J=9.7 Hz), 7.4-7.6(5H, m), 9.37(2H, br)

ESI/MS: 495(M−HCl+H)⁺, 517(M−HCl+Na)⁺

### EXAMPLE 110

2-[(2-Hydroxypropyl)amino]-N-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: 209-211° C. (diisopropyl ether)

IR (KBr): 3421, 1664 cm⁻¹

¹H NMR (DMSO-d₆, δ): 1.13(3H, d, J=6.3 Hz), 1.27(6H, d, J=6.6 Hz), 2.85-3.0(11H, m), 3.05-3.15(1H, m), 3.95-4.05(1H, m), 4.13(2H, s), 5.14(1H, 7-plet, J=6.6 Hz), 5.3-5.5(1H, br), 6.83(1H, d, J=9.7 Hz), 7.03(1H, d, J=9.7 Hz), 7.4-7.6(5H, m), 8.99(1H, brs), 9.37(1H, brs), 13.0(1H, br)

ESI/MS: 428(M−HCl+H)⁺, 450(M−HCl+Na)⁺

Elemental Analysis for C₂₁H₂₆ClN₅O₃S.1.2H₂O

Calcd. C: 51.92, H: 5.89, N: 14.42

Found C: 51.92, H: 5.78, N: 14.28

### EXAMPLE 111

N-[5-(1-Isopropyl-6-oxo-1,6-dihydro-3-pyridazinyl)-4-phenyl-1,3-thiazol-2-yl]-2-{methyl[2-(2-pyridinyl)ethyl]amino}acetamide was obtained in a manner similar to Example 100.

mp: 94-96° C. (diisopropyl ether)

IR (KBr): 1666 cm⁻¹

¹H NMR (DMSO-d₆, δ): 1.28(6H, d, J=6.6 Hz), 2.32(3H, s), 2.8-3.0(4H, m), 3.43(2H, s), 5.14(1H, 7-plet, J=6.6 Hz), 6.81(1H, d, J=9.7 Hz), 7.03(1H, d, J=9.7 Hz), 7.15-7.25(1H, m), 7.25-7.35(1H, m), 7.4-7.6(5H, m), 7.71(1H, t, J=7.6 Hz), 8.67(1H, d, J=7.6 Hz), 12.3-12.6(1H, br)

ESI/MS: 489(M+H)⁺, 511(M+Na)⁺

### EXAMPLE 112

2-[(2-Hydroxy-2-phenylethyl)amino]-N-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: 218-220° C. (diisopropyl ether)

IR (KBr): 3421, 1666, 1650 cm⁻¹

¹H NMR (DMSO-d₆, δ): 1.28(6H, d, J=6.6 Hz), 3.1-3.4(2H, m), 4.18(2H, s), 5.02(1H, dd, J=2.7 Hz and 10.2 Hz), 5.14(1H, 7-plet, J=6.6 Hz), 6.1-6.3(1H, br), 6.83(1H, d, J=9.7 Hz), 7.04(1H, d, J=9.7 Hz), 7.2-7.6(10H, m), 9.0-9.2(1H, br), 9.4-9.7 (1H, br), 13.0(1H, s)

ESI/MS: 490(M−HCl+H)⁺, 512(M−HCl+Na)⁺

Elemental Analysis for C₂₆H₂₅ClN₅O₃S.1.0H₂O

Calcd. C: 57.40, H: 5.56, N: 12.87

Found C: 57.41, H: 5.36, N: 12.77

### EXAMPLE 113

2-[(3-Hydroxypropyl)amino]-N-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: 136-142° C. (ethyl acetate)

IR (KBr): 3421, 1648 cm⁻¹

¹H NMR (DMSO-d₆, δ): 1.27(6H, d, J=6.6 Hz), 1.75-1.9(2H, m), 3.0-3.15(2H, m), 3.4-3.6(2H, m), 4.05-4.2(2H, m), 5.14(1H, 7-plet, J=6.6 Hz), 6.83(1H, d, J=9.7 Hz), 7.04(1H, d, J=9.7 Hz), 7.35-7.6(5H, m), 9.33(2H, br), 12.8-13.2(1H, br)

ESI/MS: 428(M−HCl+H)⁺, 550(M−HCl+Na)⁺

### EXAMPLE 114

2-({2-[4-(Amirosulfonyl)phenyl]ethyl}amino)-N-[5-(1-isopropyl-6-oxo-1,6-dihydro-3-pyridazinyl)-4-phenyl-1,3-thiazol-2-yl]acetamide was obtained in a manner similar to Example 100.

mp: 104-106° C. (diisopropyl ether)

IR (KBr): 3253, 3224, 1650 cm⁻¹

¹H NMR (DMSO-dr, 6): 1.27(6H, d, J=6.6 Hz), 2.75-2.9(4H, m), 3.51(2H, s), 5.13(1H, 7-plet, J=6.6 Hz), 6.81(1H, d, J=9.7 Hz), 7.01(1H, d, J=9.7 Hz), 7.2-7.35(3H, br), 7.35-7.55(7H, m), 7.74(2H, d, J=8.3 Hz)

ESI/MS: 553(M+H)⁺, 575(M+Na)⁺

### EXAMPLE 115

2-[(2,3-Dihydroxypropyl)aniino]-N-[5-(1-isopropyl-6-oxo-1,6-dihydro-3-pyridazinyl)-4-phenyl-1,3-thiazol-2-yl]acetamide was obtained in a manner similar to Example 100.

mp: 147-149° C. (diisopropyl ether)

IR (KBr): 3419, 1650 cm⁻¹

¹H NMR (DMSO-d₆, δ): 1.27(6H, d, J=6.6 Hz), 2.5-2.7(1H, m), 2.85-3.0(1H, m), 3.5-3.9(3H, m), 4.5-4.7(1H, m), 4.9-5.1(1H, m), 5.13(1H, 7-plet, J=6.6 Hz), 6.79(1H, d, J=9.7 Hz), 6.98(1H, d, J=9.7 Hz), 7.35-7.6(5H, br)

ESI/MS: 444(M+H)⁺, 466(M+Na)⁺

### EXAMPLE 116

N-[5-(1-Isopropyl-6-oxo-1,6-dihydro-3-pyridazinyl)-4-phenyl-1,3-thiazol-2-yl]-2-{[3-(4-morpholinyl)propyl]amino}acetamide dihydrochloride was obtained in a manner similar to Example 100.

mp: 211-213° C. (diisopropyl ether)

IR (KBr): 3451, 1662, 1648 cm⁻¹

¹H NMR (DMSO-dr, 6): 1.27(6H, d, J=6.6 Hz), 2.1-2.25(2H, m), 2.9-3.4(8H, m), 3.7-4.05(4H, m), 4.15(2H, s), 5.14(1H, 7-plet, J=6.6 Hz), 6.83(1H, d, J=9.7 Hz), 7.04(1H, d, J=9.7 Hz), 7.4-7.6(5H, m), 9.5-9.7(2H, br), 11.0-11.4(1H, br), 12.9-13.20(1H, br)

ESI/MS: 497(M−2HCl+H)⁺, 519(M−2HCl+Na)⁺

Elemental Analysis for C₂₅H₃₄Cl₂N₆O₃S 1.5H₂O

Calcd. C: 50.33, H: 6.25, N: 14.09

Found C: 50.38, H: 6.24, N: 13.92

### EXAMPLE 117

2-[(2-Hydroxyethyl)amino]-N-[5-(1-isopropyl-6-oxo-1,6-dihydro-3-pyridazinyl)-4-phenyl-1,3-thiazol-2-yl]acetamide was obtained in a manner similar to Example 100.

mp: 142-144° C. (diisopropyl ether)

IR (KBr): 3291, 1693, 1648 cm⁻¹

¹H NMR (DMSO-d₆, o): 1.27(6H, d, J=6.6 Hz), 2.55-2.70(2H, m), 3.4-3.55(4H, m), 5.13(1H, 7-plet, J=6.6 Hz), 6.81(1H, d, J=9.7 Hz), 7.02(1H, d, J=9.7 Hz), 7.35-7.6(5H, m) ESI/MS: 414(M+H)⁺, 436(M+Na)⁺

### EXAMPLE 118

2-[(2-(Dimethylamino)ethyl)(methyl)amino]-N-[5-(1-isopropyl-6-oxo-1,6-dihydro-3-pyridazinyl)-4-phenyl-1,3-thiazol-2-yl]acetamide was obtained in a manner sirmilar to Example 100.

mp: 155-157° C. (diisopropyl ether)

IR (KBr): 1660 cm⁻¹

¹H NMR (DMSO-d₆, δ): 1.27(6H, d, J=6.6 Hz), 2.28(6H, s), 2.40(3H, s), 2.40-2.50(2H, m), 2.55-2.65(2H, m), 3.38(2H, s), 5.14(1H, 7-plet, J=6.6 Hz), 6.81(1H, d, J=9.7 Hz), 7.04(1H, d, J=9.7 Hz), 7.35-7.6(5H, m)

ESI/MS: 455(M+H)⁺, 477(M+Na)⁺

Elemental Analysis for C₂₃H₃₀N₆O₂S.0.1H₂O

Calcd. C: 60.53, H: 6.67, N: 18.41

Found C: 60.42, H: 6.61, N: 18.27

### EXAMPLE 119

2-{[2-(Dimethylamino)ethyl]amino}-N-[5-(1-isopropyl-6-oxo-1,6-dihydro-3-pyridazinyl)-4-phenyl-1,3-thiazol-2-yl]acetamide dihydrochloride

mp: 136-142° C. (diisopropyl ether)

IR (KBr): 3421, 1673, 1648 cm⁻¹

¹H NMR (DMSO-d₆, δ): 1.27(6H, d, J=6.6 Hz), 2.85(6H, s), 3.3-3.6(4H, m), 4.21(2H, s), 5.14(1H, 7-plet, J=6.6 Hz), 6.83(1H, d, J=9.7 Hz), 7.04(1H, d, J=9.7 Hz), 7.4-7.6(5H, m), 9.81(1H, brs), 10.84(1H, brs), 13.1(1H, br)

ESI/MS: 441(M−2HCl+H)⁺, 463(M−2HCl+Na)⁺

Elemental Analysis for C₂₂H₁₀Cl₂N₆O₂S 4.0H₂O

Calcd. C: 45.13, H: 6.54, N: 14.35

Found C: 45.22, H: 6.27, N: 14.15

### EXAMPLE 120

N-[5-(1-Isopropyl-6-oxo-1,6-dihydro-3-pyridazinyl)-4-phenyl-1,3-thiazol-2-yl]-2-[(3-pyridinylmethyl)amino]acetamide was obtained in a manner similar to Example 100.

mp: 156-158° C. (diisopropyl ether)

IR (KBr): 1664 cm⁻¹

¹H NMR (DMSO-d₆, δ): 1.27(6H, d, J=6.6 Hz), 3.50(2H, s), 3.79(2H, s), 5.14(1H, 7-plet, J=6.6 Hz), 6.81(1H, d, J=9.7 Hz), 7.02(1H, d, J=9.7 Hz), 7.3-7.55(8H, m), 7.7-7.8(1H, m), 8.4-8.5(1H, m), 8.5-8.6(1H,m)

ESI/MS: 461(M+H)⁺, 483(M+Na)⁺

### EXAMPLE 121

N-[5-(1-Isopropyl-6-oxo-1,6-dihydro-3-pyridazinyl)-4-phenyl-1,3-thiazol-2-yl]-3-(1-pyrrolidinyl)propanamide hydrochloride was obtained in a manner similar to Example 100.

mp: 224-225° C. (ethayl acetate)

IR (KBr): 3421, 1666 cm⁻¹

¹H NMR (DMSO-d₆, δ): 1.27(6H, d, J=6.6 Hz), 1.8-2.1(4H, m), 2.9-3.1(4H, m), 3.4-3.6(4H, m), 5.14(1H, 7-plet, J=6.6 Hz), 6.81(1H, d, J=9.7 Hz), 7.02(1H, d, J=9.7 Hz), 7.3-7.6(5H, m), 10.7-10.9(1H, br), 12.70(1H, s)

ESI/MS: 438(M−HCl+H)⁺, 460(M−HCl+Na)⁺

### EXAMPLE 122

N-[5-(1-Isopropyl-6-oxo-1,6-dihydro-3-pyridazinyl)-4-phenyl-1,3-thiazol-2-Yl]-3-[(2-methoxyethyl)amino]propanamide was obtained in a manner similar to Example 100.

mp: 167-168° C. (ethanol)

IR (KBr): 3303, 1658 cm⁻¹

¹H NMR (DMSO-d₆, δ): 1.27(6H, d, J=6.6 Hz), 2.5-2.65(2H, m), 2.65-2.75(2H, m), 2.8-2.9(2H, m), 3.24(3H, s), 3.3-3.45(2H, m), 5.13(1H, 7-plet, J=6.6 Hz), 6.81(1H, d, J=9.7 Hz), 7.01(1H, d, J=9.7 Hz), 7.05-7.35(1H, br), 7.3-7.6(5H, m)

ESI/MS: 442(M+H)⁺, 464(M+Na)⁺

### EXAMPLE 123

N-[5-(1-Isopropyl-6-oxo-1,6-dihydro-3-pyridazinyl)-4-phenyl-1,3-thiazol-2-yl]-3-(4-morpholinyl)propanamide was obtained in a manner similar to Example 100.

mp: 197-198° C. (ethanol)

IR (KBr): 3423, 1658 cm⁻¹

¹H NMR (DMSO-d₆, δ): 1.27(6H, d, J=6.6 Hz), 2.3-2.45(4H, m), 2.6-2.7(4H, m), 3.5-3.6(4H, m), 5.13(1H, 7-plet, J=6.6 Hz), 6.81(1H, d, J=9.7 Hz), 7.01(1H, d, J=9.7 Hz), 7.3-7.6(5H, m), 12.4-12.5(1H, br)

ESI/MS: 454(M+H)⁺, 476(M+Na)⁺

### EXAMPLE 124

N-[5-(1-Isopropyl-6-oxo-1,6-dihydro-3-pyridazinyl)-4-phenyl-1,3-thiazol-2-yl]-3-[(2-pyridinylmethyl)amino]propanamide was obtained in a manner similar to Example 100.

mp: 200-201° C. (ethanol)

IR (KEBr): 3235, 1652 cm⁻¹

¹H NMR (DMSO-d₆, δ): 1.27(6H, d, J=6.6 Hz), 2.6-2.7(2H, m), 2.8-2.9(2H, m), 3.82(2H, s), 5.13(1H, 7-plet, J=6.6 Hz), 6.81(1H, d, J=9.7 Hz), 7.01(1H, d, J=9.7 Hz), 7.2-7.3(1H, m), 7.3-7.6(7H, m), 7.65-7.8(1H, m), 8.45-8.55(1H, m)

ESI/MS: 475(M+H)⁺, 479(M+Na)⁺

### EXAMPLE 125

2-{[2-(Acetylamino)ethyl]aminoN-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: 102-106° C. (ethyl acetate)

IR (KBr): 3444, 1668, 1648 cm⁻¹

¹H NMR (DMSO-d₆, S): 1.27(6H, d, J=6.6 Hz), 1.86(3H, s), 3.0-3.2(2H, m), 3.3-3.5(2H, m), 4.13(2H, s), 5.13(1H, 7-plet, J-6.6 Hz), 6.83(1H, d, J=9.7 Hz), 7.04(1H, d, J=9.7 Hz), 7.4-7.6(5H, m), 8.2-8.3(1H, m), 9.39(2H, br), 12.9-13.1(1H, br)

ESI/MS: 455(M−HCl+H)⁺, 477(M−HCl+Na)⁺

### EXAMPLE 126

2-{[3-(Dimethylanmiio)propyl]amino}-N-[5-(1-isopropyl-6-oxo-1,6-dihydro-3-pyridazinyl)-4-phenyl-1,3-thiazol-2-yl]acetamide dihydrochloride was obtained in a manner similar to Example 100.

mp: 240-242° C. (ethyl acetate)

IR (KBr): 3490, 1668, 1652 cm⁻¹

¹H NMR (DMSO-d₆, δ): 1.27(6H, d, J=6.6 Hz), 2.0-2.2(2H, m), 2.75(6H, s), 3.0-3.2(4H, m), 4.13(2H, s), 5.14(1H, 7-plet, J=6.6 Hz), 6.83(1H, d, J=9.7 Hz), 7.04(1H, d, J=9.7 Hz), 7.4-7.6(5H, m), 9.3-10.0(2H, br), 10.0-10.9(1H, br), 12.7-13.3(1H, br)

ESI/MS: 455(M−2HCl+H)⁺, 477(M−2HCl+Na)⁺

### EXAMPLE 127

2-{[2-(Diethylamino)ethyl]amino}-N-[5-(1-isopropyl-6-oxo-1,6-dihydro-3-pyridazinyl)-4-phenyl-1,3-thiazol-2-yl]acetamide dihydrochloride was obtained in a manner similar to Example 100.

mp: 157-159° C. (ethyl acetate)

IR (KBr): 3421, 1648 cm⁻¹

¹H NMR (DMSO-d₆, δ): 1.1-1.4(12H, m), 3.1-3.3(4H, m), 3.4-3.6(4H, m), 4.22(2H, s), 5.14(1H, 7-plet, J=6.6 Hz), 6.83(1H, d, J=9.7 Hz), 7.04(1H, d, J=9.7 Hz), 7.3-7.6(5H, m), 9.7-10.2(2H, br), 10.8-11.3(1H, br), 12.7-13.3(1H, br)

ESI/MS: 469(M−2HCl+H)⁺

### EXAMPLE 128

2-[[2-(Diethylamino)ethyl](methyl)amino]-N-[5-(1-isopropyl-6-o.xo-1,6-dihydro-3-pyridazinyl)-4-phenyl-1,3-thiazol-2-yl]acetamide dihydrochloride was obtained in a manner similar to Example 100.

mp: 227-229° C. (ethyl acetate)

IR (KBr): 3444, 1650 cm⁻¹

¹H NMR (DMSO-d₆, δ): 1.1-1.4(12H, m), 2.94(3H, s), 3.1-3.3(4H, m), 3.4-3.6(4H, m), 4.33(2H, s), 5.14(1H, 7-plet, J=6.6 Hz), 6.83(1H, d, J=9.7 Hz), 7.04(1H, d, J=9.7 Hz), 7.3-7.6(5H, m), 10.8-11.3(1H, br), 12.7-13.3(1H, br)

ESI/MS: 483(M−2HCl+H)⁺

### EXAMPLE 129

N-[5-(1-Isopropyl-6-oxo-1,6-dihydro-3-pyridazinyl)-4-phenyl-1,3-thiazol-2-yl]-2-{[2-(4-morpholinyl)ethyl]amino}acetamide dihydrochloride was obtained in a manner similar to Example 100.

mp: 250-252° C. (ethyl acetate)

IR (KBr): 3444, 1648 cm⁻¹

¹H NMR (DMSO-d₆, δ): 1.27(6H, d, J=6.6 Hz), 3.1-4.0(12H, m), 4.22(2H, s), 5.14(1H, 7-plet, J=6.6 Hz), 6.83(1H, d, J=9.7 Hz), 7.04(1H, d, J=9.7 Hz), 7.3-7.6(5H, m), 9.7-10.3(2H, br), 10.8-11.8(1H, br), 12.7-13.3(1H, br)

ESI/MS: 483(M−2HCl+H)⁺, 505(M−2HCl+Na)⁺

### EXAMPLE 130

2-(Isopropylamino)-N-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: >250° C. (ethyl acetate)

IR (KBr): 3423, 1666 cm⁻¹

¹H NMR (PMSO-d₆, δ): 1.1-1.3(12H, m), 3.3-3.5(1H, m), 4.11(2H, s), 5.14(1H, 7-plet, J=6.6 Hz), 6.83(1H, d, J=9.7 Hz), 7.04(1H, d, J=9.7 Hz), 7.3-7.6(5H, m), 9.2-9.3(2H, br), 12.8-13.3(1H, br)

ESI/MS: 412(M−HCl+H)⁺, 434(M−HCl+Na)⁺

### EXAMPLE 131

2-(Cyclopropylamino)-N-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: 123-125° C. (ethyl acetate)

IR (KBr): 3423, 1648 cm⁻¹

¹H NMR (DMSO-d₆, δ): 0.65-0.8(2H, m), 0.9-1.05(2H, m), 1.27(6H, d, J=6.6 Hz), 2.75-2.9(1H, m), 4.18(2H, s), 5.14(1H, 7-plet, J=6.6 Hz), 6.83(1H, d, J=9.7 Hz), 7.04(1H, d, J=9.7 Hz), 7.3-7.6(5H, m), 9.72(2H, br), 12.8-13.3(1H, br)

ESI/MS: 410(M−HCl+H)⁺, 432(M−HCl+Na)⁺

### EXAMPLE 132

N-[5-(1 Isopropyl-6-oxo-1,6-dihydro-3-pyridazinyl)-4-phenyl-1,3-thiazol-2-yl]-2-(1-piperidinyl)acetamide was obtained in a manner similar to Example 100.

mp: 189-190° C. (ethanol)

IR (KBr): 3241, 1666 cm⁻¹

¹H NMR (DMSO-d₆, δ): 1.27(6H, d, J=6.6 Hz), 1.3-1.6(6H, m), 2.4-2.6(2H, m), 3.2-3.4(4H, m), 5.14(1H, 7-plet, Js6.6 Hz), 6.80(1H, d, J=9.7 Hz), 7.01(1H, d, J=9.7 Hz), 7.3-7.6(5H, m),11.0-13.0(1H, br)

ESI/MS: 438(M+H)⁺, 460(M+Na)⁺

### EXAMPLE 133

N-[5-(1-Isopropyl-6-oxo-1,6-dihydro-3-pyridazinyl)-4-phenyl 1,3-thiazol-2-yl]-2-[(3-methoxypropyl)amino]acetamide hydrochloride was obtained in a manner similar to Example 100.

mp: >250° C. (ethyl acetate)

IR (KBr): 3444, 1666 cm⁻¹

¹H NMR (DMSO-d₆, δ): 1.27(6H, d, J=6.6 Hz), 1.8-2.0(2H, m), 3.0-3.15(2H, m), 3.26(3H, s), 3.35-3.5(2H, m), 3.8-4.1(2H, m), 5.14(1H, 7-plet, J=6.6 Hz), 6.83(1H, d, J=9.7 Hz), 7.04(1H, d, J=9.7 Hz), 7.3-7.6(5H, m), 9.37(2H, br), 12.8-13.2(1H, br)

ESI/MS: 442(M−HCl+H)⁺, 464(M−HCl+Na)⁺

### EXAMPLE 134

2-[(2-Ethoxyethyl)amino]-N-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: 252-253° C. (ethyl acetate)

IR (KBr): 3444, 1666 cm⁻¹

¹H NMR (DMSO-d₆, δ): 1.16(3H, t, J=7.0 Hz), 1.27(6H, d, J=6.6 Hz), 3.2-3.3(2H, m), 3.49(2H, q, J=7.0 Hz), 3.6-3.75(2H, m), 4.14(2H, s), 5.14(1H, 7-plet, J=6.6 Hz), 6.83(1H, d, J=9.7 Hz), 7.04(1H, d, J=9.7 Hz), 7.3-7.6(5H, m), 9.35(2H, br), 12.8-13.2(1H, br)

ESI/MS: 442(M−HCl+H)⁺, 464(M−HCl+Na)⁺

### EXAMPLE 135

N-[5-(1-Isopropyl-6-oxo-1,6-dihydro-3-pyridazinyl)-4-phenyl-1,3-thiazol-2-yl]-2-{[2-(1-piperidinyl)ethyl]amino}acetamide dihydrochloride was obtained in a manner similar to Example 100.

mp: >250° C. (ethyl acetate)

IR (KBr): 1664, 1648 cm⁻¹

¹H NMR (DMSO-d₆, δ): 1.27(6H, d, J=6.6 Hz), 1.3-2.0(6H, m), 2.8-3.1(2H, m), 3.3-3.7(6H, m), 4.21(2H, s), 5.14(1H, 7-plet, J=6.6 Hz), 6.83(1H, d, J=9.7 Hz), 7.04(1H, d, J=9.7 Hz), 7.3-7.6(5H, m), 9.7-10.0(2H, br), 10.3-10.7(1H, br), 12.8-13.2(1H, br)

ESI/MS: 481(M−2HCl+H)⁺

### EXAMPLE 136

N-[5-(1-Isopropyl-6-oxo-1,6-dihydro-3-pyridazinyl)-4-phenyl-1,3-thiazol-2-yl]-4-(1-piperidinylmethyl)benzamide was obtained in a manner similar to Example 100.

mp: 136-138° C. (isopropyl ether)

IR (KBr): 3421, 1648, 1579 cm⁻¹

¹H NMR (DMSO-d₆, δ): 1.30(6H, d, J=6.6 Hz), 1.2-1.65(6H, br), 2.2-2.4(4H, br), 3.51(2H, s), 5.15(1H, 7-plet, J=6.6 Hz), 6.82(1H, d, J=9.7 Hz), 7.03(1H, d, J=9.7 Hz), 7.3-7.6(7H, m), 8.10(2H, d, J=8.1 Hz), 12.89(1H, br)

ESI/MS: 514(M+H)⁺, 536(M+Na)⁺

Elemental Analysis for C₂₀H₃₁N₅O₂S 0.6H₂O

Calcd. C: 66.41, H: 6.19, N: 13.35

Found C: 66.65, H: 6.21, N: 12.96

### EXAMPLE 137

4-{[[2-(Dimethylamino)ethyl] (methyl)amino]methyl}-N-[5-(1-isopropyl-6-oxo-1,6-dihydro-3-pyridazinyl)-4-phenyl-1,3-thiazol-2-yl]benzamide was obtained in a manner similar to Example 100.

mp: 131-133° C. (isopropyl ether)

IR (KBr): 3442, 1648, 1579 cm⁻¹

¹H NMR (DMSO-d₆, δ): 1.30(6H, d, J=6.6 Hz), 2.16(3H, s), 2.21(6H, s), 2.4-2.6(4H, br), 3.58(2H, s), 5.15(1H, 7-plet, J=6.6 Hz), 6.82(1H, d, J=9.7 Hz), 7.03(1H, d, J=9.7 Hz), 7.4-7.6(7H, m), 8.11(2H, d, J=8.1 Hz)

ESI/MS: 531(M+H)⁺, 553(M+Na)⁺

Elemental Analysis for C₂₉H₃₄N₆O₂S 0.9H₂O

Calcd. C: 63.69, H: 6.60, N: 15.37

Found C: 63.79, H: 6.45, N: 15.20

### EXAMPLE 138

4-{[(2-Hydroxyethyl)amino]methyl}-N-[5-(1-isopropyl-6-oxo-1,6-dihydro-3-pyridazinyl)-4-phenyl-1,3-thiazol-2-yl]benzamide was obtained in a manner similar to Example 100.

mp: 111-113° C. (isopropyl ether)

IR (KBr): 3421, 1650, 1579 cm⁻¹

¹H NMR (DMSO-d₆, δ): 1.30(6H, d, J=6.6 Hz), 2.60(2H, t, J=5.8 Hz), 3.4-3.6(3H, m), 3.82(2H, s), 4.52(1H, br), 5.15(1H, 7-plet, J=6.6 Hz), 6.81(1H, d, J=9.7 Hz), 7.03(1H, d, J=9.7 Hz), 7.3-7.6(7H, m), 8.11(2H, d, J=8.1 Hz)

ESI/MS: 490(M+H)⁺, 512(M+Na)⁺

Elemental Analysis for C₂₆H₂₇NsO₃S 1.5H₂O

Calcd. C: 60.45, H: 5.85, N: 13.56

Found C: 60.43, H: 5.47, N: 13.26

### EXAMPLE 139

4-(1H-Imidazol-1-ylmethyl)-N-[5-(1-isopropyl-6-oxo-1,6-dihydro-3-pyridazinyl)-4-phenyl-1,3-thiazol-2-yl]benzamide was obtained in a manner similar to Example 100.

mp: >250° C. (isopropyl ether)

IR (KBr): 3442, 1654, 1581 cm⁻¹

¹H NMR (DMSO-d₆, δ): 1.29(6H, d, J=6.6 Hz), 5.15(1H, 7-plet, J=6.6 Hz), 5.32(2H, s), 6.82(1H, d, J=9.7 Hz), 6.94(1H, s), 7.03(1H, d, J=9.7 Hz), 7.23(1H, s), 7.3-7.6(7H, m), 7.80(1H, s), 8.12(2H, d, J=8.3 Hz)

ESI/MS: 497(M+H)⁺, 519(M+Na)⁺

### EXAMPLE 140

N-[5-(1-Isopropyl-6-oxo-1,6-dihydro-3-pyridazinyl)-4-phenyl-1,3-thiazol-2-yl]-4-({[2-(4-morpholinyl)ethyl]amino}methyl)benzamide was obtained in a manner similar to Example 100.

mp: 86-88° C. (isopropyl ether)

IR (KBr): 3442, 1652, 1579 cm⁻¹

¹H NMR (DMSO-d₆, o): 1.30(6H, d, J=6.6 Hz), 2.2-2.7(8H, m), 3.5-3.6(4H, m), 3.82(2H, s), 5.15(1H, 7-plet, J=6.6 Hz), 6.82(1H, d, J=9.7 Hz), 6.9-7.1(1H, br), 7.03(1H, d, J=9.7 Hz), 7.3-7.6(7H, m), 8.10(2H, d, J=8.1 Hz)

ESI/MS: 559(M+H)⁺, 581(M+Na)⁺

### EXAMPLE 141

4-ff[2-(Diethylamino)ethyl] (methyl)amino]methyl}-N-[5-(1-isopropyl-6-oxo-1,6-dihydro-3-pyridazinyl)-4-phenyl-1,3-thiazol-2-yl]benzamide was obtained in a manner similar to Example 100.

mp: 115-116° C. (isopropyl ether)

IR (KBr): 3421, 1650, 1581 cm⁻¹

¹H NMR (DMSO-d₆, δ): 0.96(6H, t, J=7.1 Hz), 1.30(6H, d, J=6.6 Hz), 2.17(3H, s), 2.3-2.7(8H, m), 3.59(2H, s), 5.15(1H, 7-plet, J=6.6 Hz), 6.82(1H, d, J=9.7 Hz), 7.03(1H, d, J=9.7 Hz), 7.3-7.6(7H, m), 8.11(2H, d, J=8.1 Hz)

ESI/MS: 559(M+H)⁺, 581(M+Na)⁺

Elemental Analysis for C₃₁H₃₈N₆O₂S.0.7H₂O

Calcd. C: 65.17, H: 6.95, N: 14.71

Found C: 65.22, H: 6.74, N: 14.56

### EXAMPLE 142

4-({[2-(Diethylamino)ethyl]amino}methyl)-N-[5-(1-isopropyl-6-oxo-1,6-dihydro-3-pyridazinyl)-4-phenyl-1,3-thiazol-2-yl]benzamide was obtained in a manner similar to Example 100.

mp: 158-160° C. (isopropyl ether)

IR (KBr): 3426, 1660, 1585 cm⁻¹

¹H NMR (DMSO-d₆, δ): 0.95(6H, t, J=7.1 Hz), 1.30(6H, d, J=6.6 Hz), 2.4-2.6(8H, m), 3.81(2H, s), 5.15(1H, 7-plet, J=6.6 Hz), 6.81(1H, d, J=9.7 Hz), 7.03(1H, d, J=9.7 Hz), 7.3-7.6(7H, m), 8.11(2H, d, J=8.1 Hz)

ESI/MS: 545(M+H)⁺, 567(M+Na)⁺

Elemental Analysis for C₃₀H₃₆N₆O₂S 0.1H₂O

Calcd. C: 65.93, H: 6.68, N: 15.38

Found C: 65.95, H: 6.78, N: 14.94

### EXAMPLE 143

4-({[3-(Dimethylamino)propyl]amino}methyl)-N-[5-(1-isopropyl-6-oxo-1,6-dihydro-3-pyridazinyl)-4-phenyl-1,3-thiazol-2-yl]benzamide was obtained in a manner similar to Example 100.

mp: 108-110° C. (isopropyl ether)

IR (KBr): 3424, 1652, 1581 cm⁻¹

¹H NMR (DMSO-d₆, δ): 1.29(6H, d, J=6.6 Hz), 1.45-1.7(2H, m), 2.11(6H, s), 2.1-2.4(4H, m), 3.77(2H, s), 5.15(1H, 7-plet, J=6.6 Hz), 6.79(1H, d, J=9.7 Hz), 7.02(1H, d, J=9.7 Hz), 7.3-7.6(7H, m), 8.10(2H, d, J=8.2 Hz)

ESI/MS: 531(M+H)⁺, 553(M+Na)⁺

Elemental Analysis for C₂₉H₃₄N₆O₂S 1.0H₂O

Calcd. C: 63.48, H: 6.61, N: 15.32

Found C: 63.62, H: 6.85, N: 15.16

### EXAMPLE 144

N-[5-(1-Isopropyl-6-oxo-1,6-dihydro-3-pyridazinyl)-4-phenyl-1,3-thiazol-2-yl]-4-({[2-(1-piperidinyl)ethyl]amino}methyl)benzamide was obtained in a manner similar to Example 100.

mp: 134-136° C. (isopropyl ether)

IR (KBr): 3421, 1648, 1579 cm⁻¹

¹H NMR (DMSO-ds, 6): 1.30(6H, d, J=6.6 Hz), 1.2-1.6(6H, m), 2.2-2.7(8H, m), 3.80(2H, s), 5.15(1H, 7-plet, J=6.6 Hz), 6.81(1H, d, J=9.7 Hz), 7.03(1H, d, J=9.7 Hz), 7.3-7.6(7H, m), 8.10(2H, d, J=8.1 Hz)

ESI/MS: 557(M+H)⁺, 579(M+Na)⁺

Elemental Analysis for C₃₁H₃₆N₆O₂S 0.2H₂O

Calcd. C: 66.45, H: 6.55, N: 15.00

Found C: 66.42, H: 6.53, N: 14.72

### EXAMPLE 145

4-{[(2-Ethoxyethyl)amino]methyl}-N-[5-(1-isopropyl-6-oxo-1,6-dihydro-3-pyridazinyl)-4-phenyl-1,3-thiazol-2-yl]benzamide was obtained in a manner similar to Example 100.

mp: 153-154° C. (isopropyl ether)

IR (KBr): 3423, 1656, 1583 cm⁻¹

¹H NMR (DMSO-d₆, δ): 1.11(3H, t, J=7.0 Hz), 1.30(6H, d, J=6.6 Hz), 2.6-2.7(2H, m), 3.2-3.5(4H, m), 3.81(2H, s), 5.15(1H, 7-plet, J=6.6 Hz), 6.81(1H, d, J=9.6 Hz), 7.03(1H, d, J=9.6 Hz), 7.3-7.6(7H, m), 8.10(2H, d, J=8.2 Hz)

ESI/MS: 518(M+H)⁺, 540(M+Na)⁺

Elemental Analysis for C₂₈H₃₁N₉O₃S.0.2H₂O

Calcd. C: 64.52, H: 6.07, N: 13.44

Found C: 64.48, H: 6.99, N: 13.33

### EXAMPLE 146

N-15-(1-Isopropyl-6-oxo-1,6-dihydro-3-pyridazinyl)-4-phenyl-1,3-thiazol-2-yl]-4-{L(3-methoxypropyl)amino]methyl}benzamide was obtained in a manner similar to Example 100.

mp: 169-171° C. (isopropyl ether)

IR (KBr): 1654, 1583 cm⁻¹

¹H NMR PMSO-d₆, δ): 1.30(6H, d, J=6.6 Hz), 1.67(2H, 5-plet, J=6.7 Hz), 2.4-2.65(2H, m), 3.21(3H, s), 3.25-3.45(2H, m), 3.79(2H, s), 5.15(1H, 7-plet, J=6.6 Hz), 6.81(1H, d, J=9.7 Hz), 7.03(1H, d, J=9.6 Hz), 7.3-7.6(7H, m), 8.10(2H, d, J=8.2 Hz)

ESI/MS: 518(M+H)⁺, 540(M+Na)⁺

Elemental Analysis for C₂₈H₃₁N₅O₃S.0.4H₂O

Calcd. C: 64.08, H: 6.11, N: 13.34

Found C: 64.07, H: 5.94, N: 13.24

### EXAMPLE 147

N-[5-(1-Isopropyl-6-oxo-1,6-dihydro-3-pyridazinyl)-4-phenyl-1,3-thiazol-2-yl]-4-({methyl[2-(2-pyridinyl)ethyl]amino}methyl)benzamide was obtained in a manner similar to Example 100.

mp: 172-174° C. (isopropyl ether)

IR (KBr): 3307, 1648, 1579 cm⁻¹

¹H NMR PMSO-d₆, δ): 1.30(6H, d, J=6.6 Hz), 2.22(3H, s), 2.6-2.8(2H, m), 2.8-3.0(2H, m), 3.61(2H, s), 5.15(1H, 7-plet, J=6.6 Hz), 6.83(1H, d, J=9.7 Hz), 7.04(1H, d, J=9.6 Hz), 7.1-7.8(10H, m), 8.07(2H, d, J=8.2 Hz), 8.4-8.45(1H, m), 12.88(1H, br)

ESI/MS: 565(M+H)⁺, 587(M+Na)⁺

Elemental Analysis for C₃₂H₃₂N₆O₂S

Calcd. C: 68.06, H: 5.71, N: 14.88

Found C: 68.09, H: 5.76, N: 14.66

### EXAMPLE 148

2-{[(2R)-2-Hydroxypropyl anio}-N-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: 206-208° C. (ethyl acetate-diisopropyl ether)

IR (KBr):3411, 1646, 1579 cm⁻¹

¹H NMR (DMSO-d₆, δ): 1.16(3H, d, J=5.2 Hz), 1.28(6H, d, J=6.6 Hz), 2.7-3.2(2H, s), 3.8-4.2(3H, m), 5.14(1H, 7-plet, J=6.6 Hz), 6.83(1H, d, J=9.1 Hz), 7.04(1H, d, J=9.7 Hz), 7.3-7.6(5H, m), 8.7-9.5(2H, br), 12.99(1H, br)

ESI/MS: 428(M−HCl+H)⁺, 450(M−HCl+Na)⁺

### EXAMPLE 149

2-{[(2S)-2-Hydroxypropyl]arino}-N-[5-(1-isopropyl-6-oxo-1,6-dihydro-3-pyridazinyl)-4-phenyl-1,3-thiazol-2-yl]acetamide hydrochloride was obtained in a manner similar to Example 100.

mp: 211-213° C. (ethyl acetate-diisopropyl ether)

IR (KBr): 3438, 1644, 1583 cm⁻¹

¹H NMR (DMSO-d₆, δ): 1.16(3H, d, J=5.2 Hz), 1.28(6H, d, J=6.6 Hz), 2.7-3.2(2H, s), 3.8-4.2(3H, m), 5.14(1H, 7-plet, J=6.6 Hz), 6.83(1H, d, J=9.7 Hz), 7.04(1H, d, J=9.7 Hz), 7.3-7.6(5H, m), 8.7-9.5(2H, br), 12.99(1H, br)

ESI/MS: 428(M−HCl+H)⁺, 450(M−HCl+Na)⁺

### EXAMPLE 150

2-(4-Acetyl-1-piperazinyl)-N-[5-(1-isopropyl-6-oxo-1,6-dihydro-3-pyridazinyl)-4-phenyl-1,3-thiazol-2-yl]acetamide was obtained in a manner similar to Example 100.

mp 220-222° C. (diisopropyl ether)

IR (KBr): 3451, 1698, 1656 cm⁻¹

¹H NMR (DMSO-d₆, δ): 1.27(6H, d, J=6.6 Hz), 1.99(3H, s), 2.4-2.65(4H, m), 3.2-3.6(6H, m), 5.14(1H, 7-plet, J=6.6 Hz), 6.81(1H, d, J=9.7 Hz), 7.02(1H, d, J=9.7 Hz), 7.3-7.6(5H, m), 12.2-12.6 (1H, brs)

ESI/MS: 481(M+H)⁺, 503 (M+Na)⁺

### EXAMPLE 151

N-[5-(1-Isopropyl-6-oxo-1,6-dihydro-3-pyridazinyl)-4-(4-fluorophenyl)-1,3-thiazol-2-yl]-2-(4-morpholinylacetamide hydrochloride wasobtained in a manner similar to Example 100.

mp>250° C. (diisopropyl ether)

IR (KBr): 3451, 1698, 1656 cm⁻¹

¹H NMR (DMSO-d₆, δ): 1.27(6H, d, J=6.6 Hz), 3.2-4.0(8H, m), 4.36(2H, s), 5.14(1H, 7-plet, J=6.6 Hz), 6.85(1H, d, J=9.7 Hz), 7.07(1H, d, J=9.7 Hz), 7.2-7.4(2H, m), 7.5-7.65(2H, m), 10.8-11.4 (1H, brs), 13.0-13.5(1H, br)

ESI/MS: 458(M−HCl+H)⁺, 480 (M−HCl+Na)⁺

### EXAMPLE 152

4-[(Isopropylamino)methyl]-N-[5-(1-isopropyl-6-oxo-1,6-dihydro-3-pyridazinyl)-4-phenyl-1,3-thiazol-2-yl]benzamide was obtained in a manner similar to Example 100.

mp: 139-141° C. (isopropyl ether)

IR (KBr): 3426, 1654, 1581 cm⁻¹

¹H NMR (DMSO-d₆, δ): 1.04(6H, d, J=6.2 Hz), 1.30(6H, d, J=6.6 Hz), 2.76(1H, m), 3.81(2H, s), 5.15(1H, 7-plet, J=6.6 Hz), 6.81(1H, d, J=9.7 Hz), 7.03(1H, d, J=9.7 Hz), 7.3-7.6(7H, m), 8.11(2H, d, J=8.1 Hz)

ESI/MS: 488(M+H)⁺, 510(M+Na)⁺

Elemental Analysis for C₂₇H₂₉NsO₂S.0.4H₂O

Calcd. C: 65.54, H: 6.07, N: 14.15

Found C: 65.54, H: 5.97, N: 14.06

### EXAMPLE 153

A mixture of 6-(2-amino-4-phenyl-1,3-thiazol-5-yl)-2-isopropyl-3(2H)-pyridazinone (2.0 g) and triethylamine (80.94 ml) in dichloromethane (40 ml) was stirred at 0° C. 3-Chloropropionyl chloride (0.64 ml) was added to the solution with stirring. Chloroform and 1N-hydrochloric acid were added to the reaction mixture at ambient temperature. The separated organic layer was dried over sodium sulfate. The solvent was removed in vacuo to give a yellow powder, which was objected to a column chlomatography on silica gel eluting with ethyl acetate. The solvent was removed in vacuo to afford a yellow powder, which was suspended in diisopropyl ether with stirring.

The powder was collected by filtration to afford N-[5-(1-Isopropyl-6-oxo-1,6-dihydro-3-pyridazinyl)-4-phenyl-1,3-thiazol-2-yl]-3-chloropropanamide (0.64 g) as a white powder.

¹H NMR (CDCl₃ 6): 1.3-1.45(6H, m), 1.7-1.8(1H, m), 2.1-2.2(1H, m), 3.5-3:6(1H, m), 5.3-5.6(2H, m), 6.7-6.8(1H, m), 6.9-7.0(1H, m), 7.35-7.6(5H, m), 10.7(1H, br)

ESI/MS nega: 401(M−H)⁻

### EXAMPLE 154

A mixture of 6-(1-bromo-2-oxo-2-phenylethyl)-2-isopropyl-3(2H)-pyridazinone (150 mg) and 1-hexyl-2-thiourea (108 mg) in dioxane (1 ml) was stirred overnight at 80° C. The solvent was removed in vacuo to give yellow powder, which was objected to a column chromatography on silicagel eluting with a mixture of chloroform and methanol (20:1). The solvent was removed in vacuo to afford a yellow powder, which was suspended in a mixture of ethyl acetate and methanol with stirring.

The powder was collected by filtration to afford 6-(2-amino-4-phenyl-1,3-thiazol-5-yl)-2-isopropyl-3(2H)-pyridazinone hydrobromide as a yellow powder (6.51 g).

IR(KBr):3421, 1629, 1577 cm⁻¹

¹H NMR (DMSO-d₆, δ): 1.26(6H, d, J=6.6 Hz), 4.0-5.0(2H, br), 5.10(1H, 7-plet, J=6.6 Hz), 6.80(2H, s), 7.5-7.6(5H, m) mp>250 GC fdiisopropyl ether)

ESI/MS: 313(M+H)⁺, 335 (M+Na)+

### EXAMPLE 155

A mixture of 2-isopropyl-6-(2-{[3-(1-piperidyl)ethyl]amino}-4-phenyl-1,3-thiazol-5-yl)-3(2H)-pyridazinone(50 mg) and 4N-hydrochloric acid in ethyl acetate (0.3 ml) in methanol (2 ml) was stirred at ambient temperature. The solvent was removed in vacuo to afford a yellow powder, which was suspended in diisopropyl ether with stirring. The powder was collected by filtration to afford 2-isopropyl-6-(2-{[3-(1-piperidyl)ethyl]amino}-phenyl-1,3-thiazol-5-yl)-3(2H)-pyridazinone dihydrochloride as a yellow powder (30 mg).

mp: >250° C. (diisopropyl ether)

¹H NMR (DMSO-d₆, δ): 1.25(6H, d, J=6.6 Hz), 1.2-1.5(2H, m), 1.5-1.9(4H, m), 2.8-3.1(2H, m), 3.2-3.4(2H, m), 3.4-3.6(2H, m), 3.7-3.9(2H, m), 5.10(1H, 7-plet, J=6.6 Hz), 6.74(1H, d, J=9.8 Hz), 6.87(1H, d, J=9.8 Hz), 7.3-7.6(5H, m), 8.75(1H, br), 10.46(1H, br)

### EXAMPLE 156

A mixture of 2-isopropyl-6-(2-{[2-(4-morpholinyl)ethyl]amino}-4-phenyl-1,3-thiazol-5-yl)-3(2H)-pyridazinone (50 mg) and 4N-hydrochloric acid in ethyl acetate (0.3 ml) in methanol (2 ml) was stirred at ambient temperature. The solvent was removed in vacuo to afford a yellow powder, which was suspended in diisopropyl ether with stirring. The powder was collected by filtration to afford 2-isopropyl-6-(2-{[2-(4-morpholinyl)ethyl]amino}-4-phenyl-1,3-thiazol-5-yl)-3(2H)-pyridazinone dihydrochloride as yellow powder (30 mg).

mp: 140-143° C. (isopropyl ether)

¹H NMR PMSO-d₆, δ): 1.25(6H, d, J=6.6 Hz), 3.1-4.3(12H, m), 5.10(1H, 7-plet, J=6.6 Hz), 6.74(1H, d, J=9.7 Hz), 6.88(1H, d, J=9.7 Hz), 7.3-7.6(5H, m), 8.65(1H, br), 11.16 (1H, br)

### EXAMPLE 157

A mixture of 2-isopropyl-6-(2-{[3-(4-morpholinyl)propyl]amino}-4-phenyl-1,3-thiazol-5-yl)-3(2H)-pyridazinone (50 mg) and 4N-hydrochloric acid in ethyl acetate (0.3 ml) in methanol (2 ml) was stirred at ambient temperature. The solvent was removed in vacuo to afford a yellow powder, which was suspended in diisopropyl ether with stirring. The powder was collected by filtration to afford 2-isopropyl-6-(2-{[3-(4-morpholinyl)propyl]amino}-4-phenyl-1,3-thiazol-5-yl)-3(2H)-pyridazinone dihydrochloride as yellow powder (30 mg).

mp: 150-153° C. (diisopropyl ether)

¹H NMR (DMSO-d₆, δ): 1.26(6H, d, J=6.6 Hz), 1.9-2.2(2H, m), 2.9-3.6(8H, m), 3.7-4.0(4H, m), 5.10(1H, 7-plet, J=6.6 Hz), 6.74(1H, d, J=9.7 Hz), 6.83(1H, d, J=9.7 Hz), 7.3-7.6(5H, m), 9.17(1H, br), 11.37 (1H, br)

### EXAMPLE 158

A mixture of ethyl 5-(6-oxo-1,6-dihydro-3-pyridazinyl)-4-phenyl-1,3-thiazole-2-carboxylate (164 mg) and potassium tert-butoxide (17 mg) in formamide (1.64 mL) was heated for 6 hours at 100-105° C. Water (2 mL) was added to the reaction mixture to obtain a solid. The solid was collected by filtration, dried over phosphorous petoxide and purified by a column chromatography on silica gel (n-hexane:ethyl acetate=20:80, v/v) to give 5-(6-oxo-1,6-dihydro-3-pyridazinyl)-4-phenyl-1,3-thiazole-2-carboxamide as a solid (49 mg).

m.p.: >250° C. (methanol-diisopropyl ether)

IR (KBr): 3454, 3184, 1699, 1676, 1579 cm⁻¹

ESI/MS: 321(M+Na)⁺

¹H NMR (DMSO-d₆, δ): 6.82(1H, d, J=9.94 Hz), 7.06(1H, d, J=9.94 Hz), 7.45-7.49(3H, m), 7.57-7.63(2H, m), 7.98(1H, br.s), 8.25(1H, br.s), 13.37(1H, br.s)

Elemental Analysis for C₁₄H₁₀N₄O₂S

Calcd. C: 56.37; H: 3.38; N: 18.78

Found C: 56.05; H: 3.28; N: 18.59

### EXAMPLE 159

A mixture of ethyl 5-(1-methyl-6-oxo-1,6-dihydro-3-pyridazinyl)-4-phenyl-1,3-thiazole-2-carboxylate (171 mg) and potassium tert-butoxide (17 mg) in formamide (1.71 mL) was heated for 6 hours at 100-105° C. Water (2 mL) was added to the reaction mixture to obtain a solid. The solid was collected by filtration, dried over phosphorous petoxide and recrystallized from ethanol to give 5-(1-methyl-6-oxo-1,6-dihydro-3-pyridazinyl)-4-phenyl-1,3-thiazole-2-carboxamide as a solid (126 mg).

m.p.: 231-232° C. (ethanol)

IR (KBr): 3371, 3147, 1693, 1664, 1587 cm⁻¹

ESI/MS: 335(M+Na)⁺

¹H NMR (DMSO-d₆, δ): 3.70(3H, s), 6.88(1H, d, J=9.74 Hz), 7.07(1H, d, J=9.74 Hz), 7.45-7.50(3H, m), 7.58-7.64(2H, m), 7.98(1H, br.s), 8.26(1H, br.s)

Elemental Analysis for CirH₁₂N₄O₂S

Calcd. C: 57.68; H: 3.87; N: 17.94

Found C: 57.57; H: 3.79; N: 17.90

### EXAMPLE 160

5-(1-Ethyl-6-oxo-1,6-dihydro-3-pyridazinyl)-4-phenyl-1,3-thiazole-2-carboxamide was obtained in a manner similar to Example 159.

m.p.: 231-232.5° C. (ethanol)

IR (KBr): 3363, 3153, 1693, 1660, 1585 cm⁻¹

ESI/MS: 349(M+Na)⁺

¹H NMR PMSO-d₆, δ): 1.27(3H, t, J=7.17 Hz), 4.10(2H, q, J=7.17 Hz), 6.89(1H, d, J=9.78 Hz), 7.12(1H, d, J=9.78 Hz), 7.45-7.50(3H, m), 7.56-7.63(2H, m), 7.98(1H, br.s), 8.26(1H, br.s)

Elemental Analysis for C₁₆H₁₄N₄O₂S

Calcd. C: 58.88; H: 4.32; N: 17.17

Found C: 58.99; H: 4.22; N: 17.17

### EXAMPLE 161

5-(1-Isopropyl-6-oxo-1,6-dihydro-3-pyridazinyl)-4-phenyl-1,3-thiazole-2-carboxamidewas obtained in a manner similar to Example 159.

m.p.: 222-223° C. (ethanol)

IR (KBr): 3464, 3132, 1685, 1664, 1585 cm¹

ESI/MS: 363(M+Na)⁺, 341(M+H)⁺

¹H NMR PMSO-d₆, δ): 1.24(6H, d, J=6.61 Hz), 5.13(1H, 7-plet, J=6.61 Hz), 6.88(1H, d, J=9.70 Hz), 7.15(1H, d, J=9.70 Hz), 7.43-7.50(3H, m), 7.55-7.62(2H, m), 7.97(1H, br.s), 8.25(1H, br.s)

Elemental Analysis for C₁₇H₁₆N₄O₂S

Calcd. C: 59.98; H: 4.74; N: 16.46

Found C: 60.13; H: 4.74; N: 16.45

### EXAMPLE 162

5-(1-Allyl-6-oxo-1,6-dihydro-3-pyridazinyl)-4-phenyl-1,3-thiazole-2-carboxamide was obtained in a manner similar to Example 159.

m.p.: 198-199.5° C. (ethanol)

IR (KBr): 1691, 1664 cm⁻¹

ESI/MS: 361(M+Na)⁺

¹H NMR (CDCl₃, δ): 4.78-4.82(2H, m), 5.27-5.38(2H, m), 5.76(1H, br.s), 5.93-6.11(1H, m), 6.75(1H, d, J=9.70 Hz), 6.96(1H, d, J=9.70 Hz), 7.19(1H, br.s), 7.43-7.57(5H, m)

Elemental Analysis for C₁₇H₁₄N₄O₂S

Calcd. C: 60.34; H: 4.17; N: 16.56

Found C: 60.45; H: 4.18; N: 16.63

### EXAMPLE 163

4-(2-Fluorophenyl)-5-(1-isopropyl-6-oxo-1,6-dihydro-3-pyridazinyl)-1,3-thiazole-2-carboxamide was obtained in a manner similar to Example 159.

m.p.: 213-215° C. (ethanol)

IR (KBr): 3465, 3143, 1689, 1664, 1585 cm⁻¹

ESI/MS: 381(M+Na)⁺, 359(M+H)⁺

¹H NMR (CDCl₃, δ): 1.30(6H, d, J=6.61 Hz), 5.28(1H, 7-plet, J=6.61 Hz), 5.69(1H, br.s), 6.77(1H, d, J=9.62 Hz), 7.00(1H, d, J=9.62 Hz), 7.10-7.60(5H, m)

Elemental Analysis for C₁₇HisFN₄O₂S

Calcd. C: 56.97; H: 4.22; N: 15.63

Found C: 57.18; H: 4.28; N: 15.61

### EXAMPLE 164

4-(3-Fluorophenyl)-5-(1-isopropyl-6-oxo-1,6-dihydro-3-pyridazinyl)-1,3-thiazole-2-carboxamide was obtained in a manner similar to Example 159.

m.p.: 248-250° C. (ethanol)

IR (KBr): 3473, 3134, 1687, 1653, 1585 cm⁻¹

ESI/MS: 739(2M+Na)⁺, 381(M+Na)⁺

¹H NMR (CDCl₃, δ): 1.37(6H, d, J=6.62 Hz), 5.32(1H, 7-plet, J=6.62 Hz), 5.72(1H, br.s), 6.77(1H, d, J=9.61 Hz), 7.01(1H, d, J=9.61 Hz), 7.10-7.20(2H, m), 7.26-7.50(3H, m)

Elemental Analysis for C₁₇HlsFN₄O₂S

Calcd. C: 56.97; H: 4.22; N: 15.63

Found C: 57.13; H: 4.27; N: 15.55

### EXAMPLE 165

4-(4-Fluorophenyl)-5-(1-isopropyl-6-oxo-1,6-dihydro-3-pyridazinyl)-1,3-thiazole-2-carboxamide was obtained in a manner similar to Example 159.

m.p.: 226.5-227.5° C. (ethanol)

IR (KBr): 3473, 1691, 1664, 1587 cm⁻¹

ESI/MS: 381(M+Na)⁺

¹H NMR (CDCl₃, 3): 1.23(6H, d, J=6.60 Hz), 5.12(1H, 7-plet, J=6.60 Hz), 6.90(1H, d, J=9.60 Hz), 7.19(1H, d, J=9.60 Hz), 7.24-7.36(2H, m), 7.59-7.68(2H, m), 7.97(1H, br.s), 8.27(1H, br.s)

Elemental Analysis for C₁₇H₁₅FN₄O₂S

Calcd. C: 56.97; H: 4.22; N: 15.63

Found C: 56.87; H: 4.14; N: 15.65

### EXAMPLE 166

4-(3-Chlorophenyl)-5-(1-isopropyl-6-oxo-1,6-dihydro-3-pyridazinyl)-1,3-thiazole-2-carboxamide was obtained in a manner similar to Example 159.

m.p.: 232-234.5° C. (ethanol)

IR (KBr): 3365, 3153, 1689, 1653, 1579 cm⁻¹

ESI/MS: 773 and 771(2M+Na)⁺, 399 and 337(M+Na)⁺

¹H NMR (DMSO-d₆, δ): 1.21(6H, d, J=6.58 Hz), 5.12(1H, 7-plet, J=6.58 Hz), 6.93(1H, d, J=9.66 Hz), 7.30(1H, d, J=9.66 Hz), 7.42-7.75(3H, m), 7.74(1H, s), 8.01(1H, br.s), 8.34(1H, br.s)

Elemental Analysis for C₁₇H₁₅ClN₄O₂S

Calcd. C: 54.47; H: 4.03; N: 14.95

Found C: 54.71; H: 4.09; N: 14.82

### EXAMPLE 167

A mixture of ethyl 5-(6-oxo-1-propyl-1,6-dihydro-3-pyridazinyl)-4-phenyl-1,3-thiazole-2-carboxylate (185 mg) and potassium tert-butoxide (17 mg) in formamide (1.85 mL) was heated for 6 hours at 100-105° C. Water (2 mL) was added to the reaction mixture to obtain a solid. The solid was collected by filtration, dried over phosphorous petoxide and recrystallized from a mixture of ethanol and diisopropyl ether to give 5-(6-oxo-1-propyl-1,6-dihydro-3-pyridazinyl)-4-phenyl-1,3-thiazole-2-carboxamide as a solid (124 mg).

m.p.: 201-202° C. (ethanol-diisopropyl ether)

IR (KBr): 3163, 1697, 1664, 1585 cm⁻¹

ESI/MS: 363(M+Na)⁺

¹H NMR (DMSO-d₆, δ): 0.89(3H, t, J=7.38 Hz), 1.62-1.81(2H, m), 4.04(2H, t, J=7.09 Hz), 6.90(1H, d, J=9.64 Hz), 7.12(1H, d, J=9.64 Hz), 7.45-7.50(3H, m), 7.57-7.63(2H, m), 7.98(1H, br.s), 8.26(1H, br.s)

Elemental Analysis for C₁₇H₁₆N₄O₂S

Calcd. C: 59.98; H: 4.74; N: 16.46

Found C: 60.07; H: 4.65; N: 16.43

### EXAMPLE 168

5-[1-(2-Methoxyethyl)-6-oxo-1,6-dihydro-3-pyridazinyl]-4-phenyl-1,3-thiazole-2-carboxamide was obtained in a manner similar to Example 167.

m.p.: 198-199.5° C. (ethanol-diisopropyl ether)

IR (KBr): 3403, 3161, 1684, 1658, 1589 cm⁻¹

ESI/MS: 379(M+Na)⁺

¹H NMR (DMSO-d₆, δ): 3.26(3H, s), 3.68(2H, t, J=5.55 Hz), 4.26(2H, t, J=5.55 Hz), 6.90(1H, d, J=9.64 Hz), 7.11(1H, d, J=9.64 Hz), 7.45-7.49(3H, m), 7.57-7.63(2H, m), 7.98(1H, br.s), 8.27(1H, br.s)

Elemental Analysis for C₁₇H₁₆N₄O₃S

Calcd. C: 57.29; H: 4.52; N: 15.72

Found C: 57.29; H: 4.44; N: 15.69

### EXAMPLE 169

In a sealed tube, a mixture of ethyl 5-(6-oxo-1,6-dihydro-3-pyridazinyl)-4-phenyl-1,3-thiazole-2-carboxylate (164 mg) and propylamine (1 mL) in tetrahydrofuran (4 mL) was heated for 70 hours at 50-55° C. The mixture was concentrated under reduced pressure to give a residue. The residue was crystallized from ethanol to give 5-(6-oxo-1,6-dihydro-3-pyridazinyl)-4-phenyl-N-propyl-1,3-thiazole-2-carboxamide as a solid (133 mg).

m.p.: 232-233° C. (ethanol)

IR (KBr): 3363, 1680, 1662, 1593, 1527 cm⁻¹

ESI/MS: 363(M+Na)⁺, 341(M+H)⁺

¹H NMR (DMSO-d₆, δ): 0.88(3H, t, J=7.40 Hz), 1.51-1.59(2H, m), 3.21-3.28(2H, m), 6.83(1H, d, J=9.92 Hz), 7.05(1H, d, J=9.92 Hz), 7.46-7.50(3H, m), 7.57-7.61(2H, m); 8.93(1H, t, J=6.02 Hz), 13.37(1H, s)

Elemental Analysis for C₁₇H₁₆N₄O₂S

Calcd. C: 59.98; H: 4.74; N: 16.46

Found C: 59.96; H: 4.83; N: 16.31

### EXAMPLE 170

In a sealed tube, a mixture of ethyl 5-(1-methyl-6-oxo-1,6-dihydro-3-pyridazinyl)-4-phenyl-1,3-thiazole-2-carboxylate (171 mg) and propylamine (1 mL) in tetrahydrofuran (4 mL) was heated for 70 hours at 50-55° C. The mixture was concentrated under reduced pressure to give a residue. The residue was crystallized from diisopropyl ether to give 5-(1-methyl-6-oxo-1,6-dihydro-3-pyridazinyl)-4-phenyl-N-propyl-1,3-thiazole-2-carboxamide as a solid (162 mg).

m.p.: 108-109° C. (diisopropyl ether)

IR (KBr): 3379, 1678, 1651, 1595, 1525 cm⁻¹

ESI/MS: 377(M+Na)⁺

¹H NMR (CDCl₃, δ): 1.00(3H, t, J=7.42 Hz), 1.58-1.73(2H, m), 3.38-3.50(2H, m), 3.84(3H, s), 6.72(1H, d, J=9.62 Hz), 6.94(1H, d, J=9.62 Hz), 7.31(1H, br.s), 7.41-7.56(5H, m)

Elemental Analysis for ClsH₁₈N₄O₂S

Calcd. C: 61.00; H: 5.12; N: 15.81

Found C: 61.01; H: 5.16; N: 15.72

### EXAMPLE 171

In a sealed tube, a mixture of ethyl 5-(1-ethyl-6-oxo-1,6-dihydro-3-pyridazinyl)-4-phenyl-1,3-thiazole-2-carboxylate (178 mg) and propylamine (1 mL) in tetrahydrofuran (4 mL) was heated for 70 hours at 50-55° C. The mixture was concentrated under reduced pressure to give a residue. The residue was crystallized from a mixture of ethanol and diisopropyl ether to give 5-(1-ethyl-6-oxo-1,6-dihydro-3-pyridazinyl)-4-phenyl-N-propyl-1,3-thiazole-2-carboxamide as a solid (113 mg).

m.p.: 106.5-107.5° C. (ethanol-diisopropyl ether)

IR (KBr): 3319, 1672, 1653, 1589, 1531 cm⁻¹

ESI/MS: 391(M+Na)⁺

¹H NMR (CDCl₃, δ): 1.00(3H, t, J=7.42 Hz), 1.42(3H, t, J=7.21 Hz), 1.60-1.74(2H, m), 3.38-3.50(2H, m), 4.25(2H, q, J=7.21 Hz), 6.72(1H, d, J=9.68 Hz), 6.94(1H, d, J=9.68 Hz), 7.31(1H, br.s), 7.43-7.56(5H, m)

Elemental Analysis for C₁₉H₂₀N₄O₂S

Calcd. C: 61.94; H: 5.47; N: 15.21

Found C: 61.93; H: 5.50; N: 15.20

### EXAMPLE 172

In a sealed tube, a rixture of ethyl 5-(6-oxo-1-propyl-1,6-dihydro-3-pyridazinyl)-4-phenyl-1,3-thiazole-2-carboxylate (185 mg) and propylamine (1 mL) in tetrahydrofuran (4 mL) was heated for 70 hours at 50-55° C. The mixture was concentrated under reduced pressure to give a residue. The residue was crystallized from a mixture of ethanol and diisopropyl ether to give 5-(6-oxo-1-propyl-1,6-dihydro-3-pyridazinyl)-4-phenyl-N-propyl-1,3-thiazole-2-carboxamide as a solid (123 mg).

m.p.: 121-122° C. (ethanol-diisopropyl ether)

IR (KBr): 3319, 1676, 1651, 1593, 1539 cm⁻¹

ESI/MS: 405(M+Na)⁺, 383(M+H)⁺

¹H NMR (CDCl₃, δ): 0.99(3H, t, J=7.42 Hz), 1.00(3H, t, J=7.40 Hz), 1.64-1.70(2H, m), 1.84-1.90(2H, m), 3.41-3.47(2H, m), 4.16(2H, q, J=7.36 Hz), 6.72(1H, d, J=9.72 Hz), 6.94(1H, d, J=9.72 Hz), 7.31(1H, br.s), 7.44-7.48(3H, m), 7.51-7.55(2H, m)

Elemental Analysis for C₂₀H₂₂N₄O₂S

Calcd. C: 62.81; H: 5.80; N: 14.65

Found C: 62.75; H: 5.81; N: 14.59

### EXAMPLE 173

In a sealed tube, a mixture of ethyl 5-(1-isopropyl-6-oxo-1,6-dihydro-3-pyridazinyl)-4-phenyl-1,3-thiazole-2-carboxylate (100 mg) and propylamine (0.5 mL) in tetrahydrofuran (2 mL) was heated for 70 hours at 50-55° C. The mixture was concentrated under reduced pressure to give a residue. The residue was purified by a preparative TLC on silica gel (n-hexane:ethyl acetate 50:50, v/v) and crystallized from a mixture of ethanol and diisopropyl ether to give 5-(1-isopropyl-6-oxo-1,6-dihydro-3-pyridazinyl)-4-phenyl-N-propyl-1,3-thiazole-2-carboxamide as a solid (82 mg).

m.p.: 141-142° C. (ethanol-diisopropyl ether)

IR (KBr): 3273, 1672, 1651, 1541 cm⁻¹

ESI/MS: 787(2M+Na)⁺, 405(M+Na)⁺, 383(M+H)⁺

¹H NMR (CDCl₃, δ): 1.00(3H, t, J=7.36 Hz), 1.38(6H, d, J=6.62 Hz), 1.59-1.73(2H, m), 3.39-3.50(2H, m), 5.31(1H, 7-plet, J=6.62 Hz), 6.71(1H, d, J=9.60 Hz), 6.95(1H, d, J=9.60 Hz), 7.26-7.35(1H, m), 7.43-7.57(5H, m)

Elemental Analysis for C₂₅H₂₂N₄O₂S

Calcd. C: 62.81; H: 5.80; N: 14.65

Found C: 62.85; H: 5.88; N: 14.67

### EXAMPLE 174

In a sealed tube, a mixture of ethyl 5-(1-benzyl-6-oxo-1,6-dihydro-3-pyridazinyl)-4-phenyl-1,3-thiazole-2-carboxylate (105 mg) and propylamine (0.5 mL) in tetrahydrofuran (2 mL) was heated for 70 hours at 50-55° C. The mixture was concentrated under reduced pressure to give a residue. The residue was crystallized from a mixture of ethanol and diisopropyl ether to give 5-(1-benzyl-6-oxo-1,6-dihydro-3-pyridazinyl)-4-phenyl-N-propyl-1,3-thiazole-2-carboxamide as a solid (64 mg).

m.p.: 120-121° C. (ethanol-diisopropyl ether) rR (KBr): 3350, 1674, 1664, 1589, 1537 cm⁻¹ ESI/IMS: 883(2M+Na)⁺, 453(M+Na)⁺, 431(M+H)⁺

¹H NMR (CDCl₃, δ): 1.00(3H, t, J=7.40 Hz), 1.58-1.74(2H, m), 3.38-3.50(2H, m), 5.33(2H, s), 6.71(1H, d, J=9.77 Hz), 6.91(1H, d, J=9.77 Hz), 7.26-7.53(11H, m)

Elemental Analysis for C₂₄H₂₂N₄O₂S

Calcd. C: 66.96; H: 5.15; N: 13.01

Found C: 66.84; H: 5.15; N: 12.98

### EXAMPLE 175

In a sealed tube, a mixture of ethyl 4-(4-fluorophenyl)-5-(1-isopropyl-6-oxo-1,6-dihydro-3-pyridazinyl)-1,3-thiazole-2-carboxylate (100 mg) and propylamine (0.5 mL) in tetrahydrofuran (2 mL) was heated for 70 hours at 50-55° C. The mixture was concentrated under reduced pressure to give a residue. The residue was purified by a preparative TLC on silica gel (n-hexane:ethyl acetate=50:50, v/v) and crystallized from diisopropyl ether to give 4-(4-fluorophenyl)-5-(1-isopropyl-6-oxo-1,6-dihydro-3-pyridazinyl)-N-propyl-1,3-thiazole-2-carboxamide as a solid (86 mg).

m.p.: 146.5-147.5° C. (diisopropyl ether)

IR (KBr): 3275, 1674, 1651, 1541 cm⁻¹

ESI/MS: 423(M+Na)⁺, 401(M+H)⁺

¹H NMR (CDCl₃, δ): 1.00(3H, t, J=7.40 Hz), 1.13(6H, d, J=6.62 Hz), 1.62-1.70(2H, m), 3.41-3.48(2H, m), 5.31(1H, 7-plet, J=6.62 Hz), 6.75(1H, d, J=9.68 Hz), 6.95(1H, d, J=9.68 Hz), 7.12-7.18(2H, m), 7.28(1H, t, J=5.72 Hz), 7.51-7.55(2H, m)

Elemental Analysis for C₂₀H₂₁FN₄O₂S

Calcd. C: 59.98; H: 5.29; N: 13.99

Found C: 60.24; H: 5.40; N: 13.90

### EXAMPLE 176

In a sealed tube, a ixture of ethyl 5-(6-oxo-1,6-dihydro-3-pyridazinyl)-4-phenyl-1,3-thiazole-2-carboxylate (164 mg) and isopropylamine (1 mL) in tetrahydrofuran (4 mL) was heated for 70 hours at 50-55° C. The mixture was concentrated under reduced pressure to give a residue. The residue was crystallized from ethanol to give N-isopropyl-5-(6-oxo-1,6-dihydro-3-pyridazinyl)-4-phenyl-1,3-thiazole-2-carboxamide as a solid (137 mg).

m.p.: >250° C. (ethanol)

IR (KBr): 3278, 1682, 1651, 1591, 1541 cm⁻¹

ESI/MS: 363(M+Na)⁺

¹H NMR (DMSO-d₆, δ): 1.20(6H, d, J=6.60 Hz), 4.09-415(1H, m), 6.83(1H, d, J=9.92 Hz), 7.04(1H, d, J=9.92 Hz), 7.46-7.50(3H, m), 7.59-7.62(2H, m), 8.68(1H, d, J=8.44 Hz), 13.28(1H, br.s)

Elemental Analysis for C₁₇H₁₆N₄O₂S

Calcd. C: 59.98; H: 4.74; N: 16.46

Found C: 60.06; H: 4.75; N: 16.46

### EXAMPLE 177

In a sealed tube, a mixture of ethyl 5-(1-methyl-6-oxo-1,6-dihydro-3-pyridazinyl)-4-phenyl-1,3-thiazole-2-carboxylate (171 mg) and isopropylamine (1 mL) in tetrahydrofuran (4 mL) was heated for 70 hours at 50-55° C. The mixture was concentrated under reduced pressure to give a residue. The residue was crystallized from a mixture of ethanol and diisopropyl ether to give N-isopropyl-5-(1-methyl-6-oxo-1,6-dihydro-3-pyridazinyl)-4-phenyl-1,3-thiazole-2-carboxamide as a solid (124 mg).

m.p.: 154-154.5° C. (ethanol-diisopropyl ether)

IR (KBr): 3307, 1680, 1645, 1595, 1535 cm⁻¹

ESI/MS: 377(M+Na)⁺

¹H NMR (CDCl₃, o): 1.30(6H, d, J=6.60 Hz), 3.84(3H, s), 4.25-4.30(1H, m), 6.73(1H, d, J=9.72 Hz), 6.94(1H, d, J=9.72 Hz), 7.11(1H, d, J=8.10 Hz), 7.44-7.48(3H, m), 7.51-7.55(2H, m)

Elemental Analysis for C₁₅H₁₈N₄O₂S

Calcd. C: 61.00; H: 5.12; N: 15.81

Found C: 61.00; H: 5.15; N: 15.76

### EXAMPLE 178

In a sealed tube, a mixture of ethyl 5-(1-ethyl-6-oxo-1,6-dihydro-3-pyridazinyl)-4-phenyl-1,3-thiazole-2-carboxylate (178 mg) and isopropylamine (1 mL) in tetrahydrofuran (4 mL) was heated for 70 hours at 50-55° C. The mixture was concentrated under reduced pressure to give a residue. The residue was crystallized from a mixture of ethanol and diisopropyl ether to give 5-(1-ethyl-6-oxo-1,6-dihydro-3-pyridazinyl)-N-isopropyl-4-phenyl-1,3-thiazole-2-carboxamide as a solid (104 mg).

m.p.: 152.5-153° C. (ethanol-diisopropyl ether)

IR (KBr): 3300, 1674, 1651, 1593, 1554 cm⁻¹

ESI/MS: 391(M+Na)⁺

¹H NMR (CDCl₃, δ): 1.30(6H, d, J=6.60 Hz), 1.42(3H, t, J=7.20 Hz), 4.22-4.31(3H, m), 6.72(1H, d, J=9.72 Hz), 6.93(1H, d, J=9.72 Hz), 7.11(1H, d, J=8.32 Hz), 7.44-7.48(3H, m), 7.52-7.55(2H, m)

Elemental Analysis for C₁₉H₂₀N₄O₂S

Calcd. C: 61.94; H: 5.47; N: 15.21

Found C: 62.00; H: 5.49; N: 15.21

### EXAMPLE 179

In a sealed tube, a mixture of ethyl 5-(6-oxo-1-propyl-1,6-dihydro-3-pyridazinyl)-4-phenyl-1,3-thiazole-2-carboxylate (185 mg) and isopropylamine (1 mL) in tetrahydrofuran (4 mL) was heated for 70 hours at 50-55° C. The mixture was concentrated under reduced pressure to give a residue. The residue was crystallized from a mixture of ethanol and diisopropyl ether to give N-isopropyl-5-(6-oxo-1-propyl-1,6-dihydro-3-pyridazinyl)-4-phenyl-1,3-thiazole-2-carboxamide as a solid (108 mg).

m.p.: 146.5-147.5° C. (ethanol-diisopropyl ether)

IR (KBr): 3313, 1676, 1651, 1593, 1531 cm⁻¹

ESI/MS: 787(2M+Na)⁺, 405(M+Na)⁺

¹H NMR (CDCl₃, δ): 1.00(3H, t, J=7.42 Hz), 1.29(6H, d, J=6.56 Hz), 1.83-1.91(2H, m), 4.16(2H, t, J=7.34 Hz), 4.24-4.31(1H, m), 6.72(1H, d, J=9.68 Hz), 6.92(1H, d, J=9.68 Hz), 7.11(1H, d, J=8.08 Hz), 7.44-7.48(3H, m), 7.52-7.55(2H, m)

Elemental Analysis for C₂₀H₂₂N₄O₂S

Calcd. C: 62.81; H: 5.80; N: 14.65

Found C: 62.89; H: 5.83; N: 14.62

### EXAMPLE 180

In a sealed tube, a mixture of (100 mg) and isopropylamine (0.5 mL) in tetrahydrofuran (2 mL) was heated for 70 hours at 50-55° C. The mixture was concentrated under reduced pressure to give a residue. The residue was purified by a preparative TLC on silica gel (n-hexane:ethyl acetate=50:50, v/v) and crystallized from a mixture of diisopropyl ether and n-hexane to give N-isopropyl-5-(1-isopropyl-6-oxo-1,6-dihydro-3-pyridazinyl)-4-phenyl-1,3-thiazole-2-carboxamide as a solid (86 mg).

m.p.: 131-132.5° C. (diisopropyl ether-n-hexane)

IR (KBr): 3273, 1666, 1643, 1534 cm⁻¹

ESI/MS: 787(2M+Na)⁺, 405(M+Na)⁺

¹H NMR (CDCl₃, δ): 1.28(6H, d, J=6.56 Hz), 1.38(6H, d, J=6.60 Hz), 4.22-4.34(1H, m), 5.31(1H, 7-plet, J=6.60 Hz), 6.70(1H, d, J=9.60 Hz), 6.94(1H, d, J=9.60 Hz), 7.11(1H, d, J=8.04 Hz), 7.43-7.57(5H, m)

Elemental Analysis for C₂₀H₂₂N₄O₂S

Calcd. C: 62.81; H: 5.80; N: 14.65

Found C: 63.07; H: 5.98; N: 14.63

### EXAMPLE 181

In a sealed tube, a mixture of ethyl 5-(1-allyl-6-oxo-1,6-dihydro-3-pyridazinyl)-4-phenyl-1,3-thiazole-2-carboxylate (184 mg) and isopropylamine (1 mL) in tetrahydrofuran (4 rL) was heated for 70 hours at 50-55° C. The mixture was concentrated under reduced pressure to give a residue. The residue was crystallized from ethanol to give 5-(1-allyl-6-oxo-1,6-dihydro-3-pyridazinyl)-N-isopropyl-4-phenyl-1,3-thiazole-2-carboxamide as a solid (152 mg).

m.p.:166-167° C. (ethanol)

IR (KBr): 3305, 1678, 1647, 1593, 1531 cm⁻¹

ESI/MS: 403(M+Na)⁺, 381(M+H)⁺

¹H NMR (CDCl₃, δ): 1.29(6H, d, J=6.56 Hz), 4.24-4.30(1H, m), 4.77-4.81(2H, m), 5.28-5.36(2H, m), 5.97-6.06(1H, m), 6.74(1H, d, J=9.72 Hz), 6.94(1H, d, J=9.72 Hz), 7.11(1H, d, J=8.10 Hz), 7.44-7.48(3H, m), 7.52-7.55(2H, m)

Elemental Analysis for C₂₀H₂₀N₄O₂S

Calcd. C: 63.14; H: 5.30; N: 14.73

Found C: 63.09; H: 5.32; N: 14.66

### EXAMPLE 182

In a sealed tube, a mixture of ethyl 5-(1-benzyl-6-oxo-1,6-dihydro-3-pyridazinyl)-4-phenyl-1,3-thiazole-2-carboxylate (105 mg) and isopropylamine (0.5 mL) in tetrahydrofuran (2 mL) was heated for 70 hours at 50-55° C. The mixture was concentrated under reduced pressure to give a residue. The residue was crystallized from a mixture of ethanol and diisopropyl ether to give 5-(1-benzyl-6-oxo-1,6-dihydro-3-pyridazinyl)-N-isopropyl-4-phenyl-1,3-thiazole-2-carboxamide as a solid (88 mg).

m.p.: 163.5-165° C. (ethanol-diisopropyl ether)

IR (KBr): 3288, 1674, 1649, 1593, 1539 cm⁻¹

ESI/MS: 883(2M+Na)⁺, 453(M+Na)⁺, 431(M+E)⁺

¹H NMR (CDCl₃, δ): 1.29(6H, d, J=6.60 Hz), 4.25-4.32(1H, m), 5.33(2H, s), 6.71(1H, d, J=9.72 Hz), 6.91(1H, d, J=9.72 Hz), 7.11(1H, d, J=8.10 Hz), 7.30-7.53(10H, m)

Elemental Analysis for C₂₄H₂₂N₄O₂S

Calcd. C: 66.96; H: 5.15; N: 13.01

Found C: 66.73; H: 5.13; N: 12.94

### EXAMPLE 183

In a sealed tube, a mixture of ethyl 5-[1-(2-methoxyethyl)-6-oxo-1,6-dihydro-3-pyridazinyl]-4-phenyl-1,3-thiazole-2-carboxylate (96.4 mg) and isopropylamine (0.5 mL) in tetrahydrofuran (2 mL) was heated for 70 hours at 50-55° C. The mixture was concentrated under reduced pressure to give a residue. The residue was crystallized from a mixture of ethanol and diisopropyl ether to give N-isopropyl-5-[1-(2-methoxyethyl)-6-oxo-1,6-dihydro-3-pyridazinyl]-4-phenyl-1,3-thiazole-2-carboxamide as a solid (83 mg).

m.p.: 172-172.5° C. (ethanol-diisopropyl ether)

IR (KBr): 3294, 1670, 1649, 1591, 1537 cm⁻¹

ESI/MS: 819(2M+Na)⁺, 421(M+Na)⁺, 399(M+H)⁺

¹H NMR (CDCl₃, δ): 1.29(6H, d, J=6.58 Hz), 3.40(3H, s), 3.83(2H, t, J=5.60 Hz), 4.25-4.31(1H, m), 4.40(2H, t, J=5.60 Hz), 6.72(1H, d, J=9.72 Hz), 6.93(1H, d, J=9.72 Hz), 7.11(1H, d, J=8.12 Hz), 7.45-7.48(3H, m), 7.52-7.56(2H, m)

Elemental Analysis for C₂₀H₂₂N₄O₃S

Calcd. C: 60.28; H: 5.56; N: 14.06

Found C: 60.29; H: 5.59; N: 14.04

### EXAMPLE 184

In a sealed tube, a mixture of ethyl 4-(2-fluorophenyl)-5-(1-isopropyl-6-oxo-1,6-dihydro-3-pyridazinyl)-1,3-thiazole-2-carboxylate (201 mg) and isopropylamine (1 mL) in tetrahydrofuran (4 mL) was heated for 80 hours at 50-55° C. The mixture was concentrated under reduced pressure to give a residue. The residue was crystallized from a mixture of ethanol and n-hexane to give 4-(2-fluorophenyl)-N-isopropyl-5-(1-isopropyl-6-oxo-1,6-dihydro-3-pyridazinyl)-1,3-thiazole-2-carboxamide as a solid (132 mg).

m.p.: 129-130.5° C. (ethanol-n-hexane)

IR (KBr): 3317, 1678, 1655, 1531 cm⁻¹

ESI/MS: 423(M+Na)⁺, 401(M+H)⁺

¹H NMR (CDCl₃, δ): 1.29(12H, d, J=6.62 Hz), 4.18-4.37(1H, m), 5.27(1H, 7-plet, J=6.62 Hz), 6.76(1H, d, J=9.62 Hz), 6.99(1H, d, J=9.62 Hz), 7.03-7.35(3H, m), 7.40-7.65(2H, m)

Elemental Analysis for C₂₀H₂₁FN₄O₂S

Calcd. C: 59.98; H: 5.29; N: 13.99

Found C: 60.05; H: 5.32; N: 13.97

### EXAMPLE 185

In a sealed tube, a mixture of ethyl 4-(3-fluorophenyl)-5-(1-isopropyl-6-oxo-1,6-dihydro-3-pyridazinyl)-1,3-thiazole-2-carboxylate (201 mg) and isopropylamine (1 mL) in tetrahydrofuran (4 mL) was heated for 80 hours at 50-55° C. The mixture was concentrated under reduced pressure to give a residue. The residue was crystallized from ethanol to give 4-(3-fluorophenyl)-N-isopropyl-5-(1-isopropyl-6-oxo-1,6-dihydro-3-pyridazinyl)-1,3-thiazole-2-carboxamide as a solid (133 mg).

m.p.: 103.5-105.5° C. (ethanol-n-hexane)

IR (KBr).: 3286, 1662, 1653, 1587, 1537 cm⁻¹

ESI/MS: 823(2M+Na)⁺, 423(M+Na)⁺, 401(M+H)⁺

¹H NMR (CDCl₃, &): 1.30(6H, d, J=6.60 Hz), 1.36(6H, d, J=6.60 Hz), 4.20-4.37(1H, m), 5.31(1H, 7-plet, J=6.60 Hz), 6.76(1H, d, J=9.84 Hz), 6.99(1H, d, J=9.84 Hz), 7.10-7.50(5H, m)

Elemental Analysis for C₂₀H₂₁FN₄O₂S

Calcd. C: 59.98; H: 5.29; N: 13.99

Found C: 60.00; H: 5.56 N: 13.70

### EXAMPLE 186

In a sealed tube, a mixture of ethyl 4-(4-fluorophenyl)-5-(1-isopropyl-6-oxo-1,6-dihydro-3-pyridazinyl)-1,3-thiazole-2-carboxylate (100 mg) and isopropylamine (0.5 mL) in tetrahydrofuran (2 mL) was heated for 80 hours at 50-55° C. The mixture was concentrated under reduced pressure to give a residue. The residue was crystallized from a mixture of diisopropyl ether to give 4-(4-fluorophenyl)-N-isopropyl-5-(1-isopropyl-6-oxo-1,6-dihydro-3-pyridazinyl)-1,3-thiazole-2-carboxamide as a solid (96 mg).

m.p.: 122-123.5° C. (diisopropyl ether-n-hexane)

IR (KBr) 3417, 1664, 1587, 1518 cm⁻¹

ESI/MS: 423(M+Na)⁺

¹H NMR (CDCl₃, δ): 1.30(6H, d, J=6.56 Hz), 1.37(6H, d, J=6.62 Hz), 4.25-4.31(1H, m), 5.31(1H, 7-plet, J=6.62 Hz), 6.75(1H, d, J=9.66 Hz), 6.95(1H, d, J=9.66 Hz), 7.08(1H, d, J=8.06 Hz), 7.13-7.18(2H, m), 7.51-7.56(2H, m)

Elemental Analysis for C₂₀H₂₁FN₄O₂S

Calcd. C: 59.98; H: 5.29; N: 13.99

Found C: 60.02; H: 5.40; N: 13.86

### EXAMPLE 187

In a sealed tube, a mixture of ethyl 4-(3-chlorophenyl)-5-(1-isopropyl-6-oxo-1,6-dihydro-3-pyridazinyl)-1,3-thiazole-2-carboxylate (203 mg) and isopropylamine (1 mL) in tetrahydrofuran (4 mL) was heated for 80 hours at 50-55° C. The mixture was concentrated under reduced pressure to give a residue. The residue was crystallized from a mixture of diisopropyl ether and n-hexane to give 4-(3-chlorophenyl)-N-isopropyl-5-(1-isopropyl-6-oxo-1,6-dihydro-3-pyridazinyl)-1,3-thiazole-2-carboxamide as a solid (177 mg).

m.p.: 105-106° C. (diisopropyl ether-n-hexane)

IR (KBr): 1662, 1591, 1531 cm⁻¹

ESI/MS: 857 and 855(2M+Na)⁺, 441 and 439(M+Na)⁺

¹H NMR (CDCl₃, δ): 1.31(3H, d, J=6.61 Hz), 1.36(3H, d, J=6.64 Hz), 4.19-4.38(1H, m), 5.31(1H, 7-plet, J=6.62 Hz), 6.77(1H, d, J=9.66 Hz), 7.04(1H, d, J=9.66 Hz), 7.31(1H, d, J=8.38 Hz), 7.35-7.61(3H, m), 7.61-7.63(1H, m)

Elemental Analysis for C₂₀H₂₁ClN₄O₂S

Calcd. C: 57.62; H: 5.08; N: 13.44

Found C: 57.94; H: 5.31; N: 13.54

### EXAMPLE 188

In a sealed tube, a mixture of ethyl 5-(6-oxo-1,6-dihydro-3-pyridazinyl)-4-phenyl-1,3-thiazole-2-carboxylate (164 mg) and cyclopropylamine (0.347 mL) in dioxane (0.5 mL) was heated for 40 hours at 70-75° C. The mixture was concentrated under reduced pressure to give a residue. The residue was crystallized from a mixture of ethanol and diisopropyl ether to give N-cyclopropyl-5-(6-oxo-1,6-dihydro-3-pyridazinyl)-4-phenyl-1,3-thiazole-2-carboxamide as a solid (81 mg).

m.p.: >250° C. (ethanol-diisopropyl ether)

IR (KBr):3267, 1680, 1651, 1591, 1552 cm⁻¹

ESI/MS: 361(M+Na)⁺, 339(M+H)⁺

¹H NMR (DMSO-d₆, δ): 0.70-0.72(4H, m), 2.87-2.92(1H, m), 6.83(1H, d, J=9.92 Hz), 7.04(1H, d, J=9.92 Hz), 7.45-7.49(3H, m), 7.57-7.60(2H, m), 8.93(1H, d, J=4.80 Hz), 13.38(1H, br.s)

Elemental Analysis for C₁₇H₁₄N₄O₂S

Calcd. C: 60.34; H: 4.17; N: 16.56

Found C: 60.44; H: 4.22; N: 16.59

### EXAMPLE 189

In a sealed tube, a mixture of ethyl 5-(1-methyl-6-oxo-1,6-dihydro-3-pyridazinyl)-4-phenyl-1,3-thiazole-2-carboxylate (171 mg) and cyclopropylamine (0.347 mL) in dioxane (0.5 mL) was heated for 40 hours at 70-75° C. The mixture was concentrated under reduced pressure to give a residue. The residue was crystallized from a mixture of ethanol and diisopropyl ether to give N-cyclopropyl-5-(1-methyl-6-oxo-1,6-dihydro-3-pyridazinyl)-4-phenyl-1,3-thiazole-2-carboxamide as a solid (152 mg).

m.p.: 163-164° C. (ethanol-diisopropyl ether)

IR (KBr): 1672, 1651 cm⁻¹

ESI/MS: 375(M+Na)⁺, 353(M+H)⁺

¹H NMR (CDCl₃, δ): 0.69-0.72(2H, m), 0.87-0.93(2H, m), 2.91-2.95(1H, m), 3.84(3H, s), 6.72(1H, d, J=9.72 Hz), 6.93(1H, d, J=9.72 Hz), 7.35(1H, br.s), 7.43-7.47(3H, m), 7.48-7.52(2H, m)

Elemental Analysis for CiBH₁₆N₄O₂S

Calcd. C: 61.35; H: 4.58; N: 15.90

Found C: 61.35; H: 4.70; N: 15.83

### EXAMPLE 190

In a sealed tube, a mixture of ethyl 5-(1-ethyl-6-oxo-1,6-dihydro-3-pyridazinyl)-4-phenyl-1,3-thiazole-2-carboxylate (178 mg) and cyclopropylamine (0.347 mL) in dioxane (0.5 mL) was heated for 40 hours at 70-75° C. The mixture was concentrated under reduced pressure to give a residue. The residue was crystallized from a mixture of ethanol and diisopropyl ether to give N-cyclopropyl-5-(1-ethyl-6-oxo-1,6-dihydro-3-pyridazinyl)-4-phenyl-1,3-thiazole-2-carboxamide as a solid (144 mg).

m.p.: 144-145° C. (ethanol-diisopropyl ether)

IR (KBr): 3286, 1668, 1653, 1591, 1527 cm⁻¹

ESI/MS: 389(M+Na)⁺, 367(M+H)⁺

¹H NMR (CDCl₃, δ): 0.69-0.72(2H, m), 0.87-0.91(2H, m), 1.42(2H, t, J=7.20 Hz), 2.91-2.95(1H, m), 4.25(2H, q, J=7.20 Hz), 6.72(1H, d, J=9.72 Hz), 6.93(1H, d, J=9.72 Hz), 7.35(1H, br.s), 7.43-7.47(3H, m), 7.50-7.53(2H, m)

Elemental Analysis for C₁₉H₁₈N₄O₂S

Calcd. C: 62.28; H: 4.95; N: 15.29

Found C: 62.42; H: 5.18; N: 15.29

### EXAMPLE 191

In a sealed tube, a mixture of ethyl 5-(6-oxo-1-propyl-1,6-dihydro-3-pyridazinyl)-4-phenyl-1,3-thiazole-2-carboxylate (185 mg) and cyclopropylamine (0.347 mL) in dioxane (0.5 mL) was heated for 40 hours at 70-75° C. The mixture was concentrated under reduced pressure to give a residue. The residue was crystallized from a mixture of ethanol and diisopropyl ether to give N-cyclopropyl-5-(6-oxo-1-propyl-1,6-dihydro-3-pyridazinyl)-4-phenyl-1,3-thiazole-2-carboxamide as a solid (144 mg).

m.p.: 146-147° C. (ethanol-diisopropyl ether)

IR (KBr): 3282, 1676, 1655, 1593, 1535 cm⁻¹

ESI/MS: 403(M+Na)⁺, 381(M+H)⁺

¹H NMR (CDCl₃, δ): 0.69-0.71(2H, m), 0.88-0.91(2H, m), 1.00(3H, t, J=7.42 Hz), 1.84-1.90(2H, m), 2.90-2.96(1H, m), 4.16(2H, t, J=7.36 Hz), 6.72(1H, d, J=9.72 Hz), 6.93(1H, d, J=9.72 Hz), 7.34(1H, br.s), 7.43-7.47(3H, m), 7.49-7.53(2H, m)

Elemental Analysis for C₂₀H₂₀N₄O₂S

Calcd. C: 63.14; H: 5.30; N: 14.73

Found C: 63.26; H: 5.41; N: 14.71

### EXAMPLE 192

In a sealed tube, a mixture of ethyl 5-(1-isopropyl-6-oxo-1,6-dihydro-3-pyridazinyl)-4-phenyl-1,3-thiazole-2-carboxylate (100 mg) and cyclopropylamine (0.075 mL) in dioxane (0.3 mL) was heated for 10 hours at 80-85° C. The mixture was poured into a mixture of water and chloroform. A separated organic layer was washed with brine, dried over magnesium sulfate and concentrated under reduced pressure to give a residue. The residue was purified by a preparative TLC on silica gel (n-hexane:ethyl acetate=50:50, v/v) and crystallized from a mixture of ethanol and n-hexane to give N-cyclopropyl-5-(1-isopropyl-6-oxo-1,6-dihydro-3-pyridazinyl)-4-phenyl-1,3-thiazole-2-carboxamide as a solid (71 mg).

m.p.: 127-128° C. (ethanol-n-hexane)

IR (KBr): 3228, 1666, 1643, 1590, 1533 cm⁻¹

ESI/MS: 783(2M+Na)⁺, 403(M+Na)⁺

¹H NMR (CDCl₃, δ): 0.67-0.74(2H, m), 0.85-0.95(2H, m), 1.38(6H, d, J=6.62 Hz), 2.89-2.99(1H, m), 5.31(1H, 7-plet, J=6.62 Hz), 6.70(1H, d, J=9.70 Hz), 6.93(1H, d, J=9.70 Hz), 7.33(1H, d, J=2.68 Hz), 7.42-7.55(5H, m)

Elemental Analysis for C₂₀H₂₀N₄O₂S

Calcd. C: 63.14; H: 5.30; N: 14.73

Found C: 62.89; H: 5.26; N: 14.58

### EXAMPLE 193

In a sealed tube, a mixture of ethyl 5-(1-allyl-6-oxo-1,6-dihydro-3-pyridazinyl)-4-phenyl-1,3-thiazole-2-carboxylate (184 mg) and cyclopropylamine (0.347 mL) in dioxane (0.5 mL) was heated for 40 hours at 70-75° C. The mixture was concentrated under reduced pressure to give a residue. The residue was crystallized from a mixture of ethanol and diisopropyl ether to give S-(1-allyl-6-oxo-1,6-dihydro-3-pyridazinyl)-N-cyclopropyl-4-phenyl-1,3-thiazole-2-carboxamide as a solid (142 mg).

m.p.: 167.5-168.5° C. (ethanol-diisopropyl ether)

IR (KBr): 3284, 1678, 1655, 1593, 1533 cm⁻¹

ESI/MS: 779(2M+Na)⁺, 401(M+Na)⁺, 379(M+H)⁺

¹H NMR (CDCl₃, δ): 0.67-0.72(2H, m), 0.87-0.93(2H, m), 2.90-2.96(1H, m), 4.78-4.80(2H, m), 5.28-5.36(2H, m), 5.99-6.07(1H, m), 6.74(1H, d, J-9.72 Hz), 6.94(1H, d, J=9.72 Hz), 7.34(1H, br.s), 7.42-7.47(3H, m), 7.49-7.53(2H, m)

Elemental Analysis for C₂₀H₁₈N₄O₂S

Calcd. C: 63.48; H: 4.79; N: 14.80

Found C: 63.29; H: 4.64; N: 14.74

### EXAMPLE 194

In a sealed tube, a mixture of ethyl 5-(1-benzyl-6-oxo-1,6-dihydro-3-pyridazinyl)-4-phenyl-1,3-thiazole-2-carboxylate (105 mg) and cyclopropylamine (0.174 mL) in dioxane (0.25 mL) was heated for 40 hours at 70-75° C. The mixture was concentrated under reduced pressure to give a residue. The residue was crystallized from a mixture of ethanol and diisopropyl ether to give 5-(1-benzyl-6-oxo-1,6-dihydro-3-pyridazinyl)-N-cyclopropyl-4-phenyl-1,3-thiazole-2-carboxamide as a solid (84 mg).

m.p.: 151-152.5° C. (ethanol-diisopropyl ether)

IR (KBr): 3298, 1674, 1657, 1591, 1527 cm⁻¹

ESI/MS: 879(2M+Na)⁺, 451(M+Na)⁺, 429(M+H)⁺

¹H NMR (CDCl₃, δ): 0.67-0.72(2H, m), 0.87-0.93(2H, m), 2.91-2.96(1H, m), 5.33(2H, s),6.71(1H, d, J-9.72 Hz), 6.91(1H, d, J=9.72 Hz), 7.33-7.51(11H, m)

Elemental Analysis for C₂₄H₂₀N₄O₂S

Calcd. C: 67.27; H: 4.70; N: 13.07

Found C: 67.33; H: 4.74; N: 13.09

### EXAMPLE 195

In a sealed tube, a mixture of ethyl 5-[1-(2-methoxyethyl)-6-oxo-1,6-dihydro-3-pyridazinyl]-4-phenyl-1,3-thiazole-2-carboxylate (96.8 mg) and cyclopropylamine (0.174 mL) in dioxane (0.25 mL) was heated for 40 hours at 70-75° C. The mixture was concentrated under reduced pressure to give a residue. The residue was crystallized from a mixture of ethanol and diisopropyl ether to give N-cyclopropyl-5-l 1-(2-methoxyethyl)-6-oxo-1,6-dihydro-3-pyridazinyl]-4-phenyl-1,3-thiazole-2-carboxamide as a solid (77 mg).

m.p.: 161-162.5° C. (ethanol-diisopropyl ether)

IR (KBr): 3290, 1674, 1655, 1591, 1529 cm⁻¹

ESI/MS: 815(2M+Na)⁺, 419(M+Na)⁺, 397(M+H)⁺

¹H NMR (CDCl₃, δ): 0.69-0.72(2H, m), 0.87-0.91(2H, m), 2.91-2.95(1H, m), 3.40(3H, s), 3.83(2H, t, J=5.60 Hz), 4.40(2H, t, J=5.60 Hz), 6.73(1H, d, J=9.72 Hz), 6.93(1H, d, J=9.72 Hz), 7.34(1H, br.s), 7.44-7.47(3H, m), 7.50-7.53(2H, m)

Elemental Analysis for C₂₀H₂₀N₄O₃S

Calcd. C: 60.59; H: 5.08; N: 14.13

Found C: 60.74; H: 5.04; N: 14.22

### EXAMPLE 196

In a sealed tube, a mixture of ethyl 4-(2-fluorophenyl)-5-(1-isopropyl-6-oxo-1,6-dihydro-3-pyridazinyl)-1,3-thiazole-2-carboxylate (202 mg) and cyclopropylamine (0.145 mL) in dioxane (0.3 mL) was heated for 12 hours at 80-85° C. The mixture was concentrated under reduced pressure to give a residue. The residue was crystallized from a mixture of ethanol and n-hexane to give N-cyclopropyl-4-(2-fluorophenyl)-5-(1-isopropyl-6-oxo-1,6-dihydro-3-pyridazinyl)-1,3-thiazole-2-carboxamide as a solid (117 mg).

m.p.: 133.5-135° C. (ethanol-n-hexane)

IR (KBr): 3222, 1664, 1639, 1593, 1533 cm⁻¹

ESI/MS: 421(M+Na)⁺, 399(M+H)⁺

¹H NMR (CDCl₃, δ): 0.64-0.74(2H, m), 0.76-0.95(2H, m), 1.29(6H, d, J=6.58 Hz), 2.87-2.99(1H, m), 5.27(1H, 7-plet, J=6.58 Hz), 6.75(1H, d, J=9.57 Hz), 6.99(1H, d, J=9.57 Hz), 7.11-7.21(1H, m), 7.20-7.55(4H, m)

Elemental Analysis for C₂₀HigFN₄O₂S

Calcd. C: 60.29; H: 4.81; N: 14.06

Found C: 60.57; H: 4.95; N: 14.03

### EXAMPLE 197

In a sealed tube, a mixture of ethyl 4-(3-fluorophenyl)-5-(1-isopropyl-6-oxo-1,6-dihydro-3-pyridazinyl)-1,3-thiazole-2-carboxylate (201 mg) and cyclopropylamine (0.144 mL) in dioxane (0.3 mL) was heated for 12 hours at 80-85° C. The mixture was concentrated under reduced pressure to give a residue. The residue was crystallized from a mixture of ethanol and n-hexane to give N-cyclopropyl-4-(3-fluorophenyl)-5-(1-isopropyl-6-oxo-1,6-dihydro-3-pyridazinyl)-1,3-thiazole-2-carboxamide as a solid (160 mg).

m.p.: 145.5-147° C. (ethanol-n-hexane)

IR (KBr): 3249, 1660, 1587 cm⁻¹

ESI/MS: 819(2M+Na)⁺, 421(M+Na)⁺, 399(M+H)⁺

¹H NMR (CDCl₃, δ): 0.66-0.75(2H, m), 0.86-0.97(2H, m), 1.37(6H, d, J=6.62 Hz), 2.89-2.99(1H, m), 5.31(1H, 7-plet, J=6.62 Hz), 6.76(1H, d, J=9.65 Hz), 6.98(1H, d, J=9.65 Hz), 7.10-7.47(5H, m)

Elemental Analysis for C₂₀H₁₉FN₄O₂S.0.2H₂O

Calcd. C: 59.75; H: 4.86; N: 13.94

Found C: 60.05; H: 5.12; N: 13.64

### EXAMPLE 198

In a sealed tube, a mixture of ethyl 4-(4-fluorophenyl)-5-(1-isopropyl-6-oxo-1,6-dihydro-3-pyridazinyl)-1,3-thiazole-2-carboxylate (100 mg) and cyclopropylamine (0.072 mL) in dioxane (0.3 mL) was heated for 10 hours at 80-85° C. The mixture was concentrated under reduced pressure to give a residue. The residue was purified by a preparative TLC on silica gel (n-hexane:ethyl acetate=50:50, v/v) and crystallized from a mixture of ethanol and diisopropyl ether to give N-cyclopropyl-4-(4-fluorophenyl)-5-(1-isopropyl-6-oxo-1,6-dihydro-3-pyridazinyl)-1,3-thiazole-2-carboxamide as a solid (78 mg).

m.p.: 157-158° C. (ethanol-diisopropyl ether)

IR (KBr): 3228, 1668, 1651, 1637, 1539 cm⁻¹

ESI/MS: 421(M+Na)⁺

¹H NMR (CDCl₃, δ): 0.69-0.72(2H, m), 0.87-0.92(2H, m), 1.37(6H, d, J=6.62 Hz), 2.92-2.95(1H, m), 5.31(1H, 7-plet, J=6.62 Hz), 6.74(1H, d, J=9.68 Hz), 6.94(1H, d, J=9.68 Hz), 7.12-7.17(2H, m), 7.31(1H, d, J=2.92 Hz), 7.49-7.53(2H, m)

Elemental Analysis for C₂₀H₁₉FN₄O₂S

Calcd. C: 60.29; H: 4.81; N: 14.06

Found C: 60.34; H: 4.72; N: 13.98

### EXAMPLE 199

In a sealed tube, a mixture of ethyl 4-(3-chlorophenyl)-5-(1-isopropyl-6-oxo-1,6-dihydro-3-pyridazinyl)-1,3-thiazole-2-carboxylate (203 mg) and cyclopropylamine (0.139 mL) in dioxane (0.3 mL) was heated for 12 hours at 80-85° C. The mixture was concentrated under reduced pressure to give a residue. The residue was crystallized from a mixture of ethanol and diisopropyl ether to give 4-(3-chlorophenyl)-N-cyclopropyl-5-(1-isopropyl-6-oxo-1,6-dihydro-3-pyridazinyl)-1,3-thiazole-2-carboxamide as a solid (141 mg).

m.p.: 118.5-119.5° C. (ethanol-diisopropyl ether)

IR (KBr): 3251, 1660, 1645, 1585 cm⁻¹ ES[/MS: 853 and 851(2M+Na)⁺, 439 and 437(M+Na)⁺, 415(M+H)⁺

¹H NMR (CDCl₃, δ): 0.66-0.76(2H, m), 0.86-0.97(2H, m), 1.36(6H, d, J=6.64 Hz), 2.98-2.99(1H, m), 5.31(1H, 7-plet, J=6.64 Hz), 6.77(1H, d, J=9.78 Hz), 6.99(1H, d, J=9.78 Hz), 7.32-7.59(4H, m), 7.58-7.60(1H, m)

Elemental Analysis for C₂₀H₁₉ClN₄O₂S.0.2H₂O

Calcd. C: 57.40; H: 4.67; N: 13.39

Found C: 57.47; H: 4.76; N: 13.30

### EXAMPLE 200

A mixture of ethyl 5-(6-oxo-1,6-dihydro-3-pyridazinyl)-4-phenyl-1,3-thiazole-2-carboxylate (164 mg) and 2-pyridinylmethylamine (0.155 mL) in dioxane (0.5 mL) was heated for 40 hours at 90-95° C. Water (4 mL) and chloroform (4 mL) were added to the mixture to give a solid. The solid was collected by filtration, dried over phosphorous petoxide and crystallized from ethanol to give 5-(6-oxo-1,6-dihydro-3-pyridazinyl)-4-phenyl-N-(2-pyridinylmethyl)-1,3-thiazole-2-carboxamide as a solid (48 mg).

m.p.: 221-222.5° C. (ethanol)

IR (KBr): 3226, 1674, 1595, 1529 cm⁻¹

ESI/MS: 412(M+Na)⁺, 390(M+H)⁺

¹H NMR (DMSO-ds, 5): 4.60(2H, d, J=6.06 Hz), 6.84(1H, d, J=9.80 Hz), 7.08(1H, d, J=9.80 Hz), 7.25-7.38(2H, m), 7.46-7.52(3H, m), 7.60-7.66(2H, m), 7.72-7.81(1H, m), 8.50-8.54(1H, m), 9.47(1H, t, J=6.06 Hz), 13.37(1H, br.s)

Elemental Analysis for C₂₀H is N₅O₂S

Calcd. C: 61.68; H: 3.88; N: 17.98

Found C: 61.36; H: 4.05; N: 17.79

### EXAMPLE 201

A mixture of ethyl 5-(1-methyl-6-oxo-1,6-dihydro-3-pyridazinyl)-4-phenyl-1,3-thiazole-2-carboxylate (171 mg) and 2-pyridinylmethylamine (0.155 mL) in dioxane (0.5 mL) was heated for 40 hours at 90-95° C. The mixture was concentrated under reduced pressure to give a residue. The residue was purified by a column chromatography on silica gel (n-hexane:ethyl acetate=10:90, v/v) to give 5-(1-methyl-6-oxo-1,6-dihydro-3-pyridazinyl)-4-phenyl-N-(2-pyridinylmethyl)-1,3-thiazole-2-carboxamide as a solid (189 mg).

m.p.: 189-190.5° C. (ethanol-diisopropyl ether)

IR (KBr): 3369, 1674, 1589, 1510 cm⁻¹

ESI/MS: 829(2M+Na)⁺, 426(M+Na)⁺, 404(M+H)⁺

¹H NMR (CDCl₃, δ): 3.85(3H, s), 4.79(2H, d, J=5.60 Hz), 6.73(1H, d, J=9.65 Hz), 6.96(1H, d, J=9.65 Hz), 7.22-7.27(1H, m), 7.32-7.74(7H, m), 8.28(1H, br.t, J=5.40 Hz), 8.59(1H, d, J=4.26 Hz)

Elemental Analysis for C₂₁H₁₇NSO₂S

Calcd. C: 62.52; H: 4.25; N: 17.36

Found C: 62.44; H: 4.35; N: 17.26

### EXAMPLE 202

5-(1-Ethyl-6-oxo-1,6-dihydro-3-pyridazinyl)-4-phenyl-N-(2-pyridinylmethyl)-1,3-thiazole-2-carboxamide was obtained in a manner similar to Example 201.

m.p.: 169-170.5° C. (ethanol-diisopropyl ether)

IR (KBr): 1678, 1593, 1527 cm⁻¹

ESI/MS: 440(M+Na)⁺, 418(M+H)⁺

¹H NMR (CDCl₃, 3): 1.43(3H, t, J=7.18 Hz), 4.25(2H, q, J=7.18 Hz), 4.79(2H, d, J=5.62 Hz), 6.72(1H, d, J=9.70 Hz), 6.96(1H, d, J=9.70 Hz), 7.22-7.74(8H, m), 8.27(1H, br.t, J=5.37 Hz), 8.59(1H, d, J=4.34 Hz)

Elemental Analysis for C₂₂H₁₉N₅O₂S

Calcd. C: 63.29; H: 4.59; N: 16.78

Found C: 63.15; H: 4.66; N: 16.63

### EXAMPLE 203

5-(6-Oxo-1-propyl-1,6-dihydro-3-pyridazinyl)-4-phenyl-N-(2-pyridinylmethyl)-1,3-thiazole-2-carboxamide was obtained in a manner similar to Example 201.

m.p.: 134-135.5° C. (ethanol-diisopropyl ether)

IR (KBr): 3386, 1668, 1587, 1512 cm⁻¹

ESI/MS: 885(2M+Na)⁺, 454(M+Na)⁺, 432(M+H)⁺

¹H NMR (CDCl₃, δ): 1.00(3H, t, J=7.38 Hz), 1.78-1.97(2H, m), 4.16(2H, t, J=7.32 Hz), 4.79(2H, d, J=5.62 Hz), 6.72(1H, d, J=9.68 Hz), 6.96(1H, d, J=9.68 Hz), 7.21-7.74(8H, m), 8.27(1H, br.t, J=5.49 Hz), 8.59(1H, d, J=4.50 Hz)

Elemental Analysis for C₂₃H₂₁N₅O₂S Calcd; C: 64.02; H: 4.91; N: 16.23

Found C: 64.00; H: 4.99; N: 16.06

### EXAMPLE 204

5-(1-Allyl-6-oxo-1,6-dihydro-3-pyridazinyl)-4-phenyl-N-(2-pyridinylmethyl)-1,3-thiazole-2-carboxamide was obtained in a manner similar to Example 201.

m.p.: 117-118° C. (acetone-n-hexane)

IR (KBr): 1680, 1658, 1591, 1514 cm⁻¹

ESI/MS: 881(2M+Na)⁺, 452(M+Na)⁺, 430(M+H)⁺

¹H NMR (CDCl₃, δ): 4.77-4.82(4H, m), 5.28-5.38(2H, m), 5.91-6.15(1H, m), 6.74(1H, d, J=9.60 Hz), 6.97(1H, d,J=9.60 Hz), 7.23-7.74(8H, m), 8.27(1H, br.t, J=5.56 Hz), 8.59(1H, d, J=4.92 Hz)

Elemental Analysis for C₂₃H₁₉N₅O₂S

Calcd. C: 64.32; H: 4.46; N: 16.31

Found C: 64.19; H: 4.47; N: 16.13

### EXAMPLE 205

5-(1-Benzyl-6-oxo-1,6-dihydro-3-pyridazinyl)-4-phenyl-N-(2-pyridinylmethyl)-1,3-thiazole-2-carboxamide was obtained in a manner similar to Example 201.

m.p.: 172.5-173.5° C. (ethanol-diisopropyl ether)

IR (KBr): 3346, 1678, 1589, 1527 cm⁻¹

ESI/MS: 981(2M+Na)⁺, 502(M+Na)⁺, 480(M+H)⁺

¹H NMR (CDCl₃, δ): 4.79(2H, d, J=5.68 Hz), 5.34(2H, s), 6.72(1H, d, J=9.68 Hz), 6.93(1H, d, J=9.68 Hz), 7.26-7.73(13H, m), 8.27(1H, br.t, J=8.27 Hz), 8.58(1H, d, J=4.32 Hz)

Elemental Analysis for C₂₇H₂₁N₅O₂S 0.2H₂O

Calcd. C: 67.12; H: 4.46; N: 14.49

Found C: 67.19; H: 4.40; N: 14.49

### EXAMPLE 206

5-[1-(2-Methoxyethyl)-6-oxo-1,6-dihydro-3-pyridazinyl]-4-phenyl-N-(2-pyridinylmethyl)-1,3-thiazole-2-carboxamide was obtained in a manner similar to Example 201.

m.p.: 168-169.5° C. (ethanol-diisopropyl ether)

IR (KBr): 3379, 1660, 1589, 1522 cm⁻¹

ESI/MS: 917(2M+Na)⁺, 470(M+Na)⁺, 448(M+H)⁺

¹H NMR (CDCl₃, δ): 3.40(3H, s), 3.83(2H, t, J=5.58 Hz), 4.40(2H, t, J=5.58 Hz), 4.79(2H, d, J=5.64 Hz), 6.73(1H, d, J=9.62 Hz), 6.96(1H, d, J=9.62 Hz), 7.21-7.74(8H, m), 8.27(1H, br.t, J=5.35 Hz), 8.59(1H, d, J=5.00 Hz)

Elemental Analysis for C₂₃H₂₁N.₅O₃S

Calcd. C: 61.73; H: 4.73; N: 15.65

Found C: 61.59; H: 4.80; N: 15.44

### EXAMPLE 207

4-(2-Fluorophenyl)-5-(1-isopropyl-6-oxo-1,6-dihydro-3-pyridazinyl)-N-(2-pyridinylmethyl)-1,3-thiazole-2-carboxamide was obtained in a manner similar to Example 201.

m.p.: 190-191° C. (ethanol)

IR (KBr): 3354, 1668, 1595, 1513 cm⁻¹

ESI/MS: 921(2M+Na)⁺, 472(M+Na)⁺, 450(M+H)⁺

¹H NMR (CDCb₃, 6): 1.30(6H, d, J=6.62 Hz), 4.79(2H, d, J=5.60 Hz), 5.28(1H, 7-plet, J=6.62 Hz), 6.76(1H, d, J-9.75 Hz), 7.01(1H, d, J=9.75 Hz), 7.09-7.80(7H, m), 8.18-8.24(1H, m), 8.56-8.60(1H, m)

Elemental Analysis for C₂₃H₂₀FNSO₂S

Calcd. C: 61.46; H: 4.48; N: 15.58

Found C: 61.40; H: 4.53; N: 15.47

### EXAMPLE 208

4-(3-Fluorophenyl)-5-(1-isopropyl-6-oxo-1,6-dihydro-3-pyridazinyl)-N-(2-pyridinylmethyl)-1,3-thiazole-2-carboxamide was obtained in a manner similar to Example 201.

m.p.: 188-189.5° C. (ethanol)

IR (KBr): 3384, 1668, 1587, 1512 cm⁻¹

ESI/MS: 921(2M+Na)⁺, 472(M+Na)⁺, 450(M+H)⁺

¹H NMR (CDCl₃, δ): 1.38(6H, d, J=6.62 Hz), 4.80(2H, d, J=5.56 Hz), 5.32(1H, 7-plet, J=6.62 Hz), 6.77(1H, d, J=9.74 Hz), 7.01(1H, d, J=9.74 Hz), 7.15-7.43(H, m), 7.65-7.76(1H, m), 8.25-8.31(1H, m), 8.59-8.62(1H, m)

Elemental Analysis for C₂₃H₂₀FNsO₂S

Calcd. C: 61.46; H: 4.48; N: 15.58

Found C: 61.42; H: 4.55; N: 15.49

### EXAMPLE 209

4-(3-Chlorophenyl)-5-(1-isopropyl-6-oxo-1,6-dihydro-3-pyridazinyl)-N-(2-pyridinylmethyl)-1,3-thiazole-2-carboxamide was obtained in a manner similar to Example 201.

m.p.: 168-169.5° C. (ethanol-diisopropyl ether)

IR (KBr) 3384, 1668, 1587, 1514 cm⁻¹

ESI/MS: 955 and 953(2M+Na)⁺, 490 and 488(M+Na)⁺, 468 and 466(M+H)⁺

¹H NMR (CDCl₃, δ): 1.37(6H, d, J=6.60 Hz), 4.80(2H, d, J=5.56 Hz), 5.32(1H, 7-plet, J=6.60 Hz), 6.78(1H, d, J=9.78 Hz), 7.01(1H, d, J=9.78 Hz), 7.23-7.27(1H, m), 7.33-7.43(5H, m), 7.61-7.74(2H, m), 8.24-8.30(1H, m), 8.59-8.62(1H, m)

Elemental Analysis for C₂₃H₂₀ClN₅O₂S

Calcd. C: 59.29; H: 4.33; N: 15.03

Found C: 59.38; H: 4.38; N: 14.98

### EXAMPLE 210

A mixture of ethyl 5-(1-isopropyl-6-oxo-1,6-dihydro-3-pyridazinyl)-4-phenyl-1,3-thiazole-2-carboxylate (100 mg) and 2-pyridinylmethylamine (0.112 mL) in dioxane (0.3 mL) was heated for 10 hours at 80-85° C. To the mixture was added water (3 mL) to obtain a solid. The solid was collected by filtration, dissolved in chloroform, dried over magnesium sulfate and concentrated under reduced pressure to give a solid. The solid was crystallized from a mixture of ethanol and diisopropyl ether to give 5-(1-isopropyl-6-oxo-1,6-dihydro-3-pyridazinyl)-4-phenyl-N-(2-pyridinylmethyl)-1,3-thiazole-2-carboxamide as a solid (90 mg).

m.p.: 176.5-177.5° C. (ethanol-diisopropyl ether)

IR (KBr): 3384, 1666, 1589, 1513 cm⁻¹

ESI/MS: 885(2M+Na)⁺, 454(M+Na)⁺, 432(M+H)⁺

¹H NMR (CDCl₃, δ): 1.23(6H, d, J=6.62 Hz), 4.79(2H, d, J=5.50 Hz), 5.33(1H, 7-plet, J=6.62 Hz), 6.71(1H, d, J=9.70 Hz), 6.96(1H, d, J=9.70 Hz), 7.20-7.24(1H, m), 7.35(1H, d, J=7.84 Hz), 7.44-7.50(3H, m), 7.54-7.57(2H, m), 7.66-7.69(1H, m), 8.26(1H, t, J=5.50 Hz), 8.59(1H, d, J=4.84 Hz)

Elemental Analysis for C₂₃H₂₁N₅O₂S

Calcd. C: 64.02; H: 4.91; N: 16.23

Found C: 63.81; H: 4.86; N: 16.08

### EXAMPLE 211

4-(4-Fluorophenyl)-5-(1-isopropyl-6-oxo-1,6-dihydro-3-pyridazinyl)-N-(2-pyridinylmethyl)-1,3-thiazole-2-carboxamide was obtained in a manner similar to Example 210.

m.p.: 203.5-205° C. (ethanol-diisopropyl ether)

IR (KBr): 3383, 1668, 1587, 1514 cm⁻¹

ESI/MS: 472(M+Na)⁺, 450(M+H)⁺

¹H NMR (CDCl₃, δ): 1.38(6H, d, J=6.64 Hz), 4.79(2H, d, J=5.56 Hz), 5.32(1H, 7-plet, J=6.64 Hz), 6.75(1H, d, J=9.68 Hz), 6.98(1H, d, J=9.68 Hz), 7.1 i-7.18(2H, m), 7.23(1H, dd, J=5.02, 6.79 Hz), 7.35(1H, d, J=7.82 Hz), 7.52-7.58(2H, m), 7.69(1H, dt, J=1.78, 7.68 Hz), 8.27(1H, t, J=5.56 Hz), 8.58-8.61(1H, m)

Elemental Analysis for C₂₃H₂₀FNsO₂S

Calcd. C: 61.46; H: 4.48; N: 15.58

Found C: 61.43; H: 4.58; N: 15.42

### EXAMPLE 212

A mixture of ethyl 5-(1-isopropyl-6-oxo-1,6-dihydro-3-pyridazinyl)-4-phenyl-1,3-thiazole-2-carboxylate (500 mg) and potassium tert-butoxide (152 mg) in methylformamide (5 mL) was heated for 5 hours at 95-100° C. Water (50 mL) and 1N-hydrochloric acid (1.35 mL) were added to the reaction mixture. The mixture was extracted with chloroform (20 mL×5). The organic layer was dried over magnesium sulfate and concentrated under reduced pressure to give a residue. The residue was purified by a column chromatography on silica gel (n-hexane:ethyl acetate=50:50, v/v) to give 5-(1-isopropyl-6-oxo-1,6-dihydro-3-pyridazinyl)-N-methyl-4-phenyl-1,3-thiazole-2-carboxamide as a solid (143 mg).

m.p.: 165-167.5° C. (ethanol-diisopropyl ether)

IR (KBr): 3396, 1666, 1589, 1531 cm⁻¹

ESI/MS: ³77(M+Na)⁺

¹H NMR (CDCl₃, δ): 1.38(6H, d, J=6.60 Hz), 3.05(3H, d, J=5.10 Hz), 5.31(1H, 7-plet, J=6.60 Hz), 6.71(1H, d, J=9.72 Hz), 6.95(1H, d, J=9.72 Hz), 7.25-7.35(1H, m), 7.43-7.56(5H, m)

Elemental Analysis for C₁₈H₁₈N₄O₂S

Calcd. C: 61.00; H: 5.12; N: 15.81

Found C: 60.91; H: 5.23; N: 15.75

### EXAMPLE 213

To a solution of 5-(1-isopropyl-6-oxo-1,6-dihydro-3-pyridazinyl)-N-methyl-4-phenyl-1,3-thiazole-2-carboxamide (68 mg) in dimethylformamide (0.2 mL) was added sodium hydride (60% in oil) (8.4 mg), and the mixture was stirred for 30 minutes at 50-55° C. Iodomethane (0.0241 mL) was added to the mixture, and the mixture was stirred at ambient temperature for 18 hours. Water (3 mL) was added to the mixture. The mixture was extracted with ethyl acetate (2 mL×4). The organic layer was dried over magnesium sulfate and concentrated under reduced pressure to give a residue. The residue was purified by preparative TLC on silica gel (n-hexane:ethyl acetate 50:50, v/v) to give 5-(1-isopropyl-6-oxo-1,6-dihydro-3-pyridazinyl)-N,N-dimethyl-4-phenyl-1,3-thiazole-2-carboxamide as a solid (40 mg).

m.p.: 141-144° C. (diisopropyl ether)

IR (KBr): 1664, 1626, 1587 cm⁻¹

ESI/MS: 759(2M+Na)⁺, 391(M+Na)⁺, 369(M+H)⁺

¹H NMR (CDCl₃, δ): 1.38(6H, d, J=6.63 Hz), 3.18(3H, s), 3.65(3H, s), 5.31(1H, 7-plet, J=6.63 Hz), 6.72(1H, d, J=9.70 Hz), 6.99(1H, d, J=9.70 Hz), 7.41-7.47(3H, m), 7.49-7.57(2H, m)

Elemental Analysis for C₁₉H₂₀N₄O₂S

Calcd. C: 61.94; H: 5.47; N: 15.21

Found C: 61.88; H: 5.60; N: 15.13

### EXAMPLE 214

In a sealed tube, a mixture of ethyl 5-(1-isopropyl-6-oxo-1,6-dihydro-3-pyridazinyl)-4-phenyl-1,3-thiazole-2-carboxylate (100 mg) and ethylamine (0.406 mL) in tetrahydrofuran (2 mL) was heated at 50-55° C. for 70 hours. The mixture was concentrated under reduced pressure to give a residue. The residue was purified by preparative TLC on silica gel (n-hexane:ethyl acetate=50:50, v/v) to give N-ethyl-5-(1-isopropyl-6-oxo-1,6-dihydro-3-pyridazinyl)-4-phenyl-1,3-thiazole-2-carboxamide as a solid (84 mg).

m.p.: 172-174° C. (ethanol-diisopropyl ether)

IR (KBr): 3305, 1670, 1658, 1539 cm⁻¹

ESI/MS: 759(2M+Na)⁺, 391(M+Na)⁺

¹H NMR (CDCl₃, δ): 1.28(3H, t, J=7.27 Hz), 1.38(6H, d, J=6.60 Hz), 3.45-3.60(2H, m), 5.31(1H, 7-plet, J=6.60 Hz), 6.71(1H, d, J=9.78 Hz), 6.95(1H, d, J=9.78 Hz), 7.25-7.35(1H, m), 7.42-7.57(5H, m)

Elemental Analysis for C₁₉H₂₀N₄O₂S

Calcd. C: 61.94; H: 5.47; N: 15.21

Found C: 62.07; H: 5.57; N: 15.20

### EXAMPLE 215

A mixture of ethyl 5-(1-isopropyl-6-oxo-1,6-dihydro-3-pyridazinyl)-4-phenyl-1,3-thiazole-2-carboxylate (100 mg) and butylamine (0.108 mL) in dioxane (0.3 mL) was heated for 10 hours at 80-85° C. Water (2 mL) and 1N-hydrochloric acid (0.5 mL) were added to the mixture. The mixture was extracted with chloroform (3 mL), dried over magnesium sulfate and concentrated under reduced pressure to give a residue. The residue was purified by a preparative TLC on silica gel (n-hexane:ethyl acetate=50:50, v/v) to give N-butyl-5-(1-isopropyl-6-oxo-1,6-dihydro-3-pyridazinyl)-4-phenyl-1,3-thiazole-2-carboxamide as a solid (40 mg).

m.p.: 147.5-148.5° C. (ethanol-diisopropyl ether)

IR (KBr): 3294, 1672, 1537 cm⁻¹

ESI/MS: 815(2M+Na)⁺, 419(M+Na)⁺, 397(M+H)⁺

¹H NMR (CDCl₃, δ): 0.96(3H, t, J=7.22 Hz), 1.33-1.67(4H, m), 1.38(6H, d, J=6.66 Hz), 3.42-3.53(2H, m), 5.31(1H, 7-plet), 6.70(1H, d, J=9.58 Hz), 6.94(1H, d, J=9.58 Hz), 7.26-7.32(1H, m), 7.43-7.57(5H, m)

Elemental Analysis for C₂₁H₂₄N₄O₂S.0.1H₂O

Calcd. C: 63.33; H: 6.12; N: 14.07

Found C: 63.27; H: 6.05; N: 14.02

### EXAMPLE 216

5-(1-Isopropyl-6-oxo-1,6-dihydro-3-pyridazinyl)-N-(2-metho.xyethyl)-4-phenyl-1,3-thiazole-2-carboxamide was obtained in a manner similar to Example 215.

m.p.: 131-132.5° C. (ethanol-diisopropyl ether)

IR (KBr): 3419, 1674, 1589, 1527 cm⁻¹

ESI/MS: 819(2M+Na)⁺, 421(M+Na)⁺, 399(M+H)⁺

¹H NMR (CDCl₃, δ): 1.38(6H, d, J=6.62 Hz), 3.39(3H, s), 3.58(2H, t, J=4.96 Hz), 3.65-3.70(2H, m), 5.31(1H, 7-plet, J=6.62 Hz), 6.71(1H, d, J=9.70 Hz), 6.94(1H, d, J=9.70 Hz), 7.44-7.47(3H, m), 7.52-7.60(3H, m)

Elemental Analysis for C₂₀H₂₂N₄O₃S

Calcd. C: 60.28; H: 5.56; N: 14.06

Found C: 60.11; H: 5.47; N: 14.02

### EXAMPLE 217

N-[2-(Acetylamino)ethyl]-5-(1-isopropyl-6-oxo-1,6-dihydro-3-pyridazinyl)-4-phenyl-1,3-thiazole-2-carboxamide was obtained in a manner similar to Example 215.

m.p.: 170-171.5° C. (ethanol-diisopropyl ether)

IR (KBr): 3294, 1657, 1585, 1533 cm⁻¹

ESI/MS: 873(2M+Na)⁺, 448(M+Na)⁺, 426(M+H)⁺

¹H NMR (CDCl₃, δ): 1.38(6H, d, J=6.62 Hz), 2.00(3H, s), 3.47-3.56(2H, m), 3.58-3.68(2H, m), 5.32(1H, 7-plet, J=6.62 Hz), 6.16(1H, br.s), 6.71(1H, d, J=9.63 Hz), 6.95(1H, d, J=9.63 Hz), 7.43-7.57(5H, m), 7.68(1H, t, J=5.78 Hz)

Elemental Analysis for C₂₁H₂₃N₅O₃S

Calcd. C: 59.28; H: 5.45; N: 16.46

Found C: 58.83; H: 5.36; N: 16.28

### EXAMPLE 218

2-Isopropyl-6-[4-phenyl-2-(1-piperidinylcarbonyl)-1,3-thiazol-5-yl]-3(2H)-pyridazinone was obtained in a manner similar to Example 215.

m.p.: 111-112° C. (n-hexane)

IR (KBr): 1670, 1618, 1589 cm⁻¹

ESI/MS: 839(2M+Na)⁺, 431(M+Na)⁺, 409(M+H)⁺

¹H NMR (CDCl₃, δ): 1.37(6H, d, J=6.64 Hz), 1.71(6H, br.s), 3.75(2H, br.s), 4.29(2H, br.s), 5.31(1H, 7-plet, J=6.64 Hz), 6.71(1H, d, J=9.68 Hz), 6.98(1H, d, J=9.68 Hz), 7.41-7.45(3H, m), 7.52-7.55(2H, m)

Elemental Analysis for C₂₂H₂₄N₄O₂S 0.1H₂O

Calcd. C: 64.40; H: 5.94; N: 13.65

Found C: 64.38; H: 5.82; N: 13.61

### EXAMPLE 219

6-{2-[(4-Acetyl-1-piperazinyl)carbonyl]-4-phenyl-1,3-thiazol-5-yl}-2-isopropyl-3(2H)-pyridazinone was obtained in a manner similar to Example 215.

m.p.: 78-82° C. (n-hexane)

IR (KBr): 1662, 1624, 1589 cm⁻¹

ESI/MS: 474(M+Na)⁺, 452(M+E)⁺

¹H NMR (CDCl₃, δ): 1.38(6H, d, J=6.62 Hz), 2.15(3H, s), 3.55-3.90(6H, m), 4.40-4.65(2H, m), 5.32(1H, 7-plet, J=6.62 Hz), 6.73(1H, d, J=9.68 Hz), 6.98(1H, d, J=9.68 Hz), 7.42-7.55(5H, m)

Elemental Analysis for C₂₃H₂₅N₅O₃S 0.5H₂O

Calcd. C: 59.98; H: 5.69; N: 15.21

Found C: 60.14; H: 5.65; N: 14.95

### EXAMPLE 220

N-Benzyl-5-(1-isopropyl-6-oxo-1,6-dihydro-3-pyridazinyl)-4-phenyl-1,3-thiazole-2-carboxamide was obtained in a manner similar to Example 215.

m.p.: 186-187° C. (ethanol-diisopropyl ether)

IR (KBr): 3344, 1660, 1587, 1529 cm⁻¹

ESI/MS: 883(2M+Na)⁺, 453(M+Na)⁺, 431(M+H)⁺

¹H NMR (CDCl₃, δ): 1.38(6H, d, J=6.63 Hz), 4.67(2H, d, J=6.14 Hz), 5.31(1H, 7-plet, J=6.63 Hz), 6.71(1H, d, J=9.68 Hz), 6.94(1H, d, J=9.668 Hz), 7.30-7.60(11H, m)

Elemental Analysis for C₂₄H₂₂N₄O₂S.0.1H₂O

Calcd. C: 66.68; H: 5.18; N: 12.96

Found C: 66.64; H: 5.13; N: 12.93

### EXAMPLE 221

4-(4-Fluorophenyl)-5-(1-isopropyl-6-oxo-1,6-dihydro-3-pyridazinyl)-N-[(5-methyl-2-pyrazinyl)methyl]-1,3-thiazole-2-carboxamide was obtained in a manner similar to Example 215.

m.p.: 187.5-188.5° C. (ethanol-diisopropyl ether)

IR (KBr): 1670, 1520 cm⁻¹

ESI/MS: 487(M+Na)⁺

¹H NMR (CDCl₃, δ): 1.37(6H, d, J=6.62 Hz), 2.57(3H, s), 4.79(2H, d, J=5.76 Hz), 5.31(1H, 7-plet, J=6.62 Hz), 6.75(1H, d, J=9.68 Hz), 6.97(1H, d, J=9.68 Hz), 7.12-7.17(2H, m), 7.51-7.56(2H, m), 8.06(1H, t, J=5.76 Hz), 8.43(1H, s), 8.56(1H, s)

Elemental Analysis for C₂₃H₂₁FN₆O₂S

Calcd. C: 59.47; H: 4.56; N: 18.09

Found C: 59.36; H: 4.54; N: 18.00

### EXAMPLE 222

A mixture of ethyl 5-(1-isopropyl-6-oxo-1,6-dihydro-3-pyridazinyl)-4-phenyl-1,3-thiazole-2-carboxylate (100 mg), 2-aminoacetamide hydrochloride (120 mg) and triethylamine (0.151 mL) in dioxane (0.3 mL) was heated for 10 hours at 80-85° C. To the reaction mixture, water (2 mL) and 1N-hydrochloric acid (0.5 mL) were added to give a precipitate. The precipitate was collected by filtration, dissolved in chloroform, dried over magnesium sulfate and concentrated under reduced pressure to give a residue. The residue was crystallized from ethanol to give N-(2-amino-2-oxoethyl)-5-(1-isopropyl-6-oxo-1,6-dihydro-3-pyridazinyl)-4-phenyl-1,3-thiazole-2-carboxamide as a solid (60 mg).

m.p.: 247.5-249° C. (ethanol)

IR (KBr): 3392, 3199, 1666, 1587 cm⁻¹

ESI/MS: 817(2M+Na)⁺, 420(M+Na)⁺

¹H NMR (DMSO-d₆, δ): 1.24(6H, d, J=6.66 Hz), 3.86(2H, d, J=5.95 Hz), 5.13(1H, 7-plet, J=6.66 Hz), 6.89(1H, d, J=9.62 Hz), 7.12(1H, br.s), 7.16(1H, d, J-9.62 Hz), 7.46-7.63(6H, m), 8.87(1H, t, J=5.95 Hz)

Elemental Analysis for CigHigNSO₃S

Calcd. C: 57.42; H: 4.82; N: 17.62

Found C: 57.54; H: 4.90; N: 17.25

### EXAMPLE 223

N-12-(Dimethylamino)ethyl]-5-(1-isopropyl-6-oxo-1,6-dihydro-3-pyridazinyl)-4-phenyl-1,3-thiazole-2-carboxamide was obtained in a manner similar to Example 222.

m.p.: 151-152.5° C. (ethanol-diisopropyl ether)

IR (KBr): 3408, 1668, 1587, 1514 cm⁻¹

ESI/MS: 434(M+Na)⁺, 412(M+H)⁺

¹H NMR (CDCl₃, 3): 1.38(6H, d, J=6.60 Hz), 2.53(2H, t, J=6.11 Hz), 3.51-3.61(2H, m), 5.31(1H, 7-plet, J=6.60 Hz), 6.70(1H, d, J=9.70 Hz), 6.94(1H, d, J=9.70 Hz), 7.43-7.62(6H, m)

Elemental Analysis for C₂₁H₂₅N₅O₂S

Calcd. C: 61.29; H: 6.12; N: 17.02

Found C: 61.10; H: 6.03; N: 16.84

### EXAMPLE 224

5-(1-Isopropyl-6-oxo-1,6-dihydro-3-pyridazinyl)-N-[2-(4-morpholinyl)-ethyl]-4-phenyl-1,3-thiazole-2-carboxamide was obtained in a manner similar to Example 222.

m.p.: 196.5-197.5° C. (ethanol)

IR (KBr): 3413, 1670, 1587, 1512 cm⁻¹

ESI/MS: 929(2M+Na)⁺, 476(M+Na)⁺, 454(M+H)⁺

¹H NMR (CDCl₃, δ): H1.37(6H, d, J=6.60 Hz), 2.49-2.54(4H, m), 2.61(2H, t, J=6.19 Hz), 3.54-3.64(2H, m), 3.70-376(4H, m), 5.31(1H, 7-plet, J=6.60 Hz), 6.71(1H, d, J=9.60 Hz), 6.96(1H, d, J=9.60 Hz), 7.43-7.58(5H, m), 7.68(1H, t, J=5.27 Hz)

Elemental Analysis for C₂₃H₂₇NsO₃S

Calcd. C: 60.91; H: 6.00; N: 15.44

Found C: 60.67; H: 5.90; N: 15.19

### EXAMPLE 225

N-Cyclohexyl-5-(1-isopropyl-6-oxo-1,6-dihydro-3-pyridazinyl)-4-phenyl-1,3-thiazole-2-carboxamide was obtained in a manner similar to Example 222.

m.p.: 225-226° C. (ethanol-diisopropyl ether)

IR (KBr): 3307, 1670, 1597, 1531 cm⁻¹

ESI/MS: 867(2M+Na)⁺, 445(M+Na)⁺, 423(M+H)⁺

¹H NMR (CDCl₃, δ): 1.20-1.47(5H, m), 1.38(6H, d, J=6.60 Hz), 1.60-1.80(3H, m), 2.01-2.06(2H, m), 3.91-4.01(1H, m), 5.31(1H, 7-plet, J=6.60 Hz), 6.70(1H, d, J-9.66 Hz), 6.93(1H, d, J=9.66 Hz), 7.17(1H, br.s), 7.44-7.47(3H, m), 7.52-7.56(2H, m)

Elemental Analysis for C₂₃H₂₆N₄O₂S.0.2H₂O

Calcd. C: 64.83; H: 6.24; N: 13.15

Found C: 64.79; H: 6.11; N: 13.15

### EXAMPLE 226

2-Isopropyl-6-[4-phenyl-2-(1-pyrrolidinylcarbonyl)-1,3-thiazol-5-yl]-3(2H)-pyridazinone was obtained in a manner similar to Example 222.

m.p.: 169-170.5° C. (ethanol-diisopropyl ether)

IR (KBr): 1666, 1620, 1591 cm⁻¹

ESI/MS: 811(2M+Na)⁺, 417(M+Na)⁺, 395(M+H)⁺

¹H NMR (CDCl₃, 3): 1.38(6H, d, J=6.62 Hz), 1.90-1.97(2H, m), 1.99-2.06(2H, m), 3.72(2H, t, J=6.85 Hz), 4.17(2H, t, J=6.85 Hz), 5.31(1H, 7-plet, J=6.62 Hz), 6.72(1H, d, J=9.68 Hz), 6.99(1H, d, J=9.68 Hz), 7.41-7.45(3H, m), 7.52-7.56(2H, m)

Elemental Analysis for C₂₁H₂₂N₄O₂S

Calcd. C: 63.94; H: 5.62; N: 14.20

Found C: 63.67; H: 5.52; N: 14.19

### EXAMPLE 227

2-Isopropyl-6-[2-(4-morpholinylcarbonyl)-4-phenyl-1,3-thiazol-5-yl]-3(2H)-pyridazinone was obtained in a manner similar to Example 222.

m.p.: 161.5-162.5° C. (ethanol-diisopropyl ether)

IR (KBr): 1664, 1624, 1585 cm⁻¹

ESI/MS: 843(2M+Na)⁺, 433(M+Na)⁺, 411(M+H)⁺

¹H NMR (CDCl₃, δ): 1.38(6H, d, J=6.60 Hz), 3.70-3.85(6H, m), 4.48-4.54(2H, m), 5.31(1H, 7-plet, J=6.60 Hz), 6.72(1H, d, J=9.58 Hz), 6.97(1H, d, J=9.58 Hz), 7.40-7.55(5H, m)

Elemental Analysis for C₂₁H₂₂N₄O₃S

Calcd. C: 61.45; H: 5.40; N: 13.65

Found C: 61.18; H: 5.30; N: 13.62

### EXAMPLE 228

2-Isopropyl-6-{2-[(4-methyl-1-piperazinyl)carbonyl]-4-phenyl-1,3-thiazol-5-yl)-3(2H)-pyridazinone was obtained in a manner similar to Example 222.

m.p.: 155-156.5° C. (ethanol-diisopropyl ether)

IR (KBr): 1668, 1628, 1589 cm⁻¹

ESI/MS: 869(2M+Na)⁺, 446(M+Na)⁺, 424(M+H)⁺

¹H NMR (CDCl₃, δ): 1.37(6H, d, J=6.58 Hz), 2.34(3H, s), 2.48-2.54(4H, m), 3.82-3.86(2H, m), 4.45-4.49(2H, m), 5.31(1H, 7-plet, J=6.58 Hz), 6.72(1H, d, J=9.80 Hz), 6.97(1H, d, J=9.80 Hz), 7.41-7.56(5H, m)

Elemental Analysis for C₂₂H₂₅N₅O₂S.0.1H₂₀

Calcd. C: 62.13; H: 5.97; N: 16.47

Found C: 62.03; H: 5.82; N: 16.47

### EXAMPLE 229

6-{2-[(4-Benzyl-1-piperazinyl)carbonyl]-4-phenyl-1,3-thiazol-5-yl}-2-isopropyl-3(2H)-pyridazinone was obtained in a manner similar to Example 222.

m.p.: 181-182° C. (ethanol)

IR (KBr): 1666, 1624, 1587 cm⁻¹

ESI/MS: 522(M+Na)⁺, 500(M+H)⁺

¹H NMR (CDCl₃, δ): 1.37(6H, d, J=6.60 Hz), 2.51-2.59(4H, m), 3.55(2H, s), 3.82-3.86(2H, m), 4.41-4.48(2H, m), 5.31(1H, 7-plet), 6.71(1H, d, J=9.62 Hz), 6.97(1H, d, J=9.62 Hz), 7.26-7.54(10H, m)

Elemental Analysis for C₂₈H₂₁N₅O₂S.0.2H₂O

Calcd. C: 66.83; H: 5.89; N: 13.92

Found C: 66.89; H: 5.73; N: 14.03

### EXAMPLE 230

5-(1-Isopropyl-6-oxo-1,6-dihydro-3-pyiidazinyl)-4-phenyl-N-(2-phenylethyl)-1,3-thiazole-2-carboxamide was obtained in a manner similar to Example 222.

m.p.: 115-116° C. (ethanol-diisopropyl ether)

IR (KBr): 3361, 1660, 1587, 1531 cm⁻¹

ESI/MS: 911(2M+Na)⁺, 467(M+Na)⁺, 445(M+H)+tH NMR (CDCl₃, δ): 1.38(6H, d, J=6.58 Hz), 2.96(2H, t, J=7.24 Hz), 3.67-3.79(2H, m), 5.31(1H, 7-plet, J=6.58 Hz), 6.70(1H, d, J=9.76 Hz), 6.95(1H, d, J-9.76 Hz), 7.23-7.52(11H, m)

Elemental Analysis for C₂₅H₂₄N₄O₂S

Calcd. C: 67.55; H: 5.44; N: 12.60

Found C: 76.32; H: 5.38; N: 12.55

### EXAMPLE 231

Under nitrogen atmosphere, a mixture of ethyl 5-(1-isopropyl-6-oxo-1,6-dihydro-3-pyridazinyl)-4-phenyl-1,3-thiazole-2-carboxylate (100 mg) and 1-phenylpiperazine (0.165 mL) was heated for 18 hours at 120-125° C. The rmixture was purified by a preparative TLC on silica gel (n-hexane:ethyl acetate=50:50, v/v) to give 2-isopropyl-6-(4-phenyl-2-[(4-phenyl-1-piperazinyl)carbonyl]-1,3-thiazol-5-yl}-3(2H)-pyridazinone as a solid (91 mg).

m.p.: 163.5-165° C. (ethanol)

IR (KBr): 1664, 1625, 1591 cm⁻¹

ESI/MS: 508(M+Na)⁺, 486(M+H)⁺

¹H NMR (CDCl₃, δ): 1.38(6H, d, J=6.60 Hz), 3.31(4H, br.s), 3.99(2H, br.s), 4.64(2H, br.s), 5.32(1H, 7-plet, J=6.60 Hz), 6.73(1H, d, J=9.70 Hz), 6.91-7.01(4H, m), 7.26-7.34(2H, m), 7.43-7.57(5H, m)

Elemental Analysis for C₂₇H₂₇NsO₂S 0.2H₂O

Calcd. C: 66.29; H: 5.65; N: 14.32

Found C: 66.25; H: 5.52; N: 14.37

### EXAMPLE 232

A mixture of ethyl 5-(1-isopropyl-6-oxo-1,6-dihydro-3-pyridazinyl)-4-phenyl-1,3-thiazole-2-carboxylate (100 mg) and 3-pyridinylmethylamine (0.111 mL) in dioxane (0.3 mL) was heated for 20 hours at 100-105° C. The mixture was concentrated under reduced pressure and purified by a column chromatography on silica gel (n-hexane:ethyl acetate=20:80, v/v) to give 5-(1-isopropyl-6-oxo-1,6-dihydro-3-pyridazinyl)-4-phenyl-N-(3-pyridinylmethyl)-1,3-thiazole-2-carboxamide as a solid (82 mg).

m.p.: 92.5-194° C. (ethanol-diisopropyl ether)

IR (KBr): 1670, 1589 cm⁻¹

ESI/MS: 885(2M+Na)⁺, 454(M+Na)⁺, 432(M+H)⁺

¹H NMR (CDCl₃, δ): 1.39(6H, d, J=6.60 Hz), 4.68(2H, d, J=6.26 Hz), 5.31(1H, 7-plet, J=6.60 Hz), 6.71(1H, d, J=9.66 Hz), 6.95(1H, d, J=9.66 Hz), 7.26-7.32(1H, m), 7.42-7.54(5H, m), 7.68-7.76(2H, m), 8.56(1H, dd, J=1.58, 4.80 Hz), 8.63(1H, d, J=2.06 Hz)

Elemental Analysis for C₂₃H₂₁NsO₂S

Calcd. C: 64.02; H: 4.91; N: 16.23

Found C: 63.90; H: 4.82; N: 16.15

### EXAMPLE 233

5-(1-Isopropyl-6-oxo-1,6-dihydro-3-pyridazinyl)-4-phenyl-N-(4-pyridinylmethyl)-1,3-thiazole-2-carboxamide was obtained in a manner similar to Example 232.

m.p.: 192-193° C. (ethanol-diisopropyl ether)

IR (KBr): 1670, 1589 cm⁻¹

ESI/MS: 454(M+Na)⁺, 432(M+H)⁺

¹H NMR (CDCl₃, δ): 1.39(6H, d, J=6.60 Hz), 4.68(2H, d, J=6.32 Hz), 5.32(1H, 7-plet, J=6.60 Hz), 6.72(1H, d, J=9.72 Hz), 6.95(1H, d, J=9.72 Hz), 7.26-7.30(2H, m), 7.43-7.56(5H, m), 7.74(1H, t, J=6.32 Hz), 8.57-8.61(2H, m)

Elemental Analysis for C₂₃H₂₁N₅O₂S

Calcd. C: 64.02; H: 4.91; N: 16.23

Found C: 63.74; H: 4.82; N: 16.10

### EXAMPLE 234

5-(1-Isopropyl-6-oxo-1,6-dihydro-3-pyridazinyl)-4-phenyl-N-[2-(2-pyridinyl)ethyl]-1,3-thiazole-2-carboxamide was obtained in a manner similar to Example 232.

m.p.: 144-147° C. (ethanol-diIsopropyl ether)

IR (KBr): 1666, 1587, 1520 cm⁻¹

ESI/MS: 913(2M+Na)⁺, 468(M+Na)⁺, 446(M+H)⁺

¹H NMR (CDClz, 8): 1.38(6H, d, J=6.60 Hz), 3.13(2H, t, J=6.54 Hz), 3.85-3.96(2H, m), 5.31(1H, 7-plet, J=6.60 Hz), 6.71(1H, d, J=9.64 Hz), 6.96(1H, d, J=9.64 Hz), 7.17-7.26(2H, m), 7.42-7.64(6H, m), 8.07(1H, t, J=5.88 Hz), 8.55-8.58(1H, m)

Elemental Analysis for C₂₄H₂₃N₅O₂S

Calcd. C: 64.70; H: 5.20; N: 15.72

Found C: 64.56; H: 5.15; N: 15.54

### EXAMPLE 235

4-(4-Fluorophenyl)-5-(11-isopropyl-6-oxo-1,6-dihydro-3-pyridazinyl)-N-(3-pyridinylmethyl)-1,3-thiazole-2-carboxamide was obtained in a manner similar to Example 232.

m.p.: 202-204° C. (ethanol-diisopropyl ether)

IR (KBr): 1668, 1589 cm⁻¹

ESI/MS: 921(2M+Na)⁺, 472(M+Na)⁺, 450(M+H)⁺

¹H NMR (CDCl₃, δ): 1.38(6H, d, J=6.60 Hz), 4.69(2H, d, J=6.28 Hz), 5.32(1H, 7-plet, J=6.60 Hz), 6.75(1H, d, J=9.66 Hz), 6.96(1H, d, J=9.66 Hz), 7.09-7.18(2H, m), 7.26-7.33(1H, m), 7.47-7.55(2H, m), 7.64-7.75(2H, m), 8.57(1H, dd, J=1.56, 4.80 Hz), 8.64(1H, d, J=2.08 Hz)

Elemental Analysis for C₂₃H₂₀FN₅O₂S

Calcd. C: 61.46; H: 4.48; N: 15.58

Found C: 61.24; H: 4.41; N: 15.45

### EXAMPLE 236

4-(4-Fluorophenyl)-5-(1-isopropyl-6-oxo-1,6-dihydro-3-pyridazinyl)-N-(4-pyridinylmethyl)-1,3-thiazole-2-carboxamide was obtained in a manner similar to Example 232.

m.p.: 155-157° C. (acetone-diisopropyl ether)

IR (KBr): 1668, 1589 cm⁻¹

ESI/MS: 448(M−1)⁻

¹H NMR (CDCl₃, δ): 1.38(6H, d, J=6.60 Hz), 4.68(2H, d, J=6.40 Hz), 5.32(1H, 7-plet, J=6.60 Hz), 6.76(1H, d, J=9.69 Hz), 6.97(1H, d, J=9.69 Hz), 7.10-7.19(2H, m), 7.26-7.30(2H, m), 7.48-7.56(2H, m), 7.70(1H, t, J=6.40 Hz), 8.59(2H, d, J=5.74 Hz)

Elemental Analysis for C₂₃H₂₀FN₅O₂S.0.1H₂O

Calcd. C: 61.21; H: 4.51; N: 15.52

Found C: 61.41; H: 4.55; N: 15.10

### EXAMPLE 237

4-(4-Fluorophenyl)-5-(1-isopropyl-6-oxo-1,6-dihydro-3-pyridazinyl)-N-[2-(2-pyridinyl)ethyl]-1,3-thiazole-2-carboxamide was obtained in a manner similar to Example 232.

m.p.: 144-145° C. (acetone-diisopropyl ether)

IR (KBr): 1670, 1589 cm⁻¹

ESI/MS: 486(M+Na)⁺, 464(M+H)⁺

¹H NMR (CDCl₃, δ): 1.37(6H, d, J=6.60 Hz), 3.13(2H, d, J=6.52 Hz), 3.85-3.96(2H, m), 5.31(1H, 7-plet, J=6.60 Hz), 6.75(1H, d, J=9.64 Hz), 6.97(1H, d, J=9.64 Hz), 7.09-7.24(4H, m), 7.49-7.64(3H, m), 8.10(1H, t, J=5.77 Hz), 8.54-8.58(1H, m)

Elemental Analysis for C₂₄H₂₂FN₅O₂S.H₂O

Calcd. C: 59.86; H: 5.02; N: 14.54

Found C: 59.84; H: 5.06; N: 14.14

### EXAMPLE 238

In a sealed tube, a mixture of ethyl 4-(4-fluorophenyl)-5-(1-isopropyl-6-oxo-1,6-dihydro-3-pyridazinyl)-1,3-thiazole-2-carboxylate (150 mg), O-methylhydroxylamine hydrochloride (162 mg) and potassium tert-butoxide (217 mg) in methanol (2 mL) was heatedfor 10 hours at 70-75° C. Water (9 ml) was added to the mixture to give a solid. The solid was collected by filtration, dissolved in chloroform, dried over magnesium sulfate and concentrated under reduced pressure to give a residue. The residue was purified by a preparative TLC on silica gel (n-hexane:ethyl acetate 50:50, v/v) to give 4-(4-fluorophenyl)-5-(1-isopropyl-6-oxo-1,6-dihydro-3-pyridazinyl)-N-methoxy-1,3-thiazole-2-carboxamide as a solid (32 mg).

m.p.: 164.5-166.5° C. (ethanol-diisopropyl ether)

IR (KBr): 3421, 1722, 1668, 1587 cm⁻¹

ESI/MS: 396(M+Na-15)⁺, 374(M−14)⁺

¹H NMR (CDCl₃, 3): 1.39(6H, d, J=6.62 Hz), 4.05(3H, s), 5.32(1H, 7-plet, J=6.62 Hz), 6.75(1H, d, J=9.72 Hz), 6.97(1H, d, J=9.72 Hz), 7.11-7.16(1H, m), 7.52-7.57(1H, m)

### EXAMPLE 239

4-(4-Fluorophenyl)-5-(1-isopropyl-6-oxo-1,6-dihydro-3-pyridazinyl)-N′,N′-dimethyl-1,3-thiazole-2-carbohydrazide was obtained in a manner similar to Example 238.

m.p.: 169-170.5° C. (ethanol-diisopropyl ether)

IR (KBr): 3444, 1668 cm⁻¹

ESI/MS: 825(2M+Na)⁺, 424(M+Na)⁺, 402(M+H)⁺

¹H NMR (CDCl₃, 3): 1.37(6H, d, J=6.61 Hz), 2.74(6H, s), 5.31(1H, 7-plet, J=6.61 Hz), 6.75(1H, d, J=9.68 Hz), 6.95(1H, d, J=9.68 Hz), 7.13-7.19(2H, m), 7.51-7.55(2H, m), 7.96(1H, s)

Elemental Analysis for C₁₉H₂₀FN₅O₂S

Calcd. C: 56.85; H: 5.02; N: 17.44

Found C: 56.98; H: 5.06; N: 17.47

### EXAMPLE 240

To a solution of 5-(1-isopropyl-6-oxo-1,6-dihydro-3-pyridazinyl)-4-phenyl-1,3-thiazole-2-carbonitrile (162 mg) and thioacetamide (114 mg) in dimethylformamide (1 mL) was added 4.0 M solution of hydrogen chloride in dioxane (1 mL). The mixture was stirred for 3 hours at 100-105° C. Water was added to the reaction mixtuere to give a solid. The solid was collected by filtration, dissolved in a mixture of methanol and chloroform (5:95 v/v), dried over magnesium sulfate and concentrated under reduced pressure to give a residue. The residue was purified by a column chromatography on silica gel (chloroform only) to give 5-(1-isopropyl-6-oxo-1,6-dihydro-3-pyridazinyl)-4-phenyl-1,3-thiazole-2-carbothioamide as a solid (143 mg).

m.p.: >250° C. (ethanol)

IR (KBr): 1660, 1622, 1583 cm⁻¹

ESI/MS: 379(M+Na)⁺, 357(M+H)⁺

¹H NMR (DMSO-d₆, δ): 1.24(6H, d, J=6.64 Hz), 5.13(1H, 7-plet, J=6.64 Hz), 6.88(1H, d, J=9.70 Hz), 7.14(1H, d, J=9.70 Hz), 7.42-7.50(3H, m), 7.57-7.63(2H, m), 9.91(1H, br.s), 10.27(1H, br.s)

Elemental Analysis for C₁₇H₁₆N₄OS₂H₂O

Calcd. C: 54.53; H: 4.84; N: 14.96

Found C: 54.30; H: 4.42; N: 14.56

1. A thiazole derivative of the formula (I): 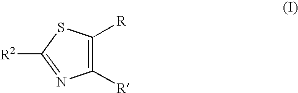
R is a 1-optionally substituted-6-oxo-1,6-dihydro-3-pyridazinyl,
R′ is an optionally substituted phenyl,
R² is a hydrogen atom,
a group represented by the formula (i): 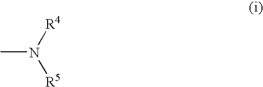
wherein
R⁴ is hydrogen atom,
a lower alkyl group or
a lower alkenyl group, and
R⁵ is hydrogen atom,
an optionally substituted lower alkyl group,
an acyl group,
a cyclo(lower)alkyl group,
a lower alkenyl group,
an optionally substituted aryl group or
a heterocyclic group, or
a group represented by the formula (ii): 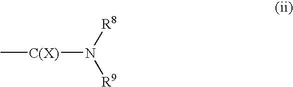
wherein
X is an oxygen or sulfur atom,
R⁸ is a hydrogen atom or
a lower alkyl group,
R⁹ is a hydrogen atom,
an optionally substituted lower alkyl group,
a cyclo(lower)alkyl group,
a lower alkoxy group or
a mono- or di-lower alkylamino group or
R⁸ and R⁹ may combine together to form an optionally substituted saturated N-containing heterocyclic group,
or a salt thereof.

2. A thiazole derivative of claim 1,
wherein the derivative is represented by the formula (1-1): 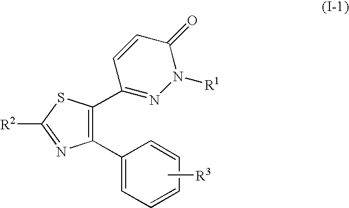
wherein
R¹ is a hydrogen atom,
an optionally substituted lower allyl group,
a lower alkenyl group, or
a cyclo(lower)allyl,
R² is as defined in claim 1, and
R³ is a hydrogen atom, a halogen atom, a hydroxy group, a lower alkyl group or a lower alkoxy group.

3. A compound of claim 2,
wherein
R¹ is a hydrogen atom;
a lower alkyl group which may be substituted with lower alkoxy, lower alkoxycarbonyl, lower alkanoyl, cyclo(lower)alkyl or aryl;
a lower alkenyl group; or
a cyclo(lower)alkyl;
R² is a hydrogen atom,
a group represented by the formula (ia): 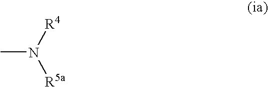
wherein
R⁴ is a hydrogen atom,
a lower alkyl group or
a lower alkenyl group, and
R^{5a} is a hydrogen atom;
a lower alkyl group which may be substituted with one or more substituents selected from amino, imino, lower alkoxy, aryl and saturated or unsaturated heterocyclic group;
a lower alkyl sulfonyl group;
a cyclo(lower)alkyl group;
a lower alkenyl group;
an aryl group which may be substituted with halo(lower)alkyl or di(lower)alkylamino;
an unsaturated heterocyclic group,
a group represented by the formula (iii): 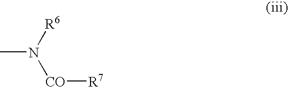
wherein
R⁶ is a hydrogen atom or
a lower alkyl group, and
R⁷ is a hydrogen atom;
a cyclo(lower)alkyl group;
a lower alkoxy group;
an aryloxy group;
a saturated or unsaturated heterocyclic group;
a mono- or di-lower alkylamino group;
an ar(lower)alkylamino group;
a lower alkyl group which may be substituted with halogen, aryl, lower alkoxy-substituted aryl, aryloxy, or a group of the formula (Iv): 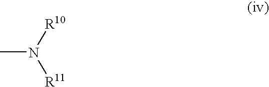
wherein
R¹⁰ is a hydrogen atom or a lower alkyl group,
R¹¹ is a lower alkyl group, a cyclo(lower)alkyl group, a hydroxy(lower)alkyl group, a lower alkoxy(lower)alkyl group, a saturated or unsaturated heterocyclic(lower)allyl group, a mono- or di-lower alkylamino(lower)alkyl group, a lower alkanoylamino(lower)alkyl group, an ar(lower)alkyl group, a hydroxy- or sulfamoyl-substituted ar(lower)alkyl group or a pyrrolidonyl(lower)alkyl group,
or R¹⁰ and R¹¹ may combine together to form a N-containing heterocyclic group which may be substituted with lower allyl or lower alkanoyl;
an arylamino group which may be substituted with lower alkyl;
an arylsulfonylamino group which may be substituted with lower alkyl; or
an aryl group which may be substituted with one or more of substituent(s) selected from the group consisting of halogen, lower alkyl, halo(lower)alkyl, lower alkoxy, halo(lower)alkoxy, and
a group of the formula (v): 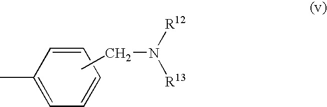
wherein
R¹² is a hydrogen atom or a lower alkyl group,
R¹³ is a lower alkyl group, a hydroxy(lower)alkyl group, a lower alkoxy(lower)alkyl group, a saturated or unsaturated heterocyclic(lower)alkyl group, or a mono- or di-lower alkylaxnmo(lower)alkyl group,
or R¹² and R¹³ may combine together to form a N-containing heterocyclic group which may be substituted with lower alkyl, and
a group represented by the formula (ii): 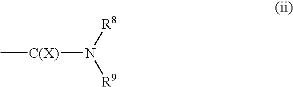
wherein
X is an oxygen or sulfur atom,
R⁸ is a hydrogen atom or
a lower alkyl group,
R⁹ is a hydrogen atom;
a lower alkyl group which may be substituted with carbamoyl, lower alkoxy, mono- or di-lower alkylamino, lower alkanoylamino, aryl, or unsubstituted or lower alkyl-substituted, saturated or unsaturated heterocyclic group;
a cyclo(lower)alkyl group;
a lower alkoxy group; or
a mono- or di-lower alkylamino group; or
R⁸ and R⁹ may combine together to form a saturated N-containing heterocyclic group which may be substituted with lower alkyl, lower alkanoyl, aryl or ar(lower)alkyl; and
R³ is a hydrogen atom, a halogen atom, a hydroxy group, a lower alkyl group or a lower alkoxy group,
or a salt thereof.

4. A compound of claim 3 wherein
R¹ is a hydrogen atom;
a lower alkyl group which may be substituted with lower alkoxy, lower alkoxycarbonyl, lower alkanoyl, cyclo(lower)alkyl or phenyl;
a lower alkenyl group; or
a cyclo(lower)alkyl;
R² is a hydrogen atom,
a group represented by the formula (ia): 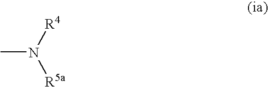
wherein
R⁴ is a hydrogen atom,
a lower alkyl group or
a lower alkenyl group, and
R^{5a} is a hydrogen atom;
a lower alkyl group which may be substituted with one or more substituents selected from amino, imino, lower alkoxy, phenyl, piperidyl, morpholinyl, pyridyl or furyl;
a lower alkyl sulfonyl group;
a cyclo(lower)alkyl group;
a lower alkenyl group;
a phenyl or naphthyl group which may be substituted with halo(lower)alkyl or di(lower)alkylamino;
a pyridyl group,
a group represented by the formula (iii): 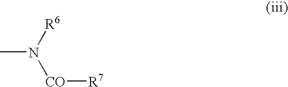
wherein
R⁶ is a hydrogen atom or
a lower alkyl group, and
R⁷ is a hydrogen atom;
a cyclo(lower)alkyl group;
a lower alkoxy group;
a phenoxy group;
a piperidyl, morpholinyl, pyridyl or carbazolyl group;
a mono- or di-lower alkylamino group;
a phenyl(lower)alkylamino group;
a lower alkyl group which may be substituted with halogen, phenyl, lower alkoxy-substituted phenyl, phenoxy, or a group of the formula (Iv): 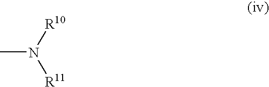
wherein
R¹⁰ is a hydrogen atom or a lower alkyl group,
R¹¹ is a lower allyl group, a cyclo(lower)allyl group, a hydroxy(lower)alkyl group, a lower alkoxy(lower)alkyl group, a piperidyl(lower)alkyl, a morpholinyl(lower)alkyl or a pyridyl(lower)alkyl group, a mono- or di-lower alkylamino(lower)alkyl group, a lower akoylamino(lower)alkyl group, a phenyl(lower)alkyl group, a hydroxy- or sulfamoyl-substituted phenyl(lower)alkyl group or a pyrrolidonyl(lower)alkyl group,
or R¹⁰ and R¹¹ may combine together to form a imidazolyl, pyrrolidinyl, piperidyl, morpholinyl or piperazinyl group which may be substituted with lower alkyl or lower alkanoyl;
an phenylamino group which may be substituted with lower alkyl;
an phenylsulfonylamino group which may be substituted with lower alkyl; or
a phenyl or naphthyl group which may be substituted with one or more of
substituent(s) selected from the group consisting of halogen, lower alkyl, halo(lower)alkyl, lower alkoxy, halo(lower)alkoxy, and
a group of the formula (v): 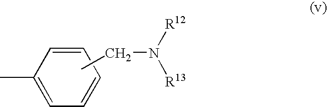
wherein
R¹² is a hydrogen atom or a lower alkyl group,
R¹³ is a lower alkyl group, a hydroxy(lower)alkyl group,
a lower alkoxy(lower)alkyl group, a piperidyl(lower)alkyl, a morpholinyl(lower)alkyl or a pyridyl(lower)alkyl group, or a mono- or di-lower alkylamino(lower)alkyl group,
or R¹² and R¹³ may combine together to form a imidazolyl, pyrrolidinyl, piperidyl, morpholinyl or piperazinyl group which may be substituted with lower allyl, and
a group represented by the formula (ii): 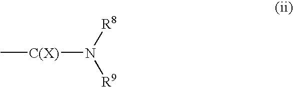
wherein
X is an oxygen or sulfur atom,
R⁸ is a hydrogen atom or
a lower alkyl group,
R⁹ is a hydrogen atom;
a lower alkyl group which may be substituted with carbamoyl, lower alkoxy, mono- or di-lower alkylamino, lower alkanoylamino, phenyl, morpholinyl, pyridyl or pyrazinyl which may be substituted with lower alkyl;
a cyclo(lower)alkyl group;
a lower alkoxy group; or
a mono- or di-lower alkylamino group; or
R⁸ and R⁹ may combine together to form a pyrrolidinyl, piperidyl, morpholinyl or piperazinyl group which may be substituted with lower alkyl, lower alkanoyl, phenyl or phenyl(lower)alkyl;
or a salt thereof.

5. A process for preparing a compound of the formula (XII-1): 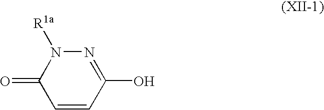
or a salt thereof
which is an intermediate for preparing the compound (I) of claim 1 comprising the steps of:
reacting a compound of the formula (XVII): 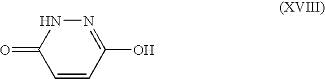
or a salt thereof with a silylating reagent, and
then with a compound of the formula (XIX):
R^{1a}—X¹ (XIX)
or a salt thereof to give a compound of the formula (XII-1) or salt thereof wherein R^{1a} is an optionally substituted lower alkyl, lower alkenyl or cyclo(lower)alkyl group, and
X¹ is a halogen atom.

6. A process of claim 5, wherein a solvent used for the reaction with the compound of the formula (XIX) is a solvent having a high inductivity.

7. A pharmaceutical composition comprising the compound of any one of claims 1 to 4 or a pharmaceutically acceptable salt thereof in admixture with a pharmaceutically acceptable carrier.

8. A pharmaceutical composition of claim 7 for treating or preventing a disease selected from the group consisting of depression, dementia, Parkinson's disease, anxiety, pain, cerebrovascular disease, heart failure, hypertension, circulatory insufficiency, post-resuscitation, asystole, bradyarrhythmia, electromechanical dissociation, hemodynamic collapse, SIRS (systemic inflammatory response syndrome), multiple organ failure, renal failure (renal insufficiency), renal toxicity, nephrosis, nephritis, edema, obesity, bronchial asthma, gout, hyperuricemia, sudden infant death syndrome, immunosuppression, diabetes, ulcer, pancreatitis, Meniere's syndrome, anemia, dialysis-induced hypotension, constipation, ischemic bowel disease, ileus, myocardial infarction, thrombosis, obstruction, arteriosclerosis obliterans, thrombophlebitis, cerebral infarction, transient ischemic attack and angina pectoris.

9. A method for preventing or treating a disease selected from the group consisting of depression, dementia, Parldnson's disease, anxiety, pain, cerebrovascular disease, heart failure, hypertension, circulatory insufficiency, post-resuscitation, asystole, bradyarrhythmia, electromechanical dissociation, hemodynamic collapse, SIRS (systemic inflainatory response syndrome), multiple organ failure, renal failure (renal insufficiency), renal toxicity, nephrosis, nephritis, edema, obesity, bronchial asthma, gout, hyperuricemia, sudden infant death syndrome, immunosuppression, diabetes, ulcer, pancreatitis, Meniere's syndrome, anemia, dialysis-induced hypotension, constipation, ischemic bowel disease, ileus, myocardial infarction, thrombosis, obstruction, arteriosclerosis obliterans, thrombophlebitis, cerebral infarction, transient ischemic attack and angina pectoris, which comprises administering the compound of claim 1 or a pharmaceutically acceptable salt thereof to a human being or an animal suffering the above disease.

10 Use of the compound of any one of claims 1 to 4 or a pharmaceutically acceptable salt thereof as a medicament.

11. Use of the compound of any one of claims 1 to 4 or a pharmaceutically acceptable salt thereof as an adenosine antagonist.

12 Use of the compound of any one of claims 1 to 4 or a pharmaceutically acceptable salt thereof as an A₁ receptor and A₂ receptor dual antagonist.

13. A process for preparing a pharmaceutical composition which comprises admixing the compound of any one of claims 1 to 4 or a pharmaceutically acceptable salt thereof with a pharmaceutically acceptable carrier.

14 Use of the compound of any one of claims 1 to 4 or a pharmaceutically acceptable salt thereof for the production of a pharmaceutical composition for the therapy of diseases on which an adenosine antagonist is therapeutically effective.
